SEQUENCE LISTING (53 sequences)

SEQ ID NO: 1 - DNA, 23 bp, synthetic construct
  source 1..23: mol_type = other DNA
tggaatcgattataagcaaaagg

SEQ ID NO: 2 - DNA, 23 bp, synthetic construct
  source 1..23: mol_type = other DNA
ggaatattcaattttcctgcaag

SEQ ID NO: 3 - DNA, 903 bp, Homo sapiens
  source 1..903: mol_type = genomic DNA
atggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctga

SEQ ID NO: 4 - DNA, 74905 bp, Homo sapiens
  source 1..74905: mol_type = genomic DNA
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttggcaccgagcgtgcccgcgggaggcgggggggggcgctgctcggtggctctgctgcgtagccggtgaacacttggcaccgatgcccgccttctgggcaaggtgccctgagcccagcccctcgccgggctgcagcccaccctcggcgcgctcagcccgcttcaccgcttcagggacggaatagaactcgcagatgcagggtgtcgctgacattttcaactttttctgcggtttccgcccgctgtctctgacccgaaagtgcccccggacggttacagaggacacttaagtggtttgcaaagcctgtggtaggggaggagggtgtagaagggccaaaccacggaacttagttttattcatttatataaagcagcactccgattctttttgcgcggcctgaaatgcatgtgaccagagaagtaattaacaaaacaatgtcaacttctaaaaccgagacattacttagatgataaggcgcagcaactcggtgaatctgtacaaaccttggaaaaaaaacacattagtctatgggaccttccagttttctcatgctcctttccagctactaacctctcctaaagggaacaaccactttttggatttgattcccaggcctcgctttcaccgggaaattatcgttgcttgtaaaacagaagaagccgggaaggcaggcagggggagctgctactttacactctgtgctttgggatagcaaaatcccgcatttaagcaatccgaggaaacgagcaaatagacctccctcgcctctccgagcacactcaacagttccggttgcaaaatgtttgcctcctgggcttcccagcgtcccgttagttgttctatttacacataattagatacttaatggagagagaaactagaagttgaggcgttcctccaggctgtattgtaaagtatgaagtgaaatccaaaatgaaatggtaatgttagaaagcaacctcattaaaaaaaaaaaaagtaacactggtcttgaagatctttcaatgtgagtacataaagatctatctcatttcttttgacagcccatagtatttcataaactagatgtaaccatttcctattgacaggaaattagcttgtttccaatttttcaatcccattcattcatccaacaagtatctgttgagcacccactatgttccagacagtgatctagctactgatgacacaagagtgaatgacgaagttctcactcatgaaattttcaccttagttgggagaaacatgatgcaatgaaaatcttcatacatacattgtgtgtacatatgggagtatttctgtaggatagatttctagtgatgaaactgctgagtaaaagggagaattatgcatattttaagttttgatttttccaaattccaggtattccatatatactccaaaatagttgtgccattttactctcccatcatcagtctatcagaggggatgctttcccacaatctcttgaatgctgaatattttcaacttttttacttaagagaaaaaagagcatctaattgttccctcagtatcagtgagtctaagcatcttgtatatgtttatttgccatttatatttttttctgtgattttcctgtccagatacttgatactttctattgagctgcttatttatttcttatgggagatttttatatattttagataatattctctctctcacacacacacacacacacacacacacacacacacacacacacacacacatacagtcttacagccacatccctgaaatcttgaccttgtgaacatgttttactggcagcactctggactcgatcattgccttgagactatttcttttttgatattctttggaaagactaacaatgacagttttattttcaaacccaacaaatcctggcatggaaatgtttgctcttgattctgcttttaaaaaaataaagaattattttctctctttctttctgcaccttatcagaaacagctaaaagaagtgagttgggccaggcactgtggctcacacctgtaatcccagcactttgggaggcccaggcaggtggatcacttaaggtcaggagtacaagacctgcctggccaacatgctgaaactccgtctctactaaaaatacaaaattagccgggtgttgtggcgcgtgcctgtaatcccagctactctggagactgaggtgggagaatcgcttgaacccaggaggaggaggtagcactgaaccaagatccagcctggccaagagagtaagactccgtctcaaaaccaaaccaaaccaaaccaaaaaaagaagtgagttggcactttcaacattctgcctggaaatctccttaccaaacctataagatcattaggtatattttctgcactttgtattgtgacaggtgacagtgttaccaaactttttaccaggacataatagggtctgcctttcttctagttgctaacaatttcccccaagtccatcttgcctgcactaacagtctcctttagacctctcctctcctgcctgtcacacattcctagtactaatgctacagtatagtagtaagggtctccagagaaacaacatttatataacataatataaatacattaatagagagaaagagattttaagaaattggcttatatcattgtgaagtcgggcaagccccaaatctgctggacaggccagcagcctggagacccagggaagagttgatgttgcagctggagtccaaaggcagtctctggcagaattctcttttacttctgggaccttggtctttctcttaaggccttcaactgattggatgaggcccaccacattatgaagggtaacatgctttacaccgagtctcctgacttaaaatctaaaaaataccttcacatcacaactagatgtgtttgaccaaatatctggataccatgcctgggcgaattgccacttaaaattaatcatcacgtacatgttttaaggttttgttacacaagacctcacttccaagtatcactttctgttttggtcatcagttgctgcataataaacaaccaccctaaaatttagtgacttaaaacaacaatcatttattgtctgccatggttctgtggtttgactgggatcagctgagtggcctgtttcacttggtgtcagctgggggtgtaggcatctgcaacattgtcttggcaggaacatccaagatgtcccacttaacacgatggctcctgggctcagctgggctggtcaggcctcccttcctctctgtgttgccacacggcctctctctatccatgtggcctctccatatggtctctccctggtggaggtgaatttcttcaaggtttctaaactctcaaaagtggagcctggcaagcccactcaaagcttcagatccacaactggcacagcttcccttccacagattctataggttaaaacaatcaccggaccagcccagattcaaaagaaggagaaacaaactccacccctccatggaggaagtagcaaaaataatgtagacagtttttaccctttacatctggatttgttagcttttctgttttcattttcttctggtctttcttttgtccttctcttacacttattttattaccttttatcaattagcttttaagatgataaaaatctaatacatgcctcctggaatgtcttcttgagcctagggacttttgtttaatgatatatcttgaggacctagaatagtgcctgaaatacaatagtcattaaatatttagctgaattaaatgaatggtatataagccagggtattaaaaataacataaacaaagttgtaataaatatacttccccagtgaacgacctaataccattactccccaaaccctcaatttctgtcttgagcatagaaactgttaatttttcctttgtgtagtaggtccttagtatttctttagaggttgtagcactttatcttcctcactgttccttttccttggttgtccttttccaaacatctctcaacaatttctcccaagtccatcttgcctgcactaacagtctcctttagaactcccctctcctgcctgtcacacagtcttagtaatagtgccacagtatagtagtaagggtctccagagaaactcactttctgcaagttttttagtgtgggtaggtgagtattgaagcttgttcttggcgttaccaggttggttctttgagttgaaccaggggcattacatgcggaatattcctgaacagatcacctctggttctgctgtctcaagggccacacacaagagctgccttctgaccaagatgtctctgggcacatgagacctgaaatacacatggccaagactcaacaaagcgtttgctgactgtcagagctgacagcatctcggtactgtgggagggagcccagtgtctggtgatagtcaggacggacccaggtgatgtcaggggtggggtggggcctgcaggagggaatggagagccagcacctaggggaagctgggaatttaggaagatatccagagagtgtaacttcagttccactaatctcacctgggtgaaaaccagccctctccatggatgatggtgattgcaggtactgacgatagctgcagactgctggtggtctaggcaagcatgcagggatgggagcaggcatttctgagagctccccttatccctgccccaagacaaggtgggggccctgtggggaaggactattttattcacttctgcatctccagttgtctaacacattgcttatcacctaacagtctcttaatagaagcttgctatattgagctgccttgagtccacatcatgctggttgaacataagtagaaattgtgggagacttaatcagagaaaatctttcatctgtcttaccttacgttctaatgatcacttctgtgcccacaaggaccatcttttctgattcatgttcatgtcgatttcttttttatttaacttcctcttcaaaatttctggcaggatttcttgggagccaattctccatttttacttccccatgcctcccattttaatgactgtaggattttctcagggtctactcagcaaaacttgttagtacaacatgagcaaaacagcaaatttatgcaaacacttagcaaagacttagctgtctgcctaatgctagggtgagacatgggagattcacaaatgaagaaacacaagcattgatctccaggatcctgcagtgggaggtatgcggaggaccaacctgggaacagagcaacgcaacaccatgtgatctgtaatgaaaaagaggcctgtacacagccaagaggtcactgaggaaggagccattgcttctgtgcagttagtgctgcgctagattctgcagggatgtaagaaatgtacccctatcaaacaagaaagactatgtgaattgctgaatatgtgagtggagtaccagcacaatgccatggagatgcagacgtgccccatgtggtggggcagagtcaggaagcactttaagaaagaaatagcatttcaggtcttcctttaaaggtagaatttcaacaagagggtgctctggagagtgtgtgttaccctgctgagaaaatcctggcggtcaggtaagatgctactgccagggaagattggccaattgattgactaaacccttaaaggtttggggatcttggggaggattctgctggtgagagggtctggacttcctcttggtctgtccacagctggaccttctcagcacacaagagactatgagggtgaccattttgcacaggacagaattccagcatgtttttcccctggagtgatggaatgaccacctgctcaacatcagtgtcctcactgagaccatgagattcagtagagtgctggaaagcttcatgcttacctgtgtcttcttaatgcttagtgttatgattgaaggcttccttcagtcctaccttttgttctggggttctaagaatcttaggtgcaggcgaggcacggtggctcacacctgtaatcccagcattttgggaggctgacgtgggtggatgacgaggtcaggggattgagaccatcctggcaaacatggtgaaaccccgtctctactaaaaatacaaaaattagccgggtgtggttgcgtgcacctgtagtcccagctactcaggaggctgaggcaggagaatcgcttgaacctgggaggcggaggttgcaatgagctgagctcacgccactacactccagcctgggtgacagagcaagactctgtcttaaaaaaaaaaaatctcaggtgcacctgagacagattgaatgtggaaggggaagtgaaacaggccttccaggtgtggggcctgggtgctgctatagttacaaatggggaagtgagactataggtctcagttacctgtggaaggaagggtagagtggagtacttacgcaaattagctaattctgggagcttggggtgctaccagggtatcagggagaatacagccagggaatagaatcttcttgaagcaaaggctgtttggaagcccccagagtggatgaaaaggctcagtgggaaacaacagatatcaggagagggagaagaagatacctatttctataccttttggccttgtgttttgcttcagacactgttcccagcaaggtcagtggaacccactgctcaaaacacacacttgctccttgttctggtgtcataatagctctgcaagcagtggtggtgttcagcctggagaacgttccttttctttttttttttatcacaataaacactcatggcttctctgcttcttcctttcttctttgtcttaggactcttgaaaaacagctgccaaatgtcagtttagatattttggagggaaaaaagttgggaatcaatgtttacaggttgcctgcaatgtgctggaaactacatagttggttctttttaaactttctctgaatcctgtcaggaaagttccagcaatcacatcttagtgggtccggaattcgtgggttcttggtctcactaacttcaagaatgaagctgtggaccctcgtggtgagtgttacagttcttaaaggtggtgtgtccagagtttgttccttctggtgttggacatgttcggagtttcttccttctggtgggctcgtggtctcgctggcttcaggagtgaagctgcagaccttcacggtgagtgttacagctcttaaggcagcgcgtctggagttgttcgttcctcccatctggagttgttcgttcctcctggtgggttcatggtctcactgtgctcaggagttaagctgcagactttcgtggtgagtgttacagctcataaaagcactgtggacccaaagagtgagcagcagcaagatttattgcaaagagcaaaagaacaaagcttccccagtgtagaagtgtagaacgggacgccaatgggttgccagtgttggctccccccagcctgcttttattcccttatctggccccacccacatcctgctgattggttcattttacagagggctgattggtctgttttacagagagctgattggtccgttttgacagggtgctgattggtgcatttacaaaccttgagctagacacaaagtgctgattggtgtgtttacaaaccttgagctagacatagagtgctgattggtgtatttacaatcccttagctagacataaagattctctaagtccctagtagattagctagacacagagcactgattggtgcatttacaaaccttgagctagacacagggtgctgattggtccgtttacaaaccttgagccagacacagagtgctgattggtgtatttacaatcccttagctagacataaatgttctccaagtccccactagactcagaagcccagctggcttcacctagccgattgtgcaccaagtcagcaggcggagctgcctgccagtcacctgctatgcacccgcactcctcagcccttggacggtggatgggacgccagggagcagggcgcggtgctcgtcggggaggctccagcggcacaggagcccacggcagggagggtggggggaggctcaggcatggtgggctacaggtcccaagccctgccccgcggggaggcagctgaggcccagcaagaattggagcgcagcgccagtgggccagcactgctgggggacttggcacaccctccacagctgctggcctgggtgctaagcccttcactgtccggggcctgctgcgctcgccggccgctcagagtgcggcctggggagcccacgcccacctggaactcgcgctggcccacgagcgcctctctctctacacctccgctcaagcagagggagccgactccggcctgggccagcccagagagggtctcccacagtgcagctgtgggctgaagggctcctcaagcacggccagagtgggcgcgcagaggccggggggcactgagagcgagcgagggccaccagcacgttgtctcctctcattagggttggggaaatggaccctgagagagattaagtaatttggtgatattctatagtcactctggctatgtaatttatggagcctagtacagaatgaaaatgtggggctcatttttcaaaaagcaggaaacagcttttcctttcttccagggtctcttcctccacctgccatgctggtgtttggttgctatttaatgttgagccctcttgggcacagggatacttgcagggacagggtgtctgctcatttttctgtagacctcaaaggtgagtcctgaggcttcagggtcactggcctccttttagggagtcacgacgccttgtctttgtacttcaggaatgattacgaatctttgtaggtaaagcggcagaatgccacgtcctctcctggttgccaggacgtgttccttgtggtttaattgccgggtctgccctgcagaccctggctgagcgacagatgaaaggagtactcagacacaggtacgcagtgaaagagcggctaggggactgccgaagagtcagcagtctcaataaactggagctgctcacttttattcagtacagacataatgccgaaagcctggagccaacgcagtctgtgggtaattaacattgttgttccgccgtgcagggagcagtctcgccagaggatgatgaaaggttggtttccggaagtaaacaagcttatttagacaaactcccctacattcccttgtacccactcctcgccctctgcgtcagggtaagagaacagctgccttcagcttattctcccccgaagctttgcagagccttctgacctttcaaaaggtcttcttctttccctatcggttctcccactactctgactgatctcctatatttgatctcaccttaacaatcacttcttagagctgggtcaggaagtatgcagcatgcacctggcactcctagtactgtgcccatgatgggcattgctgattgttcagagcatattggatgagcctggttcagcctcagaatcttccacccagtgcaccatggagatgctaccaattggttggagttgctctgagaggtgacatttccttgtgattctgcattagaaacatgttgtttgtcagccgaaacagggaaacctgacacgttatccgcccccaggaagatcccatcatcattccatgcaccttcagtcctgggagcttactttaaaaaaaagtgactgacatatgagcgcaggtccccaaacagaggggaggcaggatgagaagccagatgaagagagtcaaggtcctggggctgcttagcttggatgaatctgatgggaggtggggtgcatctgagtgttctctgctgatgaagaacagacttgttgcacgggggtaggtgtgtgctgtgtaaacacacatcagaatcaggaccccgaatagtgaataggcaagagtaacagctgaatttgcccagctcatcacaatttaacatcagttttcaaaaaggtaagagcgtggctttcatagcatgcagaatcaacacacatcaaagattgatttactcatttatgaaggaatcagcaaaatgacaaacttagttcagagaatattttgaggctctgagtagatataaaactggttaatgtttctcagggcaataaaaagctataaacgttggggatttcttttttatcagacagaaattatttgcatacttaacagaaaagatctccaagttaccatctaacttcataaggttcgaataaaacttcatagagttattaatgaatggtaaatagaaaagacaaatatatgttttaccagataattaagtaattcttggtaaacctggcaaacagtaccccagtgtgactctgaaaagacatgctgcccatctttttgccttatttccacgttttaggtatttttgtaagatatctattcaataaatatgtattgagctcctatgatgtcccagaaactcttttagatcctggagatatagcagtaaacaaaacagatgaaattcttgctcacatggaacttatattctagtaggggagacagacattgaaacagaaaaatacatagtatggcagatggtggaaagtattaaaaagagtgctgtgtagtgtttacaacttactcatttatgaaggaatcagcaagatgataaactcagttcaaagaacatcttgaggcaccgagtacatttaaaagtggttagtttctcagggcgataaaaagccataaactttggggatttcttttttaggtatggaaacctaaagtaaagaagatgctatggtttgcacatttgtcccctccaaaactcatgtttgaaatgtaatcccagaagtggcaggatgagagattggccctttaggaggtgactgggtcatgagagatctgccctcatgaatggattaatccattcatggattactgattaatacgctaatgggttaatggatcaatgggttatccttggaatgaaatggcaggctttacaaggagaggaaaagggacttgagctagcatgctcaccctcctcaccgtgtgatgccctgtcctgcctcaggactctgcggagttctggtaagcaagaaggctgtcaccagatgtgtcccctaaaccttggactttttggcctccataactttaaggaataaattcatttttaaaataaattacccagcttcaggtattctgctataagcaacagaaaatgactaaaacaggaggctttactggaaggtgtcctcttagcaaagacctaaagaaagagggagagtgaaacatagaaatatctggggagaacatcctaggtaaaaggaacagcatgtgcaaaggccttgaaaagcagcaagccgctctccctctccctctccctctccctctccctctccctctccctctccctctccccctctccctccccctccccctccctctccctctctctccacggtctccttccacggtctccctctgatgccgagccaaagctggacggtactgctgccatctcggctcactgcaacctccctgcctgattctcctgcctcagcctgccgagtgcctgcgcacgccgccacgcctgactggttttcgttttttttttttggtggagacggggttttgctgtgttggccgggctggtctccagctcctgaccgcgagtgatccgccggcctcggcctcccgaggtgccgggattgcggacggagtctcgttcactcggtgctcggtggtgcccaggctggagtgcagtggcgtgatctcggctcgctacaacctccacctcccagccgcctgccttggccccccaaagtgccgagattgcagcctctgcccggccgccaccccgtctgggaagtgaggagcgtctctgcctggccccccatcgtctgggatatgaggagcctctctgcctggctgcccagtctggagggtgaggagcgtctctgcccggccgccatcccatctaggaggcgaggagcgcctcttccccgccgccatcccatctaggaagtgaggagcgtctctgcccggccgcccatcgtctgagatgtggggagcacctctgccccgccgccctgtctgggatgtgaggagcgcctctgctgggccgcaaccctgtctgggaggtgaggagtgtctctgcccggccgccccgtctgagaggtgaggagaccctctgcctggcaaccgccccgtctgagaagtgaggagcccctccgtccggcggccaccccgtctgggaagtgaggagcgtctccgcccggcagccaccccgtccgggagggaggtgggggggggtcagccccccgcccggccagctgccccgtccgggaggtgaggggctcctctgcccggccgcccctactgggaagtgaggagcccctctgcccggccagccgccccgtccgggagggaggtgggggggtcagccccccgcccggccagccgcccagtccgggaggtgaggggcgcctctgcccggccgcccgtactgggaagtgaggagcccctctgcccggccagccaccccgtccgggaggggggagggggggtcagccccctgcccggccagccgccccgtccgggagggaggtggtgggggtcagccccccgcccggccggccgccccgtccgggaggtgaggggcgcctctgcccgtccgcccgtactgggaagtgaggacccctctgcccggccagccgccccgtccgggagggaggtgggggggggtcagccccccgcccggccagccgcccagtccgggagggaggtggggggatcagccccccgcccggccagccgccccgtccgggagggaggtgggggggtcagccccccgcccggccagccgccccgtccgggagggaggtgggggggtcagccccctgcccggccagccgccccgtccgggagggaggtggggggatcagccccccgcctggccagccgccccgtccgggaggtgaggggcgcctctgcccggccgcccctactgggaagtgaggatccctctgcccggccagccgccccgtccgggagggaggtgggagggtcagccccccgcccggccagccgccctatccaggaggtgaggggcgcctctgcccggccgcccctactgggaagtgaggagcccctctgcccggccaggaccccgtctgggaggtgtgcccagcggctcattggggatgggccatgatgacaatggcggttttgtggaatagaaaggcgggaagggtggggaaaaaattgagaaatcggatggttgccgggtctgtgtggatagaagtagacatgggagacttttcattttgttctgtactaagaaaaattcttctgccttgggatcctgttgatctgtgaccttatccccaaccctgtgctctctgaaacatgtgctgtgtccactcagggttaaatggattaagggcggtgcaagatgtgctttgttaaacagatgcttgaaggcagcatgctcgttaagagtcatcaccactccctaatctcaagtacccagggacacaaacactgcggaaggccgcagggtcctctgcctaggaaaaccagagacctttgttcacttgtttatctgctgaccttccctccactattgtcctatgaccctgccaaatccccctctgcgagaaacacccaagaatgatcaataaaaataaaataaaataaaaaaaaggaatgaatcaagaaaaaaaaagaaaagaaaagaaaagcagcaagccagccagtgtgtttggaatgttctcttatggaaaatttcaaagatatgtaaaactagggctaataatacaattaacccctacttacccattacccaacttcaacaactatcaacattctgctgttcttatttcatctatttacccattaaaaaaaaagtgtactttaaagcgaattccagagtttgtataattttgtctgtaaatctttcagtctgtatctctaaatctttcagtctgtaactctaaaaagaacattaaaaaacacaactatcataccatcatcctacctgacacaactgagtaatttttcatatcatccaatatcgatcagaagtttaattttccatgattttctttaaaatgtagttttataattgatttgttctaatcaagatttgcacattgcattaaaaatatatgtatcttaaatttcctttaatttacaaccatttccctcccccatttaagagtgcatattaatttattaaacagactgggcaattcatcttgtagaatttcccaccttctgggtttggccaattacatccttgtggtgttatttaaaatcctcctctattccctttattatctgttgactgacagcttggccaatcagaatcacttgaacgagctgattacaccccctctttctgaaacgttttcttcccttgagtctgtacacactgttggcttccaaatttatatctccaacccagacctctctccagtacttctgcttgctactgtgtttttcataactttctgatttttttagtatccaatgctagaaaatatatccattttgagaaagagacaaagtatgagctcatattgataatttcatttcaaagtaaagggaacaaagtttttatttaacatgttaattttatgcttgtttctcttttacactgaaaatcttagttctcaatgacattaatataattatgtatttacttcccatatatatgttgtatataattgttttatatatacatatgtgcatatatattattgctaatgaaacgtctactgaatgatgtaagttttctctgtgattcttttggtccttgggacacaggacttatcccactagtgatgtgtagtcaaaatactatgtaccagtgtttgataccttaattaaaggatctctctggctgctgcattgagaacagatggtagcaggacaaggacatcagcctggaagccatctggaagctctttagtgattcagataagatttgattttggcttggatggtgatggatgatgttgagaagtgggtggattctggatatgttttgaaagtaggttccatgtgatttgccatgggctaatatatggaatgtgagagaaaaagaagaaacaacaatgcttccaagattttggggcagaggtactgaaaaaatgaatttccatttatcagaaagagaaagactgtggtaaagcaagttgggaaggaaacagcagctcagttttccacattcagtaaccctcccttatctagggtttcactttctgcagtttcacttatccatggccagccacagtctgaaaatattaaatggaatattccagaattaaacaactcgtaagttttaaattgagcactcttctgagtagcgtgatgaaatctcatgatgtcctgctgtcttctgccctggatgtgaatcatccctttgtccagcatatccatgctgcatgttgtacctgcctgttacttgcctaagcaagtggatggctacttgcttagtgtccatctcagttatcagattgaaaaagcagtacatatacatagggtttagtactatctgcagtttcaggcatcactgagggggttggaacataaccctggcagataagggagactactgttttagtggagatgttgaccagaagactggttcatatgaatatgggggtcctggagagaggtctgggctggagatataaatttggaaatcaacagcgtatacagacacaaggaaagaaaacatttcagaaagagggggtataatcagcttgttcaagtgattctgattggccaagtaagatgaagattggaaattgaccaatggatcggtgacttggcaaggtcagtttgggaggagtggtagtgatgaaagcttatgtggaacaaattcaagaaaaaaacagaagagagaggaattagagattgttgtgcacagacaactcttgcaagaggttttgctggccaggagaacagaaatacaagtgaagtagtggctggatgttttccaaaacatgaaacttagttttcataggaaaaaaatggtttttctttttctacttattcaattttgtgcacaatttcattacattatataagtaaaaaccacaagcacgaacatgtttataaataggtaaataaataacaaagtagatagaaacaaaaattctcaggtgtgcaaaagagtagtttattagctgtgtagaagacagaaaacttgcttttatagagggaatgaatagtgttgattagtatagtgagtcagttaaataggtatcagttgaattttttgagacaataagtacatttagaattggctaggcattattcttccattaaaggaaactcttagtaatagattaggtcagaaaaccacaaagacagttttcttgaataagggggtggtgggaaaaaatggatgtaagatgctgagaaaaaacaggaaactcaacaatagatgaaatcaagaattagtttcgtgtgtgtgtgtgtgtgtatttcacagaaaagggctgaaaggggaggaatggttatattcacagatttttgtagttgatcttaatagggaataggagattacctttttttcagcaaaatattaccagcaaatgtctactctggaaatagaggataagaatcttctatttaaagaccaaaaaggtgaaaactgaggtcagaaatttaaagagtaaatgtagcaagatgctaacattcttgttccctctaataactcttcttttttaaaaattataaaagtaaaacatgcccattgaagaatatttggatgaatacaaaaatatagaaaagaatgtaaaatcgcttaagcccccaagtagaattctaaatagcggagaaacccacgatttaaaaaaataaataaggagatccacaaaaggaagatatatacctgcttaagtggacggcctcagcataagtagtcttacagttacaattaattttttttctattatacgatgttttaaaaaattgctaagctcagttgtctactccctgctagaatgtatcaaacatgctgcatccacatagtagacagttattctacatttttggtctttggatgttttgaagcaattagcaaagtttgatttgaagagacattataaatttccgacggcatatttttctctggccatgatccatatttgccttgatattgtccaaatgttcattttatcttatgactatcaagcaaatattataaagttatttgtgaatttggcacttcatagatgaaatattgtgaaattctgaataaaactgcttcattgattatcttggttcctaaaagagatagtttatgagttaaattatctctaacgttgtctaagttggcagtaattaaatctccatgggaattcttaataaaggcaaagaaggactgaaaacaccttgctgaaactgagagagatttaaaaccacaaaaaaaatgtaagatttagttagttgttcacgttttggggtcctgcttttatgtgttaatacttccagataaaattttttcaaaggtagttttgaaccccaggatctgaatatattaatttgtttatctctgaaaactgtatactttgtgtttcccaggtctggctttcttcatttttcctacttgagggaactccttttcttccttcaatatcctcatatcctccttcaatgtttctcagtcaatatctcctctcaatttttccatttaaaaattaaaaattaaattattaacattttaaatacactgaaatgttcatggaatagtagtacaaatacccatttacccaccacatggagttaatagacaataaaaatttgccatatttgttttacatcttttttctagtttttaaagaaataacatgttacaaataaaatcgaaatactctttccgccgattttcttttctcctgtctcagaggcaaatactgctacttgcttctcttgtatctttttagaaatattctctgcatatataagcatatatccatatatttttccacacatagtatctcttccgtgtcttagtttttttcattctacaatgtatcttgatgatcagtgtagctttgtttcattctaataattatatagattccactttacaaatttactatactttggacttgtatctatgaacgtttcgattgttttaaacctttggcaacattacagacaatgctgcaatgaaatccttctgtatgtatacatacgtggatgtgtgtataagatataattctggaagtagaattgctaagttaaagcatatgctacttaattttgaaaaaattgtcgaacttctcttcacaaggcaattatcttcttgaggtaatgagaactcctatttccccatacccatactacccagagtatcttcaaattttctgatttttgctggtctgataagggaacgttacctcagtttaatctttatttgcatttactatgtgtttttgccctgtagagtgaattttacttttctacctaacaatatatttttgtgattttaatgacattacttttgagatttacccatgctaatatataaactctggttctgcaattttaagtgctatatagtatcctattgaatgaccatactactcatttatttattattatctacttgttaattagtctgttatcttttttgtcagtttttgtgggcataaacaatgctaaagcatacatgtctacttgcgcatatgattttttcccagtgtgtttaatagaatgactaaggtagaatgactaaagttctgaggtgtggcaggtatgccatcttcacctttactgggtatgctagtgtgctgcagctgccgtgataaagtactacaagccgggtggcttcaacaatagcattgtactgtctcacagttctggaggctggaagtctaaaatcaaggtgtcagaagggtcggtgccttctgaaggtgtgagagagaatctgcttcgtgccctcttccaagcttctagtaacctcaggtgttccttgtcttgttgatggtgttgtcccagtgtcttcatattgtcttctctctgtttgtgtttgtgtttgtctctatgcccaaatttccccttttcataaggacccagtcgtatgggattagggcttaccctaatgatctcatcttaacttgatcatctgtaaaaccttatttccaaataaggtcattttctgaggtactgggtgttaggatttcaacatcgtttgggggggtaaaattcaatcaataacaacaggtataccagttaagatgtttttggctgcaactaacagaacattcaactgaaaaggtttaaaatatattgtaaattttaaacaaatgttttatagagatgagttctgactgtgttgcccaggctggtctcaaaatcctggcctcaagcaatccttccaccttggcctcccaaagtgctgggattacaggcatgagccacagagcctggccactttaaaatgttgttgacacacataacaagacatccagaagtggggtggtcataggatcggttcagcagcacagtgatggccgaggctctttccttccactcttccatctcccttgagtggctttcagcttcttgtcacaagattgctgcattatgtcctcatatacctggaacagagggcagggagaggggcaagagcacacccccatttcaagtctcttttcatcccaaagtaacatatttcccagcagccctctttcaaccctctctaggcttctccttaaatttcactggccagaattgggccacacggttacccaaaggtataaaagaggcttgagaagtgagaatttgatattttgaatcataataggagacatgctttgccaataggaaagaagggtaaaagaaatcttctggagagtacggaaccaacggcgtctgccacgttgcatatggccaaatggtcaccaaatttgttgtatcactttattttcacaaaattggtgtaaagtcctcattgctattcatcctcgtcatcattcggaattctcaaatttaaaaaatgcccatttccttgttgttttatttttttaattccctgatcattaacataagtaattatctttttatgtgtttatttgtcattcaggtttcttctgtgaaaagcctatttctgtcttttgcttgttcttatgttgaattgtctctacacattcaggattctaatcttctgtcagttatgtggattgcaaataaagtctcccagtctatggcttaactcttgacactatatttatcacgtcccttatttactaaagatttaaatatgatcaaatttatgttttcttttagggttgtgccttttgtatcttatttaagaactgatttctttaatgtgagatcacaaaagggttataaatattctaaaaatgttaaacttttgcttttcatagttagatctttgatccacttggaatttatctttgtgtgtggtgaaattaggaagcccatttcatttttccccagatggatagtcagttgtgtaagtgctgtttatcaaataatccatcctttatcgagggttttctgatgccacttctgttgcatcttgtgtttctttatgtatgtcagtttatttctggactctccaatctgttttatggtctgtttgtcgctataccactgccacgttgtttaaattactaatgctttgatatccaatagaacatgtctgctttctgttacgggagataaatttacatttttaagaagtttatataaatggaatacatatcatttactcttttgtgtctgagttgtatgtctttgtcatgatgtttttgtgattcatccatgttgtatgtattaatagcttgttcctttttattattgggtctattgtgtgtatatatcacaatttgtttatttatctattgatagacatttggcttgtagtcactttttggatacaatgaatagaggttctatgaacattagtgtacaagttattgtatggacatactgacagagcaggagcacagtcctcttggacaaacactgccactttaagttccagctccatttttagcctcatgcatctcagggaaatcacttctcttctaactacaagtagccagaaagagcaaacagtaaaccacagataaaacagctcaggcacagagggaggagggagaaaagtctcttgggtaactgccacacttcaccctcatacagtgggccccagtaaaacagtgggcgttaataaacacattattttcccttcaggtgcactaaaatagggaagctaaaagcagactcggggggtatgcctgcagctgcagaaaaatgtataaaaacagacacacaactctccctccaaaataagcacaacaaaaaacacaaaagcagtccaagcctctaataaactctcctatcctaaatccttaaaaactcttagtctgtaagagagtgtgctgttgacctagctcagccaaaagctcctcacaggttcgttttctctaaaataaacctgtcttaactggcaagccacctttcgtgttttttttcctctttctttaattcttacacatactgttttatttctcttgagtgaacacccagaaatagaatagcagagccatatggtacataagttgattagctttttgagaaaccaccaaactgttttataaggcaattgtatagttttacatgtgtagcatcagtgtgtgaatattctagttgttctacgtccttgttaacatttggtattgtcagacttttaaattttagccatctaaaaatttatagtgttattttatggtggttatagtttgcatttcccccatgactaatgatgctgaggatcatctcataggctttttgaagtatgtgttcaaatcttttgcccatctttaaaaattagggtttttgttttgtacaaatacttggaaattaagcaacatactccagaatgaccaatgggtcaatgaagaaattaagaaaaataaaaaaacttactgaaaatgatgaaaacatgtctaacaaataaaaattgatacacaacataccaaaatctatggaatacagtaaaagcagtactaggaggaaagttcatagtaatgattgcctacgtcaaaaaagtagaaagatttaaaacaacttaacagtgaacctcaggaaactataaaagcaaaacaacaacaacaaaacccccaaactccaaattagtagaaggaagtaaataataaagatcagaacagaaataaatgaaataggttggaaaagtaatacaaaagatcaacaaaatgaaaagttgttttttaaaaaaaattgactaagcattacctagactaactaagaaaaaagagggaagaaccaaataaatgaaaaaggagatgttacaattgataccacaaaaatataaaggatcgtaagagactattatgaacaccaataaattggaaagcccagaggagatggataaatttctgggcacctacaacctaccaagattgaaccaggatgagatacaaaatccgaatagaccaataacaattattgaggaacctcaataataatttttattaaacaacaataaaaagtttcccaattaaaaaaaaaagctcaggactggatggctttactgctggattctaccaaactttgaaaaataactaccaattcttctcaaactattccaaaaaattgaagggaagagaattcttccaaactcattctataaggccagaattaacctgatacaaaaccagacaaggatacaacaacaaaaaaagaattttgcaggccagtatccctgatgaacataaatggaaagttcctcaacaaaatactagcaaactgaatccaacagcacattaataagtttatttactaaaaccaggtgggattcattccagggatgcaagagtggttcaacatatgcaaatcaacaaacataatacatcccatcaacggaatgaaggacaaaaatcatatgatcaccacaatagatgcagaaaaacagttgataaaattcaacatccctccatgataaaaactctcaaacaattaggtttagaagaaggaacacacttcatcttaataaaggccatatatgacaaatccacagctaatattgtaccaaacagggaaatgttggaagttttttctctaaaaactggaacaagataaggatgcttaccctcactactctgattccacatagtactggaagttctagccagagcaattaggcaacagaaagaaataaaagacatccaaatttggaaggaataagtcaaattgaccatgtttgcagatgacatcctcttacctacagaaaaatctaaagactccaccaaaaaactcttagaattgatatacaaattcagtaaagttgtgagatacaaaatcaacatacaaaaatcagtagcatttctatacaccaataataaactatctgtaaaaggaaccccacttacaatagctaccccccaaaaaaaacctccacctaggagtaaatttaaccaaagaggtgaaagatctctagaataaagactacagaacactaataaaagaaattgaagaggacataaaaaattggatagatatcccatgttcatggattggaaaaattaatattgttaagattccatactacccaaaggaatctacagattcagtgcaatctctatcgaattttcaatggcatttttcacagaaatggaaaaaaagattcttaaatttgttaggaaccataaaagaccccaaatagccaaagcaattgtttgttttattttattttattttattttattttattttattttattttattttattttattttatttcacttcatttcattttatttttgagacagtctccctctgtcacacaggttggagtgcagttgcatgatctcagctcactgcaatctctccctcctgggttcaagcaattctcctgcttcagccacctcagtagctagaattacagacatgcactaccatgccctatttttagtagagatagggtttcaccatgttggccaggctggtctcaaactcctgacctcaagtgatccacctgcctcagcctcccaaagtgctggtattacaggcatgagcctctgctcctagccaagccaaagcaattctgagcaaaaagaactggagggatcacactactttactttaaaatataatactatagtaaaacagcattgtattggcattaaagcaacacataaatcaatggaacagaatagagaacccagaaataaattcacatatttatggcctattgattttctgcaacagcactaagaacataaactgaagaaaggacacctttttcaataaatattgctggggaaactggatattcatatgcagaagaatgaaactagagccccatctatcataatataaaaaagtaactcaaaacgaatcaaagacttaagtgtaagaccccaaattatgaaactactaagagaaaacataaggaaaatgttctgagcaaagattttatggataagacctccaaaacacagtcaacaaaggcaaaaatagactaatgggattacatcaaactaaacatttctgtgcagcaaagtaaacaatcaacagagtcatgtgacaaactacagaatgggataaaatatttgcaaacggtttctctgacaaggaattaatatctagaatatacaaggaacttaacagcaaaaagccaaataatctgatttttaaaatcggcaattgatctgaacagacgtttctcaaaagaagacataaaatggccactaagtatttgaaacagtgctctatgtgactaatgattagggaaatgcaaatcaaaatcacagtgagatattatctcacgccagctagaatggctatcatcaaaaagaagaaaaaataacaaagctagcaagaatgcagataaaaggaaacttaaaacatttcagcttttactttagattcaggggttacatgtgcaggtgtattgcatgatgttgaggtttcagaatatgattgaacccatctcccaggtggtgagcatagtacccaatatgtggttttgcaacccttccttcctccttccctccctcctcttatactccccagtgcctagcattcctatttttatgtccatgcgtacccaatgtttagctcccacttataagtgagaaatgtagtatttggttttctgtttctgcgttaacttgtttaggataatggcctccagctgcattcatgttgctgcaaaatacatgatttcattcttctttttgtggctgcatggtattccatggtgtatacataccaccacattttctttatccaatctgccattattgggcatctaggttgattccatgtctttgctactgtgaatagtgctgtaatgaacatataagtgcatgtcttttttttggtagaacaatttattttcctgtgggccatatacccagtagtgggatttctgggttgaatggtaattcagtttttattaatagttctttaagaaatctccaaagtgatattcacagcggttgaactaatttacattcccaccaacagtgtataagcgacaaagaaaattctcacacattgttggtaggaatgtaaattagtacagctattatggaaagcagtgtggaagttccttaacaggctaaaaatagaactaccatatgatccagtaatctcgctactggacctatatccaaagaaaataaaatcatatgtcgaagagatacctgcactcccatctttactgtagtttcatttataataatgaagatatggaatccaccgaagtgtctatcaacagatgaagagataaagaaaatgtgggatatatagacaatggaatgcagccataaaagagaatgaaatcctatcattggtggcaacatggatgagcctggaggacattatgttaagtgatataagccaggcacagaaagacaagtttcatatattctcacttacatgtgggagctaaaaaagttgatctcagagaagtagagagtagaatagtggttactagaagctgagaagggtagggagacagagattgatcaatgaatacaaaattatatatatggatagaggaaataagttttagttttagtgttctatagcattgtagggtgactatagtgaacaataacttattgtatattttcaagtactgagaggagaaaattttgtacattaccagcacaaagaaatgataaacctttgagataatggatatgccaattacacagattcgatcattatgcattgtatgcacgtattgaaatgtcacttcaccccataaatatgtgcaattacatgtcatttaaaagtgataagaaaaattagtttttttgatcttattattgactcgtaggaagcttatatattctgaaaagaagtccattttcagatgtgtactaaaaatattttctttcattttatgacttacctttccattttctttaatggaaacttttaaaaacttttaaaaaacaaagttttaaaaaaatctagttaaggtccagtttgttaacttttttctaatatggcttgtgacgcttattccttctgcctagaatgttcctgggatgtttgtggagctaagtcctcgcttccttcaattctttactcaaatatgtccaccctatttaatgtcaactgtccaccgtatttaatgccaccctatttaatatcactaacacctcctcccccctcactcttgacattcattctagtctattttacatttttttctcatagaactcataaatttctagcatgctttataacttacatattcattatgtttattgtttattgtccgtctttgttccagtaaaatgtaaactcctagaagaacagagacctgtgttttgttcactgatgtaccctaagtgctcacaagtgtttctagcacctagtatttgctcaataaatatttgctaggttgatgaattaatgatttctaagctttccttcagcctgaagagttttctgattgtaagattctacttagataatcctaattgtctcagtgactctcaccagtcactcacttctcccacaaggtggcagtctttaccttcaacacaggttctggtagcctcaaatttgagaattaatagctgagttaacctgcttgttttctttgagcccagacagcctgccctatgggaactgacagctgtaaaatttaaaggacgagtgtaattaccctgcaagatctgagtgcttttaggcaagaggatttagggggtgagagttttcctggagagggacacattatgaaggtgatattgcttaattgatggggactttgaaacatagttgctctttgtgagaatggtataggtttagagagaggtgctagcacagagctgtgacacctgaagtaggctgaccgcagacaaattggatttaaccaccaaatatatctgtgttttcatgtcttcctgccccgtgccctcttatctgactcactttaccccagcactggggaataactgtgccctattctggtcctgacccttttgtaccatctagggaaatgagaactcctcttggggtctcagatcctcatttctgttagaaccaatcctattctgtgggtagggccatggttgtaaatttcctgtgggaggcagcattgctttgcaaaaagaacacagtttggcatgtgaggcagctctgccacttggacaaggtgataacgctttagtctctttatttctaaaacagggaagatgctaataccccgcccatggactagtatgagatttaaatggcaggtacttggcacagtggcaggtggtgaatgctctttggtgatcatgactatccctttctcctggtagtgctgcctcctccctctgagcacctggagtcaatccaccttgggtaggtcagagaaggcagaagaaagtggtgggaggtgaactcgacggaatgatgtacagggcgatagggtgagtgagaggtctgggatctattggcaggagcagaatggtaggaaagggaaaacatgccattgaccttgaatcttgacatttgtgcccatcctatgctgtgttgagcctcaggtcaccgtttgcggaggtgagcagaaaactgctaacagatcgaggcttctccagccttctaggtaaactttcatcagtgggttagttgtcttgttcagaagctgatcacggagctttggccaagcataaacactgattatggcagtccaattgtcataatccctttgattctttaatatcaccttcaagattgtttgttattgtcaatgcccccacaacctaagaccaccaggaacacactgtaattgaaaaaggtgggtttgttgctctctgcaagaagggaggacactcagcgtagagactcatgagggtggggcaggttttatccgatgacgttagaaaggacttactgaggatttgggcttgttttaggagatttgggggaaaggttcaaggagactggcttttcctggatgctgccaggaagtagtgggatggtagtaagtctgtggtaggatgtttaaataaatttcctctactgggctggaagaatgagaaggctgaagctgtaataagtaaagaagtggcagtcactcctatcagctatgataaaaggatgtttggctattactttatggtttggatgctatttttgcttgtgttcacatcatggtctatcatggtgataggccacgtacacagtggccttgtctgatgctggtgtcccatggagttgattatgctcagctgaaggacactaaggcccaactgtgggggccaggccagctcctgagtgtcagggggggctgcactgctttgtcattatcaacatctccacatacaatacagcctgtgcatgtgaggtcccagaaggaaggagctaaagcagctagactgggatcactttactcaatttgaggagaggagtgctcttgagggaaccagaggaaggcagatgagctggtctgatctccttttctattggagctctatgcagatagactgaaaatatttgctaaaacaaagagctccatctctagaatacccttagcaggatgtccttgattaaaggattatttctgaaaactaaatccagaatccgtgaggcatgattccctggaagatcatgtaagctgtacaattctctatggaataaattggagacttcatcccttagatccctttgactgtgtgaggaaccccacgaaacctcacttagtgactttattcattcttctgggcctgaaagcatatgtctcatgcaggaaggaaggcaggaccagtggggctttgcaggttgtgccattctctccttgctttgcttgagactctttattacctgtctccctgacattatttgtagtgtgattctgggtgtgttctgtgattcttgtgactaggtatccaattttgcttaaggatgcaaggaagtgtttggggagaaagctctattggaagaggtctgtagtcctagcctccctccccaccccacatttcacatcattagacctcagcacatgggtctggggcaccaacactgtcttacctgttacacagtgtggtctttatctggatgagggatgcgaaaggatacattgtgaccaagagacctgggagaggcacaaaaataacaggtgaccaccaagggtgcttggacctgagatgtttccatttcctaagaccctccaagattctccaacatttggtatagttgcccaggcaatttaacaagaaaatcaaatttttgttatgacccccattgtaatttatgcttatcgcagaaaaattgagactataagaaggagaatagaaggtcacaaaaccactctatactagtccagggatagctattcttacaacatggattgatcagtgtggggtgatttctcctagtgttttttggacagagaagcattgaagatgccctggttttaaggtcttaggatgaaggaattatagttgaacagttcaaaatgatgttatgaattactttcagatttgtttgcttgattgcattagccttgcctggccctacggtaactatttggttccatcatggtggctgagtaggtggctctggaaaaagagctattcaagaaaagcttttctttctctaaaaatattgtagggggctcgccctctgttcttggaagcaacgtttgggatggcctcttgggaggctgtctggtgaagtgtctaggggtatgtggtctggacttggacaggacaagatgcaaattctggctgggatattctagttgtggagtgttgggcaagttacttggtcttctgagtttataggtaaactggagataataggtatgtgtgaatgaggatccaatgagatgcctgtaaaacacttagccagatggctgcgtgaagaaagcacttggtaaatgctaattgttgtggttgttataattagtacaatgattagtcattgctgattgttggctaactggcggtaagaaatgaaagtaaagtaaggcagtagcagctgagggaggtggtggagggaatcaggagacacttggaggttctggttctgcctgagtttaagtgctggggaaaactagttgaataactgctggtctaacatttaacagctgtgtgacctcgggctagtcacatttcctttataaacttcctttttctcatttgcgaaatgaaggggtttcgttaggttacttctcatcacccctggttgaccattagaatcgtttgggagacctttttagaaattcttggttctggggtcttccattttccccattctcacttggtgggtctgaaagcaggcactgcagcttttccaaagctcttcaggtgaccttgaagtgtggccagggctgagaacctctgacttccaacagcacttctggtttaggaaggagcaaatcaccggcacagaatgagctctcaggaacggctgctgagctagtaattgccgtgacactgtctccctgtcccaactgcaggcacccctagacgtctcctgatgaagacttccaattttggaacagaagaatctttgaaaaaaatattattgaacttccagaaatgattcattccttctctgctccttctttagttggaaagatctgcccccatccctgtctactgcagtcccaatccctttttatttcaacatatatatccaaaccaacaaaaaaattgactcgcacaaccaaggtgaggtgtttggctttaaggataaaataaatagtttcataaaacctgcccccagatttctcattgcctctactcattttctctaatttgtagggcacactgaaagctcggattcataagatgtagaaagggcagagagttaagttacaaactcttcctaggtctgtttcaactctaacgttctatagctctgctccgtctaaacaaggaattctgttaaatttgtaacctggactttcttgaatactgaggtaatgtttcttgaagtaggatatatatacccaagaagaataaaaataattctggaggtgtcttaattctctgtgggactcaataaaagttttggtgattatatataaacacacttatgaaagcatctggtacatgtaggtgctcagtgcacatgaatttctctttcctgccagatcttttgtagtggaaaattatcttattcttccatctttgtctgcaaagatgctgctaaggaaagatgtagaagagttttacaagggagttggagtatgggaacaggaagttcccaagaaggccactgcattaatagaattgaaacaagatcccctaaaggaaaatcgctgccaaatctcttttctctaaactatccaaaatggtgccccataaattttcattgacattgaagtacataatgtaataagcttttttttccttaaatatataatgtacggagaaaaccaggttgataatggtttcctggcatagctttcaaaggcaagtgtggatgaagtggaaatatggtgcacagatattggaaagaaaccgctgttgaactcttcacattttcatgtataacccaatgattctcaaacctaaacttgcatcagaattgcctggagggcttattaaaacatgattgttggatgccacccttgcattaattaattacacctgcaactgttctatttttaaatggtcacattttaaggtactgggctgaggacttcattatgtgagtttcaaatgggacataattcaacccctagcccaatcctagtgagtggtgagtggtatctcttcgttttgatttctacttccctagtgactaatgatgttgagcatttttatgtgtgtttattagccatttttatatctcttatgaaaaaatgtgtataaaattattggcccatttgttaattgggttatccatttattattgaattataagagctctttactactctggatgcaagtcctgaaacagaaataatatttacaaatattttaatccattctgtgaattttattttcactttcttgatggcgtcctttgaagaacgtaggattttaattttgataaagtgtaatttatgtattttttcttttgttgctgtgcttttggtatcatatttaagaaataattgcctaatccaaggttatgacaattttttattctatgtttgcctctaagaattttctaattttaccttttatatttaggtctttcattcattttgagtcaatttttgtatatggtgtaaagtatgggtcctaatttattcttttgcatgtggatatcttgttgtccctgcaccatttgttgaaaagtgttgttttttttttcccattgaatggccttggcactcttgtctaaaattaattgatggtaactgtaagactttatttctggactctttattccattgatctatatttctatcattgttactgagcaatgtgcttgctgcctgacagatagggaagccaatattatggaactggtttttgagaaaagcaaaagctttatcgtgaggttgacttgcaaggaaacaggatgcaaagctcaaatctgtctccccttctgggatctgggacaagttttatgggttagggagggcaagctggtatgcagaagcactggtagggcaggtttcaactggaagtactttaaacaagaccatttatggtaaggtatggtaagggtcttaacactggacatgcctgggctcaggtttcttgcttttaaaaatgtttgggccctcaggttccagtcatgtcttgaccattttcttctgtggtggggcaggagaggaatttttcttctgggtgttattcaaggttgaggtcttcttttctgcattgcttcggctgcatgacttaacaactttttgactttgtgcctgttaaataacttgacatactattatcatcagagtagggccagttagaactggtcctgtgattacatcattatgccagtaccaattatcttgattactgtagcattgtagtaagttttgaaatcaggaagtttgtgtctttcaactttggtcttctttttcaggatttttggctcttctgtgttccttacatttccatatgaattttaagttaaactgtcactatctgcaaaagaaggaactgggatttttatagagattacattgaagctgtaaatcagcttggagaatactgtcatcttaacaatattaagtcttctggccgggcacggtggctcacgcctgtaatcccagcactttgggaggccgaggcgggtggatcacgagctcagaagttcgagaccagcctggccaacctggtaaaaccccgtctctactaaaaataataataataaaaaactgggcatggtggcatgtgcctgtaatcccagctactcaggaggctgaggcaggagaatcatttgaaccctggaggcagaggttgcagtgagccgagatcgcaccattgcactctagcctgggcaacagggcgagattctgtctcaaaaaaacaaaaacaaaaacaatattaagtctcctgatccatgaatgtagaatgtttttccatttgttcaggtcttctttactttgtaacagtgttgtgtatttttcaatgttccagtcctgtaattctttgttatatttactcctaagaatattaattgttttgctgctattataagtggaattgtttaaattttgattttatatttttcattgatagtatatttttcattgatagtatacaattgatttttgtacactgattttgtaacctgaaaccttgctgaccatgtttactcgttctaacagtttcctttttgtggatttcttataattttctatatacagtatttcatgtcatccatgaaggggataggtttacttcttcttatctaatctggatgagtttagtttatttttcttacctaaattccttggctagaactccaatacaatgttgaatataagtaatgaaatcagacatctttggactgtacttgattttaagggggagcatccagtcttttgccattatgtataatgttagctgtggggtttaatagatgaattttatcaggttgaggaaattttatttctaatctgctcagtgtttttttcatcacaagagtgttggattttgttaatatttttgtgtgtctattgagatgatcatatggtttttgtcattctacaaaatacagcacattaaattgatggatttttacatgttaatttttttttaaattttactttaagttctgggacacatgtgcagaacgtgcaggtttgttacataggtatacatgtgccatggtggtttgctgcacctatcaacctatcatctaggttttaagccctacatgcattaggtatttgtcctaatactctccctccccttgctccccacccccgccgacaggccccggtgtgtgttgttcccctccctgtgtccatgtgttctcactgttcaactctcacttatgagtgagaagacgtggtgtctggttttctgttcctgtgttttttagctgagaatgatggcttccagcttcatccatgtccctgcaaaggacataaactcattcttttttatgactgcatagtattccatggtgtatatgtgccacattttctttattcagtccatcatttatgggcatttgggttggctctaagtctttgctattgtaaatagtgctccaataaacatatgtgtggatgtgtctttatagtacaatgatttatactcctttgggtatatacccagtaatgggattgctgggtcaaatgatatttctggatctagatccttgaggaatcgccacactatcttccacagtggttgaactaattcacactcccaccaacagtgtaaaagcattcctatttctccacagcctcaccagcatctgttgtttcctgactttttaatgatcgtcattctaactggcgtgagatggtatccattgcgattttgatttgcatttctctaatgaccagtgatgataagctttttttcatatgtttgctgggcacataaatgtcttcttttgagaagcatctgttaatacccttcgcccactttttgatggggttgtttttttcttgtaaatttgtttaagttgtagacttaggatattagatctttgtcaggtggatagattgcaaaaaatttctcccattctgtaggttgcctgttcactctgatggtagtttcttttggtgtgcagtatctctttagtttaattagatcccatttgtcaattttggcttttgttgccattgcttttggtgttttagtcatgaagtctttgcccatgcctatgtcctgaatggtattgcctagattttcgtctagggtttttatggttttaggttttacatttaagtgtttaatccatcttgagttaatttttgtataaggtgtaaagaaggggtccagtttttgttttctgtatatggctagccagttttcccagcactattaattaaataggtaatcctttctccattgcttgcttttgtcaggtttgttgaagatcaggtggttgtagacatgtggtattatttctgaggtctctgttctgtttttgttttttgttttttgttttttgttttttttttttgagatgagatctcgctctgttacccaggctggagtgcagtggcacgatctcggctcactgcaacctccgcctccctggttcaagcaattctcctacctcagcctcctgagtagctgggattacaggcatgtatcaccgcgcctggctaatttttgtatttttagtagagatggggtttcaccatgttggtcaggcttgtctcgaacttatcacctcatgatctgcctgcctcagcctcccaaagtgctgggattacaggcgtgagccaccgtgcccggccaaggtctccgttctctttcattggtctatatatctgttttggtactagtactgtagttactgtagccttgtagtacactttgtagtcaggtaacgtgatgcctccaacttcgttctttttgcttaggattgtcttggctatacgggctcttttttggttccatatgaaatttaaagtagtttttttctaattctatgaagaaagtcaatggtatcttgatgggaatagcattgaatctatcaattactttgggcaatatggccattttcacaatatttattcttcctatctatgagcatggaattttttctatttgtttgtgtcctttatttccttgagcagtggtttgtagttctccttgaagaggtccttatgtctcttgtaagttgtattcctaggtattttattctctttgtagcaattttgaatgggagttcactcgtgatttggctctctgcttgtctattattggtatataggaatgcttgtgatttttgcacactgattttatatcctgagactttgctgaagttgcttatcagcttaaggaggttttgggctgagacgttggggttttctaaatatacaatcatgtcatctgcacacagagacaatttgactttctctcttcctatatgagtacactttatttatttcttatgtctgattgccctggccagaacttccaatactatgttgaacaggagtggtgagagaggacatccttgtctcgtgccacttttcgataggaatgcttccagcttttgcccatttagtatgatatgggctatgggtttttcagaaatagctcttattattttgagatatgttccatcgatacctagtttattgagagtttttagcatgaagggatgttgaattttattgaaggacttttctgcatctattgagataatcatgtgatttttttcattggttctgattatgtgatggattatgtttattgatttgtgtatgttgaaccagccttgcatcccagagatgaagccaacttgatcgtggtggataagctttttgatgtgctgctggattcagtttgccagtattttagtgaggatttttgcatcgatgttcatcagggatattggactgaaattttctttttttattgtgtctctgccaggtttcggtattaggatgatattggcctcataaaatgacttatggaggagtccctctttttctattgtttgcaatagtgtcagaaggaatggtaccagctcctctttgtacccctggtagactgcatgttagacgagataatatgtatgaactacctggcatataatagatgcttcctaaataagattctaaaaaataattatgctccaaaaatatttttaaaatcaaataatttatgttttattttctgtgttttatctcagacatgtagactgccaaagtgtatgggatgctttcaagggtgcatttatttcaaaacatccttgcaacattactgaagaagactatcagccactaatgaagttgggaactcagaccgtaccttgcaacaaggtaattgggggcatgccattgattttaaaactggggataaaagccaatggtaacaattcataggtccaaatttttattagaatgaaggaagaggaaaaatccagacattatagtgtgagtgtggttggtaggaatggaatttgcaggccattgaggggccatgatataattaagatttaggacatctggagaagggagctaagagagagaaatagggatacagagataggaaaggggctttggccaaaaactaggcagaaaaaacctaacaccaaacccaactcgaacaaacaaattaacacgacctatataataacaaaactttcccctgacctatgataataatagtagtagtagtagtaataacagcaatgccaagttacacttgcagactgcttcttctttttcttgcttacaaaagactctcctaatccttactttcttaggccttcatagccattctctggaatgggcacatcaggtgtcagcatcccaatttcaccagtgagaaaactgagggttggtgtgtttaggtgaccagtgttgcccaagtttgacaggcttcaaagtgaccagtttaaatgtaaatggtatgagacctggagccacagaggcctggattctaatacattggttatattggaaaagctctatcagagtgcaccttttctatagccaatgtttaaggcaaaattccatgtgcctaaaattttctttgtgaagcccttaaatccatccagaaattacagcctctcattccattgttagtgagctggagtcattgtgaaacttctccattcactaggcgtgatgccctatgcagagaaggtgtttggcaaataataacccaggctgacatttgtcaaataagtgactatgcgatggatagtatgctaagcaatttacttgcatttatctcagttaatttccctagcaccccattagtttatttcagtcattatcattaccattttacaggtgtagaaagtggggcttagtgatgttttggttgctcaaggtgaaacacctgataagtgatgatgatgctgggcttcaataagggctgggattttagggcccatactttaaaccagtatccttcactgactcccattaagaatgaatagggggaggagccaagatggctgaataggaacagctccagtctgcagctcccagtgagaccaacgcagaaggtgggtgatttctgcatttccatctgagatcaggtttcctcgtgtgtctacaccaccagggccctgggtttcaggcacaaaaccgagccgctgtttgggcagacaccaagctaggtgcaggagtttttttcgtaccccagtggcgcctgaaaccccagtgagacagaactgttcactcccctggaaagggggctgaagccagggagccaagtggtctcgctcagcgggtctcactcccacggagaccagcaagctaagaaccactggcttgaaattcttgctgccagcacagcagtctgaagttgacctgggatgatggagctgggtggggggaggggcgtccgccattactgaggctttaataggcggttttcccctgacagtgctaagggggctgggaagtctggactgagtgtggcaacgtggttgtggccagactgcttctctagattcctcctcactgggcagggcatctctgaaggaaaggtaacaaccccagtcaggggcttacagacaaaacctccgtctccctgggacagagcacctggcagaaggggcagctgtgggcacagcttcagtggatttaatcattcctgcctgctggctctgaagacagcagctgatcctgacaagagggattctcccagcacagcacaccaactctgctaaaggacggattgcctcctcaagtgagtccctgacccctgtgtctcctgactgagagagaccacccaacaggggtcgatagacacctcatacaggagagctccggctggcatcaggccggtgcccctctggaatgaagcttccagaggaaggagcaggctgtcatctttgctgttctgtagcctccactcgtgataccttcaggtgcgggaggaacccaggtgaatagggtctggagtggaccccctgcacactgcagcagccctatggaagaaagggcctgactgctaaaagaaaaaacagaaagcaacaacatcaatgaaaaagaccccacaaaaacccatccaaaggtcagtagcctcaaagatcaaaggtagataaatgcaagaagatgagaaagaatcagcacaaaaatgctgaaaactcaaaaagccagtgtgcctcttctcctccagatgatcttaacacatctccaacaagggcatagaactgggctgaggcccctaaaaagagatgagttcatgtcctttgcaggcatatggatgaagctggaaaccatcattctcagcaaactatcacaagatcagaaaaccaaacaccacatgttctcactcataagtgggagttgaacagtgagaacacatggacacagggaggggaacatcacacaccagggcctgtcaggggtgggtgctaggggaggataacattaggagaaatacctaacgtaggtgacgggttgatgggtgcagcaaaccaccatggcatgtgtatacctatgtaacaaaactgcacattctgcacatgtaacccagaacttaaagtataaaaaaacaaaagatactagctacatttacccaatgttaaaaaaaaaaaaagaactgggctgaggctgaggtggatgaattgacagaagtaggcttcagaagatgcataataatgaaattcactgagctgaaggagtatattctaacccactgcaaagaagctaagaaccatgataaaacataggagctgttaaccagaataactggtttagagaggaacataaatgacctgatggagctgaaaaacacaacacgagaacttcaagatgtaaacacaagtatcaataaccaaatagaccaaacagaagaaaggatatcagagcttgaagagtatcttgctgaaataagacaggcagacaagattagagaaaaaagaatgaaaaggaacaaacaaaacctctgagaactatgggattacataaaaagaacctatgactgattggggtacctgaaagagacaggaagaatgaaaccacgttggaaaacacacttcaggatatcatccaggagaacttcttcaacctagcaagatgggccaacattcaaattcaggaaatccagagaaccccagtaagatactccatgagaagatcaaccccaagacacataatcatcagattctccaggtcacctataaagggaagccaattagactaacagcagacctctcagcagaaacctacaagccagaagagattgggggccaatattcaacattcttaaagaaaataatttccaaccttgaatttcatatctagccaaactaagttcataaatgaaggagaaataaaatctttttcagacaagcaaatgctaagggaattcgtcaccaccaggcctgccttgcaagagctcctgaaggaagcactaaatatggaaaggaaaaaccattatcagccactacagaaacacaccgaagtacacagaccaatgacactatgaagcaactacgtaaacaaatcttcacaataaccagctagcatcatgataactggatcaaattcacacataacaaattaaccttaagtgtaaatgggctaaatgtcccaactaaaagacatggaatggcaagctggatagtcaagatcaattggtgtgctgtatacaagagacccatctcacatgcaaagacacacataggctcaaaataagggatggaggaatatttaccaagcaaatgggaaacagaaaagagcaggggttgcaatcctagtttatgacaaaacagactttaaaccaacaaagatcaaaaaagaaaaagaagggtattacataaggataaaggggtaaattcaacaagaagagcaaactatcttaaatatatatgtgcccaatacaggaacaccgagattcataaaacaagttcttagagaccttcaaagagatttagatacccacacaataatagtgggagaatttaacatcccactgtcaatattagacagatcatcaagacagaaaattagcaaagatattcacgacctgaactcagctcaggatcaagtggacctgatggatatctactgaagtctccatgccaaagcaacagaatatacattattattggtgccacatggcatctactctaaaattgatcacacaattggaagtaaattactcctcagcaaatgcagaagaactaaaatcataacaaacaatctcacagaccacagcacaatcaaattagaactcaagattaagaaactcactgaaaaccatgcaattacatggaaattgaacaacctgctcctgaatgactcctgggtaaataataaaattaagccagaaattaagaagttctttgaaactaataggaaaaaagagacaatgtatcagaatctctgggatgcaactaaagcagtgttaagagggaaatttatagcactaaatgcccacatcaaaaagctaggaagatatcaaattgacatcctaacatcacaactaaaagaactagagaaccaagagaaaacaaatcccaaagctagcagaagacaagaaataaccaagctcagagcagaactgaaggagatagagacacaaaaatcccttccaaaaaaaaatgaatgcaggaggtggttttttgaaaaaaaattaatagaatagatggatcgctagctagactaataaagaaaatagagaagaatcagatagatacaataaaatgataaaggggatatcaccacagaaatacaaacaaccatcagagaatactataaatacctctatgcaaataaactagaacatctagaagaaatgaataaatttctggatacatacaccctcccaagactgaaccaggaagaagttgagttcctgaacagaccaataacaagttctataattgaggcagtaataaataccaaccaaaaaaaaaaaaaaaaaagcccaggatcagacagatttataactgaattttaccagatttacaaagaggagctgataccctttcttctgaaactgttccaaaaaattgaaaagtaaggactcctccctaactcattttatgagactagcaccatcctgataataaaaactggcagagatttaaaaaaaaaaagaaagaaagaaaacttcaggccaatatcctgaagaacatcgatacaaaaattctcaacaaaatactggcaaactgaatccagcagcacatcaaaaagcttatccaccatgatcaagttggcttcatcctcaggatgcaaggcaggttcaacgtacatgaatcaataaatgtaattcattacataaagagaactaaagacaaaaaccacatgattatctcaatagatgcggaaaaggccttcgataaaattcaccatcccttcacgttaaaaactctcaataagctaggtatcaaaggaacatacctcaaaataataagaaccatttatgacaaacccacaagcaatatcatactgagtgggcaaaagctggaagcattccccttgaaaaccggcacaagacaaggatgtcctctctcaccactcctattcaacatagtattggatgttctgaccgggacaatcaggcaagagaaagaaataaagtcttttcaaatggaaaaaaggaaataaaattgtctttgtttgcagatgacatgatcctataactagaaaaccggatcatctcagccccaaagcttcttaagctgataagcaacttcagcaaagtctcaggatacaaaatcaatgtgcaaaaatcacaagcattcctgtacaccaacaacacgcaagcagagagccaaatcatgaatgaactcccattcacaaagggaataaaatacctaggaatacagcaaacaagggaagtgaaggacctcttcatggatacctataatccactgctcaaggaaatcagaaaggacacaaacaaatagaaaaacattccttcctcatggataggaagaatcaatatcgtgaaaatggccatactgcccaaggtaatttatagattcagtgctattcccattaaactactattgacattcttcatagaattagaagaaactattttaaaattcatatggaaccaaaaaagctcatatagccaagatgatcctaagcaaaaagaacaaagctggaggcatcgtgctacccaactccaaactgcactacaaggctacagatgccaaaatagcatggtacttgtacaaaaataaacacatagaccaatagaacagagtagagatctcagaaataaaactacacatctgcagccatctaatctttggcaaacctgacaaaaacaagcaatggggaaaggaatccacatttaataactggtgcttgagaactacctagccatatgcagacaattgaaactggaccccttccttgcaactcatacaaaaattaagatgaattagagacttaaatgtataacccaaaactataaaaaccttagaagaaaatctaggcaatatcatttgggacacaggcacaggcaaagatttcatgaaattgccaaatgcaattgtaacaaaagcaaaaattgacaaatgggatctaattaaactaaagtgcttctgcacagcagaagaaactatcatcagagtgaacagaaaacctgcagaatgggagaagatttttgcaatctatccctctgacaaaggtctaatatccagaatttacaaagaacttaaacaaatttacaagaaaaaaataaacagccccatcaaaaagtgggcaaagaacatgaacagacacttctcaaaagaagacatccatgtggccaacaaacatatgaaaaaaagctcaacatcactggtcattagagaaatgcaaatcaaaaccacaatctcatgccagtcagaatggcattattaaaaagtcaagaaacagcagatgctggtgagattgtggagagatagaaatgcttttacactgttggtgggaatgtaaattagttcaaccattgtggaagatagtgtggcaattcctcaaagatctagaactagaaataccgtttgacccagcaatcccattactgggtataatagaaatcattctattataaagatatgtgcatgcatatgttcattgcagtgccattcacaatagcaaagacatggaatcaactcaaatgcccatcagtgataggctggataaagaaaatgtggtacgtatacaccatgaaatattatgcagccataaaaaggaacaagatcatgtcctttgtagggacatagatggagccagaagccacatcttcagcaaactaacacaggaacatgcaaatgctgcatgttctcacttataagtgggagctgaacagtgagaacacatggacaccaggaggggaaaaacacacactgtagcttgttggggttggggtgaggggagtgagaacattaggacaaatagctaatgcatacttggcttaatacctaggtgatgggttaataggtgcagcaaacccatggcacatatttacctatgtaacaaacctgcacatcctgcatgtataccctggatatacatgcccaggatatacattttatttaaaataaaaataaaaataatagattcataaaacagaatataattctgaactttgactccctgtacctttaagagggacccttaaatttaaaaatctattgtattttttttttagtaggggtagggaatatttagggaatttggaaggggttatatagttctttaagaatcaaatagcacatcttcctgaaaatagcacgtagacaaagtttttttggagataaccttaggaatatcgtaactctctgatgccacctccatatgtgatcctatgttgattataagattttgatcagtggctttcagacttttttgactgcaacctagaataaaagattcatttacattgtgacctagaacacacacacacacacacactctctctccgccactctcctgcacacagaaatcattgatgcttacaacaattcttactcttactatgggtgatttactttgatatgctctgttttttttttcatttacaaaactgtggattaattttttttgacatgctaaattgatctcagtaatagattgtatttattcttccttagattcttctttggagcagaataaaagatctggcccatcagttcacacaggtccagcgggacatgttcaccctggaggacacgctgctaggctaccttgctgatgacctcacatggtgtggtgaattcaacacttccagtgaggctctgggccctgtgggattgcccagggatgtggagggtgaacagagtgacttctgctggaggccctgaatgattagtgtggaggacagagccacaggcacccatcctgatgccatctatacttatattagtccatttgtgttgctattaaggaatacctgaggctgcgtaatttataaagaaaagaggtttatttgactcacagttacgcaggctgtacaagaagtagggtaccagcatccacttcgggtgaaggcctgaggctgtttccactcatggagaaggggaaggggagctggcatttacagagatcacatggtgagggaggaaagcaaggagaggtcaggggaggtgccaggctgtttgtaatgaccagctgtcctgggaactagtagagtaagaactcattactataaggacagcaccatgccattcgtgcaggatcatccctatgacccaaacacctcctactagtcccgagctccaacactgggggtcgaatttcaacataaggtttggagagttaaatatccaaactatagcactacccttaatggcaactcaggctgatataaagtagcattccctgttttcttgaaaaattgacttcagagttggggattgcccatgctccctaattcccttcttttgagtgctcacatagcctgcttccgaattcttggtattttgctctctgtaaggtcatcattcaggtccaaagaagtctagaacaggatgaggtctcagtgggacctagaccaaggttcttgctcttcagaatcatcacagtagccatggactggactcttccatctcaggcactggctttgccatcatttttcagatgtagccttatcctgcccagaaagactcaacacctcaccaggggaagggatttcctacaaccaaaaccctactgcagttttcacttcttttttttttctttttgtttatatggtggatatttttactttatatagttttattcttatttttactgtttttcattgtttgtttttaaaagcttatcttattatagcttctttgtcccaggtttgcattactttcaattacaaaaataaagcatgattatttgaaaaaaaaatacttgcacattacagaaatgcataaaagcaaaaagcaaatgtcactctgaattttcccttcacctcctacctccgcatcacttctcaaagggtaactattatcagcaatttgatatagatctttctagacttttcctatgctaatgtaaacatatatatttaaaatgtacacgcgctgttgtgcaacttgctttattcacttaaaattggtaggtataaagatagctatcctcttttaaaaggctttatcattaagaatcctattaatggatattaagttgctttagttttggttgctattatgtcattattgtaagaaacacttttgtgcgcacacacacacacacacacacacacacacacacacacacctgcatacttgaacgataaatttttataaatgaaacttcaatgttaaaggataaattgtaatagaaactggtaacatgtcatttaaaagatggtaactataccctcatcaagagtatatatatgaggccaggcacagtggctcatacctgcaatcccagcactttgggaggtggaggagggaggatcacttgagcccagtagtttaagaacggcctgggcaacatagtaagaccccatctctattttacactaaaaaaagaaaaaaaaagaatgtatatgagatagtttatttacctatatccccactaacaccaggtattgttactttaaaatttttggctcatttcagaagaaaataatacctcaatttagtgtgaagttctttgatggtgaggctactatatatacataaatggtagattttctgtttcttctgtaagctggcagttcatatattttgaccatctgtatgcggtatctattattttctaattagtagaagttctttataaattaataagagctacgatgtattaagtacttacaaagtgccagtgttccatgtgctacgtaagtagtcactcatttaatcctccacagccccatgaggtcatatggtgatcccattttagagataggaagtctgaggcatggagttaagtaatttgccagccagtaagtggcaaagcaagaagcaaagtttctcagactaacttgagaaactttgctcttaactgccatgtttttctgcccactttctggctcaagttggcgaatatatttttcttattttggactttacacggtgtttatggtctgtcttttgccacccagaaatgcagaaaacctgtctgttctcttccttatagcttctgtgtttcatatcttccttaaaaagatcttcttagagaagcattcttctgtattttcacctactatttttactttcacaatgtttaaaatattttccatatttagatctgagtcttcccacctagaatatggtaatataaatatgtttttccattaatttttttagattttacagtttttcccatgttccatgttttcctttttaaatttcccttttaacaatgactgttttattggtcattcatttaacatttagcttttaaatgtatttacagtagacttctctccttttgttttattttttaatttgttcaatttttcttgaaataaagtagagaaaatgaaataatttatttttaagaactgatttatttacagttcagagttccttatttttgccttcttttaaattgaattatgtatatgtagttttatttatctacattctaatactttggcctcaatttttaatttcttcttattttatagattatctttcaagttcctgatgtatatgtatttatttactttttattctaagttgacaatttataattgtatgtatttgtggggtgaaaaatgaatttatgaatacaatgtgggataattaaatcaaactaattaacatatccaccgcctcaaatactttttttagtttttgagacagggtctcactctgtcacccaggctgaggtgcagtggtgcaatcacaattcactgcaaccttgacctaccaggctcaggtggtcctcctaccttagcctcccaggtagctgggactacaggtgcctgccaccacacttggctaattttttgtattttttttagagacagggtttcaccatgttgcccaggctggtcttgaactcctgggctcaagcgatctaccctcttcagcctcccaaagtgttgggattacaggtgtgagccaccaggcccgacctcaaatacttattttttgtagtgagaaaatgtgaagtttactgtcttagcaatgttgaaatgtacagcacactattattaactacaatcaccatgctgtgcaataaatatttttaaaaaccctttctaactgagattttgtactctttgaccatcatctccccattccttccaacttctggtctctgtatccaccattctattatctgcttctatgaacttgattgttttagattccatatgtattaggacatgcagcatttgtctttctgtgggtggcttattttacttagcatattgttttcttgttccatctatattgtcacaaatgacagaatttctttctttttaaagtctgaatagtattccattgtgtatatataccacactttatccattcgtctattgatggactcaggttgattccatatcttggctattgtaaatagtgctgcaatgaacatgggggagcaggtatctctttgacaaactgatttgaaatcttttgggtaaatacctagaagtgggattgctggatcatatggtagtattctatttttagtttgttgaggaactttcatcacattttccataatgggtatactaatttactttcccaatagtgtacaaataaccccctttcttcacattcttgccaacacttgttatttatctttcatctttttgattatacccttctgacaggtgtgagatgatgtctcattgtggttttaatttttgtttccctattaattaggaagcttgagcattttaaaatatatttgttggccatttgtatgtcttttgaaaaatgtctattcaggtcctttgcccacctttaaattgattttttttcttgtttttgagttgtttgagttccttatgtattttgtttttgtttgttttttaatttttaatttttgtgcatacataataggtgtatatatgggatgtgtgtacatgagatgttttgatatagatatacagtgcataataattacatcatgaaaaatgtctctttccccataagcatttatcttttgtgttacaaacaatccaattttattcttttagttattttaaaacaggggtgtccaattttttggcttccctgggccacattggaagaattgtcttgggccacacataaaatacactaatactaatgatagctgatgggctgaaaaaaaatcgcaaaaaatctcctaatttctaagaaagtttatgaattgaacttatgtgttgggctgcattcaaagctgtcatgggctgcttgagacccatgggccatgggttggacaagctttttttaaaatgtacaacaaaattgttattgactacagtcaccatactgtgctatcaaataataggtcttattcattctaactattttttggtaaccatccccacctccccacaatgtcttgctacacttcccagcgtctggtaaccatttttctattctccatgtccatgagatcagttgttttgatttgttggatgctaaaataagtgagaacatcctatgtttatctttctgtgtctagcttatttcacttaacataatgacctccagttctattcatgttgttgcaaatgacaggaacacattcttttttgtggctgaatagtactccattgtgtataaataccacattttctttatccatttatctattgatggacatttaggttgtttccatatcttggctattgtgaacagtgctgcaataaacatgggagtgcagatatctcttccattgactgattttctttctgttgggtatatatccagcagtggcattgctggatcatataatagctctatttttatttttttgagaaacctcaaaactgttctccatagtggttgtactaattcacattcccaccaacagtgtacaagggttcccctttctccacatcctcatcattatttgttattgcctgacttttggatgaaagccattttagctggggtgagatgatatctcatgatagttttgatttgcatttatctgatggtcaatgatttgaacacattttcatatgcctgtttgccatttgtatgtcttcttttgagaaatatgtattcaaatcttttgcccatttttaattggattattagatttctttcctatagagttgtttgaattacttatctattctggtttttaatgccttcttgaatgggtagtttgcaaatattttctcccattctgtgggctctctcttcactttgttgattgtttcctttgctatgcagaagctttttaacttgatgtgatcctgtttgttcattttgctttcgttgcctgtgctcatggggtattgctcaataattttttttgcccagacaaatgtcatggagagtttccccagtggtttcttgtagtagtttgcagtagtttcatagtttgaggtcttagatttaaatctttaatctattttgattttatttttgtatgtgatttgagataggggtctagtttcatttttatccattgagccactctgtgccttctgattgtagagtttagtccatttacatttgacgtaaatgttatatttttaagtaaggacttactcctgccattttgttacttgttttctgtttgttttgtggtcttctcttccttctttctttcctttctgtcttcctttcagtggaggtgattttttcagtttcctgctttttattttttgtggaactgttatatgtttttgagtttgaagttaccatgaggcttaaaaatagtatcttatatcccattattttaagctgataacaacttaacacagtttgcataaagaaacaaagacagcaaacagaaagctaatacaaactctataccttaacttcattctcccactctaaaactttttgttgtttctatttatgtcttattgtactttatatgtcttgaaaagttattgtagttattatttctgattggctcatcatttagtttttctacttaagacaagagtagtttacacgtcatagttacagtgttataacattctgtggttttctgtgtacttactactgccagtgagttttgtaccttcagatgattaaattgctcattaatatccttttctttctaattgaagtactccctttagcatttcttctaggacaggtctcgtgttaattaaatccctcaccttttgtttgtctggaaaagtctgtgtttctccttcaagtttgaaggatattttcaccagatatactattctagagtaaaaacttttttttttgtctttcagcacttcaaatatgtcatgccactctcatctggcctgtaaggtttccactgaaaagtctgctgccagatgtactgaaactcccttgtatgctatttgtttcttttctcttgctgcttttaggatcctttctttatcttggacctttgggagtttgattatcaaatgctttggggcagcattcttgggttaaatctgcttggtgttctataaccttcttgtacttgggatattgatatctttctctaggtttgcaaagttctctgttattattgctttgaataaactttctacctgtatctctttttctacctcctctttgacaccaataactcttggatttgcccttttaaggcaattttctagatcctgccagtgtgcttcattgttttttattcttttttcttttgtctcctctgactgtgtattttcaaatagcctgtcttcaagctcacaaattctttcttctgcttgatcagttctgctataaaaagactctgatgcattcttcagtgtgttatttgtacttttcagctccagaatttctacttgattctttttaattatttccatctctttgttaaattcatctgatagaattcttgaatttctttcagtttcctcaacatggctattttgaattctctgtctcacatatctctgtttcttcaggattgatctctgatgtcttatttaattcatttggtgaggtcatgtattcctggatggtcttgatacttgtagatatttttctgcatctaggcattgtatttattgtagtctttacaacctgggcctgtttgtacttgtccttggaaaggctttccagatattttgaaggactcggatgttgtgatctacgttgtatctgctgtagggggccctgcaagcctagtaatgctgtgggtcttgtacacactcatggaggtaccaccttgatggtcttggacaagatctagaaggattctctggattaccaggcagagattctttttctagtccctttactttctcccagagtctctctctttctgttctgacccacataaagctggtgacacactccaccgcaactaggactttgctgggtaagacttgaagccagtacagcacttgcccagggcctgcagtaaccacttcctagctgccatctatatttgctcaaggctctggggctctacaatcagtaggtgagaaagccagccagacccgtgttcttctcttcaggttggcaagtttcccaaggccctgggttggtccagaggtgccatccagaagccagggactagagtaaaaaaccttagaagtctacctagtattgcattgtactgtgactaagctggcattcaaaccacaagacacagtccttcccatgctgtcttccccttttctaaggcaaaggagcctcacctcatggccaccaccaccacaggcccacagggagtactgccagtgtactgttaatattccaaggcccaaggactcttcagtcagcttgtggttaatgctgcctggcctgggactcacccttcaaagcagtgggctcccctctggcccagggcaggcccagaaatgctatccaagagccacatcctggaatcagggaccccaagcccagttggtgctctacctctctgtggctgtacctgaagccagcaagtcacagagtctcacccaaggcccatgacatactaattgggtatcacttctggtttttcagggcccaagggctcttcagttagtaggtgatgaattctctccagatgtcgttgtgaagataaaagaggtttattttcatgaaaacatatatactttaaagcaccttatgaaatgtatgtccattccaccatcaacatttttacctctgttgggaagataattccttttgactccacaataattatttatatctacacatgggaatgtttcttttttatttgtgtggttttggttttaaagcatttaatcattacaagactcctagaattactatatcatgtgctctctgaaggcaaagttcccatctaatttttctattttatctttctacctctaagacctaaaactcaataaatgtgcattaaggcagatatccttgggagaagtgacacagaaactatgtattcatgctctgtgtccattgtacttcactcagggttaagactgccttgatgagggcaagtgtaggaagactctgaggccatctgagagtaagtggtgaagacttaagaagtggggcaggaaacacagcaagagagagttgtcaggaagcagaaaagcagttggcaaaagcaaccactgaaggactggttttacctctaattcttcctggactggggataatcctagagggcttgtctctgtcagatgaacttttggtagcatttcccagaaccatgactcaaaacttgccactgtgttcccatctgggatttggaagataaggtaagaacttggaaagaattcaggggacacttagttaaattgggtcaaaatgtgtccattctccagcctccgtcttggcagtgacacattggaaaatggttccactatgactgaacagccagggaagagtacagcttatttatactctctgttttccactttattttctacaaactatgtcttttagaaataaactatcaatcttgcccagactggagaaaggactgcagcaacaaccctgtttcagtattctggaaaacggtttcccgcagggtaagtaccaagtagtgaaattctagagctttggagaccacagaacttaagacgttactcagtcagtgcttggttttaacacttttggattacaaatacttttaggaatgaaaatataggattcattcctgagaaaaaggttcagatgcacatgccagaaaatttacacatccaattttagaacattcttagagggtccatgggctccagttgcagaatctttgcacgtacccactctgactttggctaccaggaacctggggcttggtttaatcctctgattcaggtattagtcaatcttagatacctgggacagtcgtaacaatctacatgtatagaccccttactatgtggcaggtacggtcctcagatctttacatgaactagtaacttgcatcttcaccagaaccctgtgaagcaggtgccatgagtattgtaaccatttaacacataacgtgaaggtacaagtaaacaaggaatctactaaatgtacagaattagtaagaggcatatgtgggagtttatcccaagctgtctgactccagattcagaatctaggctgggaaaaactcaccactccaccctctacctattttttttaaaaaaattgatacataatagttttacatatttatggggtatatagtgatgtggtgatacatataaggtatagtgatgaaatcggggtaattaacatatctgtcatctcgaacatttatcatttctttgtgttgggagcattgaatatcccccttctggctagctgaaactacatattattaactgtagtcctcctacagtgttattgaacaccagaatttattcctcctatctaactataattttgtatcttttaacaaatctctacctatctcctcctcctcctacttttctaagcctatggtggcctctgttctgccttttacttccataagatcaacttaattttagcttccatatatgagtgagagtatgtagtatttaaccatctgtttctggcttacttcacttaacataatgccctccagttccttccatgttgctgcaaatgacaggaatacttttttttttttttttttttttttttttttttgagatggagtcttgctctgtcacccaggctggagtgcaatggtgtgatctcggctccctgcaaccaccacctcctaagttcaagcgattctcctacctcagcctccccagtagctgggactgcaggtgtgggccactatgcccggctaatttttgtattttcagtagagacggagtttcaccatgttggccaggctggtctcaaactcctgacctcaggtgatccgccagccttggcctcccaaggtgctaagactacagccatgggccaccatgcccggctaatttttgtattttctgtagagacagggtttcactatgttggccaggctgtctcaagctcctaacctcaggtgatctacccaccttggcctcccaaagttctgggattacaggggtgagccactgcacctggccaggaatacattttttaattcctgaatacaattccattgtgcacatatacccccatctatttctaactttactcaaagctacctgtgtatatttatttatcttgtaagttgcttcagtgttaagtggacaaggaaacatttcttttcaagtgtgttagggaaaaaaagagaaaggaaggaagaaagaaatgaaagaaaaaggtgtgagtaacaatacactaattataactttcaaaattaaacttagacatctgaggaactggggcaggtggaaatgtatttgttaagtgcatatgtcttagtccatttgtgctgctatcacaaactacctgagacttggtaatttttttaaaacaggagttttattttctcattgtgctggatacttgggaaatccaggattaaggtgccagcagattcaatgtctggtgagggctgctgtctgcttccaaaatggtgccttcttgctgtgtccttacatggcagaggcagaggggctaaaggaacctagctagttccctggagccctttcataagggtgttaatcccattcatgagggcagaacccttagggcccaattatctcctaaaggactcactacttattaccatcacattggtcttaggtatcaacataagaaacacatacattcaaatcatagcagcatatctgtgacaagccttgaagtagttcctctgtcattcccattgagtcatccccataggtagtgtgacaaatccctacattaaaggtgaagaaactaagactcagaagttaagtgactcatccaagttccctgggctaataagttacagacctaagagcctaacctaggcctccctgattccaaagccatgctcttcaattttgttctttgaatctgcttattggttctgtcttttaaatgacaggtttgatcttaactctaggttggtacctagctaaatctctgtcttagggggattcatgtaaaccctggtacgatggaaacagaaaaacagcctggaagttgacataaggagaccatgtttaaacttgggcagattcctttactcattctgatcttctgattcctcatttgtcaaatggaaatcaaaatatacttgttccatagggttactgcaatgtttaaatgagataccaccatcctataacataacccaaaatccacccacttcaaaaataattcattgagtacttactatggacagtgaacattcatgggcactttatagtttttgtttgtttgctttttctgagaatagtttccatttcactactctatggtatgttttaggacagtgctgttgctaaaatcttttaaagccaagtcacattttatatgtatcaagaacctccttgcgttccccactcagtccctggcactaggaatacagaggtgcacgtgattgaaggcgtcctgcccttgtgagctaccagcacctttattttgccaatcactcatggatgtatgtggatggacttcttttttcagacttgtccctttctttttctgataacagaggccatgtttttttttaatttttaatttttgtgggtaggttaaactcatcattataatacaatacagttggataatgtggagggaatgtaagatgctgtcagagtcagagaaggggacttgagctagtcccaggggttggggaggcctcctggagggagcatatagaacactattttgttcatttcatttttccaaagtctaacaaagattcctgctgaatgtttcttgcatggagaaataagaccctttgctcaagcatatttattcattcacttattcagtcctcctttctctctgtgcttttccaggcctaagggtcccctgttctctcctcaggttcccctcttatggtgttcccatttccctcatccctgaatcatccacctgttcccactaaatgaagcataatgtttacagtgcatgacactgagaaagcactttcatcttccccctctagacattcctcttactcctctggacttctgacttctgaaccactgaaccaccagctctatgaactataacactgaacattgttcacttagagattggagcaactgcttcaagaactctgatatgaagcataatccgtccagtggcttggaataaaaattgtgtagacctgacattcctgggctaaaaccatatgggatatccttccttaaccagctattgctaagtattgttttgaatgaaactgctggaggatggtgattaagtttgcatgatgaatggtgggcatttttttttttaagtttgcagaagctgcctgtgatgtggtccatgtgatgctcaatggatcccgcagtaaaatctttgacaaaaacaggtacacatttattttgcatcctgtttgcaagtatcctgttgcaaatatcacagtgaatatttcatctctagaaagaatatgcttttcatgtttcaggtcagttctgaagattagggccaaaaaaggtaaaaattttgaattccgtggagagagttgtctcctgtcaatgtgtttgtctgatttctcctttgccaaaaattgtctaccaggttctaatggccactgcactgtatctagcccctgctcttaacttttgcaggcctggtgtaattttctcagctttctctcccgttaccctccaccctacccattgctcaccattgttcacaccgttcccccatatgacctgcctcccctgctcccctgctcccttctgtctaaatcttcaccatccatgaagacctgccttgaccctcctctcctccaggaaaattgtgtaccccaattcagtagtaaaactactaccgggaacatcgggaactgtgctgggctcttggccttcactatctttttgcagacattgtcaacaatgtactgtagtggttgaaagcaggtactggcggtcattacatatcatctgtgtgacctcaggcagggcagtcaacctctgtgagcccctgaatatgtaccaaagagttgatggtgatgggaagattaactgagacaacagatgaaaaatgctgagctctgtgcctgacaacagagaaagtgctcaatgagaatcagctattattctcatttgctgatccttgccactgaatctggccacacctgtgccttccttggctgatctccttctatatttacagtttttactatgttgattaccttttcggcctttgttctctaatttttgttctctaatcccacataaggctgactgaaaggaggaagcatatattaatttgccttataaactctaggtgccccaaattaatttttcttctctcctgttttaatatttaattctacaaggaagcatttgtcctttcgtcttctgatcccaatttttttgggtaaaagcattaacatttcagaattttatgatctaatattatggttcaagcacttgaaacaggagtgtcagttgtcagagactaacagggaagagtttaggaatgggattagggcaggcaaccatagtctttcaaagcattgcctctcaaacttcactgagcatgtgaatcacgtggggattgttcaactgcagatcatttcagcaggttatagtggttgaaattctacatttcttttttttttttttttttttttttttgagatggcgtctcgctctgttgcccaggctggagtgcagtgacacggtctccgctcactgcaagctcagcctcccaggttcacgccattctcctccctcagcctcctgaataactgggactacaggcacccaccaccacgcccagctaattttttgtatttttagtagagacggggtttcatagtgttagccaggatggtctcgatcccctggcatcatgatctgcctgcctcggcctcccaaattgctgggattacaggcatgagccaccatgcctggctgaaattctacatttctaatgagttcccaggtgatgttcattaggttggtctaaggaccactcttcaaatagcaaatatttaaagaatcaacattaatgcacaaattaagaattttattttgagaatcttgttaaccgagggtcatgctgaataagaaaaggttattgactgatttgcaatttgatgtgtcaactctaaaggataggtcctagccagtgcctttctgcctgctggttgttgaggggggtgtggatgctttcgtttggggttgatgtttggggttctttgtttcttctattttagcacttttgggagtgtggaagtccataatttgcaaccagagaaggttcagacactagaggcctgggtgatacatggtggaagagaagattccaggtatatcttactactttgtacccaagtgttattttatgaatcagtccacaaaagaatccacagtcacaagcacgcactgggaacaaattgactcaggaaatgaaactacatgaatgtgcatgaatcccaacagcctcttaactttatctccacaaaggatatttaactgcttgacacttcagctctcctgctgacccaggagctcttagaggatttacctctactttacctctttatccaagggccttgtccagggcgtgctacaaaaacaaagagactccaaaaatgtttgtgagatcttgtaattttaatactttcttctttcttccccagagacttatgccaggatcccaccataaaagagctggaatcgattataagcaaaaggaatattcaattttcctgcaagaatatctacaggtaattaatttcttcttgaagaaaaaaatgactgtcttgtcacctgtagaatttccttttttccttagcctcctctgagcttggagggctgtgtgaatctttcttgggccttgatgatgatcacagatggcaacctctggtgatctctgtccctccttccaagccgagtccagaaggtatccaagctagtggccttcacttggctgcctttcctcatccgtctctattgatcccaagtaggacttgcctctaaagctgacacaacctttgatggcatattttttcattcccagtgtgagtggcccagtccagggttcactggcctactaggtttcaggggagcaagggaatgttttgctaagccctttctcccaagttgtaaaatccttgtgacttgacatcattttgcaagtgaagcttccttagttggatctgagtacagatgcctaacacatgacaaggcgtcacacggcagtctaccaaaatctatattttttaaattaaaaaaaaaagtatttacaaaatttttctgataatttgtgtttattagaaaacagtttaaaattacagatagatatatattttttaaagtcacatataattctagtttcaaaactgagacccctcactcatttttaagcagttgtgaccaatggtgtaggtaggtactcattggtagaagcatctttggagatttttccacgtataatagcttggaacaagattgatgcagagaggaaaagctgttcaaaggaggtagaagctgagatgctagaatattgttcctgtttccatgtcactaccttctctcactaaccacatcagaaaagcagaaggatagattctggagactctactgatggcttttgtttcccaaatgacctgaattccccatgagtcaccttgcttctatctggaaacagccagaaaaggccatgagcattctacagcagttagacaggaaaacagaaagaatgaatgaaggagcaactgtaaaagcaatcttgcggcggaggagccaagatggccgaataggaacagctccggtctacagctcccagcgtgagcgacgcagaagacgggtgatttctgcatttccatctgaggtaccgggttcatctcactagggagtgccagacagtgggcgcaggccagtgtgtgtgcacaccgtgcgcgagccgaagcagggcgaggcattgcctcacctgggaagcgcaaggggtcagggagttccctttccgagtcaaagaaaggggtgacggacgcacctggaaaatcgggtcactcccacccgaatattgcgcttttcagaccggcttaagaaacggcgcaccgcgagactatatcccacacctggctcagagggtcctacgcccacggaatctcgctgattgctagcacagcagtctgagatcaaactgcaaggcggcaacgaggctgggggaggggcgcccgccattgcccaggcttgattaggtaaacaaagcagccaggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcttcaagtgggtccctgacccctgacccccgagcagcctaactgggaggcaccccccagcaggggcacactgacacctcacatggcagagtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacatgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcaggttaccctcaaaggaaagcccatcagactaacagcggatctctcggcagaaaccctacaagccagaagagagtgggggccaatattcaacattcttaaagaaaagaattttcaacccaaaatttcatatccagccaaactaagcttcataagtgaaggagaaataaaatactttatagacaagcaaatgctgagagattttgtcaccaccaggcctgccctaaaagagctcctgaaggaagagctaaacatggaaaggaacaaccggtaccagccgctgcaaaatcatgccaaaatgtaaagaccatcgagactaggaagaaactgcatcaactaatgagcaaaatcaccagctaacatcataatgacaggatcaaattcacacataacaatattaactttaaatataaatggactaaattctgcaattaaaagacacagactggcaagttggataaagagtcaagacccatcagtgtgctgtattcaggaaacccatctcacgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctcagtgacctacaaagagacttagactcccacacattaataatgggagactttaacaccccactgtcaacattagacagatcaacgagacagaaagtcaacaaggatacccaggaattgaactcagctctgcaccaagcagacctaatagacatctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagatgaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatagaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacgcttcatgctaaaaactctcaataaattaggtattgatgggacgtatttcaaaataataagagctatctatgacaaacccacagccaatatcatactgaatgggcaaaaactggaagcattccctttgaaaactggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcacaattgcttcaaagagaataaaatacctaggaatccaacttacaagggatgtgaaggacctcttcaaggagaactacaaaccactgctcaaggaaataaaagaggacacaaacaaatggaagaactgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactacctgacttcaaactatactacaaggctccagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaattttcgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatataaccaaatgagtataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacatatgtaactaacctgcacaatgtgcacatgtaccctaaaacttagagtataataaaaaaaataaaaaataaaaaacaactctcagaagcaaaaaaaaaaaaaaaaaaaaaaaaaaaagcaatcttgcagatatctgacgagtctaagctgttcaaagatatgttgcatggagaaaatagaatagtagaaacctagacaaagactgggaaataaagatggtcttatccccaatactcttttaccttttttgtcttatgaaacattaacctttttctcataaatgaccagaagacctttatattataattcgtcaactcccctcatttgtgtctgctttaggctccaagtgagctcactcattctccatctggaaagaaaatatgggcatggcttccatttggacttgtacagacagtggcccataatgggaaccaggtgacacatcacaagggcaggttctgacacctcttccttccagaagcccaggggtgctggcagctgcttctgaggatctctctcttccttggctcatatttagcaaaatcaaatttaaagaaccccattcctcgctatccaccatccccctattcatgtgccagccactcctattggatcctgttgctttagctaatttttatgaaaataatagtcattcacctgttaggtacttatctaaggtttgtttcaaagcaagtttggtccccttgctgagggtctccagctttttcccagactctgcctctgaccctggattcaacattccctcaggaagcttcggaagagaggaaagcaaattagccacagaagctgtgggggtccgtggccttggttgctgctcctgctgtttttttgaccagcaggtggcatggatagctcccctcccgacatgtcactgcaggagaggagtttatatggatgctaagtggtctgtgcaccttgtcgtcgctaaaaaaggggcttcctccattagcgaattggacgacagatgtatcctacggtctcttgatttccttttttgctttcttgtcatagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa

SEQ ID NO: 5 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 6 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQALEAWVIHGGREDSRDLCQDPTIKELESIISKRNIQFSCKNIYRPDKFLQCVKNPEDSSCTSEI

SEQ ID NO: 7 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLFAWVIHGGREDSRDLCQDPTIKELESIISKRNIQFSCKNIYRPDKFLQCVKNPEDSSCTSEI

SEQ ID NO: 8 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIRFSCKNIYRPDKFLQCVKNPEDSSCTSEI

SEQ ID NO: 9 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFFCKNIYRPDKFLQCVKNPEDSSCTSEI

SEQ ID NO: 10 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCFNIYRPDKFLQCVKNPEDSSCTSEI

SEQ ID NO: 11 - protein, 300 aa, synthetic construct
MANCEFSPVSGDKPCCRLSRRAQLCLGVSILVLILVVVLAVVVPRWRQQWSGPGTTKRFPETVLARCVKYTEIHPEFRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 12 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMFHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 13 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRFVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 14 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHFDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 15 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMFLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 16 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKFGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 17 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLFTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 18 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGFQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 19 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTFTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 20 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQFVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 21 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTFPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 22 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVFCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 23 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQFEKVQTLEAWVIHGGREDSRDLCQDPTIKELESIISKRNIQFSCKNIYRPDKFLQCVKNPEDSSCTSEI

SEQ ID NO: 24 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPFKVQTLEAWVIHGGREDSRDLCQDPTIKELESIISKRNIQFSCKNIYRPDKFLQCVKNPEDSSCTSEI

SEQ ID NO: 25 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEFVQTLEAWVIHGGREDSRDLCQDPTIKELESIISKRNIQFSCKNIYRPDKFLQCVKNPEDSSCTSEI

SEQ ID NO: 26 - DNA, 918 bp, Homo sapiens
  source 1..918: mol_type = genomic DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 27 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 28 - protein, 305 aa, synthetic construct
MWPLVAALLLGSACCGSAFLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 29 - protein, 305 aa, synthetic construct
MWPLVAALLLGSACCGSAQFLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 30 - protein, 305 aa, synthetic construct
MWPLVAALLLGSACCGSAQLFFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 31 - protein, 305 aa, synthetic construct
MWPLVAALLLGSACCGSAQLLFNKTKSVEFTFCNDTVVIPCFVTNMEAQNTFEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 32 - protein, 305 aa, synthetic construct
MWPLVAALLLGSACCGSAQLLFNKTKSVEFTFCNDTVVIPCFVTNMEAQNTTFVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 33 - protein, 305 aa, synthetic construct
MWPLVAALLLGSACCGSAQLLFNKTKSVEFTFCNDTVVIPCFVTNMEAQNTTEFYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 34 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCFVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 35 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEFTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 36 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVFELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 37 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTFLTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 38 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTEFTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 39 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELFREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 40 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTFEGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 41 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTRFGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 42 - DNA, 186 bp, Homo sapiens
  source 1..186: mol_type = genomic DNA
atgaagcgcttcctcttcctcctactcaccatcagcctcctggttatggtacagatacaaactggactctcaggacaaaacgacaccagccaaaccagcagcccctcagcatccagcaacataagcggaggcattttccttttcttcgtggccaatgccataatccacctcttctgcttcagttga

SEQ ID NO: 43 - protein, 61 aa, Homo sapiens
MKRFLFLLLTISLLVMVQIQTGLSGQNDTSQTSSPSASSNISGGIFLFFVANAIIHLFCFS

SEQ ID NO: 44 - protein, 12 aa, Homo sapiens
GQNDTSQTSSPS

SEQ ID NO: 45 - protein, 12 aa, synthetic construct
GQNDTSFTSSPS

SEQ ID NO: 46 - protein, 12 aa, synthetic construct
GQNDTSQFSSPS

SEQ ID NO: 47 - protein, 12 aa, synthetic construct
GQNDTSQTFSPS

SEQ ID NO: 48 - protein, 12 aa, synthetic construct
GQNDTSQTSFPS

SEQ ID NO: 49 - protein, 12 aa, synthetic construct
GQNDTSQTSSFS

SEQ ID NO: 50 - protein, 12 aa, synthetic construct
GQNDTSQTSSPF

SEQ ID NO: 51 - DNA, 23 bp, synthetic construct
  source 1..23: mol_type = other DNA
ttttcctgcaagaatatctacag

SEQ ID NO: 52 - DNA, 23 bp, synthetic construct
  source 1..23: mol_type = other DNA
tgtacttgacgcatcgcgccagg

SEQ ID NO: 53 - protein, 6 aa, Homo sapiens
QTSSPS

(19) United States
(12) Patent Application Publication
(10) Pub. No.: US 2025/0205282 A1
(43) Pub. Date: Jun. 26, 2025

(54) GENETICALLY ENGINEERED ANTIBODY RESISTANT (GEAR) CELLS FOR ADOPTIVE CELLULAR THERAPY

(71) Applicant: VyGen-Bio, Inc., Tampa, FL (US)

(72) Inventors: Arnika WAGNER, Grödinge (SE); Evren ALICI, Huddinge (SE)

(73) Assignee: VyGen-Bio, Inc., Tampa, FL (US)

(21) Appl. No.: 18/852,070

(22) PCT Filed: Mar. 28, 2023
(86) PCT No.: PCT/US2023/016624

§ 371 Date: Sep. 27, 2024

Related U.S. Application Data
(60) Provisional application No. 63/324,172, filed on Mar. 28, 2022.

(51) Int. Cl. A61K 35/17; A61K 40/15; A61K 40/31; A61K 40/42; C07K 16/28; C12N 5/0783

(52) U.S. Cl. CPC A61K 35/17; A61K 40/15; A61K 40/31; A61K 40/4207; A61K 40/4211; A61K 40/4212; A61K 40/4221; A61K 40/4222; C07K 16/2803; C07K 16/2863; C07K 16/2878; C07K 16/2887; C07K 16/2893; C07K 16/2896; C12N 5/0636; A61K 2239/13; A61K 2239/21; C12N 2510/00

(57) ABSTRACT

The method entails modification of non-malignant cells for transplantation or therapy to avoid recognition and attack by monoclonal antibodies and antibody-derived therapeutics. Many therapies that use monoclonal antibodies or antibody-derived therapeutics not only bind to the intended target epitope on malignant cells, but also to the same target epitope on healthy non-malignant cells that express the target antigen. This phenomenon is termed on-target off-tumor effect. This can cause rejection, immune cell attack or opsonization of non-malignant cells, which in turn can cause severe side effects which often hampers the therapeutic effect. Similarly, cytokines in cytokine therapy can bind to receptors on bystander cells and cause unintended effects. The methods of the present invention change the antigen epitope or the cytokine receptor on non-malignant and bystander cells for adoptive cell therapy, thereby disrupting the binding of the therapeutic agent—antibody or cytokine—to the target antigen or receptor on non-malignant cells.

### PRIORITY AND INCORPORATION BY REFERENCE

This application claims priority to U.S. provisional patent application No. 63/324,172 filed on Mar. 28, 2022, the contents of which are incorporated herein by reference. All references cited herein are expressly incorporated by reference.

### FIELD OF THE INVENTION

The present invention relates to mutant proteins that are targets of biologicals, cells expressing such mutant proteins, methods of making such mutant proteins and cells, and therapies (e.g., adoptive therapies, therapies in which target antigens for autoantibodies can cause rejection of an organ (e.g., after transplantation or in an autoimmune disease) involving use of such mutant proteins and cells. The proteins or cells can be made in or ex vivo and the resulting cells used as a therapeutic.

### BACKGROUND OF THE INVENTION

In modern medicine patients are increasingly treated with monoclonal antibodies as a therapeutic treatment. Many therapies that use monoclonal antibodies or antibody-derived therapeutics not only bind to the intended target epitope on malignant cells, but also to the same target epitope on healthy non-malignant cells if they also express the target antigen. This phenomenon is termed on-target off-tumor effect. It can cause rejection, immune cell attack or opsonization of non-malignant cells, which in turn can cause severe side effects, and in many cases, this even hampers the therapeutic effect. Along the same lines, cytokines in cytokine therapy can bind to receptors on bystander cells and cause unintended effects. The current invention relates to methods to change the antigen epitope or the cytokine receptor on non-malignant and bystander cells for adoptive cell therapy, with the goal of disrupting the binding of the therapeutic agent (antibody or cytokine) to the target antigen or receptor on non-malignant cells.

B cell malignancies are blood cancers. They comprise a diverse collection of diseases that arise from the B cell lineage of hematopoietic cells. The treatments for B cell malignancies include radiation and chemotherapy, autologous or allogeneic stem cell transplantation, immunomodulatory drugs, proteasome inhibitors, infusion of antibodies and antibody-derived therapeutics such as CAR T cells, bi- or trispecific engagers, small molecules that activate or inhibit specific parts of the immune system or the cancer cells metabolism. B cell malignancies include multiple myeloma (“MM”), B cell chronic lymphocytic leukemia (“CLL”)/small lymphocytic lymphoma (“SLL”), precursor B-cell lymphoblastic leukemia/lymphoma, B cell ALL, Burkitt Lymphoma, acute promyelocytic leukemia, acute lymphoblastic leukemia, mature B-cell neoplasms, mantle cell lymphoma (MCL), follicular lymphoma, diffuse large B cell lymphoma, Hodgkin's lymphoma, primary effusion lymphoma, AIDS-related Non-Hodgkin's Lymphoma, cutaneous follicle center lymphoma, marginal zone B-cell lymphoma (MALT type, nodal and splenic type), hairy cell leukemia, diffuse large B-cell lymphoma (DLBCL), plasmacytoma, plasma cell leukemia, post-transplant lymphoproliferative disorder, Waldenstrom's macroglobulinemia, plasma cell leukemias, anaplastic large-cell lymphoma (ALCL) and hairy cell leukemia.

Approximate incidence and number of newly diagnosed B cell malignancies per year in the US are shown in Table 1.

TABLE 1
B Cell Malignancies

| | Incidence | Newly diagnosed |
|---|---|---|
| Malignancy | (per 100,000) | per year |
| Multiple Myeloma | 7.1 | 34920 |
| B cell Chronic lymphocytic | 6.75 | 21 250 |
| leukaemia (CLL) | | |
| Small lymphoblastic | 3.65 | |
| leukaemia (SLL) | | |
| Precursor B cell | | 6660 |
| lymphoblastic | | |
| leukaemia/lymphoma | | |
| Burkitt lymphoma | | 1200 |
| Acute premyelocytic | | 600-800 |
| leukemia | | |
| Mantle cells lymphoma | 0.5 | |
| (MCL) | | |
| Follicular lymphoma | 2.7 | 15000 |
| Diffuse large B cells | 5.6 | 18,000 |
| lymphoma (DLBCL) | | |
| Hodgkin's lymphoma | 2.6 | 8540 |
| Cutaneous follicle center | 0.3 | |
| lymphoma | | |
| Plasmacytoma | 0.35 | |
| Waldenstrom | | 1,000-1,5000 |
| macrobulinemia | | |
| Hairy cell leukaemia | | 600-800 |

MM is a monoclonal plasma cell malignancy representing 10% of all hematological cancers. MM is the third most common haematological malignancy with an approximate incidence of 160,000 and a mortality of 106,000 globally. The cause of MM is unknown, but risk factors include obesity and a familial predisposition exists. MM can develop from monoclonal gammopathy of undetermined significance (MGUS) that progresses to smoldering myeloma (SM) and eventually to MM. Some cases of MM progress to the very aggressive plasma cell leukemia (1). The standard treatment for MM is high-dose chemotherapy conditioning with autologous stem cell transplantation (SCT) and immunomodulatory drugs (IMiD), proteasome inhibitors (PIs) as well as monoclonal antibodies (mAbs) (2). Relapses often occur, resulting in a median survival time of six years with a 10-year survival rate of up to 50% (3).

Treatment with monoclonal antibodies specific for an antigen expressed by malignant cells, has been introduced in the MM setting. Numerous new types of monoclonal antibodies and antibody-derived therapeutics (as depicted in FIG. 1) such as bi- or trispecific engagers and CAR T or CAR NK cells are under preclinical investigation. Daratumumab (“Dara”), an anti-CD38 antibody, has been approved for the treatment of patients with relapsed MM and other anti-CD38 antibodies have recently been approved or are currently in development (isatuximab and MOR-202, which is described in U.S. Pat. No. 8,263,746). Dara recognizes CD38 overexpressed on MM cells and other B cell malignancies (4). It induces tumor cell death mainly by binding to the Fc receptor on NK cells, which kill the target cells via antibody-dependent cellular cytotoxicity (ADCC) and by induction of complement-dependent cytotoxicity (4, 5). Recently, Dara was introduced also as front-line treatment in newly diagnosed MM patients.

Besides Dara, there are numerous promising new antibody drugs in testing for MM. Isatuximab is indicted for relapsed or refractory MM (RRMM) in combination with pomalidomide and dexamethasone or carfilzomib and dexamethasone (31, 32). When added to standard MM treatment regimen of pomalidomide and low-dose dexamethasone, isatuximab significantly improved progression-free survival in patients with RRMM (6). Clinical trials to use isatuximab as single-agent treatment are underway (7). Another anti-CD38 monoclonal antibody, Mezagitamab (TAK-079), is being tested currently in a phase Ib clinical trial (33).

Other antibodies or anti-body-derived therapeutics under development for the treatment of MM include anti-BCMA CAR T cells, BCMA bispecific T cell engager (BIKE), anti-SLAMF7/CRACC mAb Elotuzumab, anti-B-cell activating factor (tabalumab) molecules, antibody-drug conjugates targeting B-cell maturation antigen (GSK2857916), alpha emitter coupled to anti-CD38 antibody (Astatine-211), CD138 (Indatuximab ravtansin), anti-GPRRC5D CAR T cells or GPRC5D-bispecific engager. In addition, monoclonal antibodies directed against molecules involved stress sensing such as ligands to the NK cell receptor NKG2D or involved in MM-induced bone destruction, such as RANK-L (denosumab) or DDKK1 (BHQ880) will probably become interesting therapeutics.

Antibodies and antibody-derived therapeutics (as depicted in FIG. 1) represent a new class of drugs that stimulate tumor cell death by intrinsic mechanisms or by stimulating immune-competent cells to kill or phagocytose neoplastic plasma cells. The safety profiles are manageable, and usually relate to the target of the antibody or related to the intravenous injection. These drugs may be combined with other agents without adding significant toxicities and may lead to increase of duration and depth of the response. In fact, many therapies using antibodies or antibody-derived products are run as combination trials together with IMiDs or PIs.

CD38 is expressed by cancer cells in many B cell malignancies. This molecule is an important prognostic factor in many B cell cancers including B cell CLL (8). CD38 is expressed at high levels on malignant plasma cells and it is implicated in various hematological malignancies. It is also expressed on non-malignant blood cells including T cells, B cells and NK cells, and low levels on monocytes and osteoclast progenitors. CD38 is encoded on chromosome 4 in humans. CD38 mediates proliferation of lymphocytes and cytokine secretion and activation (9, 10).

CD38 is an ectoenzyme, that regulates intracellular Ca2+ levels. It catalyzes the synthesis and hydrolysis of cyclic ADP-ribose (cADPR) from NAD+ to ADP-Ribose and NAADP from NADP+. CD38 is also expressed in many malignant hematological diseases, including multiple myeloma, leukemias and lymphomas, such as T- and B-cell acute lymphocytic leukemia, B-cell chronic lymphocytic leukemia, primary systemic amyloidosis, Waldenstrom macroglobulinemia, mantle-cell lymphoma, pro-lymphocytic/myelocytic leukemia, acute myeloid leukemia, chronic myeloid leukemia, follicular lymphoma, Burkitt's lymphoma, large granular lymphocytic (LGL) leukemia, NK-cell leukemia and plasma-cell leukemia.

CD38-knockout (CD38KO) NK cell have been generated by several investigators (11, 12). In culture conditions and immune-compromised mice, these CD38-KO NK cells seem to retain most effector functions. However, several studies highlight the importance of CD38 for NK cell functions in vitro and in vivo (13-18). In fact, CD38 is a triggering molecule for activated NK cells, where antibody against CD38 triggered NK cell-mediated cytotoxicity and CD38-dependent lysis of target cells (15). Furthermore, CD38 can be used by NK cells for conjugation with the target cell, which is known to facilitate target cell lysis (15). In addition, CD38 crosslinking induces Ca2+ mobilization in NK cells (14, 17) and downstream phosphorylation events of key activating pathways of cytotoxic cells (17). In mice, CD38+NK cells are necessary for tumor cell in vivo killing (14) and protection against bacterial (16) and viral (19) infections.

Recently, two clinical phase I trials have been initiated using CD38-KO NK cells for adoptive therapy. The first one uses iPSC-derived CD38-KO BCMA-CAR NK cells in combination with Daratumumab, sponsored by Fate Therapeutics (FT538). The other phase I trial is using CD38-KO mbIL21-expanded NK cells in combination with Isatuximab, sponsored by Kiadis and Sanofi (KDS-1001/SAR445419). However, given the different enzymatic activities of CD38, and the vast functions in critical biological pathways such as proliferation, survival, metabolism, calcium signaling, response to hypoxia, migration and homing, CD38-KO NK cells are expected to show deficiencies in some or most of these pathways in vivo. Homing to the tumor and persistence in the tumor microenvironment which is characterized by hypoxia and low levels of metabolites, are perceived as critical to the success of cellular therapies.

In addition to CD38, targets for antibody therapy contemplated herein include CD19, CD20, CD22, CD25, CD28, CD30, CD33, CD52, CD117, PDGFRA (platelet-derived growth factor receptor alpha), Her2, FFR3, and CEACAM-1.

### SUMMARY OF THE INVENTION

Disclosed herein is a cell comprising an otherwise wild-type protein having at least one mutation in the binding site of a therapeutic agent, the at least one mutation being configured to disrupt specific binding of the therapeutic agent to the protein while retaining the physiological function of the wild-type protein.

Also disclosed herein is such a cell wherein the therapeutic agent is a therapeutic antibody.

Also disclosed herein is such a cell wherein the at least one mutation is induced with gene editing.

Also disclosed herein is such a cell wherein the at least one mutation is at least one amino acid substitution for a naturally occurring amino acid in the binding site.

In an embodiment such a cell may be a primate cell, preferably a human cell, more preferably a primary human cell.

In an embodiment the cell may be selected from an immunocyte (e.g., plasma cell, B cell, macrophage, NK cell, dendritic cell, neutrophil, monocyte, T cell), stem cell (e.g, hematopoietic stem cell, induced pluripotent stem cell), or a somatic cell.

Also disclosed herein is such a cell as above which is allogeneic and/or otherwise configured for adoptive therapy in a subject, preferably a human subject.

Also disclosed herein is such a cell as above wherein the therapeutic agent binding site is present in a protein expressed on the surface of the cell, the protein selected from CD38, SLAMF7, CD19, CD20, CD22, CD25, CD28, CD30, CD33, CD47, CD52, or PDGFRA.9.

In an embodiment the therapeutic agent binding site is present in CD38, the therapeutic agent is an antibody, and the antibody is daratumumab or isatuximab.

In an embodiment the therapeutic agent binding site is present in SLAMF7, the therapeutic agent is an antibody, and the antibody is elotuzumab.

In an embodiment the therapeutic agent binding site is present in CD19, the therapeutic agent is an antibody, and the antibody is blinatumomab.

In an embodiment the therapeutic agent binding site is present in CD19, and the therapeutic agent is a CD19-CAR-T cell or NK cell, such as Abecma, Breyanzi, Kymriah, Tecartus or Yescarta.

In an embodiment the therapeutic agent binding site is present in CD20, the therapeutic agent is an antibody, and the antibody is ibritumomab, tiuxetan, obinutuzumab, ocrelizumab, ofatumumab, or rituximab.

In an embodiment the therapeutic agent binding site is present in CD22, the therapeutic agent is an antibody, and the antibody is inotuzumab.

In an embodiment the therapeutic agent binding site is present in CD30, the therapeutic agent is an antibody, and the antibody is brentuximab.

In an embodiment the therapeutic agent binding site is present in CD33, the therapeutic agent is an antibody, and the antibody is gemtuzumab or ozogamicin.

In an embodiment the therapeutic agent binding site is present in CD52, the therapeutic agent is an antibody, and the antibody is alemtuzumab or ANT1034.

In an embodiment the therapeutic agent binding site is present in CD47, the therapeutic agent is an antibody, and the antibody is Ti-061 or CC-90002 or magrolimab or AK117 or AO-176 or CPO107 JMT601 (CPO107) or DSP107 or Evorpacept (ALX148) or HX009 or IBI188 or 1B1322 or IMC-002 or IMM0306 or OSE-172PF-07257876 or SHR-1603 or SHR2150 or SRF231 or STI-6643 or TG-1801 or TJ011133 or TTI-621 or ZL-1201.

In an embodiment the therapeutic agent binding site is present in PDGFRA, the therapeutic agent is an antibody, and the antibody is olaratumab.

Also disclosed herein is a method of treating a patient comprising administering to the patient: (i) a cell comprising an otherwise wild-type protein having at least one mutation in the binding site of a therapeutic agent, the at least one mutation being configured to disrupt specific binding of the therapeutic agent to the protein while retaining the physiological function of the wild-type protein; and (ii) the therapeutic agent.

Also disclosed herein is such a method in which the at least one mutation comprises at least one amino acid substitution in the binding site.

Also disclosed herein is such a method wherein the cell is selected from an immunocyte (e.g., plasma cell, B cell, macrophage, NK cell, dendritic cell, neutrophil, monocyte, T cell), stem cell (e.g, hematopoietic stem cell, induced pluripotent stem cell), or a somatic cell.

Also disclosed herein is such a method wherein the therapeutic agent binding site is present in a protein expressed on the surface of the cell selected from CD38, SLAMF7, CD19, CD20, CD22, CD25, CD28, CD30, CD33, CD47, CD52, or PDGFRA.

Also disclosed herein is such a method wherein the therapeutic agent binding site is present in CD38, the therapeutic agent is an antibody, and the antibody is Daratumumab or Isatuximab or TAK-079.

Also disclosed herein is such a method wherein the therapeutic agent binding site is present in SLAMF7, the therapeutic agent is an antibody, and the antibody is Elotuzumab.

Also disclosed herein is such a method wherein the therapeutic agent binding site is present in CD19, the therapeutic agent is an antibody, and the antibody is Blinatumomab.

Also disclosed herein is such a method wherein the therapeutic agent binding site is present in CD20, the therapeutic agent is an antibody, and the antibody is ibritumomab tiuxetan, obinutuzumab, ocrelizumab, ofatumumab, rituximab, or rituximab/hyaluronidase.

Also disclosed herein is such a method wherein the therapeutic agent binding site is present in CD22, the therapeutic agent is an antibody, and the antibody is inotuzumab

Also disclosed herein is such a method wherein the therapeutic agent binding site is present in CD30, the therapeutic agent is an antibody, and the antibody is Brentuximab.

Also disclosed herein is such a method wherein the therapeutic agent binding site is present in CD33, the therapeutic agent is an antibody, and the antibody is gemtuzumab ozogamicin.

Also disclosed herein is such a method wherein the therapeutic agent binding site is present in CD52, the therapeutic agent is an antibody, and the antibody is alemtuzumab or ANT1034

Also disclosed herein is such a method wherein the therapeutic agent binding site is present in CD47, the therapeutic agent is an antibody, and the antibody is Ti-061 or CC-90002 or magrolimab or AK117 or AO-176 or CPO107 JMT601 (CPO107) or DSP107 or Evorpacept (ALX148) or HX009 or IBI188 or IBI322 or IMC-002 or IMM0306 or OSE-172PF-07257876 or SHR-1603 or SHR2150 or SRF231 or STI-6643 or TG-1801 or TJ011133 or TTI-621 or ZL-1201.

Also disclosed herein is such a method wherein the therapeutic agent binding site is present in PDGFRA, the therapeutic agent is an antibody, and the antibody is olaratumab.

Also disclosed herein is such a method wherein protein expressed on the surface of the cell is functional for all purposes except therapeutic agent binding.

Also disclosed herein is such a method wherein the CD38 on the surface of the cell is functional for all purposes except daratumumab binding.

Also disclosed herein is such a method wherein the binding site of daratumumab on the cell is modified so that it is no longer recognized by daratumumab.

Also disclosed herein is such a method having at least one amino acid substitution in the daratumumab binding site.

Also disclosed herein is such a method wherein the at least one amino acid substitution is made to amino acids 233-246 or 267-286 of SEQ ID NO 5.

Also disclosed herein is such a method wherein the at least one amino substitution is made to amino acids 237, 239, 272, 274 and/or 276 of SEQ ID NO: 5.

Also disclosed herein is such a method wherein the at least one amino acid substitution is selected from the following: T237A, E239F, Q272R, S274F, and/or K276F.

Also disclosed herein is such a method wherein the CD38 on the surface of the cell comprises SEQ ID NO: 6, 7, 8, 9, or 10.

Also disclosed herein is such a method wherein the CD38 on the surface of the cell is functional for all purposes except isatuximab binding.

Also disclosed herein is such a method wherein the binding site of isatuximab on the cell is modified so that it is no longer recognized by isatuximab.

Also disclosed herein is such a method having at least one amino acid substitution in the isatuximab binding site.

Also disclosed herein is such a method wherein the amino acid substitution is made to one or more of amino acids 77-80, 111-118, or 232-234 of SEQ ID NO 5.

Also disclosed herein is such a method wherein the amino acid substitution is made to amino acids 77, 78, 79, 80, 111, 112, 113, 114, 115, 116,117, 118, 232, 233 and/or 234 of SEQ II) NO: 5.

Also disclosed herein is such a method wherein the amino acid substitution is selected from the following: M77F, R78F, H79F, V80F, K111F, L112F, G113F, T114F, Q115F, T116F, V117F, P118F, P232F, E233F and/or K234F.

Also disclosed herein is such a method wherein the CD38 on the surface of the cell comprises SEQ ID NO: 11, 12, 13, 14, 15, 16, 17, 18, 19, 20, 21, 22, 23, 24, or 25.

Also disclosed herein is such a method wherein the therapeutic agent binding site is present in CD47, the therapeutic agent is an antibody, and the antibody is Magrolimab.

Also disclosed herein is such a method wherein the amino acid substitution is made to a region of CD47 comprising amino acids 1-3, 34-36 and/or 97-104 of SEQ ID NO: 27.

Also disclosed herein is such a method wherein the amino acid substitution is made to one or more of amino acids, 1, 2, 3, 34, 35, 36, 97, 98, 99, 100, 101, 102, 103, and/or 104 of SEQ ID NO 27.

Also disclosed herein is such a method wherein the amino acid substitution is selected from the following: Q1F, L2F, L3F, T34F, E35F, V36F, E97F, V98F, T99F, E100F, L101F, T102F, R103F and/or E104F.

Also disclosed herein is such a method wherein the CD47 on the surface of the cell comprises SEQ ID NO 28, 29, 30, 31, 32, 33, 34, 35, 36, 37, 38, 39, 40, and/or 41.

Also disclosed herein is such a method wherein the therapeutic agent binding site is present in CD52, the therapeutic agent is an antibody, and the antibody is alemtuzumab.

Also disclosed herein is such a method wherein the amino acid substitution is made to a region of CD52 comprising amino acids 31-36 of SEQ ID NO: 43.

Also disclosed herein is such a method wherein the amino acid substitution is made to amino acids 31, 32, 33, 34, 35 and/or 36 of SEQ ID NO: 43.

Also disclosed herein is such a method wherein the amino acid substitution is selected from the following: Q31F, T32F, S33F, S34F, P35F and/or S36F.

Also disclosed herein is such a method wherein the CD52 on the surface of the cell comprises SEQ ID NO: 45, 46, 47, 48, 49 and/or 50.

Herein is also disclosed an adoptive cell therapy method comprising administering the cell as mentioned above to patient in need thereof.

Also disclosed herein is such an adoptive cell therapy wherein the therapeutic agent binding site is present in CD38 expressed on the surface of the cell and the cell is functional for all purposes except daratumumab binding.

Also disclosed herein is such an adoptive cell therapy wherein the therapeutic agent binding site is a binding site of daratumumab and the daratumumab binding site is modified so that it is no longer recognized by daratumumab.

Also disclosed herein is such an adoptive cell therapy wherein the daratumumab binding site comprises at least one amino acid substitution.

Also disclosed herein is such an adoptive cell therapy wherein the at least one amino acid substitution is made within an extracellular region subsequence of amino acids present in SEQ ID NO: 5, preferably within amino acids 233-246 or 267-286 of SEQ ID NO 5.

Also disclosed herein is such an adoptive cell therapy wherein the at least one amino acid substitution is made to amino acids 237, 239, 272, 274 and/or 276 of SEQ ID NO: 5.

Also disclosed herein is such an adoptive cell therapy wherein the amino acid substitution is selected from the following: T237A, E239F, Q272R, S274F, K276F.

Also disclosed herein is such an adoptive cell therapy of wherein the therapeutic agent binding site is present in CD38 expressed on the surface of the cell and the cell is functional for all purposes except isatuximab binding.

Also disclosed herein is such an adoptive cell therapy wherein the therapeutic agent binding site is a binding site of isatuximab and the isatuximab binding site is modified so that it is no longer recognized by isatuximab.

Also disclosed herein is such an adoptive cell therapy wherein the isatuximab binding site comprises at least one amino acid substitution.

Also disclosed herein is such an adoptive cell therapy wherein the at least one amino acid substitution is made within an extracellular region subsequence of amino acids present in SEQ ID NO: 5.

Also disclosed herein is such an adoptive cell therapy wherein the amino acid substitution is made to a region of CD38 comprising amino acids 77-80, 111-118, or 232-234 of SEQ ID NO 5.

Also disclosed herein is such an adoptive cell therapy wherein the amino acid substitution is made to amino acids 77, 78, 79, 80, 111, 112, 113, 114, 115, 116, 117, 118, 232, 233 and/or 234.

Also disclosed herein is such an adoptive cell therapy wherein the amino acid substitution is selected from the following: M77F, R78F, H79F, V80F, K11F, L112F, GI 13F, T114F, Q115F, T116F, V117F. P118F, P232F, E233F and/or K234F.

Also disclosed herein is such an adoptive cell therapy of wherein the CD38 on the surface of the cell comprises SEQ ID NO: 11, 12, 13, 14, 15, 16, 17, 18, 19, 20, 21, 22, 23, 24, or 25.

The present disclosure also includes a cell configured for adoptive therapy, which comprises a cell surface and/or transmembrane protein having at least one mutation in the binding site of a therapeutic antibody, the at least one mutation being configured to disrupt specific binding of the therapeutic antibody to the cell surface and/or transmembrane protein.

In an embodiment the cell may comprise SEQ II) NO: 6, 7, 8, 9, and/or 10.

In another embodiment the cell may comprise SEQ ID NO: 11, 12, 13, 14, 15, 16, 17, 18, 19, 20, 21, 22, 23, 24, and/or 25.

In another embodiment the cell may comprise SEQ ID NO: 28, 29, 30, 31, 32, 33, 34, 35, 36, 37, 38, 39, 40, and/or 41.

In another embodiment the cell may comprise SEQ ID NO: 45, 46, 47, 48, 49, and/or 50.

Also contemplated herein is a cell as above wherein the at least one mutation is introduced by knock-out (KO) of the native protein, with knock-in (KI) of the modified native protein, CRISPR editing of the native protein at the desired nucleotides, and/or editing using TALENs (transcription activator-like effector nucleases) or ZFNs (Zinc Finger Nucleases).

In an embodiment the CRISPR editing may comprise introducing a guide RNA represented by SEQ ID NO 1 and/or SEQ ID NO 2.

In an embodiment the cell may comprise an otherwise wild-type protein having at least one mutation in the binding site of a first therapeutic antibody, the at least one mutation being configured to disrupt specific binding of the therapeutic antibody to the protein while retaining the physiological function of the wild-type protein.

In an embodiment, the cell may be configured to retain binding sites to a second therapeutic antibody.

Also disclosed herein is a therapeutic method comprising administering an antibody to a subject in need thereof, wherein a cell as above has been administered to the subject prior to administering the antibody, and wherein the antibody is a monoclonal antibody, CAR T, a BIKE or a TRIKE.

Also disclosed herein is such a method wherein the method is a treatment for a malignant hematological disease.

Also disclosed herein is such a method wherein the malignant hematological disease is selected from: multiple myeloma, leukemias and lymphomas, such as T- and B-cell acute lymphocytic leukemia, B-cell chronic lymphocytic leukemia, primary systemic amyloidosis, Waldenstrom macroglobulinemia, mantle-cell lymphoma, pro-lymphocytic/myelocytic leukemia, acute myeloid leukemia, chronic myeloid leukemia, follicular lymphoma, Burkitt's lymphoma, large granular lymphocytic (LGL) leukemia, NK-cell leukemia or plasma-cell leukemia.

The present disclosure also includes a therapy cell comprising at least one mutation to more than one therapeutic antibody target site such that the therapeutic antibodies no longer bind to the cell.

In an embodiment the mutation may be induced with gene editing.

In an embodiment the antibody target sites may be selected from sites present in CD38, SLAMF7, CD19, CD20, CD22, CD25, CD28, CD30, CD33, CD47, CD52, and/or PDGFRA.

Also disclosed herein is a cell comprising an exogenous nucleotide sequence which encodes a mutant form of a human cell-surface or transmembrane protein, the mutant form engineered to lack an epitope that allows for specific binding to a therapeutic antibody but otherwise having all the same functional capabilities as the corresponding wild-type cell-surface or transmembrane protein.

Also disclosed herein is such a cell, in which the exogenous nucleotide sequence encodes a mutant form of human CD38, SLAMF7, CD19, CD20, CD22, CD25, CD28, CD30, CD33, CD47, CD52, or PDGFRA, or a variant thereof having at least 80% sequence identity thereto.

Also disclosed herein is a method of producing a cell for adoptive therapy which may comprise: (a) obtaining an allogeneic and/or stem cell comprising a nucleic acid sequence encoding a protein expressed on the surface of the allogenic and/or stem cell, wherein the protein comprises a therapeutic antibody binding site; (b) identifying the amino acid residues of the therapeutic antibody binding site; and (c) introducing one or more mutations into the nucleic acid sequence encoding the protein so that specific binding of a therapeutic antibody to the binding site is disrupted while maintaining all other functions of the expressed protein.

Also disclosed herein is such a method wherein the therapeutic antibody binding site is selected from a site present in CD38, SLAMF7, CD19, CD20, CD22, CD25, CD28, CD30, CD33, CD47, CD52, or PDGFRA.

Also disclosed herein is such a method wherein the therapeutic antibody is daratumumab, and an amino acid substitution is made to amino acids 233-246 or 267-286 of CD38 as represented by SEQ ID NO 5.

Also disclosed herein is such method wherein the amino substitution is made to amino acids 237, 239, 272, 274 and/or 276 of SEQ ID NO: 5.

Also disclosed herein is such a method wherein the amino acid substitution is selected from the following: T237A, E239F, Q272R, S274F, and/or K276F.

Also disclosed herein is such a method wherein the therapeutic antibody is isatuximab, and an amino acid substitution is made to a region of CD38 comprising amino acids 77-80, 111-118, or 232-234 of SEQ ID NO 5.

Also disclosed herein is such a method wherein the amino acid substitution is made to amino acids 77, 78, 79, 80, 111, 112, 113, 114, 115, 116, 117, 118, 232, 233 and/or 234.

Also disclosed herein is such a method wherein the amino acid substitution is selected from the following: M77F, R78F, H79F, V80F, K11 IF, L112F, G113F, Ti 14F, Q115F, T116F, V117F, P118F, P232F, E233F and/or K234F.

One embodiment of the invention described herein comprises a set of methods to genetically change the epitope of target antigens on non-malignant cells in order to avoid recognition by antibodies. These antibodies may be part of a treatment regimen, or may be autoantibodies e.g. from autoimmune diseases. Recognition of the target antigen would lead to the undesired destruction of the non-malignant cells, therefore the genetic modification could lead to persistence and function of the non-malignant cells.

The invention comprises the modification of the natural sequence of a protein to a sequence that is sufficiently changed so that it is no longer recognized by therapeutic or autoantibodies or antibody-derived therapeutics. This modification should lead to decreased side effects on non-malignant cells, an effect also referred to as “on-target-off-tumor” effect. This in turn would lead to an increased concentration of antibody that would be available to the intended target cells, which could in turn lead to either increased desired effect, or the potential to decrease the administered dose. This modification is made without affecting protein function significantly, to ensure functional integrity of the modified cell.

The modification can be introduced by different methods of genetic engineering of cells, e.g., but not limited to, CRISPR editing, knock-out and knock-in strategies, use of TALENs, Zinc Finger Nucleases etc.

In some embodiments the protein modification is introduced into cells outside the body and the cells expanded and administered to the patient. In other embodiments the protein modification is performed in vivo.

While the examples herein are presently focused on modification of the recognition site of anti-CD38 monoclonal antibodies in the context of treating multiple myeloma (as depicted in FIG. 2), one of skill in the art will recognize that the methods disclosed herein can be used to prevent antibody recognition on any cell.

Several of these targets can be combined, to enable dual or sequential targeting of several tumor associated antigens in the same patients, e.g. by mAbs, bispecifics, CAR T or CAR NK cells. This could not only improve existing therapies, but furthermore enable the use of existing therapies in new indications, e.g. the use of CD19-CAR T cells in MM. To date, this is not used, as this therapy eliminates all progenitor cells of the B cell lineage, leading to life-long B cell aplasia. Since this is a serious side effect, CD19 CAR T cell therapy has only been used in those settings where the benefit outweighs the side effects, while in MM, other treatments exist.

### BRIEF DESCRIPTION OF THE DRAWINGS

FIG. 1 is a schematic figure showing the different potential immune therapy approaches that could benefit from this invention. Any type of immunotherapy, where the recognition domain of an antibody is used, could potentially be improved with GEAR modified cells.

FIG. 2 shows the beta-sheets that comprises the binding site for Daratumumab.

FIG. 3 shows Representation of the extracellular domain of CD38 (PDB Accession number: 1YH3) showing the proximity of E239F and S274F.

FIG. 4 shows in silico analysis of CD38 mutations E239F and S274F showing a reduced hydrogen bond and increased hydrophobic contact.

FIG. 5 shows the assays that shall be done to confirm functionality of the cells containing modified CD38.

FIG. 6 shows that S274F and E239F modifications prevent Daratumumab from binding CD38. NK92 CD38KO were transduced with different mutated versions of CD38 and sorted cells were incubated 30 min with 55 μg/ml of Daratumumab in PBS in the fridge, or with HIT2 as positive control. Shown is the percentage of binding cells N=4 represented mean+/−SD, statistics with one-way Anova of Dara samples against CD38KO Dara, ns is non-significant, **** is p<0.0001.

FIG. 7 shows that S274F and E239F modifications prevent Daratumumab from binding CD38. NK92 CD38KO were transduced with different mutated versions of CD38 and sorted cells were incubated 30 min with 55 μg/ml of Daratumumab in PBS in the fridge, or with HIT2 as positive control. Shown is one representative experiment.

FIG. 8 shows that S274F and E239F modifications prevent Daratumumab from binding CD38. NK92 CD38KO were transduced with different mutated versions of CD38 and sorted cells were incubated 30 min with 55 μg/ml of Daratumumab in PBS in the fridge, or with HIT2 as positive control. Shown is the mean fluorescence intensity (MFI) of Daratumumab samples N=3 represented mean+/−SD, statistics with one-way ANOVA of Dara samples against CD38CO Dara, ns is non-significant, *** is p<0,001, **** is p<0,0001. WT is significantly different as CD38 is not overexpressed.

FIG. 9 shows vector design used in generating NK cells with modified CD38. A codon-optimized sequence has been used to avoid degradation from Cas9, and single amino acids mutation have been designed for E239F and S274F, the other mutations tested were created on the same principle.

FIG. 10 shows the workflow for modification of antigens on NK cells. Modification of the target antigen, CD38, with subsequent expansion of cell product, safety analysis, and infusion to patient.

FIG. 11 is a schematic showing the steps of the present invention for a hematopoietic stem cell having a modified CD38.

FIG. 12 is a schematic showing the steps of the present invention for a hematopoietic stem cell having a modified CD19.

FIG. 13 shows that Daratumumab disrupts HIT2 binding on CD38. The modifications S274F and E239F however are not recognized by Daratumumab, hence these do not disrupt binding of HIT2. HIT2 has a different epitope then Daratumumab. Daratumumab and HIT2 were either incubated alone or at the same time with NK92 cells with different types of CD38. Signal intensity is represented as geometric mean (MFI) for incubation alone VS co-incubation. Shown is the MFI of HIT2 N=3+/−SD, T-Test between HIT2 alone and with daratumumab.

FIG. 14 shows that HIT2 does not disrupt Daratumumab binding on CD38. HIT2 has a different epitope then Daratumumab. Detumomab and HIT2 were either incubate alone or at the same time with NK92 cells with different types of CD38. Signal intensity is represented as geometric mean (MFI) for incubation alone VS co-incubation. Shown is the MFI of Daratumumab, N=3 Mean+/−SD T-test between Daratumumab alone and with HIT2.

FIG. 15 shows that Isatuximab disrupts HIT2 binding on CD38. HIT2 has a different epitope then Isatuximab. The modifications S274F and E239F do not affect binding by Isatuximab, hence these modifications do not alter the competition between Isatuximab and HIT2. Signal intensity is represented as geometric mean (MFI) for incubation alone VS co-incubation. MFI of HIT2 N=3+/−SD, unpaired T-Test between HIT2 alone and with Isatuximab.

FIG. 16 shows that HIT2 does not disrupt Isatuximab binding on CD38. HIT2 has a different epitope then Isatuximab. Signal intensity is represented as geometric mean (MFI) for incubation alone VS co-incubation. MFI of Isatuximab, N=3 Mean+/−SD, unpaired T-Test between HIT2 alone and with Isatuximab.

FIG. 17 shows Degranulation of NK92 cells modified with the different CD38 constructs. Degranulation is measured by CD107 and tested against PMA/ionomycin (P/I) stimulation and in co-incubation with K562 target cells. Overexpression of CD38 slightly decreases degranulation, however, there is no difference between codon-optimized (so WT amino acid sequence) and S274F or E239F modifications. 100 000 NK92 were seeded and stimulated with PMA/ionomycin (50 ng/ml and 500 ng/ml respectively) and K562 at a 1:1 ratio. Δ% CD107a indicates the difference in percentage of cells expressing CD107a before activation (medium as unstimulated control) subtracted from the percentage of CD107a of stimulated sample.

FIG. 18 shows IFNgamma release of NK92 cells modified with the different CD38 constructs. IFNgamma release is measured by intracellular staining and tested against PMA/ionomycin (P/I) stimulation and in co-incubation with K562 target cells. Overexpression of CD38 slightly decreases IFNgamma release, however, there is no difference between codon-optimized (so WT amino acid sequence) and S274F or E239F modifications. 100 000 NK92 were seeded and stimulated with PMA/ionomycin (50 ng/ml and 500 ng/ml respectively) and K562 at a 1:1 ratio. Δ% IFNgamma indicates the difference in percentage of cells expressing IFNgamma before activation (medium as unstimulated control) subtracted from the percentage of IFNgamma of stimulated sample.

FIG. 19 shows the % of live K562 during the same assay when exposed to the NK cells with the different CD38 constructs.

FIG. 20 shows Transduced primary NK cells transduced in ex vivo expanded PBMCs degranulate at day 16 post isolation. Transduced NK cells were gated on CD56+/CD3−/GFP+ from PBMC. 100 000 PBMCs were seeded and stimulated with PMA/ionomycin (50 ng/ml and 500 ng/ml respectively) or K562, Δ% CD107a indicate the difference in percentage of cells expressing CD107a before activation (medium as unstimulated control) subtracted from the percentage of CD107a of stimulated sample. Data represented as mean+/−SD of 2 donors. There was not difference between primary NK cells expressing WT CD38 and those transduced with any of the CD38 overexpression constructs.

FIG. 21 shows Transduced primary NK cells transduced in ex vivo expanded PBMCs release IFNgamma at day 16 post isolation. Transduced NK cells were gated on CD56+/CD3−/GFP+ from PBMC. 100 000 PBMCs were seeded and stimulated with PMA/ionomycin (50 ng/ml and 500 ng/ml respectively) or K562, Δ% IFNgamma indicate the difference in percentage of cells expressing CD107a before activation (medium as unstimulated control) subtracted from the percentage of IFNgamma of stimulated sample. Data represented as mean+/−SD of 2 donors. There was not difference between primary NK cells expressing WT CD38 and those transduced with any of the CD38 overexpression constructs.

FIG. 22 shows Transduced primary NK cells isolated from PBMCs prior to transduction degranulate at day 16 post isolation. Transduced NK cells were gated on CD56+/CD3−/GFP+ from PBMC. 100 000 PBMCs were seeded and stimulated with PMA/ionomycin (50 ng/ml and 500 ng/ml respectively) or K562, Δ% CD107a indicate the difference in percentage of cells expressing CD107a before activation (medium as unstimulated control) subtracted from the percentage of CD107a of stimulated sample. Data represented as mean+/−SD of 2 donors. There was not difference between primary NK cells expressing WT CD38 and those transduced with any of the CD38 overexpression constructs.

FIG. 23 shows Transduced primary NK cells isolated from PBMCs prior to transduction release IFNgamma at day 16 post isolation. Transduced NK cells were gated on CD56+/CD3−/GFP+ from PBMC. 100 000 PBMCs were seeded and stimulated with PMA/ionomycin (50 ng/ml and 500 ng/ml respectively) or K562, Δ% IFNgamma indicate the difference in percentage of cells expressing CD107a before activation (medium as unstimulated control) subtracted from the percentage of IFNgamma of stimulated sample. Data represented as mean+/−SD of 2 donors. There was not difference between primary NK cells expressing WT CD38 and those transduced with any of the CD38 overexpression constructs.

FIG. 24 shows that NAD+/NADH ratio in modified NK92 cells is influenced by CD38. NAD+/NADH assay has been performed to measure the absorbance of NADH and NADtotal in 1 million cells per sample. Concentration in pmol/μl has been calculated with the standard curve and [NAD+] has been calculated as follow: [NADtotal]−[NADH]. Data represent the mean+/−SD of [NAD+]/[NADH] ratio in NK92 for 3 technical replicate (2 for CD38-CO=codon optimized).

FIG. 25 shows CD38 expression as mean fluorescence intensity (MFI) on NK cells from 4 healthy donors and NK92 cell line before (medium) and after (K562) stimulation with K562 target cells. CD38 expression increases during stimulation of NK cells and NK92 cell line.

FIG. 26 shows CD38 expression on NK in ex vivo expanded PBMC cultured for 14 days.

FIG. 27 shows CD38 expression on NK cells purified from PBMC at d0 and cultured for 14 days.

### DETAILED DESCRIPTION OF THE INVENTION

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless indicated to the contrary, the numerical parameters set forth in the following specification and attached claims are approximations that may vary depending upon the desired properties sought to be obtained and thus may be modified by the term “about”. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein. Applicant also contemplates ranges derived from data points and express ranges disclosed herein.

The art describes a strategy to improve antibody-mediated immunotherapy, and to generate antibody-resistant cells for adoptive transfer or transplantation. Many clinically relevant antibodies target not only the tumor cells, but also non-malignant cells that express the antigen. This can lead to recognition, attack, and depletion of bystander cells. This depletion can cause severe side-effects and even limit the efficacy of the therapeutic antibody.

An example of this phenomenon is seen in the treatment of multiple myeloma with the antibodies Daratumumab or Elotuzumab. Both antibodies target a protein antigen on malignant MM cells, thereby leading to recognition and destruction of the target cells (malignant MM cell) by the immune system. NK cells play a critical part in the immune recognition, as they express CD16, a receptor for the Fc-region (non-variable region) of antibodies. When NK cells recognize malignant cells via CD16-bound antibody, e.g. Daratumumab binding to CD38 on MM cells, they can kill the malignant cell via antibody-dependent cellular cytotoxicity (ADCC). However, the protein antigen of both antibodies is also expressed on NK cells, and in the case of CD38, the target protein of Daratumumab, it is even upregulated upon NK cell activation. In addition, a recent study shows that only NK cells expressing CD38 can become activated and perform effector functions (13). In addition, ample scientific literature highlights the importance of CD38 for NK cell functions (13-18). Furthermore, these functions of CD38 may have played a role in the previously mentioned clinical trial using CD38-KO NK cells.

Along the same line, many antibodies that are used in therapy against tumors, also recognize the antigen on non-malignant cells, an effect also referred to as “on-target off-tumor effect”. A non-exhaustive list of these antibodies can be found in Table 2 below.

TABLE 2
Therapeutic Antibodies Presently Approved for Treatment of Blood Cancers

| | | Brand | | | Approval | | | |
|---|---|---|---|---|---|---|---|---|
| Target | Antibody | name | Company | Indication | date | Expression | Type | Route |
| CD38 | Daratumu- | Darzalex | Janssen | Multiple | Nov. 16, 2015 | Hemato- | fully human | Intravenous |
| | mab | | Biotech | myeloma | | poietic cells | | |
| | Isatuximab | | | Multiple | | | | |
| | | | | myeloma | | | | |
| SLAMF7 | Elotuzumab | Empliciti | Bristol- | Multiple | Nov. 30, 2015 | NK cells, | Humanized | Intravenous |
| | | | Myers | myeloma | | T cells, B | | |
| | | | Squibb | | | cells, DCs, | | |
| | | | | | | NKT cells, | | |
| | | | | | | monocytes | | |
| CD19 | Blinatumo- | Blincyto | Amgen | Precursor | Dec. 3, 2014 | B cells, | mouse, | Intravenous |
| | mab | | | B-cell acute | | follicular | bispecific | |
| | | | | lymphoblastic | | DCs | (CD19, CD3) | |
| | | | | leukemia | | | | |
| CD20 | ibritumo- | Zevalin | Spectrum | Relapsed or | Feb. 19, 2002 | B cells | murine, | Intravenous |
| | mabtiuxetan | | Pharmaceuticals | refractory | | | radioimmuno- | |
| | | | | low-grade, | | | therapy | |
| | | | | follicular, or | | | | |
| | | | | transformed | | | | |
| | | | | B-cell non- | | | | |
| | | | | Hodgkin's | | | | |
| | | | | lymphoma | | | | |
| | Obinutuzu- | Gazyva | Genentech | Chronic | Nov. 1, 2013 | B cells | Humanized | Intravenous |
| | mab | | | lymphocytic | | | | |
| | | | | leukemia | | | | |
| | Ocrelizumab | Ocrevus | Genentech | Multiple | Mar. 28, 2017 | B cells | Humanized | Intravenous |
| | | | | sclerosis | | | | |
| | Ofatumumab | Arzerra | Glaxo Grp | Chronic | Oct. 26, 2009 | B cells | fully human | Intravenous |
| | | | | lymphocytic | | | | |
| | | | | leukemia | | | | |
| | Rituximab | Rituxan | Genentech | B-cell non- | Nov. 26, 1997 | B cells | Chimeric | Intravenous |
| | | | | Hodgkin's | | | | |
| | | | | lymphoma | | | | |
| | Rituximab | Rituxan | Genentech | Follicular | Jun. 22, 2017 | B cells | chimeric, co- | Subcutaneous |
| | and | Hycela | | lymphoma; | | | formulated | |
| | hyaluron- | | | diffuse | | | | |
| | idase | | | large B-cell | | | | |
| | | | | lymphoma, | | | | |
| | | | | chronic | | | | |
| | | | | lymphocytic | | | | |
| | | | | leukemia | | | | |
| CD22 | inotuzumab | Besponsa | Wyeth | Precursor | Aug. 17, 2017 | B cells | humanized, | Intravenous |
| | ozogamicin | | | B-cell acute | | | antibody | |
| | | | | lymphoblastic | | | -drug | |
| | | | | leukemia | | | conjugate | |
| CD30 | Brentuximab | Adcentris | Seattle | Hodgkin | Sep. 19, 2011 | T cells, | chimeric, | Intravenous |
| | | | Genetics | lymphoma; | | B cells | antibody | |
| | | | | anaplastic | | | -drug | |
| | | | | large-cell | | | conjugate | |
| | | | | lymphoma | | | | |
| CD33 | gemtuzumab | Mylotarg | Wyeth | Acute | Sep. 1, 2017 | All cells | humanized, | Intravenous |
| | ozogamicin | | | myeloid | | of myeloid | antibody | |
| | | | | leukemia | | lineage, | -drug | |
| | | | | | | some | conjugate | |
| | | | | | | lymphocytes | | |
| CD52 | Alemtuzumab | Campath, | Genzyme | B-cell | 5/7/20012014 | Mature | Humanized | Intravenous |
| | | Lemtrada | | chronic | for MS | lymphocytes, | | |
| | | | | lymphocytic | | monocytes, | | |
| | | | | leukemia (CLL), | | DCs, mature | | |
| | | | | cutaneous T-cell | | sperm cells | | |
| | | | | lymphoma, T-cell | | | | |
| | | | | lymphoma, | | | | |
| | | | | multiple | | | | |
| | | | | sclerosis; | | | | |
| | | | | antirejection | | | | |
| | | | | agent in some | | | | |
| | | | | conditioning | | | | |
| | | | | regimens | | | | |
| | | | | for bone marrow | | | | |
| | | | | transplantation, | | | | |
| | | | | kidny | | | | |
| | | | | transplanstation | | | | |
| | | | | and islet cell | | | | |
| | | | | transplantation | | | | |
| PDGFRA | Olaratumab | Lartruvo | Eli Lilly | Soft tissue | Oct. 19, 2016 | Hematopoietic | fully human | Intravenous |
| | | | | sarcoma | | and non- | | |
| | | | | | | hematopoietic | | |
| | | | | | | cells | | |

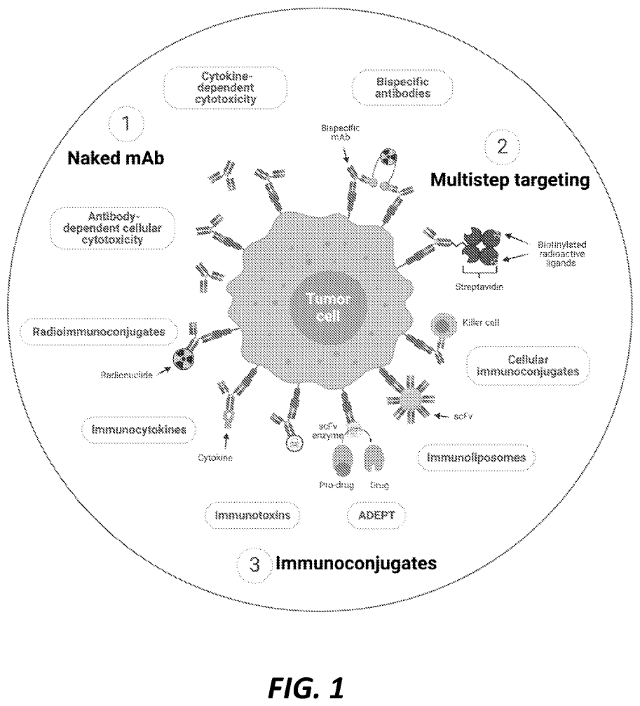

The present invention can be applied not only to therapy with monoclonal antibodies, but also to any and all therapies where the antigen-specificity of mAbs is used (as depicted in FIG. 1). Such therapies include antibody-mediated drug delivery, where toxins, pro-drugs, cytokines or radionuclides are transported to malignant cells via antibodies, bispecific antibodies, CAR-modified cells and many more. Many of these therapies are applied after the patient has undergone multiple prior lines of treatment.

The present invention can also be applied to several proteins in the same cell, to enable dual or sequential use of several therapy approaches, e.g. mAbs, bispecifics, CAR cells. Unless otherwise expressly specific that the features of a, particular embodiment are incompatible with the features of another embodiment, certain embodiments can include a combination of compatible features described herein in connection with one or more embodiments.

Proteins contemplated for mutation and/or expression on the surface of an adoptive therapy cell include but are not limited to CD38, CD19, CD20, CD22, CD25, CD 28, CD30, CD33, CD47, CD52, CD117, PDGFRA (platelet-derived growth factor receptor alpha), Her2, FFR3, and CEACAM-1, as well as homologous variants thereof. Embodiments of CD38 proteins as mentioned above include proteins represented by SEQ ID NOs: 6, 7, 8, 9, 10, 11, 12, 13, 14, 15, 16, 17, 18, 19, 20, 21, 22, 23, 24, or 25, and variants thereof. Embodiments of CD47 proteins as mentioned above include proteins represented by SEQ ID NOs: 28, 29, 30, 31, 32, 33, 34, 35, 36, 37, or 38. Embodiments of CD52 proteins as mentioned above include proteins represented by SEQ ID NOs: 42, 43, 44, 45, 46, or 47.

Such a protein having an amino acid sequence homologous to the amino acid sequence represented by SEQ ID NO: 6, 7, 8, 9, 10, 11, 12, 13, 14, 15, 16, 17, 18, 19, 20, 21, 22, 23, 24, or 25, includes a protein having the same amino acid sequence as the amino acid sequence represented by SEQ ID NO: 6, 7, 8, 9, 10, 11, 12, 13, 14, 15, 16, 17, 18, 19, 20, 21, 22, 23, 24, or 25, except that one or several amino acids are deleted, substituted, inserted, and/or added. In the case of substitution, insertion, or addition, conservative mutations resulting from conservative substitution, insertion, or addition of one or several amino acids are possible. Similarly, such a protein having an amino acid sequence homologous to the amino acid sequence represented by SEQ ID NO: 28, 29, 30, 31, 32, 33, 34, 35, 36, 37, or 38 includes a protein having the same amino acid sequence as the amino acid sequence represented by SEQ ID NO: 28, 29, 30, 31, 32, 33, 34, 35, 36, 37, or 38, except that one or several amino acids are deleted, substituted, inserted, and/or added. Similarly, such a protein having an amino acid sequence homologous to the amino acid sequence represented by SEQ ID NO: 42, 43, 44, 45, 46, or 47 includes a protein having the same amino acid sequence as the amino acid sequence represented by SEQ ID NO: 42, 43, 44, 45, 46, or 47, except that one or several amino acids are deleted, substituted, inserted, and/or added.

“One or several amino acids” herein means 1 to 50, preferably 1 to 20, more preferably 1 to 10, still more preferably 1 to 5 or 1 to 3 or 1 to 2, amino acids.

Moreover, a protein having an amino acid sequence homologous to the amino acid sequence represented by SEQ ID NO: 6, 7, 8, 9, 10, 11, 12, 13, 14, 15, 16, 17, 18, 19, 20, 21, 22, 23, 24, or 25, includes a protein having an amino acid sequence with an identity of not less than 70% to the amino acid sequence represented by SEQ ID NO: 6, 7, 8, 9, 10, 11, 12, 13, 14, 15, 16, 17, 18, 19, 20, 21, 22, 23, 24, or 25, in its full-length form. The protein includes a protein having an amino acid sequence with an identity of preferably not less than 80%, more preferably not less than 90%, and still more preferably not less than 95% to the above-described amino acid sequence in its full-length form. Similarly, such a protein having an amino acid sequence homologous to the amino acid sequence represented by SEQ ID NO: 28, 29, 30, 31, 32, 33, 34, 35, 36, 37, or 38 includes a protein with an identity of not less than 70%, 80%, 90%, or 95% to the amino acid sequence represented by SEQ ID NO: 28, 29, 30, 31, 32, 33, 34, 35, 36, 37, or 38, in its full-length form. Similarly, such a protein having an amino acid sequence homologous to the amino acid sequence represented by SEQ ID NO: 42, 43, 44, 45, 46, or 47, includes a protein with an identity of not less than 70%, 80%, 90%, or 95% to the amino acid sequence represented by SEQ ID NO: 42, 43, 44, 45, 46, or 47, in its full-length form.

“Sequence identity” may refer, in nucleotide sequences or amino acid sequences, the percentage of identical nucleotides or amino acids shared between two sequences, which percentage is determined by aligning those two sequences in an optimal pairwise alignment, optionally by using a conventional or commercially available algorithm.

For any method disclosed herein that includes discrete steps, the steps may be conducted in any feasible order. And, as appropriate, any combination of two or more steps may be conducted simultaneously.

An example for combination of two or more features would be to modify both CD19 and CD38 in HSCs prior to transplant.

One of the most common treatments is bone marrow (“BM”) transplantation or stem cell therapy (“SCT”). Often, patients relapse after SCT. If the editing strategy described herein is applied to the hematopoietic stem cells prior to infusion or implantation, these patients can afterwards be treated with a mAb therapy. An example is the B cell surface marker CD19, which is uniformly expressed by all mature B cells, including most B cell clones in B cell malignancies. If the CD19 gene is already edited in the stem cells, these will generate B cells that are genetically edited to be antibody resistant to subsequent therapy with e.g. CD19-CAR T cells.

The present invention can be used to engineer cells that are resistant to any monoclonal antibody. Such engineered cells can be used to address off-target-effects of therapeutic antibodies that have failed in clinical trials for safety reasons due to the off-target-effects.

Cell types for which this invention is applicable includes: NK cells, T cells, B cells, macrophages, hepatocytes, cardiomyocytes, hematopoietic stem cells, pancreatic cells, MSCs.

Disease conditions include: multiple myeloma (“MM”), B cell chronic lymphocytic leukemia (“CLL”)/small lymphocytic lymphoma (“SLL”), precursor B-cell lymphoblastic leukemia/lymphoma, B cell ALL, Burkitt Lymphoma, acute promyelocytic leukemia, acute lymphoblastic leukemia, mature B-cell neoplasms, mantle cell lymphoma (MCL), follicular lymphoma, diffuse large B cell lymphoma, Hodgkin's lymphoma, primary effusion lymphoma, AIDS-related Non-Hodgkin's Lymphoma, cutaneous follicle center lymphoma, marginal zone B-cell lymphoma (MALT type, nodal and splenic type), hairy cell leukemia, diffuse large B-cell lymphoma (DLBCL), plasmacytoma, plasma cell leukemia, post-transplant lymphoproliferative disorder, Waldenstrom's macroglobulinemia, plasma cell leukemias, anaplastic large-cell lymphoma (ALCL) and hairy cell leukemia.

### Definitions

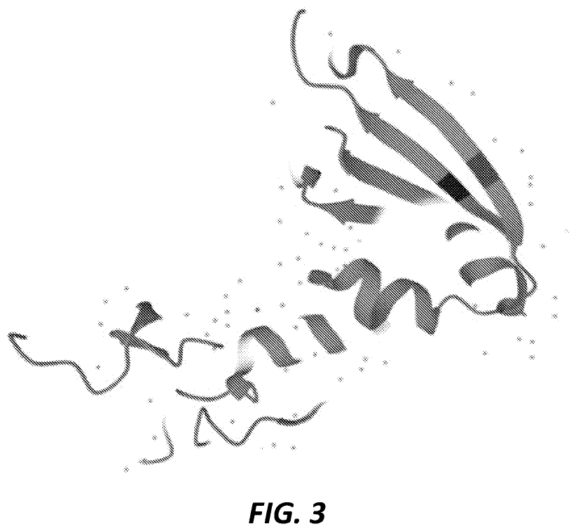

CD38 refers to a CD38 protein, preferably the human CD38 protein (synonyms: ADP-Ribosyl Cyclase 1, ADP-Ribosyl Cyclase/Cyclic ADP-Ribose Hydrolase 1, 2′-Phospho-Cyclic-ADP-Ribose Transferase, 2′-Phospho-ADP-Ribosyl Cyclase, Cyclic ADP-Ribose Hydrolase 1, NAD(+) Nucleosidase, CD38 Antigen (P45), ADPRC 1, 2′-Phospho-ADP-Ribosyl Cyclase/2′-Phospho-Cyclic-ADP-Ribose Transferase, Ecto-Nicotinamide Adenine Dinucleotide Glycohydrolase, cADPr Hydrolase 1, EC 24.99.20, EC 3.2.2.6, T10). The extracellular domain of CD38 is shown in FIG. 3.

SLAMF7 refers to a SLAMF7 protein, preferably the human SLAMF7 protein (synonyms: SLAM Family Member 7, Membrane Protein FOAP-12, CD2 Subset 1, Protein 19A, CRACC, CS1, Novel LY9 (Lymphocyte Antigen 9) Like Protein, CD2-Like Receptor Activating Cytotoxic Cells, CD2-Like Receptor-Activating Cytotoxic Cells, 19A24 Protein, CD319 Antigen, Novel Ly9, CD319, 19A).

CD19 refers to a CD19 protein, preferably the human CD19 protein (synonyms: B-Lymphocyte Surface Antigen B4, T-Cell Surface Antigen Leu-12, Differentiation Antigen CD19, B-Lymphocyte Antigen CD19, CD19 Antigen, CVID3 B4).

CD20 refers to a CD20 protein, preferably the human CD20 protein (synonyms: MS4A1, Membrane Spanning 4-Domains A1, Bp35, FMC7, CD20, B1, Membrane-Spanning 4-Domains, Subfamily A, Member 1, Leukocyte Surface Antigen Leu-16, B-Lymphocyte Antigen CD20, CD20 Antigen, Membrane-Spanning 4-Domains Subfamily A Member 1, B-Lymphocyte Cell-Surface Antigen B1, B-Lymphocyte Surface Antigen B1, CD20 Receptor, LEU-16, CVID5, S7).

CD47 refers to a CD47 protein, preferably the human CD47 protein (synonyms: IAP, Antigenic Surface Determinant Protein OA3, Leukocyte Surface Antigen CD47, MER6, OA3, CD47 Antigen (Rh-Related Antigen, Integrin-Associated Signal Transducer) Antigen Identified By Monoclonal Antibody 1D8, Integrin Associated Protein, Rh-Related Antigen, CD47 Glycoprotein, Integrin-Associated Signal Transducer, Integrin-Associated Protein, Protein MER6, CD47 Antigen).

CD52 refers to a CD52 protein, preferably the human CD52 protein (synonyms: HE5, EDDM5, CDW52, Human Epididymis-Specific Protein 5, CD52 Antigen (CAMPATH-1 Antigen), Epididymal Secretory Protein E5, Cambridge Pathology 1 Antigen, CAMPATH-1 Antigen, Epididymis Secretory Sperm Binding Protein Li 171mP, CDW52 Antigen (CAMPATH-1 Antigen), CD52 Antigen, HEL-S-171mP, CDw52, He5).

CD22 refers to a CD22 protein, preferably the human CD22 protein (synonyms: CD22 Molecule, SIGLEC2, CD22 Antigen, SIGLEC-2, Sialic Acid-Binding Ig-Like Lectin 2, B-Lymphocyte Cell Adhesion Molecule, T-Cell Surface Antigen Leu-14, B-Cell Receptor CD22, BL-CAM, Sialic Acid Binding Ig-Like Lectin 2, Siglec-2).

CD25 refers to a CD25 protein, preferably the human CD25 protein (synonyms: IL2RA, Interleukin 2 Receptor Subunit Alpha, CD25, Interleukin-2 Receptor Subunit Alpha, Interleukin 2 Receptor, Alpha, IL-2 Receptor Subunit Alpha, IL-2R Subunit Alpha, TAC Antigen, IDDM10, IL2R, P55, Insulin-Dependent Diabetes Mellitus 10, CD25 Antigen, IL-2-RA, IL2-RA, IMD41, TCGFR).

CD28 refers to a CD28 protein, preferably the human CD28 protein (synonyms: CD28 Molecule, T-Cell-Specific Surface Glycoprotein CD28, T-Cell-Specific Surface Glycoprotein, CD28 Antigen (Tp44), CD28 Antigen, Tp44, TP44).

CD30 refers to a CD30 protein, preferably the human CD30 protein (synonyms: TNFRSF8, TNF Receptor Superfamily Member 8, D1S166E, CD30, Tumor Necrosis Factor Receptor Superfamily Member 8, Lymphocyte Activation Antigen CD30, CD30L Receptor, Ki-1 Antigen, KI-1, Tumor Necrosis Factor Receptor Superfamily, Member 8, Cytokine Receptor CD30, CD30 Antigen, Ki-1).

CD33 refers to a CD33 protein, preferably the human CD33 protein (synonyms: CD33 Molecule, SIGLEC3, SIGLEC-3, P67, Sialic Acid-Binding Ig-Like Lectin 3, Myeloid Cell Surface Antigen CD33, CD33 Antigen (Gp67), FLJ00391, Gp67, Sialic Acid Binding Ig-Like Lectin 3, CD33 Molecule Transcript, CD33 Antigen, Siglec-3).

CD 117 refers to a CD 117 protein, preferably the human CD 117 protein (synonyms. KIT, KIT Proto-Oncogene, Receptor Tyrosine Kinase, SCFR, V-Kit Hardy-Zuckerman 4 Feline Sarcoma Viral Oncogene Homology, Mast/Stem Cell Growth Factor Receptor Kit, CD117, C-Kit, PBT, Tyrosine-Protein Kinase Kit, Piebald Trait Protein, Proto-Oncogene C-Kit, EC 2.7.10.1, P145 C-Kit, V-Kit Hardy-Zuckerman 4 Feline Sarcoma Viral Oncogene-Like Protein, Proto-Oncogene Tyrosine-Protein Kinase Kit, C-Kit Protooncogene, Piebald Trait, CD117 Antigen, EC 2.7.10, MASTC).

PDGFRA refers to a PDGFRA protein, preferably the human PDGFRA protein (synonyms: Platelet Derived Growth Factor Receptor Alpha, PDGFR2, Platelet-Derived Growth Factor Receptor, Alpha Polypeptide, Alpha-Type Platelet-Derived Growth Factor Receptor, Platelet-Derived Growth Factor Receptor Alpha, Platelet-Derived Growth Factor Receptor 2, CD140 Antigen-Like Family Member A, CD140a Antigen, PDGF-R-Alpha, EC 2.7.10.1, PDGFR-2, CD140a, GAS9, Alpha Platelet-Derived Growth Factor Receptor, Platelet-Derived Growth Factor Alpha Receptor, PDGFR-Alpha, RHEPDGFRA, EC 2.7.10, CD140A).

“Antibody” as used herein is meant in the broad sense and includes immunoglobulin molecules that are produced by plasma cells and recognize a unique antigen, via a fragment-antigen binding (Fab) variable region. Immunoglobulins of all subtypes, with or without the Fc region are included. “Antibody” as used herein includes those of all species, as well as nanobodies and VHH domains.

“Monoclonal antibody” or “monoclonal” as used herein refers to antibody molecules with monovalent affinity in that they bind to the same epitope on an antigen. Monoclonal antibodies are produced by identical immune cells that are clones of a unique parent cell.

“Antibody-derived therapeutics” as used herein refers to antibodies that act by themselves, and those that are coupled to a payload such as antibody-drug conjugates, CAR T or NK cells, bispecific antibodies etc.

“Bispecific antibody” or BIKEs are antibodies designed to recognize two different epitopes or antigens.

“Tri-specific antibody” or TRIKEs are antibodies designed to recognize three different epitopes or antigens.

“Autoantibody” as used herein refers to antibodies that can be formed against the body's own antigens. These autoantibodies can lead to the recognition and destruction of healthy cells by the immune system which leads to autoimmune diseases. Examples of autoimmune diseases that have an autoantibody-component include Type I Diabetes, autoimmune hepatitis or Grave's disease.

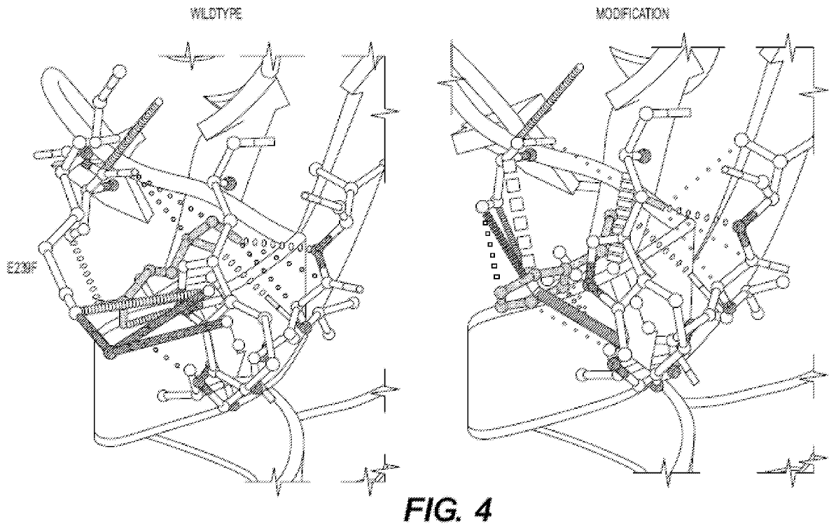

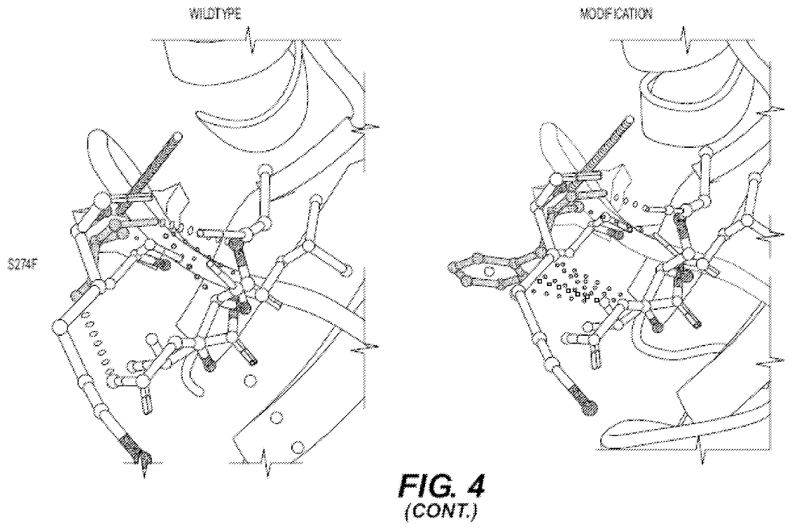

“Epitope” or “antigenic determinant” as used herein means the part of the antigen that is specifically recognized by an antibody. An epitope may consist of contiguous or non-contiguous amino acids that form the three-dimensional structure of the epitope. An epitope may be mutated so as to longer recognize the antibody. The epitope of Daratumumab on CD38, including amino acids involved in binding disruption are disclosed herein. FIG. 4 shows in silico mutation predictions studies using freeware DynaMute.

“Variant” as used herein refers to a nucleotide sequence or polypeptide sequence that differs from a reference nucleotide sequence or polypeptide sequence by one or more modifications for example, substitution, insertion or deletion.

“On-target off-tumor effect” refers to antibodies or antibody-derived therapeutics that not only bind to the intended target epitope on malignant cells, but also to the same target epitope on healthy non-malignant cells if they express the target antigen. On target-off tumor affected cells are often called “bystander” cells.

“CAR-T or CAR-NK cells” are chimeric antigen receptor cells having receptor proteins that have been engineered to give the cells the ability to target a specific protein and have cytotoxic cell activating functions on the receptor.

“Effector cell” is a cell that carries out a function in response to stimulation.

Antibodies and antibody-derived therapeutics used in the methods of the invention disclosed herein, including in the numbered embodiments listed below, may also be selected de novo from publications, clinical studies and in silico analyses.

The present invention is illustrated by the following examples. It is to be understood that the particular examples, materials, amounts, and procedures are to be interpreted broadly in accordance with the scope and spirit of the invention as set forth herein.

### Example 1: Generation of Constructs for Producing Adoptive GEAR Cell

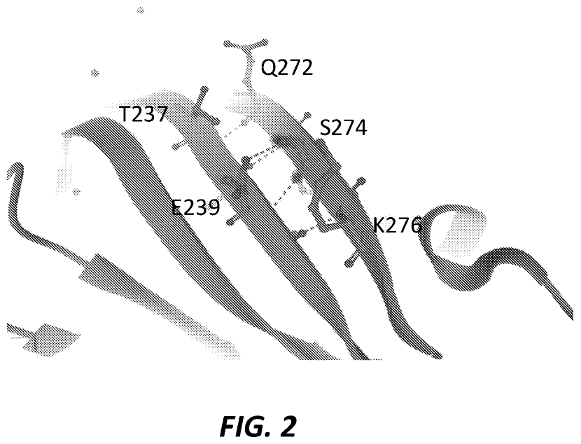

As an example of a cell comprising one or more antibody binding sites which are modified so that they no longer bind the relevant antibody or have a significantly reduced affinity for the antibody, the modified epitope of Daratumumab on CD38 is shown in FIG. 2.

Referring to FIGS. 1-2 and 10-12, cells for adoptive therapy shall be engineered to be resistant to antibody-mediated effects such as antibody-dependent cellular cytotoxicity (ADCC) or antibody-dependent cellular phagocytosis (ADCP). In order to achieve this goal, the cells of the cellular product shall be modified for one or several specific surface proteins. These surface proteins themselves are potential targets for subsequent antibody therapies.

The epitope bound by an antibody on a specific protein of interest is identified and the epitope binding site is engineered with specific amino acid substitutions such that the antibody no longer binds to the engineered binding site. The epitope engineering is performed in a manner which preserves the function of the underlying protein. As an example, the modified epitope of Daratumumab on CD38 is shown in FIG. 2.

The cells of interest can be modified in multiple ways. Genetic modifications can be introduced by different techniques, such as knock-out (KG) of the native protein, with knock-in (KI) of the modified protein, CRISPR (clustered regularly interspaced short palindromic repeats) editing of the native protein at the desired nucleotides, editing using TALENs (transcription activator-like effector nucleases) or ZFNs (Zinc Finger Nucleases).

These nucleases can be delivered by electroporation, viral vector gene transfer, piggy-back or sleeping beauty delivery systems, synthetic or biological nanoparticles, extracellular vesicles or exosomes, and many more technologies.

### Knockout and Insertion

The engineered protein is translated into a cDNA which can be inserted into cells.

The DNA coding sequence for the antibody binding epitope is identified.

The DNA encoding the protein with the engineered epitope binding site is inserted into the cell using different techniques, such as knock-out (KG) of the native protein, with knock-in (KI) of the modified CD38. KI of modified CD38 include single triplet-nucleotide modifications of the human CD38 exon sequence, and codon-optimized (CO) nucleotide sequence encoding for the amino acid sequence of the modified CD38 molecule (ie, single amino acid modification or modification with two or more amino acids).

### CRISPR-Mediated Gene Editing

Genetic editing approaches include CRISPR (clustered regularly interspaced short palindromic repeats) editing of the native protein at the desired nucleotides, editing using TALENs (transcription activator-like effector nucleases) or ZNFs (Zink-finger nucleases).

These nucleases can be delivered by electroporation, viral vector gene transfer, piggy-back or sleeping beauty delivery systems and many more technologies.

Gene editing approaches identify regions of DNA to be modified and through the design of guide RNAs (“gRNA”) that target the Cas9 towards the DNA sequence in the gene that encodes the antibody-binding epitope.

These gRNAs will be tested for targeting efficiency (use them to generate a knockout for the protein of interest in a relevant cell line).

Homology-directed repair (“HDR”) templates are designed for those gRNAs that show Cas9 cutting activity. The HDR templates will perform editing of several amino acids that are in the vicinity of the induced double-strand break in cells. HDR templates can be in the form of short dsDNA or ssDNA molecules, or in form of plasmids, thus providing the option to insert larger edits if necessary.

### Process of Finding and Replacing the Relevant Amino Acid Substitutions

The process can be divided into three separate steps: 1) identification of the relevant amino acid, 2) modification of the nt sequence to generate the modified amino acid sequence, and 3) screening of all resulting modified proteins (one amino acid at a time) for abrogation of binding to the antibody.

The epitope of the antibody in question has to be identified, either by literature research, by checking public databases such as Uniprot and others for information, by crystallization of the antibody with its antigen, or by mutational studies where single amino acids are iteratively substituted and the binding of the antibody is measured.

After epitope identification, the amino acids in that region have to be identified, using public databases such as NCBI, Uniprot or many others. Based on the three-dimensional structure of the antigen in question, amino acids that are accessible to binding, and are therefore likely to be relevant for binding, can be identified (as seen in FIG. 2).

These relevant amino acids can now be replaced by such amino acids that have opposing physico-chemical properties. Often, the native amino acid is replaced by Phenylalanine (F), as the size and structure alone can disrupt specific binding by the therapeutic agent and can confer different physico-chemical contacts (see FIG. 2). However, amino acid substitutions are not limited to replacement by F; substitutions of amino acids include all changes from the WT sequence, meaning replacement by any other amino acid is possible and expected to result in altered or disrupted specific binding by the therapeutic agent.

Relevant amino acids are those where replacement of one single amino acid, or replacement of two or more amino acids in combination, leads to a change in binding/recognition of the antibody.

Substitutions of amino acids include all changes from the wt sequence, that means replacement by any other amino acid.

The amino acid substitution or substitutions is/are done on the basis of nucleotide sequence, where either the simplest sequence change (ie as little nucleotides as necessary), or replacement of the entire codon by another codon can be done. It is recommended to use codons that are most frequently used in humans, ie to codon-optimize the sequence at this stage. This has to be done for every amino acid that shall be screened.

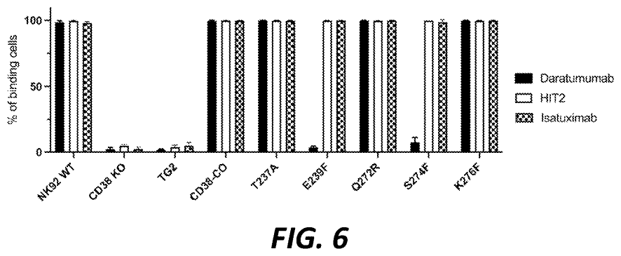

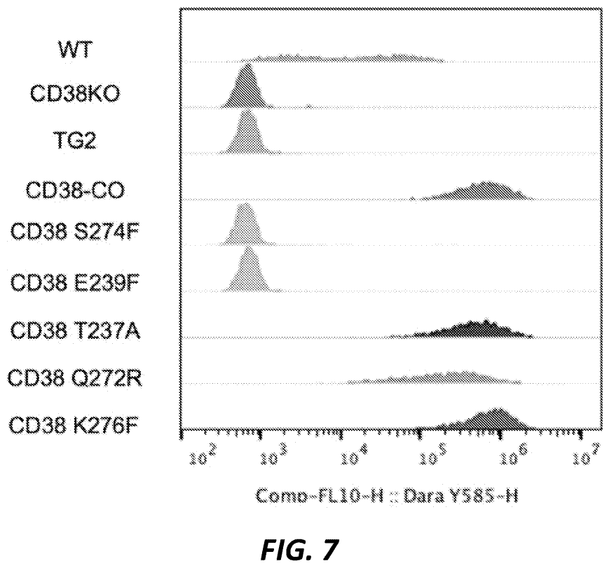

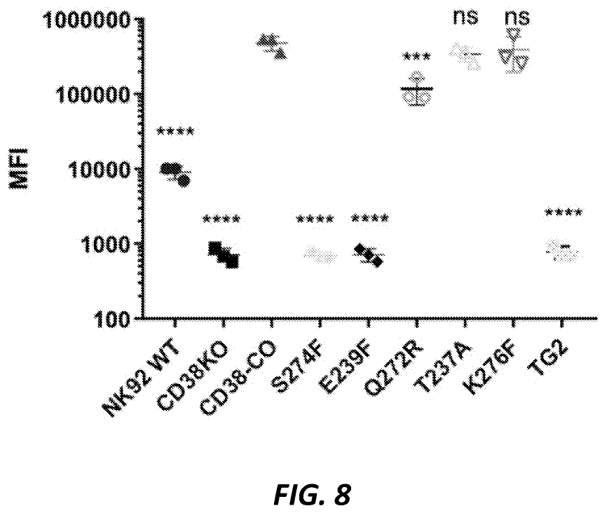

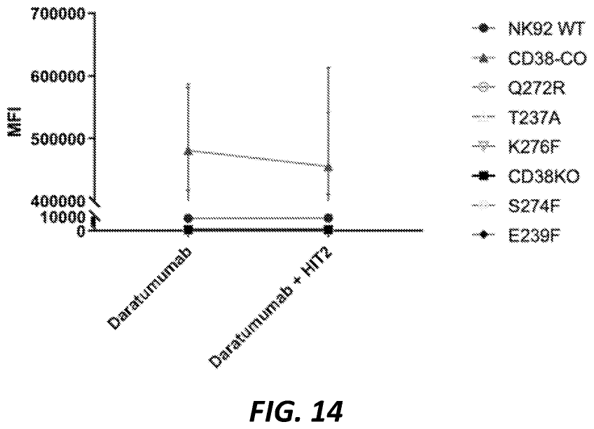

Screening of every version of the resulting protein can be done by binding assays to the antibody. Many different techniques exist to accomplish this. We have expressed all modified molecules on the surface of cells, and have identified substitutions that abrogate antibody binding by performing a staining with the antibody as described further below and shown in FIGS. 6-8 and 13-16.

As antibody epitopes contain a finite number of amino acids, there usually is a finite number of amino acid substitutions that needs to be screened. Amino acids in the vicinity of the epitope that are important for the three-dimensional structure can also be taken into account, but are given a lower priority in our algorithm due to a lower likelihood to achieve both an abrogation of antibody binding but remaining overall structure and functionality of the protein. In addition to screening replacement of any single amino acid, substitution of combinations of two or more amino acids can lead to a change in antibody binding and should be taken into account.

Substitutions can be screened in freewares such as DynaMute, which predict three-dimensional structure (as seen in FIG. 4), but these softwares still need to be improved substantially to be sued as replacement of a functional screening.

### Testing of Modified Cells

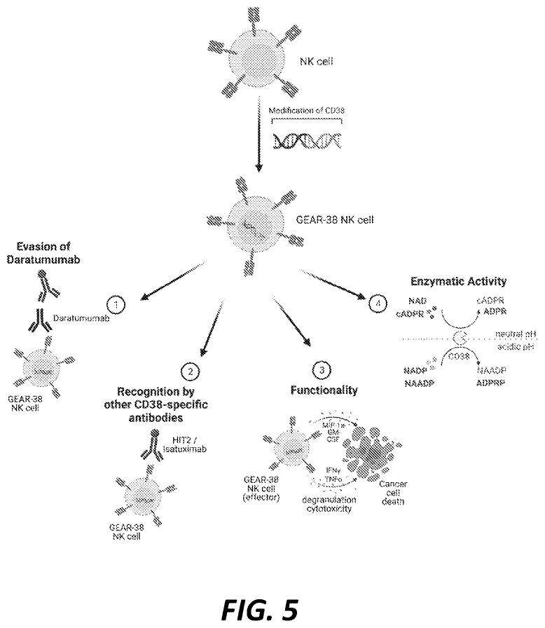

The resulting cells are tested with functional assays as shown in FIG. 5, ie recognition by the antibody it shall be shielded from, recognition of other antibodies specific for the same molecule, functionality such as killing/degranulation, cytokine production, potentially proliferation and exhaustion, and in ceases of enzymes, enzymatic activity, as are relevant for the particular clinical application.

Cells modified via either knockout/knockin strategies or gene editing strategies are then expanded as needed for treatment. Appropriate quality controls are in place to ensure sterility, phenotype and overall safety of the cells (mycoplasma, endotoxin etc.).

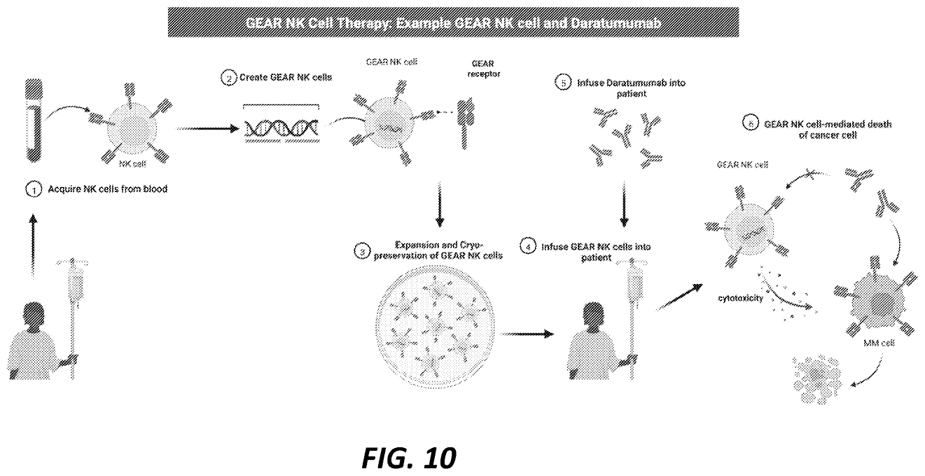

The expanded cells after appropriate quality control are administered to a patient in need thereof along with the corresponding therapeutic antibody as outlined in FIGS. 10-12.

The cells may be administered before, simultaneously with or after the administration of the antibody. The cells may be administered once or multiple times. Cells may be administered with each antibody administration, less frequent than antibody administration or more frequent than antibody administration.

Patients can be tested during therapy to determine the presence of engineered cells and dosing adjusted based on test results.

### Example 2: CD38-GEAR NK Cells

Our present efforts are focused on NK cell adoptive transfer and subsequent or concurrent anti-CD38 treatment. For the treatment of plasma cell malignancies, such as multiple myeloma and other diseases, antibody therapies have become critical. These include monoclonal antibodies, antibody-drug conjugates, bi- or tri-specific antibodies, or CAR cells, where the antibody-recognition domain is genetically introduced into T cells or NK cells. Often, these antibodies target not only the antigen on malignant cells, but also on healthy bystander cells, a process termed on-target-off-tumor effects. In the case of anti-CD38 treatment, such as daratumumab or isatuximab, the malignant cells expressing high levels of CD38 are targeted and eliminated, but also the cells expressing intermediate levels of CD38 are depleted. This has been shown for NK cells (21, 22). This is unfortunate, as NK cells are also crucial mediators of the ADCC effects of anti-CD38 antibodies. Furthermore, CD38+NK cells activated by daratumumab release IFNgamma which is crucial for increasing phagocytosis of CD38+MM cells by monocytes and for induction of a Th1-mediated immune response against MM cells (13) The treatment with daratumumab is beneficial, despite the rapid depletion of the NK effector cells. However, a prolonged presence of functional NK cells would increase the effects, and potentially even lower the required doses of daratumumab.

NK cells can be given as cellular product in adoptive cellular therapy, where they exhibit an antitumor effect (23-25). However, these cells still express CD38, and hence are susceptible to anti-CD38 targeted treatment. This makes combination treatment of daratumumab and NK cell adoptive cell therapy complicated or may even prevent this particular treatment combination.

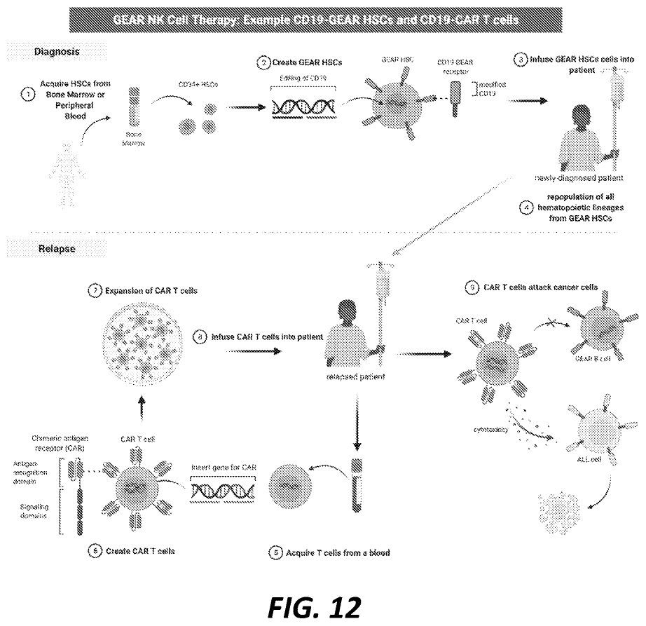

Following the process of Example 1, and referring to FIG. 12, we can improve the feasibility of treating MM with NK cells and daratumumab. We can genetically engineer the NK cell product to be resistant to daratumumab-mediated depletion. This will be achieved by changing one or several amino acids in the daratumumab-binding site of CD38 (as shown in FIG. 2), so that the antibody cannot or only weakly recognizes CD38 on the NK cells (as shown in FIGS. 6-8, and FIGS. 13-14).

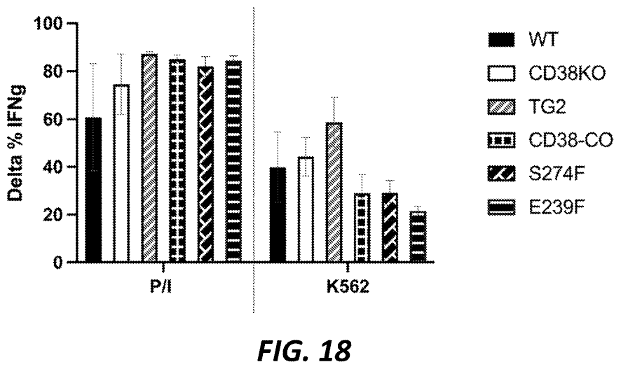

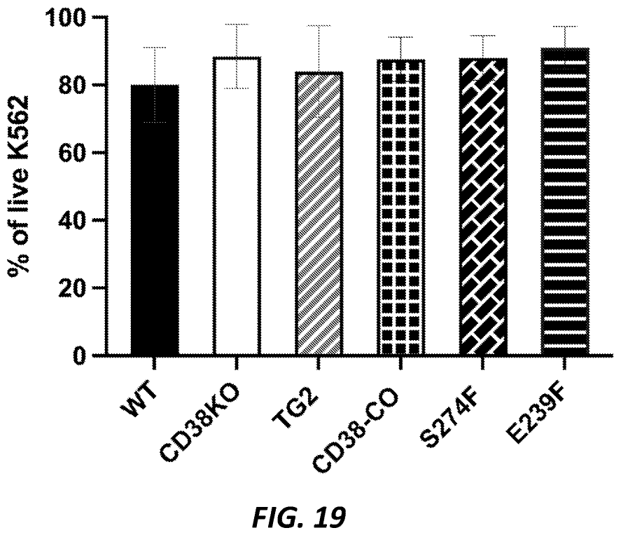

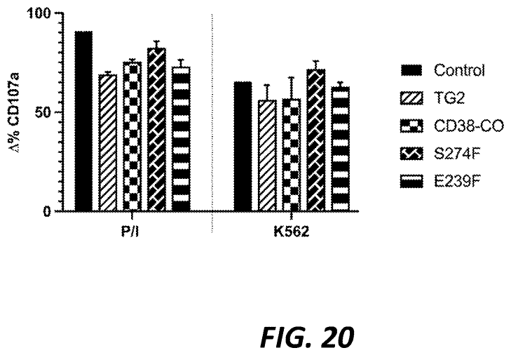

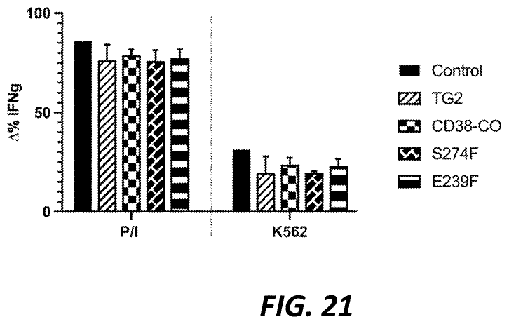

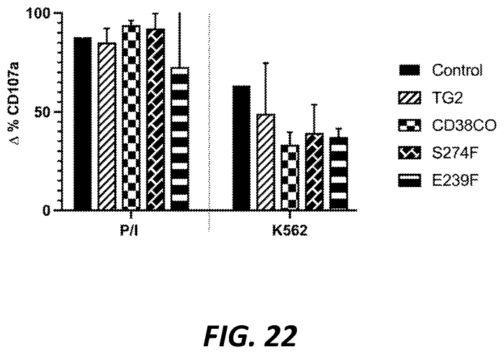

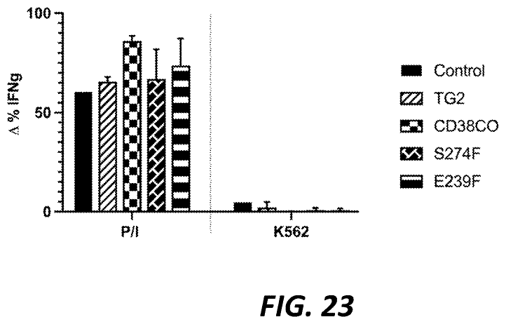

FIG. 2 is a drawing of CD38 showing the Dara binding site. This Figure is based on the structure available from the pdb database, based on the publication by Liu et al. and (20) https://www.rcsb.org/structure/1YH3). FIG. 2 shows proposed and tested substitutions in the Dara binding site to inhibit binding. All these substitutions have been tested and results are shown in FIGS. 6-8 and 13-24.

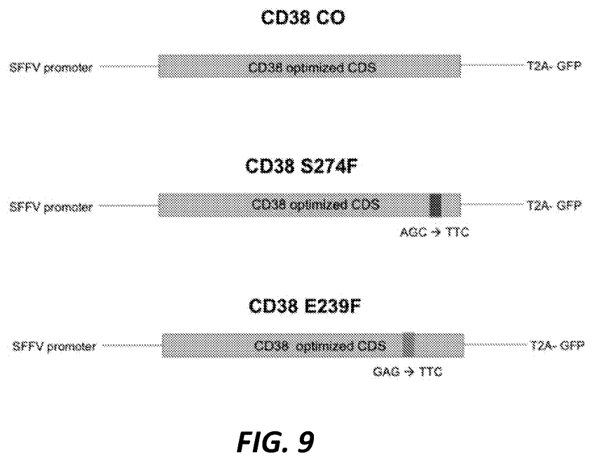

Genetic modifications can be introduced by different techniques, such as knock-out (KO) of the native CD38 (with gRNAs TGGAATCGATTATAAGCAAAAGG (SEQ ID NO: 1), GGAATATTCAATTTTCCTGCAAG (SEQ ID NO: 2), or TTITCCTGCAAGAATATCTACAG (SEQ ID NO: 51)), with knock-in (KI) of the modified CD38 (e.g. with plasmids depicted in FIG. 9), CRISPR (clustered regularly interspaced short palindromic repeats) editing of the native CD38 at the desired nucleotides (using similar gRNAs targeting CD38 in addition to homology-directed-repair templates encoding the amino acid substitutions shown in FIG. 2), editing using TALENs (transcription activator-like effector nucleases) or ZFNs (Zinc Finger Nucleases). These nucleases can be delivered by electroporation, viral vector gene transfer, piggy-back or sleeping beauty delivery systems, synthetic or biological nanoparticles, extracellular vesicles or exosomes, and many more technologies.

The sequence of editing steps can vary, and by codon-optimizing (CO) the nucleotide sequence of the modified CD38 it can be assured that the newly introduced gene will not be targeted by knock-out or editing strategies.

NK cells can be harvested from peripheral blood of healthy donors or patients (as depicted in FIG. 10) These cells can then be expanded from PBMC in an in vitro culture system under GMP conditions. Several different expansion protocols exist and are used in current clinical trials of adoptive NK cell therapy (26, 27), cytokine cocktail to induce memory-like NK cells (28), expansion from iPSC (29), expansion from PBMC (25, 30). Automated, fully-enclosed blood culture systems such as products form Miltenyi, GE and others are available and heavily used for expansion of NK cells in GMP facilities.

After expansion (15-25 days), the cells are quality controlled which entails a sterility assessment and a functionality assessment.

Sterility tests are performed throughout the expansion and manufacturing process and a sample representative of the final cell product is tested for sterility and microbiological contamination. The tests include Mycoplasma, Staphylococcus aureus, Pseudomonas aeruginosa, Bacillus subtilis, Aspergillus brasiliensis, Candida albicans and Clostridium sporogenes. Additional testing for isolates representative of the manufacturing environment can be additionally implemented.

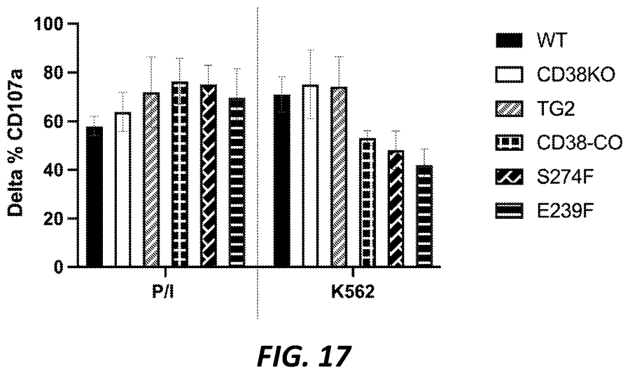

To test the functionality, the NK cells are stimulated in a standard 4 h in vitro stimulation assay using K562 erythroid leukemia cells to stimulate NK cells. Degranulation as surrogate for killing, and IFNgamma production as surrogate for cytokine secretion are assessed using Flow Cytometry (31, 32). This assay has been used to generate data shown in FIGS. 17-24. These figures confirm that modified NK cells harbouring the modified CD38 molecule are functional in terms of degranulation, an accepted marker for killing, and IFNgamma release, the most commonly tested cytokine of NK cells. Other functionality assays include testing of direct killing of target cells using a standard 4 h ⁵¹Chromium release assay, or flow cytometry-based or microscopy-based killing assays.

The cell product is frozen and cryo-preserved until needed as depicted in FIG. 10.

Genetic modification can be performed at any time during the expansion culture. At early timepoints during the culture, the frequency of NK cells among PBMC is low, leading to genetic alterations primarily of non-NK cells in the culture. At later timepoints, the number of cells is quite high, as huge amounts of cells are needed for adoptive cell therapy (0.5-50*10e6 cells/kg bodyweight), which leads to big amounts of reagents needed for genetic modifications. In our hands, introducing the genetic modifications between day 3 and day 14 has shown the best results. After introduction of the genetic modification, either editing of the native CD38 or KO of the native CD38 and KI of the modified CD38, the cells can be further expanded until the desired numbers and the release criteria are reached (as depicted in FIG. 10).

The modifications of the CD38 antigen comprise changes of one or several amino acids that would change the recognition of and the binding to an anti-CD38 antibody, see FIG. 2. In this instance we are focused on daratumumab. These are predicted to be in the daratumumab epitope, ie. amino acids 233-246, 267-286, but could potentially also be found in parts of the sequence that is in 3D proximity to the binding site. The substitutions would be from the native amino acids to those that have different physico-chemical properties, e.g. different electric charge or structure, which both could abolish/compromise binding to daratumumab. Referring to FIG. 2, examples of such substitutions are: T237A, E239F, Q272R, S274F, K276F. Additional substitutions that change the three-dimensional structure of the epitope, alone or in combination, could lead to similar results.

Once these substitutions have been introduced into the cells, an assessment of binding to Daratumumab and other CD38 antibodies shows that two of these modifications, S274F and E239F, lead to abrogation of Daratumumab recognition (FIGS. 6-8 and 13-14). In addition, functionality has been assessed by one or more of the described assays for NK cell activation or target cell killing. Cells will be expanded under standard expansion conditions, and quality and release criteria assessed as for unmodified cell product.

The CD38GEAR NK cell product can then be administered to the patient, using the same procedures and follow-up criteria as for any unmodified NK cell product.

### Procedures

The binding site for daratumumab including proposed amino acid substitutions is shown in FIG. 2. We have identified substitutions that eliminate or reduce binding in the daratumumab epitope, within the amino acid sequence 233-246, 267-286. As shown in FIG. 2, examples of such substitutions are: T237A, E239F, Q272R, S274F, K276F. Additional substitutions, alone or in combination, could lead to similar results. Modifications S274F and E239F have completely abrogated recognition by Daratumumab, shown in FIGS. 6-8 and 13-14, thus shielding the cells from Daratumumab-dependent ADCC-mediated killing of the cells that bear this modified molecule. While these modifications shield from Daratumumab recognition, they do not lead to a KO of CD38, as other CD38-specific antibodies, such as clone HIT2, are able to bind and detect the molecules on the surface of the cells, shown in FIGS. 4-6 and 13-14.

In an embodiment, the CD38 amino acid sequence may be an amino acid sequence which has at least 75%, 80%, 85%, 90%, 91%, 92%, 93%, 94%, 95%, 96%, 97%, 98%, or 99% identity to the CD38 amino acid sequence transcribed and translated from the somatic cell genome, and which is capable of functioning as wild-type CD38 as described herein. In an embodiment, an exemplary CD38 nucleotide sequence may be a nucleotide sequence which has at least 75%, 80%, 85%, 90%, 91%, 92%, 93%, 94%, 95%, 96%, 97%, 98%, or 99% identity to the CD38 nucleotide sequence of a somatic cell and which is capable of being transcribed and translated into a CD38 protein as described herein.

| | gRNAs: |
|---|---|
| (SEQ ID NO: 1) | |
| | TGGAATCGATTATAAGCAAAAGG, |
| (SEQ ID NO: 2) | |
| | GGAATATTCAATTTTCCTGCAAG, |
| | or |
| (SEQ ID NO: 51) | |
| | TTTTCCTGCAAGAATATCTACAG |

Human CD38 has the nucleotide (cDNA) sequence SEQ ID NO: 3 Nucleotides that encode the Daratumumab epitope are in bold. Nucleotides that encode the Isatuximab epitope are underlined.

Nucleotide Sequence (903 nt):

(SEQ ID NO: 3)
ATGGCCAACTGCGAGTTCAGCCCGGTGTCCGGGGACAAACCCTGCTGCCG
GCTCTCTAGGAGAGCCCAACTCTGTCTTGGCGTCAGTATCCTGGTCCTGA
TCCTCGTCGTGGTGCTCGCGGTGGTCGTCCCGAGGTGGCGCCAGCAGTGG
AGCGGTCCGGGCACCACCAAGCGCTTTCCCGAGACCGTCCTGGCGCGATG
CGTCAAGTACACTGAAATTCATCCTGAGATGAGACATGTAGACTGCCAAA
GTGTATGGGATGCTTTCAAGGGTGCATTTATTTCAAAACATCCTTGCAAC
ATTACTGAAGAAGACTATCAGCCACTAATGAAGTTGGGAACTCAGACCGT
ACCTTGCAACAAGATTCTTCTTTGGAGCAGAATAAAAGATCTGGCCCATC
AGTTCACACAGGTCCAGCGGGACATGTTCACCCTGGAGGACACGCTGCTA
GGCTACCTTGCTGATGACCTCACATGGTGTGGTGAATTCAACACTTCCAA
AATAAACTATCAATCTTGCCCAGACTGGAGAAAGGACTGCAGCAACAACC
CTGTTTCAGTATTCTGGAAAACGGTTTCCCGCAGGTTTGCAGAAGCTGCC
TGTGATGTGGTCCATGTGATGCTCAATGGATCCCGCAGTAAAATCTTTGA
CAAAAACAGCACTTTTGGGAGTGTGGAAGTCCATAATTTGCAACCAGAGA
AGGTTCAGACACTAGAGGCCTGGGTGATACATGGTGGAAGAGAAGATTCC
AGAGACTTATGCCAGGATCCCACCATAAAAGAGCTGGAATCGATTATAAG
CAAAAGGAATATTCAATTTTCCTGCAAGAATATCTACAGACCTGACAAGT
TTCTTCAGTGTGTGAAAAATCCTGAGGATTCATCTTGCACATCTGAGATC
TGA

Below is the human genomic CD38 nt sequence (SEQ ID NO: 4). Exons are bold. The nucleotide sequence that encodes the amino acids comprising the Daratumumab epitope are bold and underlined.

CD38 sequence NCBI 20220206>NC_000004.12:15778328-15853232 Homo sapiens chromosome 4, GRCh38.p13 Primary Assembly

(SEQ ID NO: 4)
GCAGTTTCAGAACCCAGCCAGCCTCTCTCTTGCTGCCTAGCCTCCTGCCGGCCTCATCTTCGCCCAGCCAAC
CCCGCCTGGAGCCCTATGGCCAACTGCGAGTTCAGCCCGGTGTCCGGGGACAAACCCTGCTGCCGGCTCTCT
AGGAGAGCCCAACTCTGTCTTGGCGTCAGTATCCTGGTCCTGATCCTCGTCGTGGTGCTCGCGGTGGTCGTC
CCGAGGTGGCGCCAGCAGTGGAGCGGTCCGGGCACCACCAAGCGCTTTCCCGAGACCGTCCTGGCGCGATGC
GTCAAGTACACTGAAATTCATCCTGAGATGAGGTGGGTTGGCGACTAAGGCGCACCGGTGGGCACTGCGGGG
ACAGCAGGGCCCCGCGCGCAGGGAAGCCGCCCGGATCGCCCGGAACCGGGCATCTTCCGTGGCGGGTCAGCC
GAGAGCCCGCCGGGTGGTGCTGAGTAGGGAGTCCCGGGCTCGGGGCTCCGCGGGCCGCTTTCAGGAGCAGCT
GGCCTTGGCACCGAGCGTGCCCGCGGGAGGCGGGGGGGGGCGCTGCTCGGTGGCTCTGCTGCGTAGCCGGTG
AACACTTGGCACCGATGCCCGCCTTCTGGGCAAGGTGCCCTGAGCCCAGCCCCTCGCCGGGCTGCAGCCCAC
CCTCGGCGCGCTCAGCCCGCTTCACCGCTTCAGGGACGGAATAGAACTCGCAGATGCAGGGTGTCGCTGACA
TTTTCAACTTTTTCTGCGGTTTCCGCCCGCTGTCTCTGACCCGAAAGTGCCCCCGGACGGTTACAGAGGACA
CTTAAGTGGTTTGCAAAGCCTGTGGTAGGGGAGGAGGGTGTAGAAGGGCCAAACCACGGAACTTAGTTTTAT
TCATTTATATAAAGCAGCACTCCGATTCTTTTTGCGCGGCCTGAAATGCATGTGACCAGAGAAGTAATTAAC
AAAACAATGTCAACTTCTAAAACCGAGACATTACTTAGATGATAAGGCGCAGCAACTCGGTGAATCTGTACA
AACCTTGGAAAAAAAACACATTAGTCTATGGGACCTTCCAGTTTTCTCATGCTCCTTTCCAGCTACTAACCT
CTCCTAAAGGGAACAACCACTTTTTGGATTTGATTCCCAGGCCTCGCTTTCACCGGGAAATTATCGTTGCTT
GTAAAACAGAAGAAGCCGGGAAGGCAGGCAGGGGGAGCTGCTACTTTACACTCTGTGCTTTGGGATAGCAAA
ATCCCGCATTTAAGCAATCCGAGGAAACGAGCAAATAGACCTCCCTCGCCTCTCCGAGCACACTCAACAGTT
CCGGTTGCAAAATGTTTGCCTCCTGGGCTTCCCAGCGTCCCGTTAGTTGTTCTATTTACACATAATTAGATA
CTTAATGGAGAGAGAAACTAGAAGTTGAGGCGTTCCTCCAGGCTGTATTGTAAAGTATGAAGTGAAATCCAA
AATGAAATGGTAATGTTAGAAAGCAACCTCATTAAAAAAAAAAAAAGTAACACTGGTCTTGAAGATCTTTCA
ATGTGAGTACATAAAGATCTATCTCATTTCTTTTGACAGCCCATAGTATTTCATAAACTAGATGTAACCATT
TCCTATTGACAGGAAATTAGCTTGTTTCCAATTTTTCAATCCCATTCATTCATCCAACAAGTATCTGTTGAG
CACCCACTATGTTCCAGACAGTGATCTAGCTACTGATGACACAAGAGTGAATGACGAAGTTCTCACTCATGA
AATTTTCACCTTAGTTGGGAGAAACATGATGCAATGAAAATCTTCATACATACATTGTGTGTACATATGGGA
GTATTTCTGTAGGATAGATTTCTAGTGATGAAACTGCTGAGTAAAAGGGAGAATTATGCATATTTTAAGTTT
TGATTTTTCCAAATTCCAGGTATTCCATATATACTCCAAAATAGTTGTGCCATTTTACTCTCCCATCATCAG
TCTATCAGAGGGGATGCTTTCCCACAATCTCTTGAATGCTGAATATTTTCAACTTTTTTACTTAAGAGAAAA
AAGAGCATCTAATTGTTCCCTCAGTATCAGTGAGTCTAAGCATCTTGTATATGTTTATTTGCCATTTATATT
TTTTTCTGTGATTTTCCTGTCCAGATACTTGATACTTTCTATTGAGCTGCTTATTTATTTCTTATGGGAGAT
TTTTATATATTTTAGATAATATTCTCTCTCTCACACACACACACACACACACACACACACACACACACACAC
ACACACACATACAGTCTTACAGCCACATCCCTGAAATCTTGACCTTGTGAACATGTTTTACTGGCAGCACTC
TGGACTCGATCATTGCCTTGAGACTATTTCTTTTTTGATATTCTTTGGAAAGACTAACAATGACAGTTTTAT
TTTCAAACCCAACAAATCCTGGCATGGAAATGTTTGCTCTTGATTCTGCTTTTAAAAAAATAAAGAATTATT
TTCTCTCTTTCTTTCTGCACCTTATCAGAAACAGCTAAAAGAAGTGAGTTGGGCCAGGCACTGTGGCTCACA
CCTGTAATCCCAGCACTTTGGGAGGCCCAGGCAGGTGGATCACTTAAGGTCAGGAGTACAAGACCTGCCTGG
CCAACATGCTGAAACTCCGTCTCTACTAAAAATACAAAATTAGCCGGGTGTTGTGGCGCGTGCCTGTAATCC
CAGCTACTCTGGAGACTGAGGTGGGAGAATCGCTTGAACCCAGGAGGAGGAGGTAGCACTGAACCAAGATCC
AGCCTGGCCAAGAGAGTAAGACTCCGTCTCAAAACCAAACCAAACCAAACCAAAAAAAGAAGTGAGTTGGCA
CTTTCAACATTCTGCCTGGAAATCTCCTTACCAAACCTATAAGATCATTAGGTATATTTTCTGCACTTTGTA
TTGTGACAGGTGACAGTGTTACCAAACTTTTTACCAGGACATAATAGGGTCTGCCTTTCTTCTAGTTGCTAA
CAATTTCCCCCAAGTCCATCTTGCCTGCACTAACAGTCTCCTTTAGACCTCTCCTCTCCTGCCTGTCACACA
TTCCTAGTACTAATGCTACAGTATAGTAGTAAGGGTCTCCAGAGAAACAACATTTATATAACATAATATAAA
TACATTAATAGAGAGAAAGAGATTTTAAGAAATTGGCTTATATCATTGTGAAGTCGGGCAAGCCCCAAATCT
GCTGGACAGGCCAGCAGCCTGGAGACCCAGGGAAGAGTTGATGTTGCAGCTGGAGTCCAAAGGCAGTCTCTG
GCAGAATTCTCTTTTACTTCTGGGACCTTGGTCTTTCTCTTAAGGCCTTCAACTGATTGGATGAGGCCCACC
ACATTATGAAGGGTAACATGCTTTACACCGAGTCTCCTGACTTAAAATCTAAAAAATACCTTCACATCACAA
CTAGATGTGTTTGACCAAATATCTGGATACCATGCCTGGGCGAATTGCCACTTAAAATTAATCATCACGTAC
ATGTTTTAAGGTTTTGTTACACAAGACCTCACTTCCAAGTATCACTTTCTGTTTTGGTCATCAGTTGCTGCA
TAATAAACAACCACCCTAAAATTTAGTGACTTAAAACAACAATCATTTATTGTCTGCCATGGTTCTGTGGTT
TGACTGGGATCAGCTGAGTGGCCTGTTTCACTTGGTGTCAGCTGGGGGTGTAGGCATCTGCAACATTGTCTT
GGCAGGAACATCCAAGATGTCCCACTTAACACGATGGCTCCTGGGCTCAGCTGGGCTGGTCAGGCCTCCCTT
CCTCTCTGTGTTGCCACACGGCCTCTCTCTATCCATGTGGCCTCTCCATATGGTCTCTCCCTGGTGGAGGTG
AATTTCTTCAAGGTTTCTAAACTCTCAAAAGTGGAGCCTGGCAAGCCCACTCAAAGCTTCAGATCCACAACT
GGCACAGCTTCCCTTCCACAGATTCTATAGGTTAAAACAATCACCGGACCAGCCCAGATTCAAAAGAAGGAG
AAACAAACTCCACCCCTCCATGGAGGAAGTAGCAAAAATAATGTAGACAGTTTTTACCCTTTACATCTGGAT
TTGTTAGCTTTTCTGTTTTCATTTTCTTCTGGTCTTTCTTTTGTCCTTCTCTTACACTTATTTTATTACCTT
TTATCAATTAGCTTTTAAGATGATAAAAATCTAATACATGCCTCCTGGAATGTCTTCTTGAGCCTAGGGACT
TTTGTTTAATGATATATCTTGAGGACCTAGAATAGTGCCTGAAATACAATAGTCATTAAATATTTAGCTGAA
TTAAATGAATGGTATATAAGCCAGGGTATTAAAAATAACATAAACAAAGTTGTAATAAATATACTTCCCCAG
TGAACGACCTAATACCATTACTCCCCAAACCCTCAATTTCTGTCTTGAGCATAGAAACTGTTAATTTTTCCT
TTGTGTAGTAGGTCCTTAGTATTTCTTTAGAGGTTGTAGCACTTTATCTTCCTCACTGTTCCTTTTCCTTGG
TTGTCCTTTTCCAAACATCTCTCAACAATTTCTCCCAAGTCCATCTTGCCTGCACTAACAGTCTCCTTTAGA
ACTCCCCTCTCCTGCCTGTCACACAGTCTTAGTAATAGTGCCACAGTATAGTAGTAAGGGTCTCCAGAGAAA
CTCACTTTCTGCAAGTTTTTTAGTGTGGGTAGGTGAGTATTGAAGCTTGTTCTTGGCGTTACCAGGTTGGTT
CTTTGAGTTGAACCAGGGGCATTACATGCGGAATATTCCTGAACAGATCACCTCTGGTTCTGCTGTCTCAAG
GGCCACACACAAGAGCTGCCTTCTGACCAAGATGTCTCTGGGCACATGAGACCTGAAATACACATGGCCAAG
ACTCAACAAAGCGTTTGCTGACTGTCAGAGCTGACAGCATCTCGGTACTGTGGGAGGGAGCCCAGTGTCTGG
TGATAGTCAGGACGGACCCAGGTGATGTCAGGGGTGGGGTGGGGCCTGCAGGAGGGAATGGAGAGCCAGCAC
CTAGGGGAAGCTGGGAATTTAGGAAGATATCCAGAGAGTGTAACTTCAGTTCCACTAATCTCACCTGGGTGA
AAACCAGCCCTCTCCATGGATGATGGTGATTGCAGGTACTGACGATAGCTGCAGACTGCTGGTGGTCTAGGC
AAGCATGCAGGGATGGGAGCAGGCATTTCTGAGAGCTCCCCTTATCCCTGCCCCAAGACAAGGTGGGGGCCC
TGTGGGGAAGGACTATTTTATTCACTTCTGCATCTCCAGTTGTCTAACACATTGCTTATCACCTAACAGTCT
CTTAATAGAAGCTTGCTATATTGAGCTGCCTTGAGTCCACATCATGCTGGTTGAACATAAGTAGAAATTGTG
GGAGACTTAATCAGAGAAAATCTTTCATCTGTCTTACCTTACGTTCTAATGATCACTTCTGTGCCCACAAGG
ACCATCTTTTCTGATTCATGTTCATGTCGATTTCTTTTTTATTTAACTTCCTCTTCAAAATTTCTGGCAGGA
TTTCTTGGGAGCCAATTCTCCATTTTTACTTCCCCATGCCTCCCATTTTAATGACTGTAGGATTTTCTCAGG
GTCTACTCAGCAAAACTTGTTAGTACAACATGAGCAAAACAGCAAATTTATGCAAACACTTAGCAAAGACTT
AGCTGTCTGCCTAATGCTAGGGTGAGACATGGGAGATTCACAAATGAAGAAACACAAGCATTGATCTCCAGG
ATCCTGCAGTGGGAGGTATGCGGAGGACCAACCTGGGAACAGAGCAACGCAACACCATGTGATCTGTAATGA
AAAAGAGGCCTGTACACAGCCAAGAGGTCACTGAGGAAGGAGCCATTGCTTCTGTGCAGTTAGTGCTGCGCT
AGATTCTGCAGGGATGTAAGAAATGTACCCCTATCAAACAAGAAAGACTATGTGAATTGCTGAATATGTGAG
TGGAGTACCAGCACAATGCCATGGAGATGCAGACGTGCCCCATGTGGTGGGGCAGAGTCAGGAAGCACTTTA
AGAAAGAAATAGCATTTCAGGTCTTCCTTTAAAGGTAGAATTTCAACAAGAGGGTGCTCTGGAGAGTGTGTG
TTACCCTGCTGAGAAAATCCTGGCGGTCAGGTAAGATGCTACTGCCAGGGAAGATTGGCCAATTGATTGACT
AAACCCTTAAAGGTTTGGGGATCTTGGGGAGGATTCTGCTGGTGAGAGGGTCTGGACTTCCTCTTGGTCTGT
CCACAGCTGGACCTTCTCAGCACACAAGAGACTATGAGGGTGACCATTTTGCACAGGACAGAATTCCAGCAT
GTTTTTCCCCTGGAGTGATGGAATGACCACCTGCTCAACATCAGTGTCCTCACTGAGACCATGAGATTCAGT
AGAGTGCTGGAAAGCTTCATGCTTACCTGTGTCTTCTTAATGCTTAGTGTTATGATTGAAGGCTTCCTTCAG
TCCTACCTTTTGTTCTGGGGTTCTAAGAATCTTAGGTGCAGGCGAGGCACGGTGGCTCACACCTGTAATCCC
AGCATTTTGGGAGGCTGACGTGGGTGGATGACGAGGTCAGGGGATTGAGACCATCCTGGCAAACATGGTGAA
ACCCCGTCTCTACTAAAAATACAAAAATTAGCCGGGTGTGGTTGCGTGCACCTGTAGTCCCAGCTACTCAGG
AGGCTGAGGCAGGAGAATCGCTTGAACCTGGGAGGCGGAGGTTGCAATGAGCTGAGCTCACGCCACTACACT
CCAGCCTGGGTGACAGAGCAAGACTCTGTCTTAAAAAAAAAAAATCTCAGGTGCACCTGAGACAGATTGAAT
GTGGAAGGGGAAGTGAAACAGGCCTTCCAGGTGTGGGGCCTGGGTGCTGCTATAGTTACAAATGGGGAAGTG
AGACTATAGGTCTCAGTTACCTGTGGAAGGAAGGGTAGAGTGGAGTACTTACGCAAATTAGCTAATTCTGGG
AGCTTGGGGTGCTACCAGGGTATCAGGGAGAATACAGCCAGGGAATAGAATCTTCTTGAAGCAAAGGCTGTT
TGGAAGCCCCCAGAGTGGATGAAAAGGCTCAGTGGGAAACAACAGATATCAGGAGAGGGAGAAGAAGATACC
TATTTCTATACCTTTTGGCCTTGTGTTTTGCTTCAGACACTGTTCCCAGCAAGGTCAGTGGAACCCACTGCT
CAAAACACACACTTGCTCCTTGTTCTGGTGTCATAATAGCTCTGCAAGCAGTGGTGGTGTTCAGCCTGGAGA
ACGTTCCTTTTCTTTTTTTTTTTATCACAATAAACACTCATGGCTTCTCTGCTTCTTCCTTTCTTCTTTGTC
TTAGGACTCTTGAAAAACAGCTGCCAAATGTCAGTTTAGATATTTTGGAGGGAAAAAAGTTGGGAATCAATG
TTTACAGGTTGCCTGCAATGTGCTGGAAACTACATAGTTGGTTCTTTTTAAACTTTCTCTGAATCCTGTCAG
GAAAGTTCCAGCAATCACATCTTAGTGGGTCCGGAATTCGTGGGTTCTTGGTCTCACTAACTTCAAGAATGA
AGCTGTGGACCCTCGTGGTGAGTGTTACAGTTCTTAAAGGTGGTGTGTCCAGAGTTTGTTCCTTCTGGTGTT
GGACATGTTCGGAGTTTCTTCCTTCTGGTGGGCTCGTGGTCTCGCTGGCTTCAGGAGTGAAGCTGCAGACCT
TCACGGTGAGTGTTACAGCTCTTAAGGCAGCGCGTCTGGAGTTGTTCGTTCCTCCCATCTGGAGTTGTTCGT
TCCTCCTGGTGGGTTCATGGTCTCACTGTGCTCAGGAGTTAAGCTGCAGACTTTCGTGGTGAGTGTTACAGC
TCATAAAAGCACTGTGGACCCAAAGAGTGAGCAGCAGCAAGATTTATTGCAAAGAGCAAAAGAACAAAGCTT
CCCCAGTGTAGAAGTGTAGAACGGGACGCCAATGGGTTGCCAGTGTTGGCTCCCCCCAGCCTGCTTTTATTC
CCTTATCTGGCCCCACCCACATCCTGCTGATTGGTTCATTTTACAGAGGGCTGATTGGTCTGTTTTACAGAG
AGCTGATTGGTCCGTTTTGACAGGGTGCTGATTGGTGCATTTACAAACCTTGAGCTAGACACAAAGTGCTGA
TTGGTGTGTTTACAAACCTTGAGCTAGACATAGAGTGCTGATTGGTGTATTTACAATCCCTTAGCTAGACAT
AAAGATTCTCTAAGTCCCTAGTAGATTAGCTAGACACAGAGCACTGATTGGTGCATTTACAAACCTTGAGCT
AGACACAGGGTGCTGATTGGTCCGTTTACAAACCTTGAGCCAGACACAGAGTGCTGATTGGTGTATTTACAA
TCCCTTAGCTAGACATAAATGTTCTCCAAGTCCCCACTAGACTCAGAAGCCCAGCTGGCTTCACCTAGCCGA
TTGTGCACCAAGTCAGCAGGCGGAGCTGCCTGCCAGTCACCTGCTATGCACCCGCACTCCTCAGCCCTTGGA
CGGTGGATGGGACGCCAGGGAGCAGGGCGCGGTGCTCGTCGGGGAGGCTCCAGCGGCACAGGAGCCCACGGC
AGGGAGGGTGGGGGGAGGCTCAGGCATGGTGGGCTACAGGTCCCAAGCCCTGCCCCGCGGGGAGGCAGCTGA
GGCCCAGCAAGAATTGGAGCGCAGCGCCAGTGGGCCAGCACTGCTGGGGGACTTGGCACACCCTCCACAGCT
GCTGGCCTGGGTGCTAAGCCCTTCACTGTCCGGGGCCTGCTGCGCTCGCCGGCCGCTCAGAGTGCGGCCTGG
GGAGCCCACGCCCACCTGGAACTCGCGCTGGCCCACGAGCGCCTCTCTCTCTACACCTCCGCTCAAGCAGAG
GGAGCCGACTCCGGCCTGGGCCAGCCCAGAGAGGGTCTCCCACAGTGCAGCTGTGGGCTGAAGGGCTCCTCA
AGCACGGCCAGAGTGGGCGCGCAGAGGCCGGGGGGCACTGAGAGCGAGCGAGGGCCACCAGCACGTTGTCTC
CTCTCATTAGGGTTGGGGAAATGGACCCTGAGAGAGATTAAGTAATTTGGTGATATTCTATAGTCACTCTGG
CTATGTAATTTATGGAGCCTAGTACAGAATGAAAATGTGGGGCTCATTTTTCAAAAAGCAGGAAACAGCTTT
TCCTTTCTTCCAGGGTCTCTTCCTCCACCTGCCATGCTGGTGTTTGGTTGCTATTTAATGTTGAGCCCTCTT
GGGCACAGGGATACTTGCAGGGACAGGGTGTCTGCTCATTTTTCTGTAGACCTCAAAGGTGAGTCCTGAGGC
TTCAGGGTCACTGGCCTCCTTTTAGGGAGTCACGACGCCTTGTCTTTGTACTTCAGGAATGATTACGAATCT
TTGTAGGTAAAGCGGCAGAATGCCACGTCCTCTCCTGGTTGCCAGGACGTGTTCCTTGTGGTTTAATTGCCG
GGTCTGCCCTGCAGACCCTGGCTGAGCGACAGATGAAAGGAGTACTCAGACACAGGTACGCAGTGAAAGAGC
GGCTAGGGGACTGCCGAAGAGTCAGCAGTCTCAATAAACTGGAGCTGCTCACTTTTATTCAGTACAGACATA
ATGCCGAAAGCCTGGAGCCAACGCAGTCTGTGGGTAATTAACATTGTTGTTCCGCCGTGCAGGGAGCAGTCT
CGCCAGAGGATGATGAAAGGTTGGTTTCCGGAAGTAAACAAGCTTATTTAGACAAACTCCCCTACATTCCCT
TGTACCCACTCCTCGCCCTCTGCGTCAGGGTAAGAGAACAGCTGCCTTCAGCTTATTCTCCCCCGAAGCTTT
GCAGAGCCTTCTGACCTTTCAAAAGGTCTTCTTCTTTCCCTATCGGTTCTCCCACTACTCTGACTGATCTCC
TATATTTGATCTCACCTTAACAATCACTTCTTAGAGCTGGGTCAGGAAGTATGCAGCATGCACCTGGCACTC
CTAGTACTGTGCCCATGATGGGCATTGCTGATTGTTCAGAGCATATTGGATGAGCCTGGTTCAGCCTCAGAA
TCTTCCACCCAGTGCACCATGGAGATGCTACCAATTGGTTGGAGTTGCTCTGAGAGGTGACATTTCCTTGTG
ATTCTGCATTAGAAACATGTTGTTTGTCAGCCGAAACAGGGAAACCTGACACGTTATCCGCCCCCAGGAAGA
TCCCATCATCATTCCATGCACCTTCAGTCCTGGGAGCTTACTTTAAAAAAAAGTGACTGACATATGAGCGCA
GGTCCCCAAACAGAGGGGAGGCAGGATGAGAAGCCAGATGAAGAGAGTCAAGGTCCTGGGGCTGCTTAGCTT
GGATGAATCTGATGGGAGGTGGGGTGCATCTGAGTGTTCTCTGCTGATGAAGAACAGACTTGTTGCACGGGG
GTAGGTGTGTGCTGTGTAAACACACATCAGAATCAGGACCCCGAATAGTGAATAGGCAAGAGTAACAGCTGA
ATTTGCCCAGCTCATCACAATTTAACATCAGTTTTCAAAAAGGTAAGAGCGTGGCTTTCATAGCATGCAGAA
TCAACACACATCAAAGATTGATTTACTCATTTATGAAGGAATCAGCAAAATGACAAACTTAGTTCAGAGAAT
ATTTTGAGGCTCTGAGTAGATATAAAACTGGTTAATGTTTCTCAGGGCAATAAAAAGCTATAAACGTTGGGG
ATTTCTTTTTTATCAGACAGAAATTATTTGCATACTTAACAGAAAAGATCTCCAAGTTACCATCTAACTTCA
TAAGGTTCGAATAAAACTTCATAGAGTTATTAATGAATGGTAAATAGAAAAGACAAATATATGTTTTACCAG
ATAATTAAGTAATTCTTGGTAAACCTGGCAAACAGTACCCCAGTGTGACTCTGAAAAGACATGCTGCCCATC
TTTTTGCCTTATTTCCACGTTTTAGGTATTTTTGTAAGATATCTATTCAATAAATATGTATTGAGCTCCTAT
GATGTCCCAGAAACTCTTTTAGATCCTGGAGATATAGCAGTAAACAAAACAGATGAAATTCTTGCTCACATG
GAACTTATATTCTAGTAGGGGAGACAGACATTGAAACAGAAAAATACATAGTATGGCAGATGGTGGAAAGTA
TTAAAAAGAGTGCTGTGTAGTGTTTACAACTTACTCATTTATGAAGGAATCAGCAAGATGATAAACTCAGTT
CAAAGAACATCTTGAGGCACCGAGTACATTTAAAAGTGGTTAGTTTCTCAGGGCGATAAAAAGCCATAAACT
TTGGGGATTTCTTTTTTAGGTATGGAAACCTAAAGTAAAGAAGATGCTATGGTTTGCACATTTGTCCCCTCC
AAAACTCATGTTTGAAATGTAATCCCAGAAGTGGCAGGATGAGAGATTGGCCCTTTAGGAGGTGACTGGGTC
ATGAGAGATCTGCCCTCATGAATGGATTAATCCATTCATGGATTACTGATTAATACGCTAATGGGTTAATGG
ATCAATGGGTTATCCTTGGAATGAAATGGCAGGCTTTACAAGGAGAGGAAAAGGGACTTGAGCTAGCATGCT
CACCCTCCTCACCGTGTGATGCCCTGTCCTGCCTCAGGACTCTGCGGAGTTCTGGTAAGCAAGAAGGCTGTC
ACCAGATGTGTCCCCTAAACCTTGGACTTTTTGGCCTCCATAACTTTAAGGAATAAATTCATTTTTAAAATA
AATTACCCAGCTTCAGGTATTCTGCTATAAGCAACAGAAAATGACTAAAACAGGAGGCTTTACTGGAAGGTG
TCCTCTTAGCAAAGACCTAAAGAAAGAGGGAGAGTGAAACATAGAAATATCTGGGGAGAACATCCTAGGTAA
AAGGAACAGCATGTGCAAAGGCCTTGAAAAGCAGCAAGCCGCTCTCCCTCTCCCTCTCCCTCTCCCTCTCCC
TCTCCCTCTCCCTCTCCCTCTCCCCCTCTCCCTCCCCCTCCCCCTCCCTCTCCCTCTCTCTCCACGGTCTCC
TTCCACGGTCTCCCTCTGATGCCGAGCCAAAGCTGGACGGTACTGCTGCCATCTCGGCTCACTGCAACCTCC
CTGCCTGATTCTCCTGCCTCAGCCTGCCGAGTGCCTGCGCACGCCGCCACGCCTGACTGGTTTTCGTTTTTT
TTTTTTGGTGGAGACGGGGTTTTGCTGTGTTGGCCGGGCTGGTCTCCAGCTCCTGACCGCGAGTGATCCGCC
GGCCTCGGCCTCCCGAGGTGCCGGGATTGCGGACGGAGTCTCGTTCACTCGGTGCTCGGTGGTGCCCAGGCT
GGAGTGCAGTGGCGTGATCTCGGCTCGCTACAACCTCCACCTCCCAGCCGCCTGCCTTGGCCCCCCAAAGTG
CCGAGATTGCAGCCTCTGCCCGGCCGCCACCCCGTCTGGGAAGTGAGGAGCGTCTCTGCCTGGCCCCCCATC
GTCTGGGATATGAGGAGCCTCTCTGCCTGGCTGCCCAGTCTGGAGGGTGAGGAGCGTCTCTGCCCGGCCGCC
ATCCCATCTAGGAGGCGAGGAGCGCCTCTTCCCCGCCGCCATCCCATCTAGGAAGTGAGGAGCGTCTCTGCC
CGGCCGCCCATCGTCTGAGATGTGGGGAGCACCTCTGCCCCGCCGCCCTGTCTGGGATGTGAGGAGCGCCTC
TGCTGGGCCGCAACCCTGTCTGGGAGGTGAGGAGTGTCTCTGCCCGGCCGCCCCGTCTGAGAGGTGAGGAGA
CCCTCTGCCTGGCAACCGCCCCGTCTGAGAAGTGAGGAGCCCCTCCGTCCGGCGGCCACCCCGTCTGGGAAG
TGAGGAGCGTCTCCGCCCGGCAGCCACCCCGTCCGGGAGGGAGGTGGGGGGGGGTCAGCCCCCCGCCCGGCC
AGCTGCCCCGTCCGGGAGGTGAGGGGCTCCTCTGCCCGGCCGCCCCTACTGGGAAGTGAGGAGCCCCTCTGC
CCGGCCAGCCGCCCCGTCCGGGAGGGAGGTGGGGGGGTCAGCCCCCCGCCCGGCCAGCCGCCCAGTCCGGGA
GGTGAGGGGCGCCTCTGCCCGGCCGCCCGTACTGGGAAGTGAGGAGCCCCTCTGCCCGGCCAGCCACCCCGT
CCGGGAGGGGGGAGGGGGGGTCAGCCCCCTGCCCGGCCAGCCGCCCCGTCCGGGAGGGAGGTGGTGGGGGTC
AGCCCCCCGCCCGGCCGGCCGCCCCGTCCGGGAGGTGAGGGGCGCCTCTGCCCGTCCGCCCGTACTGGGAAG
TGAGGACCCCTCTGCCCGGCCAGCCGCCCCGTCCGGGAGGGAGGTGGGGGGGGGTCAGCCCCCCGCCCGGCC
AGCCGCCCAGTCCGGGAGGGAGGTGGGGGGATCAGCCCCCCGCCCGGCCAGCCGCCCCGTCCGGGAGGGAGG
TGGGGGGGTCAGCCCCCCGCCCGGCCAGCCGCCCCGTCCGGGAGGGAGGTGGGGGGGTCAGCCCCCTGCCCG
GCCAGCCGCCCCGTCCGGGAGGGAGGTGGGGGGATCAGCCCCCCGCCTGGCCAGCCGCCCCGTCCGGGAGGT
GAGGGGCGCCTCTGCCCGGCCGCCCCTACTGGGAAGTGAGGATCCCTCTGCCCGGCCAGCCGCCCCGTCCGG
GAGGGAGGTGGGAGGGTCAGCCCCCCGCCCGGCCAGCCGCCCTATCCAGGAGGTGAGGGGCGCCTCTGCCCG
GCCGCCCCTACTGGGAAGTGAGGAGCCCCTCTGCCCGGCCAGGACCCCGTCTGGGAGGTGTGCCCAGCGGCT
CATTGGGGATGGGCCATGATGACAATGGCGGTTTTGTGGAATAGAAAGGCGGGAAGGGTGGGGAAAAAATTG
AGAAATCGGATGGTTGCCGGGTCTGTGTGGATAGAAGTAGACATGGGAGACTTTTCATTTTGTTCTGTACTA
AGAAAAATTCTTCTGCCTTGGGATCCTGTTGATCTGTGACCTTATCCCCAACCCTGTGCTCTCTGAAACATG
TGCTGTGTCCACTCAGGGTTAAATGGATTAAGGGCGGTGCAAGATGTGCTTTGTTAAACAGATGCTTGAAGG
CAGCATGCTCGTTAAGAGTCATCACCACTCCCTAATCTCAAGTACCCAGGGACACAAACACTGCGGAAGGCC
GCAGGGTCCTCTGCCTAGGAAAACCAGAGACCTTTGTTCACTTGTTTATCTGCTGACCTTCCCTCCACTATT
GTCCTATGACCCTGCCAAATCCCCCTCTGCGAGAAACACCCAAGAATGATCAATAAAAATAAAATAAAATAA
AAAAAAGGAATGAATCAAGAAAAAAAAAGAAAAGAAAAGAAAAGCAGCAAGCCAGCCAGTGTGTTTGGAATG
TTCTCTTATGGAAAATTTCAAAGATATGTAAAACTAGGGCTAATAATACAATTAACCCCTACTTACCCATTA
CCCAACTTCAACAACTATCAACATTCTGCTGTTCTTATTTCATCTATTTACCCATTAAAAAAAAAGTGTACT
TTAAAGCGAATTCCAGAGTTTGTATAATTTTGTCTGTAAATCTTTCAGTCTGTATCTCTAAATCTTTCAGTC
TGTAACTCTAAAAAGAACATTAAAAAACACAACTATCATACCATCATCCTACCTGACACAACTGAGTAATTT
TTCATATCATCCAATATCGATCAGAAGTTTAATTTTCCATGATTTTCTTTAAAATGTAGTTTTATAATTGAT
TTGTTCTAATCAAGATTTGCACATTGCATTAAAAATATATGTATCTTAAATTTCCTTTAATTTACAACCATT
TCCCTCCCCCATTTAAGAGTGCATATTAATTTATTAAACAGACTGGGCAATTCATCTTGTAGAATTTCCCAC
CTTCTGGGTTTGGCCAATTACATCCTTGTGGTGTTATTTAAAATCCTCCTCTATTCCCTTTATTATCTGTTG
ACTGACAGCTTGGCCAATCAGAATCACTTGAACGAGCTGATTACACCCCCTCTTTCTGAAACGTTTTCTTCC
CTTGAGTCTGTACACACTGTTGGCTTCCAAATTTATATCTCCAACCCAGACCTCTCTCCAGTACTTCTGCTT
GCTACTGTGTTTTTCATAACTTTCTGATTTTTTTAGTATCCAATGCTAGAAAATATATCCATTTTGAGAAAG
AGACAAAGTATGAGCTCATATTGATAATTTCATTTCAAAGTAAAGGGAACAAAGTTTTTATTTAACATGTTA
ATTTTATGCTTGTTTCTCTTTTACACTGAAAATCTTAGTTCTCAATGACATTAATATAATTATGTATTTACT
TCCCATATATATGTTGTATATAATTGTTTTATATATACATATGTGCATATATATTATTGCTAATGAAACGTC
TACTGAATGATGTAAGTTTTCTCTGTGATTCTTTTGGTCCTTGGGACACAGGACTTATCCCACTAGTGATGT
GTAGTCAAAATACTATGTACCAGTGTTTGATACCTTAATTAAAGGATCTCTCTGGCTGCTGCATTGAGAACA
GATGGTAGCAGGACAAGGACATCAGCCTGGAAGCCATCTGGAAGCTCTTTAGTGATTCAGATAAGATTTGAT
TTTGGCTTGGATGGTGATGGATGATGTTGAGAAGTGGGTGGATTCTGGATATGTTTTGAAAGTAGGTTCCAT
GTGATTTGCCATGGGCTAATATATGGAATGTGAGAGAAAAAGAAGAAACAACAATGCTTCCAAGATTTTGGG
GCAGAGGTACTGAAAAAATGAATTTCCATTTATCAGAAAGAGAAAGACTGTGGTAAAGCAAGTTGGGAAGGA
AACAGCAGCTCAGTTTTCCACATTCAGTAACCCTCCCTTATCTAGGGTTTCACTTTCTGCAGTTTCACTTAT
CCATGGCCAGCCACAGTCTGAAAATATTAAATGGAATATTCCAGAATTAAACAACTCGTAAGTTTTAAATTG
AGCACTCTTCTGAGTAGCGTGATGAAATCTCATGATGTCCTGCTGTCTTCTGCCCTGGATGTGAATCATCCC
TTTGTCCAGCATATCCATGCTGCATGTTGTACCTGCCTGTTACTTGCCTAAGCAAGTGGATGGCTACTTGCT
TAGTGTCCATCTCAGTTATCAGATTGAAAAAGCAGTACATATACATAGGGTTTAGTACTATCTGCAGTTTCA
GGCATCACTGAGGGGGTTGGAACATAACCCTGGCAGATAAGGGAGACTACTGTTTTAGTGGAGATGTTGACC
AGAAGACTGGTTCATATGAATATGGGGGTCCTGGAGAGAGGTCTGGGCTGGAGATATAAATTTGGAAATCAA
CAGCGTATACAGACACAAGGAAAGAAAACATTTCAGAAAGAGGGGGTATAATCAGCTTGTTCAAGTGATTCT
GATTGGCCAAGTAAGATGAAGATTGGAAATTGACCAATGGATCGGTGACTTGGCAAGGTCAGTTTGGGAGGA
GTGGTAGTGATGAAAGCTTATGTGGAACAAATTCAAGAAAAAAACAGAAGAGAGAGGAATTAGAGATTGTTG
TGCACAGACAACTCTTGCAAGAGGTTTTGCTGGCCAGGAGAACAGAAATACAAGTGAAGTAGTGGCTGGATG
TTTTCCAAAACATGAAACTTAGTTTTCATAGGAAAAAAATGGTTTTTCTTTTTCTACTTATTCAATTTTGTG
CACAATTTCATTACATTATATAAGTAAAAACCACAAGCACGAACATGTTTATAAATAGGTAAATAAATAACA
AAGTAGATAGAAACAAAAATTCTCAGGTGTGCAAAAGAGTAGTTTATTAGCTGTGTAGAAGACAGAAAACTT
GCTTTTATAGAGGGAATGAATAGTGTTGATTAGTATAGTGAGTCAGTTAAATAGGTATCAGTTGAATTTTTT
GAGACAATAAGTACATTTAGAATTGGCTAGGCATTATTCTTCCATTAAAGGAAACTCTTAGTAATAGATTAG
GTCAGAAAACCACAAAGACAGTTTTCTTGAATAAGGGGGTGGTGGGAAAAAATGGATGTAAGATGCTGAGAA
AAAACAGGAAACTCAACAATAGATGAAATCAAGAATTAGTTTCGTGTGTGTGTGTGTGTGTATTTCACAGAA
AAGGGCTGAAAGGGGAGGAATGGTTATATTCACAGATTTTTGTAGTTGATCTTAATAGGGAATAGGAGATTA
CCTTTTTTTCAGCAAAATATTACCAGCAAATGTCTACTCTGGAAATAGAGGATAAGAATCTTCTATTTAAAG
ACCAAAAAGGTGAAAACTGAGGTCAGAAATTTAAAGAGTAAATGTAGCAAGATGCTAACATTCTTGTTCCCT
CTAATAACTCTTCTTTTTTAAAAATTATAAAAGTAAAACATGCCCATTGAAGAATATTTGGATGAATACAAA
AATATAGAAAAGAATGTAAAATCGCTTAAGCCCCCAAGTAGAATTCTAAATAGCGGAGAAACCCACGATTTA
AAAAAATAAATAAGGAGATCCACAAAAGGAAGATATATACCTGCTTAAGTGGACGGCCTCAGCATAAGTAGT
CTTACAGTTACAATTAATTTTTTTTCTATTATACGATGTTTTAAAAAATTGCTAAGCTCAGTTGTCTACTCC
CTGCTAGAATGTATCAAACATGCTGCATCCACATAGTAGACAGTTATTCTACATTTTTGGTCTTTGGATGTT
TTGAAGCAATTAGCAAAGTTTGATTTGAAGAGACATTATAAATTTCCGACGGCATATTTTTCTCTGGCCATG
ATCCATATTTGCCTTGATATTGTCCAAATGTTCATTTTATCTTATGACTATCAAGCAAATATTATAAAGTTA
TTTGTGAATTTGGCACTTCATAGATGAAATATTGTGAAATTCTGAATAAAACTGCTTCATTGATTATCTTGG
TTCCTAAAAGAGATAGTTTATGAGTTAAATTATCTCTAACGTTGTCTAAGTTGGCAGTAATTAAATCTCCAT
GGGAATTCTTAATAAAGGCAAAGAAGGACTGAAAACACCTTGCTGAAACTGAGAGAGATTTAAAACCACAAA
AAAAATGTAAGATTTAGTTAGTTGTTCACGTTTTGGGGTCCTGCTTTTATGTGTTAATACTTCCAGATAAAA
TTTTTTCAAAGGTAGTTTTGAACCCCAGGATCTGAATATATTAATTTGTTTATCTCTGAAAACTGTATACTT
TGTGTTTCCCAGGTCTGGCTTTCTTCATTTTTCCTACTTGAGGGAACTCCTTTTCTTCCTTCAATATCCTCA
TATCCTCCTTCAATGTTTCTCAGTCAATATCTCCTCTCAATTTTTCCATTTAAAAATTAAAAATTAAATTAT
TAACATTTTAAATACACTGAAATGTTCATGGAATAGTAGTACAAATACCCATTTACCCACCACATGGAGTTA
ATAGACAATAAAAATTTGCCATATTTGTTTTACATCTTTTTTCTAGTTTTTAAAGAAATAACATGTTACAAA
TAAAATCGAAATACTCTTTCCGCCGATTTTCTTTTCTCCTGTCTCAGAGGCAAATACTGCTACTTGCTTCTC
TTGTATCTTTTTAGAAATATTCTCTGCATATATAAGCATATATCCATATATTTTTCCACACATAGTATCTCT
TCCGTGTCTTAGTTTTTTTCATTCTACAATGTATCTTGATGATCAGTGTAGCTTTGTTTCATTCTAATAATT
ATATAGATTCCACTTTACAAATTTACTATACTTTGGACTTGTATCTATGAACGTTTCGATTGTTTTAAACCT
TTGGCAACATTACAGACAATGCTGCAATGAAATCCTTCTGTATGTATACATACGTGGATGTGTGTATAAGAT
ATAATTCTGGAAGTAGAATTGCTAAGTTAAAGCATATGCTACTTAATTTTGAAAAAATTGTCGAACTTCTCT
TCACAAGGCAATTATCTTCTTGAGGTAATGAGAACTCCTATTTCCCCATACCCATACTACCCAGAGTATCTT
CAAATTTTCTGATTTTTGCTGGTCTGATAAGGGAACGTTACCTCAGTTTAATCTTTATTTGCATTTACTATG
TGTTTTTGCCCTGTAGAGTGAATTTTACTTTTCTACCTAACAATATATTTTTGTGATTTTAATGACATTACT
TTTGAGATTTACCCATGCTAATATATAAACTCTGGTTCTGCAATTTTAAGTGCTATATAGTATCCTATTGAA
TGACCATACTACTCATTTATTTATTATTATCTACTTGTTAATTAGTCTGTTATCTTTTTTGTCAGTTTTTGT
GGGCATAAACAATGCTAAAGCATACATGTCTACTTGCGCATATGATTTTTTCCCAGTGTGTTTAATAGAATG
ACTAAGGTAGAATGACTAAAGTTCTGAGGTGTGGCAGGTATGCCATCTTCACCTTTACTGGGTATGCTAGTG
TGCTGCAGCTGCCGTGATAAAGTACTACAAGCCGGGTGGCTTCAACAATAGCATTGTACTGTCTCACAGTTC
TGGAGGCTGGAAGTCTAAAATCAAGGTGTCAGAAGGGTCGGTGCCTTCTGAAGGTGTGAGAGAGAATCTGCT
TCGTGCCCTCTTCCAAGCTTCTAGTAACCTCAGGTGTTCCTTGTCTTGTTGATGGTGTTGTCCCAGTGTCTT
CATATTGTCTTCTCTCTGTTTGTGTTTGTGTTTGTCTCTATGCCCAAATTTCCCCTTTTCATAAGGACCCAG
TCGTATGGGATTAGGGCTTACCCTAATGATCTCATCTTAACTTGATCATCTGTAAAACCTTATTTCCAAATA
AGGTCATTTTCTGAGGTACTGGGTGTTAGGATTTCAACATCGTTTGGGGGGGTAAAATTCAATCAATAACAA
CAGGTATACCAGTTAAGATGTTTTTGGCTGCAACTAACAGAACATTCAACTGAAAAGGTTTAAAATATATTG
TAAATTTTAAACAAATGTTTTATAGAGATGAGTTCTGACTGTGTTGCCCAGGCTGGTCTCAAAATCCTGGCC
TCAAGCAATCCTTCCACCTTGGCCTCCCAAAGTGCTGGGATTACAGGCATGAGCCACAGAGCCTGGCCACTT
TAAAATGTTGTTGACACACATAACAAGACATCCAGAAGTGGGGTGGTCATAGGATCGGTTCAGCAGCACAGT
GATGGCCGAGGCTCTTTCCTTCCACTCTTCCATCTCCCTTGAGTGGCTTTCAGCTTCTTGTCACAAGATTGC
TGCATTATGTCCTCATATACCTGGAACAGAGGGCAGGGAGAGGGGCAAGAGCACACCCCCATTTCAAGTCTC
TTTTCATCCCAAAGTAACATATTTCCCAGCAGCCCTCTTTCAACCCTCTCTAGGCTTCTCCTTAAATTTCAC
TGGCCAGAATTGGGCCACACGGTTACCCAAAGGTATAAAAGAGGCTTGAGAAGTGAGAATTTGATATTTTGA
ATCATAATAGGAGACATGCTTTGCCAATAGGAAAGAAGGGTAAAAGAAATCTTCTGGAGAGTACGGAACCAA
CGGCGTCTGCCACGTTGCATATGGCCAAATGGTCACCAAATTTGTTGTATCACTTTATTTTCACAAAATTGG
TGTAAAGTCCTCATTGCTATTCATCCTCGTCATCATTCGGAATTCTCAAATTTAAAAAATGCCCATTTCCTT
GTTGTTTTATTTTTTTAATTCCCTGATCATTAACATAAGTAATTATCTTTTTATGTGTTTATTTGTCATTCA
GGTTTCTTCTGTGAAAAGCCTATTTCTGTCTTTTGCTTGTTCTTATGTTGAATTGTCTCTACACATTCAGGA
TTCTAATCTTCTGTCAGTTATGTGGATTGCAAATAAAGTCTCCCAGTCTATGGCTTAACTCTTGACACTATA
TTTATCACGTCCCTTATTTACTAAAGATTTAAATATGATCAAATTTATGTTTTCTTTTAGGGTTGTGCCTTT
TGTATCTTATTTAAGAACTGATTTCTTTAATGTGAGATCACAAAAGGGTTATAAATATTCTAAAAATGTTAA
ACTTTTGCTTTTCATAGTTAGATCTTTGATCCACTTGGAATTTATCTTTGTGTGTGGTGAAATTAGGAAGCC
CATTTCATTTTTCCCCAGATGGATAGTCAGTTGTGTAAGTGCTGTTTATCAAATAATCCATCCTTTATCGAG
GGTTTTCTGATGCCACTTCTGTTGCATCTTGTGTTTCTTTATGTATGTCAGTTTATTTCTGGACTCTCCAAT
CTGTTTTATGGTCTGTTTGTCGCTATACCACTGCCACGTTGTTTAAATTACTAATGCTTTGATATCCAATAG
AACATGTCTGCTTTCTGTTACGGGAGATAAATTTACATTTTTAAGAAGTTTATATAAATGGAATACATATCA
TTTACTCTTTTGTGTCTGAGTTGTATGTCTTTGTCATGATGTTTTTGTGATTCATCCATGTTGTATGTATTA
ATAGCTTGTTCCTTTTTATTATTGGGTCTATTGTGTGTATATATCACAATTTGTTTATTTATCTATTGATAG
ACATTTGGCTTGTAGTCACTTTTTGGATACAATGAATAGAGGTTCTATGAACATTAGTGTACAAGTTATTGT
ATGGACATACTGACAGAGCAGGAGCACAGTCCTCTTGGACAAACACTGCCACTTTAAGTTCCAGCTCCATTT
TTAGCCTCATGCATCTCAGGGAAATCACTTCTCTTCTAACTACAAGTAGCCAGAAAGAGCAAACAGTAAACC
ACAGATAAAACAGCTCAGGCACAGAGGGAGGAGGGAGAAAAGTCTCTTGGGTAACTGCCACACTTCACCCTC
ATACAGTGGGCCCCAGTAAAACAGTGGGCGTTAATAAACACATTATTTTCCCTTCAGGTGCACTAAAATAGG
GAAGCTAAAAGCAGACTCGGGGGGTATGCCTGCAGCTGCAGAAAAATGTATAAAAACAGACACACAACTCTC
CCTCCAAAATAAGCACAACAAAAAACACAAAAGCAGTCCAAGCCTCTAATAAACTCTCCTATCCTAAATCCT
TAAAAACTCTTAGTCTGTAAGAGAGTGTGCTGTTGACCTAGCTCAGCCAAAAGCTCCTCACAGGTTCGTTTT
CTCTAAAATAAACCTGTCTTAACTGGCAAGCCACCTTTCGTGTTTTTTTTCCTCTTTCTTTAATTCTTACAC
ATACTGTTTTATTTCTCTTGAGTGAACACCCAGAAATAGAATAGCAGAGCCATATGGTACATAAGTTGATTA
GCTTTTTGAGAAACCACCAAACTGTTTTATAAGGCAATTGTATAGTTTTACATGTGTAGCATCAGTGTGTGA
ATATTCTAGTTGTTCTACGTCCTTGTTAACATTTGGTATTGTCAGACTTTTAAATTTTAGCCATCTAAAAAT
TTATAGTGTTATTTTATGGTGGTTATAGTTTGCATTTCCCCCATGACTAATGATGCTGAGGATCATCTCATA
GGCTTTTTGAAGTATGTGTTCAAATCTTTTGCCCATCTTTAAAAATTAGGGTTTTTGTTTTGTACAAATACT
TGGAAATTAAGCAACATACTCCAGAATGACCAATGGGTCAATGAAGAAATTAAGAAAAATAAAAAAACTTAC
TGAAAATGATGAAAACATGTCTAACAAATAAAAATTGATACACAACATACCAAAATCTATGGAATACAGTAA
AAGCAGTACTAGGAGGAAAGTTCATAGTAATGATTGCCTACGTCAAAAAAGTAGAAAGATTTAAAACAACTT
AACAGTGAACCTCAGGAAACTATAAAAGCAAAACAACAACAACAAAACCCCCAAACTCCAAATTAGTAGAAG
GAAGTAAATAATAAAGATCAGAACAGAAATAAATGAAATAGGTTGGAAAAGTAATACAAAAGATCAACAAAA
TGAAAAGTTGTTTTTTAAAAAAAATTGACTAAGCATTACCTAGACTAACTAAGAAAAAAGAGGGAAGAACCA
AATAAATGAAAAAGGAGATGTTACAATTGATACCACAAAAATATAAAGGATCGTAAGAGACTATTATGAACA
CCAATAAATTGGAAAGCCCAGAGGAGATGGATAAATTTCTGGGCACCTACAACCTACCAAGATTGAACCAGG
ATGAGATACAAAATCCGAATAGACCAATAACAATTATTGAGGAACCTCAATAATAATTTTTATTAAACAACA
ATAAAAAGTTTCCCAATTAAAAAAAAAAGCTCAGGACTGGATGGCTTTACTGCTGGATTCTACCAAACTTTG
AAAAATAACTACCAATTCTTCTCAAACTATTCCAAAAAATTGAAGGGAAGAGAATTCTTCCAAACTCATTCT
ATAAGGCCAGAATTAACCTGATACAAAACCAGACAAGGATACAACAACAAAAAAAGAATTTTGCAGGCCAGT
ATCCCTGATGAACATAAATGGAAAGTTCCTCAACAAAATACTAGCAAACTGAATCCAACAGCACATTAATAA
GTTTATTTACTAAAACCAGGTGGGATTCATTCCAGGGATGCAAGAGTGGTTCAACATATGCAAATCAACAAA
CATAATACATCCCATCAACGGAATGAAGGACAAAAATCATATGATCACCACAATAGATGCAGAAAAACAGTT
GATAAAATTCAACATCCCTCCATGATAAAAACTCTCAAACAATTAGGTTTAGAAGAAGGAACACACTTCATC
TTAATAAAGGCCATATATGACAAATCCACAGCTAATATTGTACCAAACAGGGAAATGTTGGAAGTTTTTTCT
CTAAAAACTGGAACAAGATAAGGATGCTTACCCTCACTACTCTGATTCCACATAGTACTGGAAGTTCTAGCC
AGAGCAATTAGGCAACAGAAAGAAATAAAAGACATCCAAATTTGGAAGGAATAAGTCAAATTGACCATGTTT
GCAGATGACATCCTCTTACCTACAGAAAAATCTAAAGACTCCACCAAAAAACTCTTAGAATTGATATACAAA
TTCAGTAAAGTTGTGAGATACAAAATCAACATACAAAAATCAGTAGCATTTCTATACACCAATAATAAACTA
TCTGTAAAAGGAACCCCACTTACAATAGCTACCCCCCAAAAAAAACCTCCACCTAGGAGTAAATTTAACCAA
AGAGGTGAAAGATCTCTAGAATAAAGACTACAGAACACTAATAAAAGAAATTGAAGAGGACATAAAAAATTG
GATAGATATCCCATGTTCATGGATTGGAAAAATTAATATTGTTAAGATTCCATACTACCCAAAGGAATCTAC
AGATTCAGTGCAATCTCTATCGAATTTTCAATGGCATTTTTCACAGAAATGGAAAAAAAGATTCTTAAATTT
GTTAGGAACCATAAAAGACCCCAAATAGCCAAAGCAATTGTTTGTTTTATTTTATTTTATTTTATTTTATTT
TATTTTATTTTATTTTATTTTATTTTATTTTATTTCACTTCATTTCATTTTATTTTTGAGACAGTCTCCCTC
TGTCACACAGGTTGGAGTGCAGTTGCATGATCTCAGCTCACTGCAATCTCTCCCTCCTGGGTTCAAGCAATT
CTCCTGCTTCAGCCACCTCAGTAGCTAGAATTACAGACATGCACTACCATGCCCTATTTTTAGTAGAGATAG
GGTTTCACCATGTTGGCCAGGCTGGTCTCAAACTCCTGACCTCAAGTGATCCACCTGCCTCAGCCTCCCAAA
GTGCTGGTATTACAGGCATGAGCCTCTGCTCCTAGCCAAGCCAAAGCAATTCTGAGCAAAAAGAACTGGAGG
GATCACACTACTTTACTTTAAAATATAATACTATAGTAAAACAGCATTGTATTGGCATTAAAGCAACACATA
AATCAATGGAACAGAATAGAGAACCCAGAAATAAATTCACATATTTATGGCCTATTGATTTTCTGCAACAGC
ACTAAGAACATAAACTGAAGAAAGGACACCTTTTTCAATAAATATTGCTGGGGAAACTGGATATTCATATGC
AGAAGAATGAAACTAGAGCCCCATCTATCATAATATAAAAAAGTAACTCAAAACGAATCAAAGACTTAAGTG
TAAGACCCCAAATTATGAAACTACTAAGAGAAAACATAAGGAAAATGTTCTGAGCAAAGATTTTATGGATAA
GACCTCCAAAACACAGTCAACAAAGGCAAAAATAGACTAATGGGATTACATCAAACTAAACATTTCTGTGCA
GCAAAGTAAACAATCAACAGAGTCATGTGACAAACTACAGAATGGGATAAAATATTTGCAAACGGTTTCTCT
GACAAGGAATTAATATCTAGAATATACAAGGAACTTAACAGCAAAAAGCCAAATAATCTGATTTTTAAAATC
GGCAATTGATCTGAACAGACGTTTCTCAAAAGAAGACATAAAATGGCCACTAAGTATTTGAAACAGTGCTCT
ATGTGACTAATGATTAGGGAAATGCAAATCAAAATCACAGTGAGATATTATCTCACGCCAGCTAGAATGGCT
ATCATCAAAAAGAAGAAAAAATAACAAAGCTAGCAAGAATGCAGATAAAAGGAAACTTAAAACATTTCAGCT
TTTACTTTAGATTCAGGGGTTACATGTGCAGGTGTATTGCATGATGTTGAGGTTTCAGAATATGATTGAACC
CATCTCCCAGGTGGTGAGCATAGTACCCAATATGTGGTTTTGCAACCCTTCCTTCCTCCTTCCCTCCCTCCT
CTTATACTCCCCAGTGCCTAGCATTCCTATTTTTATGTCCATGCGTACCCAATGTTTAGCTCCCACTTATAA
GTGAGAAATGTAGTATTTGGTTTTCTGTTTCTGCGTTAACTTGTTTAGGATAATGGCCTCCAGCTGCATTCA
TGTTGCTGCAAAATACATGATTTCATTCTTCTTTTTGTGGCTGCATGGTATTCCATGGTGTATACATACCAC
CACATTTTCTTTATCCAATCTGCCATTATTGGGCATCTAGGTTGATTCCATGTCTTTGCTACTGTGAATAGT
GCTGTAATGAACATATAAGTGCATGTCTTTTTTTTGGTAGAACAATTTATTTTCCTGTGGGCCATATACCCA
GTAGTGGGATTTCTGGGTTGAATGGTAATTCAGTTTTTATTAATAGTTCTTTAAGAAATCTCCAAAGTGATA
TTCACAGCGGTTGAACTAATTTACATTCCCACCAACAGTGTATAAGCGACAAAGAAAATTCTCACACATTGT
TGGTAGGAATGTAAATTAGTACAGCTATTATGGAAAGCAGTGTGGAAGTTCCTTAACAGGCTAAAAATAGAA
CTACCATATGATCCAGTAATCTCGCTACTGGACCTATATCCAAAGAAAATAAAATCATATGTCGAAGAGATA
CCTGCACTCCCATCTTTACTGTAGTTTCATTTATAATAATGAAGATATGGAATCCACCGAAGTGTCTATCAA
CAGATGAAGAGATAAAGAAAATGTGGGATATATAGACAATGGAATGCAGCCATAAAAGAGAATGAAATCCTA
TCATTGGTGGCAACATGGATGAGCCTGGAGGACATTATGTTAAGTGATATAAGCCAGGCACAGAAAGACAAG
TTTCATATATTCTCACTTACATGTGGGAGCTAAAAAAGTTGATCTCAGAGAAGTAGAGAGTAGAATAGTGGT
TACTAGAAGCTGAGAAGGGTAGGGAGACAGAGATTGATCAATGAATACAAAATTATATATATGGATAGAGGA
AATAAGTTTTAGTTTTAGTGTTCTATAGCATTGTAGGGTGACTATAGTGAACAATAACTTATTGTATATTTT
CAAGTACTGAGAGGAGAAAATTTTGTACATTACCAGCACAAAGAAATGATAAACCTTTGAGATAATGGATAT
GCCAATTACACAGATTCGATCATTATGCATTGTATGCACGTATTGAAATGTCACTTCACCCCATAAATATGT
GCAATTACATGTCATTTAAAAGTGATAAGAAAAATTAGTTTTTTTGATCTTATTATTGACTCGTAGGAAGCT
TATATATTCTGAAAAGAAGTCCATTTTCAGATGTGTACTAAAAATATTTTCTTTCATTTTATGACTTACCTT
TCCATTTTCTTTAATGGAAACTTTTAAAAACTTTTAAAAAACAAAGTTTTAAAAAAATCTAGTTAAGGTCCA
GTTTGTTAACTTTTTTCTAATATGGCTTGTGACGCTTATTCCTTCTGCCTAGAATGTTCCTGGGATGTTTGT
GGAGCTAAGTCCTCGCTTCCTTCAATTCTTTACTCAAATATGTCCACCCTATTTAATGTCAACTGTCCACCG
TATTTAATGCCACCCTATTTAATATCACTAACACCTCCTCCCCCCTCACTCTTGACATTCATTCTAGTCTAT
TTTACATTTTTTTCTCATAGAACTCATAAATTTCTAGCATGCTTTATAACTTACATATTCATTATGTTTATT
GTTTATTGTCCGTCTTTGTTCCAGTAAAATGTAAACTCCTAGAAGAACAGAGACCTGTGTTTTGTTCACTGA
TGTACCCTAAGTGCTCACAAGTGTTTCTAGCACCTAGTATTTGCTCAATAAATATTTGCTAGGTTGATGAAT
TAATGATTTCTAAGCTTTCCTTCAGCCTGAAGAGTTTTCTGATTGTAAGATTCTACTTAGATAATCCTAATT
GTCTCAGTGACTCTCACCAGTCACTCACTTCTCCCACAAGGTGGCAGTCTTTACCTTCAACACAGGTTCTGG
TAGCCTCAAATTTGAGAATTAATAGCTGAGTTAACCTGCTTGTTTTCTTTGAGCCCAGACAGCCTGCCCTAT
GGGAACTGACAGCTGTAAAATTTAAAGGACGAGTGTAATTACCCTGCAAGATCTGAGTGCTTTTAGGCAAGA
GGATTTAGGGGGTGAGAGTTTTCCTGGAGAGGGACACATTATGAAGGTGATATTGCTTAATTGATGGGGACT
TTGAAACATAGTTGCTCTTTGTGAGAATGGTATAGGTTTAGAGAGAGGTGCTAGCACAGAGCTGTGACACCT
GAAGTAGGCTGACCGCAGACAAATTGGATTTAACCACCAAATATATCTGTGTTTTCATGTCTTCCTGCCCCG
TGCCCTCTTATCTGACTCACTTTACCCCAGCACTGGGGAATAACTGTGCCCTATTCTGGTCCTGACCCTTTT
GTACCATCTAGGGAAATGAGAACTCCTCTTGGGGTCTCAGATCCTCATTTCTGTTAGAACCAATCCTATTCT
GTGGGTAGGGCCATGGTTGTAAATTTCCTGTGGGAGGCAGCATTGCTTTGCAAAAAGAACACAGTTTGGCAT
GTGAGGCAGCTCTGCCACTTGGACAAGGTGATAACGCTTTAGTCTCTTTATTTCTAAAACAGGGAAGATGCT
AATACCCCGCCCATGGACTAGTATGAGATTTAAATGGCAGGTACTTGGCACAGTGGCAGGTGGTGAATGCTC
TTTGGTGATCATGACTATCCCTTTCTCCTGGTAGTGCTGCCTCCTCCCTCTGAGCACCTGGAGTCAATCCAC
CTTGGGTAGGTCAGAGAAGGCAGAAGAAAGTGGTGGGAGGTGAACTCGACGGAATGATGTACAGGGCGATAG
GGTGAGTGAGAGGTCTGGGATCTATTGGCAGGAGCAGAATGGTAGGAAAGGGAAAACATGCCATTGACCTTG
AATCTTGACATTTGTGCCCATCCTATGCTGTGTTGAGCCTCAGGTCACCGTTTGCGGAGGTGAGCAGAAAAC
TGCTAACAGATCGAGGCTTCTCCAGCCTTCTAGGTAAACTTTCATCAGTGGGTTAGTTGTCTTGTTCAGAAG
CTGATCACGGAGCTTTGGCCAAGCATAAACACTGATTATGGCAGTCCAATTGTCATAATCCCTTTGATTCTT
TAATATCACCTTCAAGATTGTTTGTTATTGTCAATGCCCCCACAACCTAAGACCACCAGGAACACACTGTAA
TTGAAAAAGGTGGGTTTGTTGCTCTCTGCAAGAAGGGAGGACACTCAGCGTAGAGACTCATGAGGGTGGGGC
AGGTTTTATCCGATGACGTTAGAAAGGACTTACTGAGGATTTGGGCTTGTTTTAGGAGATTTGGGGGAAAGG
TTCAAGGAGACTGGCTTTTCCTGGATGCTGCCAGGAAGTAGTGGGATGGTAGTAAGTCTGTGGTAGGATGTT
TAAATAAATTTCCTCTACTGGGCTGGAAGAATGAGAAGGCTGAAGCTGTAATAAGTAAAGAAGTGGCAGTCA
CTCCTATCAGCTATGATAAAAGGATGTTTGGCTATTACTTTATGGTTTGGATGCTATTTTTGCTTGTGTTCA
CATCATGGTCTATCATGGTGATAGGCCACGTACACAGTGGCCTTGTCTGATGCTGGTGTCCCATGGAGTTGA
TTATGCTCAGCTGAAGGACACTAAGGCCCAACTGTGGGGGCCAGGCCAGCTCCTGAGTGTCAGGGGGGGCTG
CACTGCTTTGTCATTATCAACATCTCCACATACAATACAGCCTGTGCATGTGAGGTCCCAGAAGGAAGGAGC
TAAAGCAGCTAGACTGGGATCACTTTACTCAATTTGAGGAGAGGAGTGCTCTTGAGGGAACCAGAGGAAGGC
AGATGAGCTGGTCTGATCTCCTTTTCTATTGGAGCTCTATGCAGATAGACTGAAAATATTTGCTAAAACAAA
GAGCTCCATCTCTAGAATACCCTTAGCAGGATGTCCTTGATTAAAGGATTATTTCTGAAAACTAAATCCAGA
ATCCGTGAGGCATGATTCCCTGGAAGATCATGTAAGCTGTACAATTCTCTATGGAATAAATTGGAGACTTCA
TCCCTTAGATCCCTTTGACTGTGTGAGGAACCCCACGAAACCTCACTTAGTGACTTTATTCATTCTTCTGGG
CCTGAAAGCATATGTCTCATGCAGGAAGGAAGGCAGGACCAGTGGGGCTTTGCAGGTTGTGCCATTCTCTCC
TTGCTTTGCTTGAGACTCTTTATTACCTGTCTCCCTGACATTATTTGTAGTGTGATTCTGGGTGTGTTCTGT
GATTCTTGTGACTAGGTATCCAATTTTGCTTAAGGATGCAAGGAAGTGTTTGGGGAGAAAGCTCTATTGGAA
GAGGTCTGTAGTCCTAGCCTCCCTCCCCACCCCACATTTCACATCATTAGACCTCAGCACATGGGTCTGGGG
CACCAACACTGTCTTACCTGTTACACAGTGTGGTCTTTATCTGGATGAGGGATGCGAAAGGATACATTGTGA
CCAAGAGACCTGGGAGAGGCACAAAAATAACAGGTGACCACCAAGGGTGCTTGGACCTGAGATGTTTCCATT
TCCTAAGACCCTCCAAGATTCTCCAACATTTGGTATAGTTGCCCAGGCAATTTAACAAGAAAATCAAATTTT
TGTTATGACCCCCATTGTAATTTATGCTTATCGCAGAAAAATTGAGACTATAAGAAGGAGAATAGAAGGTCA
CAAAACCACTCTATACTAGTCCAGGGATAGCTATTCTTACAACATGGATTGATCAGTGTGGGGTGATTTCTC
CTAGTGTTTTTTGGACAGAGAAGCATTGAAGATGCCCTGGTTTTAAGGTCTTAGGATGAAGGAATTATAGTT
GAACAGTTCAAAATGATGTTATGAATTACTTTCAGATTTGTTTGCTTGATTGCATTAGCCTTGCCTGGCCCT
ACGGTAACTATTTGGTTCCATCATGGTGGCTGAGTAGGTGGCTCTGGAAAAAGAGCTATTCAAGAAAAGCTT
TTCTTTCTCTAAAAATATTGTAGGGGGCTCGCCCTCTGTTCTTGGAAGCAACGTTTGGGATGGCCTCTTGGG
AGGCTGTCTGGTGAAGTGTCTAGGGGTATGTGGTCTGGACTTGGACAGGACAAGATGCAAATTCTGGCTGGG
ATATTCTAGTTGTGGAGTGTTGGGCAAGTTACTTGGTCTTCTGAGTTTATAGGTAAACTGGAGATAATAGGT
ATGTGTGAATGAGGATCCAATGAGATGCCTGTAAAACACTTAGCCAGATGGCTGCGTGAAGAAAGCACTTGG
TAAATGCTAATTGTTGTGGTTGTTATAATTAGTACAATGATTAGTCATTGCTGATTGTTGGCTAACTGGCGG
TAAGAAATGAAAGTAAAGTAAGGCAGTAGCAGCTGAGGGAGGTGGTGGAGGGAATCAGGAGACACTTGGAGG
TTCTGGTTCTGCCTGAGTTTAAGTGCTGGGGAAAACTAGTTGAATAACTGCTGGTCTAACATTTAACAGCTG
TGTGACCTCGGGCTAGTCACATTTCCTTTATAAACTTCCTTTTTCTCATTTGCGAAATGAAGGGGTTTCGTT
AGGTTACTTCTCATCACCCCTGGTTGACCATTAGAATCGTTTGGGAGACCTTTTTAGAAATTCTTGGTTCTG
GGGTCTTCCATTTTCCCCATTCTCACTTGGTGGGTCTGAAAGCAGGCACTGCAGCTTTTCCAAAGCTCTTCA
GGTGACCTTGAAGTGTGGCCAGGGCTGAGAACCTCTGACTTCCAACAGCACTTCTGGTTTAGGAAGGAGCAA
ATCACCGGCACAGAATGAGCTCTCAGGAACGGCTGCTGAGCTAGTAATTGCCGTGACACTGTCTCCCTGTCC
CAACTGCAGGCACCCCTAGACGTCTCCTGATGAAGACTTCCAATTTTGGAACAGAAGAATCTTTGAAAAAAA
TATTATTGAACTTCCAGAAATGATTCATTCCTTCTCTGCTCCTTCTTTAGTTGGAAAGATCTGCCCCCATCC
CTGTCTACTGCAGTCCCAATCCCTTTTTATTTCAACATATATATCCAAACCAACAAAAAAATTGACTCGCAC
AACCAAGGTGAGGTGTTTGGCTTTAAGGATAAAATAAATAGTTTCATAAAACCTGCCCCCAGATTTCTCATT
GCCTCTACTCATTTTCTCTAATTTGTAGGGCACACTGAAAGCTCGGATTCATAAGATGTAGAAAGGGCAGAG
AGTTAAGTTACAAACTCTTCCTAGGTCTGTTTCAACTCTAACGTTCTATAGCTCTGCTCCGTCTAAACAAGG
AATTCTGTTAAATTTGTAACCTGGACTTTCTTGAATACTGAGGTAATGTTTCTTGAAGTAGGATATATATAC
CCAAGAAGAATAAAAATAATTCTGGAGGTGTCTTAATTCTCTGTGGGACTCAATAAAAGTTTTGGTGATTAT
ATATAAACACACTTATGAAAGCATCTGGTACATGTAGGTGCTCAGTGCACATGAATTTCTCTTTCCTGCCAG
ATCTTTTGTAGTGGAAAATTATCTTATTCTTCCATCTTTGTCTGCAAAGATGCTGCTAAGGAAAGATGTAGA
AGAGTTTTACAAGGGAGTTGGAGTATGGGAACAGGAAGTTCCCAAGAAGGCCACTGCATTAATAGAATTGAA
ACAAGATCCCCTAAAGGAAAATCGCTGCCAAATCTCTTTTCTCTAAACTATCCAAAATGGTGCCCCATAAAT
TTTCATTGACATTGAAGTACATAATGTAATAAGCTTTTTTTTCCTTAAATATATAATGTACGGAGAAAACCA
GGTTGATAATGGTTTCCTGGCATAGCTTTCAAAGGCAAGTGTGGATGAAGTGGAAATATGGTGCACAGATAT
TGGAAAGAAACCGCTGTTGAACTCTTCACATTTTCATGTATAACCCAATGATTCTCAAACCTAAACTTGCAT
CAGAATTGCCTGGAGGGCTTATTAAAACATGATTGTTGGATGCCACCCTTGCATTAATTAATTACACCTGCA
ACTGTTCTATTTTTAAATGGTCACATTTTAAGGTACTGGGCTGAGGACTTCATTATGTGAGTTTCAAATGGG
ACATAATTCAACCCCTAGCCCAATCCTAGTGAGTGGTGAGTGGTATCTCTTCGTTTTGATTTCTACTTCCCT
AGTGACTAATGATGTTGAGCATTTTTATGTGTGTTTATTAGCCATTTTTATATCTCTTATGAAAAAATGTGT
ATAAAATTATTGGCCCATTTGTTAATTGGGTTATCCATTTATTATTGAATTATAAGAGCTCTTTACTACTCT
GGATGCAAGTCCTGAAACAGAAATAATATTTACAAATATTTTAATCCATTCTGTGAATTTTATTTTCACTTT
CTTGATGGCGTCCTTTGAAGAACGTAGGATTTTAATTTTGATAAAGTGTAATTTATGTATTTTTTCTTTTGT
TGCTGTGCTTTTGGTATCATATTTAAGAAATAATTGCCTAATCCAAGGTTATGACAATTTTTTATTCTATGT
TTGCCTCTAAGAATTTTCTAATTTTACCTTTTATATTTAGGTCTTTCATTCATTTTGAGTCAATTTTTGTAT
ATGGTGTAAAGTATGGGTCCTAATTTATTCTTTTGCATGTGGATATCTTGTTGTCCCTGCACCATTTGTTGA
AAAGTGTTGTTTTTTTTTTCCCATTGAATGGCCTTGGCACTCTTGTCTAAAATTAATTGATGGTAACTGTAA
GACTTTATTTCTGGACTCTTTATTCCATTGATCTATATTTCTATCATTGTTACTGAGCAATGTGCTTGCTGC
CTGACAGATAGGGAAGCCAATATTATGGAACTGGTTTTTGAGAAAAGCAAAAGCTTTATCGTGAGGTTGACT
TGCAAGGAAACAGGATGCAAAGCTCAAATCTGTCTCCCCTTCTGGGATCTGGGACAAGTTTTATGGGTTAGG
GAGGGCAAGCTGGTATGCAGAAGCACTGGTAGGGCAGGTTTCAACTGGAAGTACTTTAAACAAGACCATTTA
TGGTAAGGTATGGTAAGGGTCTTAACACTGGACATGCCTGGGCTCAGGTTTCTTGCTTTTAAAAATGTTTGG
GCCCTCAGGTTCCAGTCATGTCTTGACCATTTTCTTCTGTGGTGGGGCAGGAGAGGAATTTTTCTTCTGGGT
GTTATTCAAGGTTGAGGTCTTCTTTTCTGCATTGCTTCGGCTGCATGACTTAACAACTTTTTGACTTTGTGC
CTGTTAAATAACTTGACATACTATTATCATCAGAGTAGGGCCAGTTAGAACTGGTCCTGTGATTACATCATT
ATGCCAGTACCAATTATCTTGATTACTGTAGCATTGTAGTAAGTTTTGAAATCAGGAAGTTTGTGTCTTTCA
ACTTTGGTCTTCTTTTTCAGGATTTTTGGCTCTTCTGTGTTCCTTACATTTCCATATGAATTTTAAGTTAAA
CTGTCACTATCTGCAAAAGAAGGAACTGGGATTTTTATAGAGATTACATTGAAGCTGTAAATCAGCTTGGAG
AATACTGTCATCTTAACAATATTAAGTCTTCTGGCCGGGCACGGTGGCTCACGCCTGTAATCCCAGCACTTT
GGGAGGCCGAGGCGGGTGGATCACGAGCTCAGAAGTTCGAGACCAGCCTGGCCAACCTGGTAAAACCCCGTC
TCTACTAAAAATAATAATAATAAAAAACTGGGCATGGTGGCATGTGCCTGTAATCCCAGCTACTCAGGAGGC
TGAGGCAGGAGAATCATTTGAACCCTGGAGGCAGAGGTTGCAGTGAGCCGAGATCGCACCATTGCACTCTAG
CCTGGGCAACAGGGCGAGATTCTGTCTCAAAAAAACAAAAACAAAAACAATATTAAGTCTCCTGATCCATGA
ATGTAGAATGTTTTTCCATTTGTTCAGGTCTTCTTTACTTTGTAACAGTGTTGTGTATTTTTCAATGTTCCA
GTCCTGTAATTCTTTGTTATATTTACTCCTAAGAATATTAATTGTTTTGCTGCTATTATAAGTGGAATTGTT
TAAATTTTGATTTTATATTTTTCATTGATAGTATATTTTTCATTGATAGTATACAATTGATTTTTGTACACT
GATTTTGTAACCTGAAACCTTGCTGACCATGTTTACTCGTTCTAACAGTTTCCTTTTTGTGGATTTCTTATA
ATTTTCTATATACAGTATTTCATGTCATCCATGAAGGGGATAGGTTTACTTCTTCTTATCTAATCTGGATGA
GTTTAGTTTATTTTTCTTACCTAAATTCCTTGGCTAGAACTCCAATACAATGTTGAATATAAGTAATGAAAT
CAGACATCTTTGGACTGTACTTGATTTTAAGGGGGAGCATCCAGTCTTTTGCCATTATGTATAATGTTAGCT
GTGGGGTTTAATAGATGAATTTTATCAGGTTGAGGAAATTTTATTTCTAATCTGCTCAGTGTTTTTTTCATC
ACAAGAGTGTTGGATTTTGTTAATATTTTTGTGTGTCTATTGAGATGATCATATGGTTTTTGTCATTCTACA
AAATACAGCACATTAAATTGATGGATTTTTACATGTTAATTTTTTTTTAAATTTTACTTTAAGTTCTGGGAC
ACATGTGCAGAACGTGCAGGTTTGTTACATAGGTATACATGTGCCATGGTGGTTTGCTGCACCTATCAACCT
ATCATCTAGGTTTTAAGCCCTACATGCATTAGGTATTTGTCCTAATACTCTCCCTCCCCTTGCTCCCCACCC
CCGCCGACAGGCCCCGGTGTGTGTTGTTCCCCTCCCTGTGTCCATGTGTTCTCACTGTTCAACTCTCACTTA
TGAGTGAGAAGACGTGGTGTCTGGTTTTCTGTTCCTGTGTTTTTTAGCTGAGAATGATGGCTTCCAGCTTCA
TCCATGTCCCTGCAAAGGACATAAACTCATTCTTTTTTATGACTGCATAGTATTCCATGGTGTATATGTGCC
ACATTTTCTTTATTCAGTCCATCATTTATGGGCATTTGGGTTGGCTCTAAGTCTTTGCTATTGTAAATAGTG
CTCCAATAAACATATGTGTGGATGTGTCTTTATAGTACAATGATTTATACTCCTTTGGGTATATACCCAGTA
ATGGGATTGCTGGGTCAAATGATATTTCTGGATCTAGATCCTTGAGGAATCGCCACACTATCTTCCACAGTG
GTTGAACTAATTCACACTCCCACCAACAGTGTAAAAGCATTCCTATTTCTCCACAGCCTCACCAGCATCTGT
TGTTTCCTGACTTTTTAATGATCGTCATTCTAACTGGCGTGAGATGGTATCCATTGCGATTTTGATTTGCAT
TTCTCTAATGACCAGTGATGATAAGCTTTTTTTCATATGTTTGCTGGGCACATAAATGTCTTCTTTTGAGAA
GCATCTGTTAATACCCTTCGCCCACTTTTTGATGGGGTTGTTTTTTTCTTGTAAATTTGTTTAAGTTGTAGA
CTTAGGATATTAGATCTTTGTCAGGTGGATAGATTGCAAAAAATTTCTCCCATTCTGTAGGTTGCCTGTTCA
CTCTGATGGTAGTTTCTTTTGGTGTGCAGTATCTCTTTAGTTTAATTAGATCCCATTTGTCAATTTTGGCTT
TTGTTGCCATTGCTTTTGGTGTTTTAGTCATGAAGTCTTTGCCCATGCCTATGTCCTGAATGGTATTGCCTA
GATTTTCGTCTAGGGTTTTTATGGTTTTAGGTTTTACATTTAAGTGTTTAATCCATCTTGAGTTAATTTTTG
TATAAGGTGTAAAGAAGGGGTCCAGTTTTTGTTTTCTGTATATGGCTAGCCAGTTTTCCCAGCACTATTAAT
TAAATAGGTAATCCTTTCTCCATTGCTTGCTTTTGTCAGGTTTGTTGAAGATCAGGTGGTTGTAGACATGTG
GTATTATTTCTGAGGTCTCTGTTCTGTTTTTGTTTTTTGTTTTTTGTTTTTTGTTTTTTTTTTTTGAGATGA
GATCTCGCTCTGTTACCCAGGCTGGAGTGCAGTGGCACGATCTCGGCTCACTGCAACCTCCGCCTCCCTGGT
TCAAGCAATTCTCCTACCTCAGCCTCCTGAGTAGCTGGGATTACAGGCATGTATCACCGCGCCTGGCTAATT
TTTGTATTTTTAGTAGAGATGGGGTTTCACCATGTTGGTCAGGCTTGTCTCGAACTTATCACCTCATGATCT
GCCTGCCTCAGCCTCCCAAAGTGCTGGGATTACAGGCGTGAGCCACCGTGCCCGGCCAAGGTCTCCGTTCTC
TTTCATTGGTCTATATATCTGTTTTGGTACTAGTACTGTAGTTACTGTAGCCTTGTAGTACACTTTGTAGTC
AGGTAACGTGATGCCTCCAACTTCGTTCTTTTTGCTTAGGATTGTCTTGGCTATACGGGCTCTTTTTTGGTT
CCATATGAAATTTAAAGTAGTTTTTTTCTAATTCTATGAAGAAAGTCAATGGTATCTTGATGGGAATAGCAT
TGAATCTATCAATTACTTTGGGCAATATGGCCATTTTCACAATATTTATTCTTCCTATCTATGAGCATGGAA
TTTTTTCTATTTGTTTGTGTCCTTTATTTCCTTGAGCAGTGGTTTGTAGTTCTCCTTGAAGAGGTCCTTATG
TCTCTTGTAAGTTGTATTCCTAGGTATTTTATTCTCTTTGTAGCAATTTTGAATGGGAGTTCACTCGTGATT
TGGCTCTCTGCTTGTCTATTATTGGTATATAGGAATGCTTGTGATTTTTGCACACTGATTTTATATCCTGAG
ACTTTGCTGAAGTTGCTTATCAGCTTAAGGAGGTTTTGGGCTGAGACGTTGGGGTTTTCTAAATATACAATC
ATGTCATCTGCACACAGAGACAATTTGACTTTCTCTCTTCCTATATGAGTACACTTTATTTATTTCTTATGT
CTGATTGCCCTGGCCAGAACTTCCAATACTATGTTGAACAGGAGTGGTGAGAGAGGACATCCTTGTCTCGTG
CCACTTTTCGATAGGAATGCTTCCAGCTTTTGCCCATTTAGTATGATATGGGCTATGGGTTTTTCAGAAATA
GCTCTTATTATTTTGAGATATGTTCCATCGATACCTAGTTTATTGAGAGTTTTTAGCATGAAGGGATGTTGA
ATTTTATTGAAGGACTTTTCTGCATCTATTGAGATAATCATGTGATTTTTTTCATTGGTTCTGATTATGTGA
TGGATTATGTTTATTGATTTGTGTATGTTGAACCAGCCTTGCATCCCAGAGATGAAGCCAACTTGATCGTGG
TGGATAAGCTTTTTGATGTGCTGCTGGATTCAGTTTGCCAGTATTTTAGTGAGGATTTTTGCATCGATGTTC
ATCAGGGATATTGGACTGAAATTTTCTTTTTTTATTGTGTCTCTGCCAGGTTTCGGTATTAGGATGATATTG
GCCTCATAAAATGACTTATGGAGGAGTCCCTCTTTTTCTATTGTTTGCAATAGTGTCAGAAGGAATGGTACC
AGCTCCTCTTTGTACCCCTGGTAGACTGCATGTTAGACGAGATAATATGTATGAACTACCTGGCATATAATA
GATGCTTCCTAAATAAGATTCTAAAAAATAATTATGCTCCAAAAATATTTTTAAAATCAAATAATTTATGTT
TTATTTTCTGTGTTTTATCTCAGACATGTAGACTGCCAAAGTGTATGGGATGCTTTCAAGGGTGCATTTATT
TCAAAACATCCTTGCAACATTACTGAAGAAGACTATCAGCCACTAATGAAGTTGGGAACTCAGACCGTACCT
TGCAACAAGGTAATTGGGGGCATGCCATTGATTTTAAAACTGGGGATAAAAGCCAATGGTAACAATTCATAG
GTCCAAATTTTTATTAGAATGAAGGAAGAGGAAAAATCCAGACATTATAGTGTGAGTGTGGTTGGTAGGAAT
GGAATTTGCAGGCCATTGAGGGGCCATGATATAATTAAGATTTAGGACATCTGGAGAAGGGAGCTAAGAGAG
AGAAATAGGGATACAGAGATAGGAAAGGGGCTTTGGCCAAAAACTAGGCAGAAAAAACCTAACACCAAACCC
AACTCGAACAAACAAATTAACACGACCTATATAATAACAAAACTTTCCCCTGACCTATGATAATAATAGTAG
TAGTAGTAGTAATAACAGCAATGCCAAGTTACACTTGCAGACTGCTTCTTCTTTTTCTTGCTTACAAAAGAC
TCTCCTAATCCTTACTTTCTTAGGCCTTCATAGCCATTCTCTGGAATGGGCACATCAGGTGTCAGCATCCCA
ATTTCACCAGTGAGAAAACTGAGGGTTGGTGTGTTTAGGTGACCAGTGTTGCCCAAGTTTGACAGGCTTCAA
AGTGACCAGTTTAAATGTAAATGGTATGAGACCTGGAGCCACAGAGGCCTGGATTCTAATACATTGGTTATA
TTGGAAAAGCTCTATCAGAGTGCACCTTTTCTATAGCCAATGTTTAAGGCAAAATTCCATGTGCCTAAAATT
TTCTTTGTGAAGCCCTTAAATCCATCCAGAAATTACAGCCTCTCATTCCATTGTTAGTGAGCTGGAGTCATT
GTGAAACTTCTCCATTCACTAGGCGTGATGCCCTATGCAGAGAAGGTGTTTGGCAAATAATAACCCAGGCTG
ACATTTGTCAAATAAGTGACTATGCGATGGATAGTATGCTAAGCAATTTACTTGCATTTATCTCAGTTAATT
TCCCTAGCACCCCATTAGTTTATTTCAGTCATTATCATTACCATTTTACAGGTGTAGAAAGTGGGGCTTAGT
GATGTTTTGGTTGCTCAAGGTGAAACACCTGATAAGTGATGATGATGCTGGGCTTCAATAAGGGCTGGGATT
TTAGGGCCCATACTTTAAACCAGTATCCTTCACTGACTCCCATTAAGAATGAATAGGGGGAGGAGCCAAGAT
GGCTGAATAGGAACAGCTCCAGTCTGCAGCTCCCAGTGAGACCAACGCAGAAGGTGGGTGATTTCTGCATTT
CCATCTGAGATCAGGTTTCCTCGTGTGTCTACACCACCAGGGCCCTGGGTTTCAGGCACAAAACCGAGCCGC
TGTTTGGGCAGACACCAAGCTAGGTGCAGGAGTTTTTTTCGTACCCCAGTGGCGCCTGAAACCCCAGTGAGA
CAGAACTGTTCACTCCCCTGGAAAGGGGGCTGAAGCCAGGGAGCCAAGTGGTCTCGCTCAGCGGGTCTCACT
CCCACGGAGACCAGCAAGCTAAGAACCACTGGCTTGAAATTCTTGCTGCCAGCACAGCAGTCTGAAGTTGAC
CTGGGATGATGGAGCTGGGTGGGGGGAGGGGCGTCCGCCATTACTGAGGCTTTAATAGGCGGTTTTCCCCTG
ACAGTGCTAAGGGGGCTGGGAAGTCTGGACTGAGTGTGGCAACGTGGTTGTGGCCAGACTGCTTCTCTAGAT
TCCTCCTCACTGGGCAGGGCATCTCTGAAGGAAAGGTAACAACCCCAGTCAGGGGCTTACAGACAAAACCTC
CGTCTCCCTGGGACAGAGCACCTGGCAGAAGGGGCAGCTGTGGGCACAGCTTCAGTGGATTTAATCATTCCT
GCCTGCTGGCTCTGAAGACAGCAGCTGATCCTGACAAGAGGGATTCTCCCAGCACAGCACACCAACTCTGCT
AAAGGACGGATTGCCTCCTCAAGTGAGTCCCTGACCCCTGTGTCTCCTGACTGAGAGAGACCACCCAACAGG
GGTCGATAGACACCTCATACAGGAGAGCTCCGGCTGGCATCAGGCCGGTGCCCCTCTGGAATGAAGCTTCCA
GAGGAAGGAGCAGGCTGTCATCTTTGCTGTTCTGTAGCCTCCACTCGTGATACCTTCAGGTGCGGGAGGAAC
CCAGGTGAATAGGGTCTGGAGTGGACCCCCTGCACACTGCAGCAGCCCTATGGAAGAAAGGGCCTGACTGCT
AAAAGAAAAAACAGAAAGCAACAACATCAATGAAAAAGACCCCACAAAAACCCATCCAAAGGTCAGTAGCCT
CAAAGATCAAAGGTAGATAAATGCAAGAAGATGAGAAAGAATCAGCACAAAAATGCTGAAAACTCAAAAAGC
CAGTGTGCCTCTTCTCCTCCAGATGATCTTAACACATCTCCAACAAGGGCATAGAACTGGGCTGAGGCCCCT
AAAAAGAGATGAGTTCATGTCCTTTGCAGGCATATGGATGAAGCTGGAAACCATCATTCTCAGCAAACTATC
ACAAGATCAGAAAACCAAACACCACATGTTCTCACTCATAAGTGGGAGTTGAACAGTGAGAACACATGGACA
CAGGGAGGGGAACATCACACACCAGGGCCTGTCAGGGGTGGGTGCTAGGGGAGGATAACATTAGGAGAAATA
CCTAACGTAGGTGACGGGTTGATGGGTGCAGCAAACCACCATGGCATGTGTATACCTATGTAACAAAACTGC
ACATTCTGCACATGTAACCCAGAACTTAAAGTATAAAAAAACAAAAGATACTAGCTACATTTACCCAATGTT
AAAAAAAAAAAAAGAACTGGGCTGAGGCTGAGGTGGATGAATTGACAGAAGTAGGCTTCAGAAGATGCATAA
TAATGAAATTCACTGAGCTGAAGGAGTATATTCTAACCCACTGCAAAGAAGCTAAGAACCATGATAAAACAT
AGGAGCTGTTAACCAGAATAACTGGTTTAGAGAGGAACATAAATGACCTGATGGAGCTGAAAAACACAACAC
GAGAACTTCAAGATGTAAACACAAGTATCAATAACCAAATAGACCAAACAGAAGAAAGGATATCAGAGCTTG
AAGAGTATCTTGCTGAAATAAGACAGGCAGACAAGATTAGAGAAAAAAGAATGAAAAGGAACAAACAAAACC
TCTGAGAACTATGGGATTACATAAAAAGAACCTATGACTGATTGGGGTACCTGAAAGAGACAGGAAGAATGA
AACCACGTTGGAAAACACACTTCAGGATATCATCCAGGAGAACTTCTTCAACCTAGCAAGATGGGCCAACAT
TCAAATTCAGGAAATCCAGAGAACCCCAGTAAGATACTCCATGAGAAGATCAACCCCAAGACACATAATCAT
CAGATTCTCCAGGTCACCTATAAAGGGAAGCCAATTAGACTAACAGCAGACCTCTCAGCAGAAACCTACAAG
CCAGAAGAGATTGGGGGCCAATATTCAACATTCTTAAAGAAAATAATTTCCAACCTTGAATTTCATATCTAG
CCAAACTAAGTTCATAAATGAAGGAGAAATAAAATCTTTTTCAGACAAGCAAATGCTAAGGGAATTCGTCAC
CACCAGGCCTGCCTTGCAAGAGCTCCTGAAGGAAGCACTAAATATGGAAAGGAAAAACCATTATCAGCCACT
ACAGAAACACACCGAAGTACACAGACCAATGACACTATGAAGCAACTACGTAAACAAATCTTCACAATAACC
AGCTAGCATCATGATAACTGGATCAAATTCACACATAACAAATTAACCTTAAGTGTAAATGGGCTAAATGTC
CCAACTAAAAGACATGGAATGGCAAGCTGGATAGTCAAGATCAATTGGTGTGCTGTATACAAGAGACCCATC
TCACATGCAAAGACACACATAGGCTCAAAATAAGGGATGGAGGAATATTTACCAAGCAAATGGGAAACAGAA
AAGAGCAGGGGTTGCAATCCTAGTTTATGACAAAACAGACTTTAAACCAACAAAGATCAAAAAAGAAAAAGA
AGGGTATTACATAAGGATAAAGGGGTAAATTCAACAAGAAGAGCAAACTATCTTAAATATATATGTGCCCAA
TACAGGAACACCGAGATTCATAAAACAAGTTCTTAGAGACCTTCAAAGAGATTTAGATACCCACACAATAAT
AGTGGGAGAATTTAACATCCCACTGTCAATATTAGACAGATCATCAAGACAGAAAATTAGCAAAGATATTCA
CGACCTGAACTCAGCTCAGGATCAAGTGGACCTGATGGATATCTACTGAAGTCTCCATGCCAAAGCAACAGA
ATATACATTATTATTGGTGCCACATGGCATCTACTCTAAAATTGATCACACAATTGGAAGTAAATTACTCCT
CAGCAAATGCAGAAGAACTAAAATCATAACAAACAATCTCACAGACCACAGCACAATCAAATTAGAACTCAA
GATTAAGAAACTCACTGAAAACCATGCAATTACATGGAAATTGAACAACCTGCTCCTGAATGACTCCTGGGT
AAATAATAAAATTAAGCCAGAAATTAAGAAGTTCTTTGAAACTAATAGGAAAAAAGAGACAATGTATCAGAA
TCTCTGGGATGCAACTAAAGCAGTGTTAAGAGGGAAATTTATAGCACTAAATGCCCACATCAAAAAGCTAGG
AAGATATCAAATTGACATCCTAACATCACAACTAAAAGAACTAGAGAACCAAGAGAAAACAAATCCCAAAGC
TAGCAGAAGACAAGAAATAACCAAGCTCAGAGCAGAACTGAAGGAGATAGAGACACAAAAATCCCTTCCAAA
AAAAAATGAATGCAGGAGGTGGTTTTTTGAAAAAAAATTAATAGAATAGATGGATCGCTAGCTAGACTAATA
AAGAAAATAGAGAAGAATCAGATAGATACAATAAAATGATAAAGGGGATATCACCACAGAAATACAAACAAC
CATCAGAGAATACTATAAATACCTCTATGCAAATAAACTAGAACATCTAGAAGAAATGAATAAATTTCTGGA
TACATACACCCTCCCAAGACTGAACCAGGAAGAAGTTGAGTTCCTGAACAGACCAATAACAAGTTCTATAAT
TGAGGCAGTAATAAATACCAACCAAAAAAAAAAAAAAAAAAGCCCAGGATCAGACAGATTTATAACTGAATT
TTACCAGATTTACAAAGAGGAGCTGATACCCTTTCTTCTGAAACTGTTCCAAAAAATTGAAAAGTAAGGACT
CCTCCCTAACTCATTTTATGAGACTAGCACCATCCTGATAATAAAAACTGGCAGAGATTTAAAAAAAAAAAG
AAAGAAAGAAAACTTCAGGCCAATATCCTGAAGAACATCGATACAAAAATTCTCAACAAAATACTGGCAAAC
TGAATCCAGCAGCACATCAAAAAGCTTATCCACCATGATCAAGTTGGCTTCATCCTCAGGATGCAAGGCAGG
TTCAACGTACATGAATCAATAAATGTAATTCATTACATAAAGAGAACTAAAGACAAAAACCACATGATTATC
TCAATAGATGCGGAAAAGGCCTTCGATAAAATTCACCATCCCTTCACGTTAAAAACTCTCAATAAGCTAGGT
ATCAAAGGAACATACCTCAAAATAATAAGAACCATTTATGACAAACCCACAAGCAATATCATACTGAGTGGG
CAAAAGCTGGAAGCATTCCCCTTGAAAACCGGCACAAGACAAGGATGTCCTCTCTCACCACTCCTATTCAAC
ATAGTATTGGATGTTCTGACCGGGACAATCAGGCAAGAGAAAGAAATAAAGTCTTTTCAAATGGAAAAAAGG
AAATAAAATTGTCTTTGTTTGCAGATGACATGATCCTATAACTAGAAAACCGGATCATCTCAGCCCCAAAGC
TTCTTAAGCTGATAAGCAACTTCAGCAAAGTCTCAGGATACAAAATCAATGTGCAAAAATCACAAGCATTCC
TGTACACCAACAACACGCAAGCAGAGAGCCAAATCATGAATGAACTCCCATTCACAAAGGGAATAAAATACC
TAGGAATACAGCAAACAAGGGAAGTGAAGGACCTCTTCATGGATACCTATAATCCACTGCTCAAGGAAATCA
GAAAGGACACAAACAAATAGAAAAACATTCCTTCCTCATGGATAGGAAGAATCAATATCGTGAAAATGGCCA
TACTGCCCAAGGTAATTTATAGATTCAGTGCTATTCCCATTAAACTACTATTGACATTCTTCATAGAATTAG
AAGAAACTATTTTAAAATTCATATGGAACCAAAAAAGCTCATATAGCCAAGATGATCCTAAGCAAAAAGAAC
AAAGCTGGAGGCATCGTGCTACCCAACTCCAAACTGCACTACAAGGCTACAGATGCCAAAATAGCATGGTAC
TTGTACAAAAATAAACACATAGACCAATAGAACAGAGTAGAGATCTCAGAAATAAAACTACACATCTGCAGC
CATCTAATCTTTGGCAAACCTGACAAAAACAAGCAATGGGGAAAGGAATCCACATTTAATAACTGGTGCTTG
AGAACTACCTAGCCATATGCAGACAATTGAAACTGGACCCCTTCCTTGCAACTCATACAAAAATTAAGATGA
ATTAGAGACTTAAATGTATAACCCAAAACTATAAAAACCTTAGAAGAAAATCTAGGCAATATCATTTGGGAC
ACAGGCACAGGCAAAGATTTCATGAAATTGCCAAATGCAATTGTAACAAAAGCAAAAATTGACAAATGGGAT
CTAATTAAACTAAAGTGCTTCTGCACAGCAGAAGAAACTATCATCAGAGTGAACAGAAAACCTGCAGAATGG
GAGAAGATTTTTGCAATCTATCCCTCTGACAAAGGTCTAATATCCAGAATTTACAAAGAACTTAAACAAATT
TACAAGAAAAAAATAAACAGCCCCATCAAAAAGTGGGCAAAGAACATGAACAGACACTTCTCAAAAGAAGAC
ATCCATGTGGCCAACAAACATATGAAAAAAAGCTCAACATCACTGGTCATTAGAGAAATGCAAATCAAAACC
ACAATCTCATGCCAGTCAGAATGGCATTATTAAAAAGTCAAGAAACAGCAGATGCTGGTGAGATTGTGGAGA
GATAGAAATGCTTTTACACTGTTGGTGGGAATGTAAATTAGTTCAACCATTGTGGAAGATAGTGTGGCAATT
CCTCAAAGATCTAGAACTAGAAATACCGTTTGACCCAGCAATCCCATTACTGGGTATAATAGAAATCATTCT
ATTATAAAGATATGTGCATGCATATGTTCATTGCAGTGCCATTCACAATAGCAAAGACATGGAATCAACTCA
AATGCCCATCAGTGATAGGCTGGATAAAGAAAATGTGGTACGTATACACCATGAAATATTATGCAGCCATAA
AAAGGAACAAGATCATGTCCTTTGTAGGGACATAGATGGAGCCAGAAGCCACATCTTCAGCAAACTAACACA
GGAACATGCAAATGCTGCATGTTCTCACTTATAAGTGGGAGCTGAACAGTGAGAACACATGGACACCAGGAG
GGGAAAAACACACACTGTAGCTTGTTGGGGTTGGGGTGAGGGGAGTGAGAACATTAGGACAAATAGCTAATG
CATACTTGGCTTAATACCTAGGTGATGGGTTAATAGGTGCAGCAAACCCATGGCACATATTTACCTATGTAA
CAAACCTGCACATCCTGCATGTATACCCTGGATATACATGCCCAGGATATACATTTTATTTAAAATAAAAAT
AAAAATAATAGATTCATAAAACAGAATATAATTCTGAACTTTGACTCCCTGTACCTTTAAGAGGGACCCTTA
AATTTAAAAATCTATTGTATTTTTTTTTTAGTAGGGGTAGGGAATATTTAGGGAATTTGGAAGGGGTTATAT
AGTTCTTTAAGAATCAAATAGCACATCTTCCTGAAAATAGCACGTAGACAAAGTTTTTTTGGAGATAACCTT
AGGAATATCGTAACTCTCTGATGCCACCTCCATATGTGATCCTATGTTGATTATAAGATTTTGATCAGTGGC
TTTCAGACTTTTTTGACTGCAACCTAGAATAAAAGATTCATTTACATTGTGACCTAGAACACACACACACAC
ACACACTCTCTCTCCGCCACTCTCCTGCACACAGAAATCATTGATGCTTACAACAATTCTTACTCTTACTAT
GGGTGATTTACTTTGATATGCTCTGTTTTTTTTTTCATTTACAAAACTGTGGATTAATTTTTTTTGACATGC
TAAATTGATCTCAGTAATAGATTGTATTTATTCTTCCTTAGATTCTTCTTTGGAGCAGAATAAAAGATCTGG
CCCATCAGTTCACACAGGTCCAGCGGGACATGTTCACCCTGGAGGACACGCTGCTAGGCTACCTTGCTGATG
ACCTCACATGGTGTGGTGAATTCAACACTTCCAGTGAGGCTCTGGGCCCTGTGGGATTGCCCAGGGATGTGG
AGGGTGAACAGAGTGACTTCTGCTGGAGGCCCTGAATGATTAGTGTGGAGGACAGAGCCACAGGCACCCATC
CTGATGCCATCTATACTTATATTAGTCCATTTGTGTTGCTATTAAGGAATACCTGAGGCTGCGTAATTTATA
AAGAAAAGAGGTTTATTTGACTCACAGTTACGCAGGCTGTACAAGAAGTAGGGTACCAGCATCCACTTCGGG
TGAAGGCCTGAGGCTGTTTCCACTCATGGAGAAGGGGAAGGGGAGCTGGCATTTACAGAGATCACATGGTGA
GGGAGGAAAGCAAGGAGAGGTCAGGGGAGGTGCCAGGCTGTTTGTAATGACCAGCTGTCCTGGGAACTAGTA
GAGTAAGAACTCATTACTATAAGGACAGCACCATGCCATTCGTGCAGGATCATCCCTATGACCCAAACACCT
CCTACTAGTCCCGAGCTCCAACACTGGGGGTCGAATTTCAACATAAGGTTTGGAGAGTTAAATATCCAAACT
ATAGCACTACCCTTAATGGCAACTCAGGCTGATATAAAGTAGCATTCCCTGTTTTCTTGAAAAATTGACTTC
AGAGTTGGGGATTGCCCATGCTCCCTAATTCCCTTCTTTTGAGTGCTCACATAGCCTGCTTCCGAATTCTTG
GTATTTTGCTCTCTGTAAGGTCATCATTCAGGTCCAAAGAAGTCTAGAACAGGATGAGGTCTCAGTGGGACC
TAGACCAAGGTTCTTGCTCTTCAGAATCATCACAGTAGCCATGGACTGGACTCTTCCATCTCAGGCACTGGC
TTTGCCATCATTTTTCAGATGTAGCCTTATCCTGCCCAGAAAGACTCAACACCTCACCAGGGGAAGGGATTT
CCTACAACCAAAACCCTACTGCAGTTTTCACTTCTTTTTTTTTTCTTTTTGTTTATATGGTGGATATTTTTA
CTTTATATAGTTTTATTCTTATTTTTACTGTTTTTCATTGTTTGTTTTTAAAAGCTTATCTTATTATAGCTT
CTTTGTCCCAGGTTTGCATTACTTTCAATTACAAAAATAAAGCATGATTATTTGAAAAAAAAATACTTGCAC
ATTACAGAAATGCATAAAAGCAAAAAGCAAATGTCACTCTGAATTTTCCCTTCACCTCCTACCTCCGCATCA
CTTCTCAAAGGGTAACTATTATCAGCAATTTGATATAGATCTTTCTAGACTTTTCCTATGCTAATGTAAACA
TATATATTTAAAATGTACACGCGCTGTTGTGCAACTTGCTTTATTCACTTAAAATTGGTAGGTATAAAGATA
GCTATCCTCTTTTAAAAGGCTTTATCATTAAGAATCCTATTAATGGATATTAAGTTGCTTTAGTTTTGGTTG
CTATTATGTCATTATTGTAAGAAACACTTTTGTGCGCACACACACACACACACACACACACACACACACACA
CACCTGCATACTTGAACGATAAATTTTTATAAATGAAACTTCAATGTTAAAGGATAAATTGTAATAGAAACT
GGTAACATGTCATTTAAAAGATGGTAACTATACCCTCATCAAGAGTATATATATGAGGCCAGGCACAGTGGC
TCATACCTGCAATCCCAGCACTTTGGGAGGTGGAGGAGGGAGGATCACTTGAGCCCAGTAGTTTAAGAACGG
CCTGGGCAACATAGTAAGACCCCATCTCTATTTTACACTAAAAAAAGAAAAAAAAAGAATGTATATGAGATA
GTTTATTTACCTATATCCCCACTAACACCAGGTATTGTTACTTTAAAATTTTTGGCTCATTTCAGAAGAAAA
TAATACCTCAATTTAGTGTGAAGTTCTTTGATGGTGAGGCTACTATATATACATAAATGGTAGATTTTCTGT
TTCTTCTGTAAGCTGGCAGTTCATATATTTTGACCATCTGTATGCGGTATCTATTATTTTCTAATTAGTAGA
AGTTCTTTATAAATTAATAAGAGCTACGATGTATTAAGTACTTACAAAGTGCCAGTGTTCCATGTGCTACGT
AAGTAGTCACTCATTTAATCCTCCACAGCCCCATGAGGTCATATGGTGATCCCATTTTAGAGATAGGAAGTC
TGAGGCATGGAGTTAAGTAATTTGCCAGCCAGTAAGTGGCAAAGCAAGAAGCAAAGTTTCTCAGACTAACTT
GAGAAACTTTGCTCTTAACTGCCATGTTTTTCTGCCCACTTTCTGGCTCAAGTTGGCGAATATATTTTTCTT
ATTTTGGACTTTACACGGTGTTTATGGTCTGTCTTTTGCCACCCAGAAATGCAGAAAACCTGTCTGTTCTCT
TCCTTATAGCTTCTGTGTTTCATATCTTCCTTAAAAAGATCTTCTTAGAGAAGCATTCTTCTGTATTTTCAC
CTACTATTTTTACTTTCACAATGTTTAAAATATTTTCCATATTTAGATCTGAGTCTTCCCACCTAGAATATG
GTAATATAAATATGTTTTTCCATTAATTTTTTTAGATTTTACAGTTTTTCCCATGTTCCATGTTTTCCTTTT
TAAATTTCCCTTTTAACAATGACTGTTTTATTGGTCATTCATTTAACATTTAGCTTTTAAATGTATTTACAG
TAGACTTCTCTCCTTTTGTTTTATTTTTTAATTTGTTCAATTTTTCTTGAAATAAAGTAGAGAAAATGAAAT
AATTTATTTTTAAGAACTGATTTATTTACAGTTCAGAGTTCCTTATTTTTGCCTTCTTTTAAATTGAATTAT
GTATATGTAGTTTTATTTATCTACATTCTAATACTTTGGCCTCAATTTTTAATTTCTTCTTATTTTATAGAT
TATCTTTCAAGTTCCTGATGTATATGTATTTATTTACTTTTTATTCTAAGTTGACAATTTATAATTGTATGT
ATTTGTGGGGTGAAAAATGAATTTATGAATACAATGTGGGATAATTAAATCAAACTAATTAACATATCCACC
GCCTCAAATACTTTTTTTAGTTTTTGAGACAGGGTCTCACTCTGTCACCCAGGCTGAGGTGCAGTGGTGCAA
TCACAATTCACTGCAACCTTGACCTACCAGGCTCAGGTGGTCCTCCTACCTTAGCCTCCCAGGTAGCTGGGA
CTACAGGTGCCTGCCACCACACTTGGCTAATTTTTTGTATTTTTTTTAGAGACAGGGTTTCACCATGTTGCC
CAGGCTGGTCTTGAACTCCTGGGCTCAAGCGATCTACCCTCTTCAGCCTCCCAAAGTGTTGGGATTACAGGT
GTGAGCCACCAGGCCCGACCTCAAATACTTATTTTTTGTAGTGAGAAAATGTGAAGTTTACTGTCTTAGCAA
TGTTGAAATGTACAGCACACTATTATTAACTACAATCACCATGCTGTGCAATAAATATTTTTAAAAACCCTT
TCTAACTGAGATTTTGTACTCTTTGACCATCATCTCCCCATTCCTTCCAACTTCTGGTCTCTGTATCCACCA
TTCTATTATCTGCTTCTATGAACTTGATTGTTTTAGATTCCATATGTATTAGGACATGCAGCATTTGTCTTT
CTGTGGGTGGCTTATTTTACTTAGCATATTGTTTTCTTGTTCCATCTATATTGTCACAAATGACAGAATTTC
TTTCTTTTTAAAGTCTGAATAGTATTCCATTGTGTATATATACCACACTTTATCCATTCGTCTATTGATGGA
CTCAGGTTGATTCCATATCTTGGCTATTGTAAATAGTGCTGCAATGAACATGGGGGAGCAGGTATCTCTTTG
ACAAACTGATTTGAAATCTTTTGGGTAAATACCTAGAAGTGGGATTGCTGGATCATATGGTAGTATTCTATT
TTTAGTTTGTTGAGGAACTTTCATCACATTTTCCATAATGGGTATACTAATTTACTTTCCCAATAGTGTACA
AATAACCCCCTTTCTTCACATTCTTGCCAACACTTGTTATTTATCTTTCATCTTTTTGATTATACCCTTCTG
ACAGGTGTGAGATGATGTCTCATTGTGGTTTTAATTTTTGTTTCCCTATTAATTAGGAAGCTTGAGCATTTT
AAAATATATTTGTTGGCCATTTGTATGTCTTTTGAAAAATGTCTATTCAGGTCCTTTGCCCACCTTTAAATT
GATTTTTTTTCTTGTTTTTGAGTTGTTTGAGTTCCTTATGTATTTTGTTTTTGTTTGTTTTTTAATTTTTAA
TTTTTGTGCATACATAATAGGTGTATATATGGGATGTGTGTACATGAGATGTTTTGATATAGATATACAGTG
CATAATAATTACATCATGAAAAATGTCTCTTTCCCCATAAGCATTTATCTTTTGTGTTACAAACAATCCAAT
TTTATTCTTTTAGTTATTTTAAAACAGGGGTGTCCAATTTTTTGGCTTCCCTGGGCCACATTGGAAGAATTG
TCTTGGGCCACACATAAAATACACTAATACTAATGATAGCTGATGGGCTGAAAAAAAATCGCAAAAAATCTC
CTAATTTCTAAGAAAGTTTATGAATTGAACTTATGTGTTGGGCTGCATTCAAAGCTGTCATGGGCTGCTTGA
GACCCATGGGCCATGGGTTGGACAAGCTTTTTTTAAAATGTACAACAAAATTGTTATTGACTACAGTCACCA
TACTGTGCTATCAAATAATAGGTCTTATTCATTCTAACTATTTTTTGGTAACCATCCCCACCTCCCCACAAT
GTCTTGCTACACTTCCCAGCGTCTGGTAACCATTTTTCTATTCTCCATGTCCATGAGATCAGTTGTTTTGAT
TTGTTGGATGCTAAAATAAGTGAGAACATCCTATGTTTATCTTTCTGTGTCTAGCTTATTTCACTTAACATA
ATGACCTCCAGTTCTATTCATGTTGTTGCAAATGACAGGAACACATTCTTTTTTGTGGCTGAATAGTACTCC
ATTGTGTATAAATACCACATTTTCTTTATCCATTTATCTATTGATGGACATTTAGGTTGTTTCCATATCTTG
GCTATTGTGAACAGTGCTGCAATAAACATGGGAGTGCAGATATCTCTTCCATTGACTGATTTTCTTTCTGTT
GGGTATATATCCAGCAGTGGCATTGCTGGATCATATAATAGCTCTATTTTTATTTTTTTGAGAAACCTCAAA
ACTGTTCTCCATAGTGGTTGTACTAATTCACATTCCCACCAACAGTGTACAAGGGTTCCCCTTTCTCCACAT
CCTCATCATTATTTGTTATTGCCTGACTTTTGGATGAAAGCCATTTTAGCTGGGGTGAGATGATATCTCATG
ATAGTTTTGATTTGCATTTATCTGATGGTCAATGATTTGAACACATTTTCATATGCCTGTTTGCCATTTGTA
TGTCTTCTTTTGAGAAATATGTATTCAAATCTTTTGCCCATTTTTAATTGGATTATTAGATTTCTTTCCTAT
AGAGTTGTTTGAATTACTTATCTATTCTGGTTTTTAATGCCTTCTTGAATGGGTAGTTTGCAAATATTTTCT
CCCATTCTGTGGGCTCTCTCTTCACTTTGTTGATTGTTTCCTTTGCTATGCAGAAGCTTTTTAACTTGATGT
GATCCTGTTTGTTCATTTTGCTTTCGTTGCCTGTGCTCATGGGGTATTGCTCAATAATTTTTTTTGCCCAGA
CAAATGTCATGGAGAGTTTCCCCAGTGGTTTCTTGTAGTAGTTTGCAGTAGTTTCATAGTTTGAGGTCTTAG
ATTTAAATCTTTAATCTATTTTGATTTTATTTTTGTATGTGATTTGAGATAGGGGTCTAGTTTCATTTTTAT
CCATTGAGCCACTCTGTGCCTTCTGATTGTAGAGTTTAGTCCATTTACATTTGACGTAAATGTTATATTTTT
AAGTAAGGACTTACTCCTGCCATTTTGTTACTTGTTTTCTGTTTGTTTTGTGGTCTTCTCTTCCTTCTTTCT
TTCCTTTCTGTCTTCCTTTCAGTGGAGGTGATTTTTTCAGTTTCCTGCTTTTTATTTTTTGTGGAACTGTTA
TATGTTTTTGAGTTTGAAGTTACCATGAGGCTTAAAAATAGTATCTTATATCCCATTATTTTAAGCTGATAA
CAACTTAACACAGTTTGCATAAAGAAACAAAGACAGCAAACAGAAAGCTAATACAAACTCTATACCTTAACT
TCATTCTCCCACTCTAAAACTTTTTGTTGTTTCTATTTATGTCTTATTGTACTTTATATGTCTTGAAAAGTT
ATTGTAGTTATTATTTCTGATTGGCTCATCATTTAGTTTTTCTACTTAAGACAAGAGTAGTTTACACGTCAT
AGTTACAGTGTTATAACATTCTGTGGTTTTCTGTGTACTTACTACTGCCAGTGAGTTTTGTACCTTCAGATG
ATTAAATTGCTCATTAATATCCTTTTCTTTCTAATTGAAGTACTCCCTTTAGCATTTCTTCTAGGACAGGTC
TCGTGTTAATTAAATCCCTCACCTTTTGTTTGTCTGGAAAAGTCTGTGTTTCTCCTTCAAGTTTGAAGGATA
TTTTCACCAGATATACTATTCTAGAGTAAAAACTTTTTTTTTTGTCTTTCAGCACTTCAAATATGTCATGCC
ACTCTCATCTGGCCTGTAAGGTTTCCACTGAAAAGTCTGCTGCCAGATGTACTGAAACTCCCTTGTATGCTA
TTTGTTTCTTTTCTCTTGCTGCTTTTAGGATCCTTTCTTTATCTTGGACCTTTGGGAGTTTGATTATCAAAT
GCTTTGGGGCAGCATTCTTGGGTTAAATCTGCTTGGTGTTCTATAACCTTCTTGTACTTGGGATATTGATAT
CTTTCTCTAGGTTTGCAAAGTTCTCTGTTATTATTGCTTTGAATAAACTTTCTACCTGTATCTCTTTTTCTA
CCTCCTCTTTGACACCAATAACTCTTGGATTTGCCCTTTTAAGGCAATTTTCTAGATCCTGCCAGTGTGCTT
CATTGTTTTTTATTCTTTTTTCTTTTGTCTCCTCTGACTGTGTATTTTCAAATAGCCTGTCTTCAAGCTCAC
AAATTCTTTCTTCTGCTTGATCAGTTCTGCTATAAAAAGACTCTGATGCATTCTTCAGTGTGTTATTTGTAC
TTTTCAGCTCCAGAATTTCTACTTGATTCTTTTTAATTATTTCCATCTCTTTGTTAAATTCATCTGATAGAA
TTCTTGAATTTCTTTCAGTTTCCTCAACATGGCTATTTTGAATTCTCTGTCTCACATATCTCTGTTTCTTCA
GGATTGATCTCTGATGTCTTATTTAATTCATTTGGTGAGGTCATGTATTCCTGGATGGTCTTGATACTTGTA
GATATTTTTCTGCATCTAGGCATTGTATTTATTGTAGTCTTTACAACCTGGGCCTGTTTGTACTTGTCCTTG
GAAAGGCTTTCCAGATATTTTGAAGGACTCGGATGTTGTGATCTACGTTGTATCTGCTGTAGGGGGCCCTGC
AAGCCTAGTAATGCTGTGGGTCTTGTACACACTCATGGAGGTACCACCTTGATGGTCTTGGACAAGATCTAG
AAGGATTCTCTGGATTACCAGGCAGAGATTCTTTTTCTAGTCCCTTTACTTTCTCCCAGAGTCTCTCTCTTT
CTGTTCTGACCCACATAAAGCTGGTGACACACTCCACCGCAACTAGGACTTTGCTGGGTAAGACTTGAAGCC
AGTACAGCACTTGCCCAGGGCCTGCAGTAACCACTTCCTAGCTGCCATCTATATTTGCTCAAGGCTCTGGGG
CTCTACAATCAGTAGGTGAGAAAGCCAGCCAGACCCGTGTTCTTCTCTTCAGGTTGGCAAGTTTCCCAAGGC
CCTGGGTTGGTCCAGAGGTGCCATCCAGAAGCCAGGGACTAGAGTAAAAAACCTTAGAAGTCTACCTAGTAT
TGCATTGTACTGTGACTAAGCTGGCATTCAAACCACAAGACACAGTCCTTCCCATGCTGTCTTCCCCTTTTC
TAAGGCAAAGGAGCCTCACCTCATGGCCACCACCACCACAGGCCCACAGGGAGTACTGCCAGTGTACTGTTA
ATATTCCAAGGCCCAAGGACTCTTCAGTCAGCTTGTGGTTAATGCTGCCTGGCCTGGGACTCACCCTTCAAA
GCAGTGGGCTCCCCTCTGGCCCAGGGCAGGCCCAGAAATGCTATCCAAGAGCCACATCCTGGAATCAGGGAC
CCCAAGCCCAGTTGGTGCTCTACCTCTCTGTGGCTGTACCTGAAGCCAGCAAGTCACAGAGTCTCACCCAAG
GCCCATGACATACTAATTGGGTATCACTTCTGGTTTTTCAGGGCCCAAGGGCTCTTCAGTTAGTAGGTGATG
AATTCTCTCCAGATGTCGTTGTGAAGATAAAAGAGGTTTATTTTCATGAAAACATATATACTTTAAAGCACC
TTATGAAATGTATGTCCATTCCACCATCAACATTTTTACCTCTGTTGGGAAGATAATTCCTTTTGACTCCAC
AATAATTATTTATATCTACACATGGGAATGTTTCTTTTTTATTTGTGTGGTTTTGGTTTTAAAGCATTTAAT
CATTACAAGACTCCTAGAATTACTATATCATGTGCTCTCTGAAGGCAAAGTTCCCATCTAATTTTTCTATTT
TATCTTTCTACCTCTAAGACCTAAAACTCAATAAATGTGCATTAAGGCAGATATCCTTGGGAGAAGTGACAC
AGAAACTATGTATTCATGCTCTGTGTCCATTGTACTTCACTCAGGGTTAAGACTGCCTTGATGAGGGCAAGT
GTAGGAAGACTCTGAGGCCATCTGAGAGTAAGTGGTGAAGACTTAAGAAGTGGGGCAGGAAACACAGCAAGA
GAGAGTTGTCAGGAAGCAGAAAAGCAGTTGGCAAAAGCAACCACTGAAGGACTGGTTTTACCTCTAATTCTT
CCTGGACTGGGGATAATCCTAGAGGGCTTGTCTCTGTCAGATGAACTTTTGGTAGCATTTCCCAGAACCATG
ACTCAAAACTTGCCACTGTGTTCCCATCTGGGATTTGGAAGATAAGGTAAGAACTTGGAAAGAATTCAGGGG
ACACTTAGTTAAATTGGGTCAAAATGTGTCCATTCTCCAGCCTCCGTCTTGGCAGTGACACATTGGAAAATG
GTTCCACTATGACTGAACAGCCAGGGAAGAGTACAGCTTATTTATACTCTCTGTTTTCCACTTTATTTTCTA
CAAACTATGTCTTTTAGAAATAAACTATCAATCTTGCCCAGACTGGAGAAAGGACTGCAGCAACAACCCTGT
TTCAGTATTCTGGAAAACGGTTTCCCGCAGGGTAAGTACCAAGTAGTGAAATTCTAGAGCTTTGGAGACCAC
AGAACTTAAGACGTTACTCAGTCAGTGCTTGGTTTTAACACTTTTGGATTACAAATACTTTTAGGAATGAAA
ATATAGGATTCATTCCTGAGAAAAAGGTTCAGATGCACATGCCAGAAAATTTACACATCCAATTTTAGAACA
TTCTTAGAGGGTCCATGGGCTCCAGTTGCAGAATCTTTGCACGTACCCACTCTGACTTTGGCTACCAGGAAC
CTGGGGCTTGGTTTAATCCTCTGATTCAGGTATTAGTCAATCTTAGATACCTGGGACAGTCGTAACAATCTA
CATGTATAGACCCCTTACTATGTGGCAGGTACGGTCCTCAGATCTTTACATGAACTAGTAACTTGCATCTTC
ACCAGAACCCTGTGAAGCAGGTGCCATGAGTATTGTAACCATTTAACACATAACGTGAAGGTACAAGTAAAC
AAGGAATCTACTAAATGTACAGAATTAGTAAGAGGCATATGTGGGAGTTTATCCCAAGCTGTCTGACTCCAG
ATTCAGAATCTAGGCTGGGAAAAACTCACCACTCCACCCTCTACCTATTTTTTTTAAAAAAATTGATACATA
ATAGTTTTACATATTTATGGGGTATATAGTGATGTGGTGATACATATAAGGTATAGTGATGAAATCGGGGTA
ATTAACATATCTGTCATCTCGAACATTTATCATTTCTTTGTGTTGGGAGCATTGAATATCCCCCTTCTGGCT
AGCTGAAACTACATATTATTAACTGTAGTCCTCCTACAGTGTTATTGAACACCAGAATTTATTCCTCCTATC
TAACTATAATTTTGTATCTTTTAACAAATCTCTACCTATCTCCTCCTCCTCCTACTTTTCTAAGCCTATGGT
GGCCTCTGTTCTGCCTTTTACTTCCATAAGATCAACTTAATTTTAGCTTCCATATATGAGTGAGAGTATGTA
GTATTTAACCATCTGTTTCTGGCTTACTTCACTTAACATAATGCCCTCCAGTTCCTTCCATGTTGCTGCAAA
TGACAGGAATACTTTTTTTTTTTTTTTTTTTTTTTTTTTTTTTTGAGATGGAGTCTTGCTCTGTCACCCAGG
CTGGAGTGCAATGGTGTGATCTCGGCTCCCTGCAACCACCACCTCCTAAGTTCAAGCGATTCTCCTACCTCA
GCCTCCCCAGTAGCTGGGACTGCAGGTGTGGGCCACTATGCCCGGCTAATTTTTGTATTTTCAGTAGAGACG
GAGTTTCACCATGTTGGCCAGGCTGGTCTCAAACTCCTGACCTCAGGTGATCCGCCAGCCTTGGCCTCCCAA
GGTGCTAAGACTACAGCCATGGGCCACCATGCCCGGCTAATTTTTGTATTTTCTGTAGAGACAGGGTTTCAC
TATGTTGGCCAGGCTGTCTCAAGCTCCTAACCTCAGGTGATCTACCCACCTTGGCCTCCCAAAGTTCTGGGA
TTACAGGGGTGAGCCACTGCACCTGGCCAGGAATACATTTTTTAATTCCTGAATACAATTCCATTGTGCACA
TATACCCCCATCTATTTCTAACTTTACTCAAAGCTACCTGTGTATATTTATTTATCTTGTAAGTTGCTTCAG
TGTTAAGTGGACAAGGAAACATTTCTTTTCAAGTGTGTTAGGGAAAAAAAGAGAAAGGAAGGAAGAAAGAAA
TGAAAGAAAAAGGTGTGAGTAACAATACACTAATTATAACTTTCAAAATTAAACTTAGACATCTGAGGAACT
GGGGCAGGTGGAAATGTATTTGTTAAGTGCATATGTCTTAGTCCATTTGTGCTGCTATCACAAACTACCTGA
GACTTGGTAATTTTTTTAAAACAGGAGTTTTATTTTCTCATTGTGCTGGATACTTGGGAAATCCAGGATTAA
GGTGCCAGCAGATTCAATGTCTGGTGAGGGCTGCTGTCTGCTTCCAAAATGGTGCCTTCTTGCTGTGTCCTT
ACATGGCAGAGGCAGAGGGGCTAAAGGAACCTAGCTAGTTCCCTGGAGCCCTTTCATAAGGGTGTTAATCCC
ATTCATGAGGGCAGAACCCTTAGGGCCCAATTATCTCCTAAAGGACTCACTACTTATTACCATCACATTGGT
CTTAGGTATCAACATAAGAAACACATACATTCAAATCATAGCAGCATATCTGTGACAAGCCTTGAAGTAGTT
CCTCTGTCATTCCCATTGAGTCATCCCCATAGGTAGTGTGACAAATCCCTACATTAAAGGTGAAGAAACTAA
GACTCAGAAGTTAAGTGACTCATCCAAGTTCCCTGGGCTAATAAGTTACAGACCTAAGAGCCTAACCTAGGC
CTCCCTGATTCCAAAGCCATGCTCTTCAATTTTGTTCTTTGAATCTGCTTATTGGTTCTGTCTTTTAAATGA
CAGGTTTGATCTTAACTCTAGGTTGGTACCTAGCTAAATCTCTGTCTTAGGGGGATTCATGTAAACCCTGGT
ACGATGGAAACAGAAAAACAGCCTGGAAGTTGACATAAGGAGACCATGTTTAAACTTGGGCAGATTCCTTTA
CTCATTCTGATCTTCTGATTCCTCATTTGTCAAATGGAAATCAAAATATACTTGTTCCATAGGGTTACTGCA
ATGTTTAAATGAGATACCACCATCCTATAACATAACCCAAAATCCACCCACTTCAAAAATAATTCATTGAGT
ACTTACTATGGACAGTGAACATTCATGGGCACTTTATAGTTTTTGTTTGTTTGCTTTTTCTGAGAATAGTTT
CCATTTCACTACTCTATGGTATGTTTTAGGACAGTGCTGTTGCTAAAATCTTTTAAAGCCAAGTCACATTTT
ATATGTATCAAGAACCTCCTTGCGTTCCCCACTCAGTCCCTGGCACTAGGAATACAGAGGTGCACGTGATTG
AAGGCGTCCTGCCCTTGTGAGCTACCAGCACCTTTATTTTGCCAATCACTCATGGATGTATGTGGATGGACT
TCTTTTTTCAGACTTGTCCCTTTCTTTTTCTGATAACAGAGGCCATGTTTTTTTTTAATTTTTAATTTTTGT
GGGTAGGTTAAACTCATCATTATAATACAATACAGTTGGATAATGTGGAGGGAATGTAAGATGCTGTCAGAG
TCAGAGAAGGGGACTTGAGCTAGTCCCAGGGGTTGGGGAGGCCTCCTGGAGGGAGCATATAGAACACTATTT
TGTTCATTTCATTTTTCCAAAGTCTAACAAAGATTCCTGCTGAATGTTTCTTGCATGGAGAAATAAGACCCT
TTGCTCAAGCATATTTATTCATTCACTTATTCAGTCCTCCTTTCTCTCTGTGCTTTTCCAGGCCTAAGGGTC
CCCTGTTCTCTCCTCAGGTTCCCCTCTTATGGTGTTCCCATTTCCCTCATCCCTGAATCATCCACCTGTTCC
CACTAAATGAAGCATAATGTTTACAGTGCATGACACTGAGAAAGCACTTTCATCTTCCCCCTCTAGACATTC
CTCTTACTCCTCTGGACTTCTGACTTCTGAACCACTGAACCACCAGCTCTATGAACTATAACACTGAACATT
GTTCACTTAGAGATTGGAGCAACTGCTTCAAGAACTCTGATATGAAGCATAATCCGTCCAGTGGCTTGGAAT
AAAAATTGTGTAGACCTGACATTCCTGGGCTAAAACCATATGGGATATCCTTCCTTAACCAGCTATTGCTAA
GTATTGTTTTGAATGAAACTGCTGGAGGATGGTGATTAAGTTTGCATGATGAATGGTGGGCATTTTTTTTTT
TAAGTTTGCAGAAGCTGCCTGTGATGTGGTCCATGTGATGCTCAATGGATCCCGCAGTAAAATCTTTGACAA
AAACAGGTACACATTTATTTTGCATCCTGTTTGCAAGTATCCTGTTGCAAATATCACAGTGAATATTTCATC
TCTAGAAAGAATATGCTTTTCATGTTTCAGGTCAGTTCTGAAGATTAGGGCCAAAAAAGGTAAAAATTTTGA
ATTCCGTGGAGAGAGTTGTCTCCTGTCAATGTGTTTGTCTGATTTCTCCTTTGCCAAAAATTGTCTACCAGG
TTCTAATGGCCACTGCACTGTATCTAGCCCCTGCTCTTAACTTTTGCAGGCCTGGTGTAATTTTCTCAGCTT
TCTCTCCCGTTACCCTCCACCCTACCCATTGCTCACCATTGTTCACACCGTTCCCCCATATGACCTGCCTCC
CCTGCTCCCCTGCTCCCTTCTGTCTAAATCTTCACCATCCATGAAGACCTGCCTTGACCCTCCTCTCCTCCA
GGAAAATTGTGTACCCCAATTCAGTAGTAAAACTACTACCGGGAACATCGGGAACTGTGCTGGGCTCTTGGC
CTTCACTATCTTTTTGCAGACATTGTCAACAATGTACTGTAGTGGTTGAAAGCAGGTACTGGCGGTCATTAC
ATATCATCTGTGTGACCTCAGGCAGGGCAGTCAACCTCTGTGAGCCCCTGAATATGTACCAAAGAGTTGATG
GTGATGGGAAGATTAACTGAGACAACAGATGAAAAATGCTGAGCTCTGTGCCTGACAACAGAGAAAGTGCTC
AATGAGAATCAGCTATTATTCTCATTTGCTGATCCTTGCCACTGAATCTGGCCACACCTGTGCCTTCCTTGG
CTGATCTCCTTCTATATTTACAGTTTTTACTATGTTGATTACCTTTTCGGCCTTTGTTCTCTAATTTTTGTT
CTCTAATCCCACATAAGGCTGACTGAAAGGAGGAAGCATATATTAATTTGCCTTATAAACTCTAGGTGCCCC
AAATTAATTTTTCTTCTCTCCTGTTTTAATATTTAATTCTACAAGGAAGCATTTGTCCTTTCGTCTTCTGAT
CCCAATTTTTTTGGGTAAAAGCATTAACATTTCAGAATTTTATGATCTAATATTATGGTTCAAGCACTTGAA
ACAGGAGTGTCAGTTGTCAGAGACTAACAGGGAAGAGTTTAGGAATGGGATTAGGGCAGGCAACCATAGTCT
TTCAAAGCATTGCCTCTCAAACTTCACTGAGCATGTGAATCACGTGGGGATTGTTCAACTGCAGATCATTTC
AGCAGGTTATAGTGGTTGAAATTCTACATTTCTTTTTTTTTTTTTTTTTTTTTTTTTTGAGATGGCGTCTCG
CTCTGTTGCCCAGGCTGGAGTGCAGTGACACGGTCTCCGCTCACTGCAAGCTCAGCCTCCCAGGTTCACGCC
ATTCTCCTCCCTCAGCCTCCTGAATAACTGGGACTACAGGCACCCACCACCACGCCCAGCTAATTTTTTGTA
TTTTTAGTAGAGACGGGGTTTCATAGTGTTAGCCAGGATGGTCTCGATCCCCTGGCATCATGATCTGCCTGC
CTCGGCCTCCCAAATTGCTGGGATTACAGGCATGAGCCACCATGCCTGGCTGAAATTCTACATTTCTAATGA
GTTCCCAGGTGATGTTCATTAGGTTGGTCTAAGGACCACTCTTCAAATAGCAAATATTTAAAGAATCAACAT
TAATGCACAAATTAAGAATTTTATTTTGAGAATCTTGTTAACCGAGGGTCATGCTGAATAAGAAAAGGTTAT
TGACTGATTTGCAATTTGATGTGTCAACTCTAAAGGATAGGTCCTAGCCAGTGCCTTTCTGCCTGCTGGTTG
TTGAGGGGGGTGTGGATGCTTTCGTTTGGGGTTGATGTTTGGGGTTCTTTGTTTCTTCTATTTTAGCACTTT
TGGGAGTGTGGAAGTCCATAATTTGCAACCAGAGAAGGTTCAGACACTAGAGGCCTGGGTGATACATGGTGG
AAGAGAAGATTCCAGGTATATCTTACTACTTTGTACCCAAGTGTTATTTTATGAATCAGTCCACAAAAGAAT
CCACAGTCACAAGCACGCACTGGGAACAAATTGACTCAGGAAATGAAACTACATGAATGTGCATGAATCCCA
ACAGCCTCTTAACTTTATCTCCACAAAGGATATTTAACTGCTTGACACTTCAGCTCTCCTGCTGACCCAGGA
GCTCTTAGAGGATTTACCTCTACTTTACCTCTTTATCCAAGGGCCTTGTCCAGGGCGTGCTACAAAAACAAA
GAGACTCCAAAAATGTTTGTGAGATCTTGTAATTTTAATACTTTCTTCTTTCTTCCCCAGAGACTTATGCCA
GGATCCCACCATAAAAGAGCTGGAATCGATTATAAGCAAAAGGAATATTCAATTTTCCTGCAAGAATATCTA
CAGGTAATTAATTTCTTCTTGAAGAAAAAAATGACTGTCTTGTCACCTGTAGAATTTCCTTTTTTCCTTAGC
CTCCTCTGAGCTTGGAGGGCTGTGTGAATCTTTCTTGGGCCTTGATGATGATCACAGATGGCAACCTCTGGT
GATCTCTGTCCCTCCTTCCAAGCCGAGTCCAGAAGGTATCCAAGCTAGTGGCCTTCACTTGGCTGCCTTTCC
TCATCCGTCTCTATTGATCCCAAGTAGGACTTGCCTCTAAAGCTGACACAACCTTTGATGGCATATTTTTTC
ATTCCCAGTGTGAGTGGCCCAGTCCAGGGTTCACTGGCCTACTAGGTTTCAGGGGAGCAAGGGAATGTTTTG
CTAAGCCCTTTCTCCCAAGTTGTAAAATCCTTGTGACTTGACATCATTTTGCAAGTGAAGCTTCCTTAGTTG
GATCTGAGTACAGATGCCTAACACATGACAAGGCGTCACACGGCAGTCTACCAAAATCTATATTTTTTAAAT
TAAAAAAAAAAGTATTTACAAAATTTTTCTGATAATTTGTGTTTATTAGAAAACAGTTTAAAATTACAGATA
GATATATATTTTTTAAAGTCACATATAATTCTAGTTTCAAAACTGAGACCCCTCACTCATTTTTAAGCAGTT
GTGACCAATGGTGTAGGTAGGTACTCATTGGTAGAAGCATCTTTGGAGATTTTTCCACGTATAATAGCTTGG
AACAAGATTGATGCAGAGAGGAAAAGCTGTTCAAAGGAGGTAGAAGCTGAGATGCTAGAATATTGTTCCTGT
TTCCATGTCACTACCTTCTCTCACTAACCACATCAGAAAAGCAGAAGGATAGATTCTGGAGACTCTACTGAT
GGCTTTTGTTTCCCAAATGACCTGAATTCCCCATGAGTCACCTTGCTTCTATCTGGAAACAGCCAGAAAAGG
CCATGAGCATTCTACAGCAGTTAGACAGGAAAACAGAAAGAATGAATGAAGGAGCAACTGTAAAAGCAATCT
TGCGGCGGAGGAGCCAAGATGGCCGAATAGGAACAGCTCCGGTCTACAGCTCCCAGCGTGAGCGACGCAGAA
GACGGGTGATTTCTGCATTTCCATCTGAGGTACCGGGTTCATCTCACTAGGGAGTGCCAGACAGTGGGCGCA
GGCCAGTGTGTGTGCACACCGTGCGCGAGCCGAAGCAGGGCGAGGCATTGCCTCACCTGGGAAGCGCAAGGG
GTCAGGGAGTTCCCTTTCCGAGTCAAAGAAAGGGGTGACGGACGCACCTGGAAAATCGGGTCACTCCCACCC
GAATATTGCGCTTTTCAGACCGGCTTAAGAAACGGCGCACCGCGAGACTATATCCCACACCTGGCTCAGAGG
GTCCTACGCCCACGGAATCTCGCTGATTGCTAGCACAGCAGTCTGAGATCAAACTGCAAGGCGGCAACGAGG
CTGGGGGAGGGGCGCCCGCCATTGCCCAGGCTTGATTAGGTAAACAAAGCAGCCAGGAAGCTCGAACTGGGT
GGAGCCCACCACAGCTCAAGGAGGCCTGCCTGCCTCTGTAGGCTCCACCTCTGGGGGCAGGGCACAGACAAA
CAAAAAGACAGCAGTAACCTCTGCAGACTTAAGTGTCCCTGTCTGACAGCTTTGAAGAGAGCAGTGGTTCTC
CCAGCACGCAGCTGGAGATCTGAGAACGGGCAGACTGCCTCTTCAAGTGGGTCCCTGACCCCTGACCCCCGA
GCAGCCTAACTGGGAGGCACCCCCCAGCAGGGGCACACTGACACCTCACATGGCAGAGTATTCCAACAGACC
TGCAGCTGAGGGTCCTGTCTGTTAGAAGGAAAACTAACAACCAGAAAGGACATCTACACCGAAAACCCATCT
GTACATCACCATCATCAAAGACCAAAAGTAGATAAAACCACAAAGATGGGGAAAAAACAGAACAGAAAAACT
GGAAACTCTAAAACGCAGAGCGCCTCTCCTCCTCCAAAGGAACGCAGTTCCTCACCAGCAACAGAACAAAGC
TGGATGGAGAATGATTTTGACGAGCTGAGAGAAGAAGGCTTCAGACGATCAAATTACTCTGAGCTACGGGAG
GACATTCAAACCAAAGGCAAAGAAGTTGAAAACTTTGAAAAAAATTTAGAAGAATGTATAACTAGAATAACC
AATACAGAGAAGTGCTTAAAGGAGCTGATGGAGCTGAAAACCAAGGCTCGAGAACTACATGAAGAATGCAGA
AGCCTCAGGAGCCGATGCGATCAACTGGAAGAAAGGGTATCAGCAATGGAAGATGAAATGAATGAAATGAAG
CGAGAAGGGAAGTTTAGAGAAAAAAGAATAAAAAGAAATGAGCAAAGCCTCCAAGAAATATGGGACTATGTG
AAAAGACCAAATCTACGTCTGATTGGTGTACCTGAAAGTGATGTGGAGAATGGAACCAAGTTGGAAAACACT
CTGCAGGATATTATCCAGGAGAACTTCCCCAATCTAGCAAGGCAGGCCAACGTTCAGATTCAGGAAATACAG
AGAACGCCACAAAGATACTCCTCGAGAAGAGCAACTCCAAGACACATAATTGTCAGATTCACCAAAGTTGAA
ATGAAGGAAAAAATGTTAAGGGCAGCCAGAGAGAAAGGTCAGGTTACCCTCAAAGGAAAGCCCATCAGACTA
ACAGCGGATCTCTCGGCAGAAACCCTACAAGCCAGAAGAGAGTGGGGGCCAATATTCAACATTCTTAAAGAA
AAGAATTTTCAACCCAAAATTTCATATCCAGCCAAACTAAGCTTCATAAGTGAAGGAGAAATAAAATACTTT
ATAGACAAGCAAATGCTGAGAGATTTTGTCACCACCAGGCCTGCCCTAAAAGAGCTCCTGAAGGAAGAGCTA
AACATGGAAAGGAACAACCGGTACCAGCCGCTGCAAAATCATGCCAAAATGTAAAGACCATCGAGACTAGGA
AGAAACTGCATCAACTAATGAGCAAAATCACCAGCTAACATCATAATGACAGGATCAAATTCACACATAACA
ATATTAACTTTAAATATAAATGGACTAAATTCTGCAATTAAAAGACACAGACTGGCAAGTTGGATAAAGAGT
CAAGACCCATCAGTGTGCTGTATTCAGGAAACCCATCTCACGTGCAGAGACACACATAGGCTCAAAATAAAA
GGATGGAGGAAGATCTACCAAGCCAATGGAAAACAAAAAAAGGCAGGGGTTGCAATCCTAGTCTCTGATAAA
ACAGACTTTAAACCAACAAAGATCAAAAGAGACAAAGAAGGCCATTACATAATGGTAAAGGGATCAATTCAA
CAAGAGGAGCTAACTATCCTAAATATTTATGCACCCAATACAGGAGCACCCAGATTCATAAAGCAAGTCCTC
AGTGACCTACAAAGAGACTTAGACTCCCACACATTAATAATGGGAGACTTTAACACCCCACTGTCAACATTA
GACAGATCAACGAGACAGAAAGTCAACAAGGATACCCAGGAATTGAACTCAGCTCTGCACCAAGCAGACCTA
ATAGACATCTACAGAACTCTCCACCCCAAATCAACAGAATATACATTTTTTTCAGCACCACACCACACCTAT
TCCAAAATTGACCACATAGTTGGAAGTAAAGCTCTCCTCAGCAAATGTAAAAGAACAGAAATTATAACAAAC
TATCTCTCAGACCACAGTGCAATCAAACTAGAACTCAGGATTAAGAATCTCACTCAAAGCCGCTCAACTACA
TGGAAACTGAACAACCTGCTCCTGAATGACTACTGGGTACATAACGAAATGAAGGCAGAAATAAAGATGTTC
TTTGAAACCAACGAGAACAAAGACACCACATACCAGAATCTCTGGGACGCATTCAAAGCAGTGTGTAGAGGG
AAATTTATAGCACTAAATGCCTACAAGAGAAAGCAGGAAAGATCCAAAATTGACACCCTAACATCACAATTA
AAAGAACTAGAAAAGCAAGAGCAAACACATTCAAAAGCTAGCAGAAGGCAAGAAATAACTAAAATCAGAGCA
GAACTGAAGGAAATAGAGACACAAAAAACCCTTCAAAAAATCAATGAATCCAGGAGCTGGTTTTTTGAAAGG
ATCAACAAAATTGATAGACCGCTAGCAAGACTAATAAAGAAAAAAAGAGAGATGAATCAAATAGACACAATA
AAAAATGATAAAGGGGATATCACCACCGATCCCACAGAAATACAAACTACCATCAGAGAATACTACAAACAC
CTCTACGCAAATAAACTAGAAAATCTAGAAGAAATGGATACATTCCTCGACACATACACTCTCCCAAGACTA
AACCAGGAAGAAGTTGAATCTCTGAATAGACCAATAACAGGCTCTGAAATTGTGGCAATAATCAATAGTTTA
CCAACCAAAAAGAGTCCAGGACCAGATGGATTCACAGCCGAATTCTACCAGAGGTACAAGGAGGAACTGGTA
CCATTCCTTCTGAAACTATTCCAATCAATAGAAAAAGAGGGAATCCTCCCTAACTCATTTTATGAGGCCAGC
ATCATTCTGATACCAAAGCCGGGCAGAGACACAACCAAAAAAGAGAATTTTAGACCAATATCCTTGATGAAC
ATTGATGCAAAAATCCTCAATAAAATACTGGCAAACCGAATCCAGCAGCACATCAAAAAGCTTATCCACCAT
GATCAAGTGGGCTTCATCCCTGGGATGCAAGGCTGGTTCAATATACGCAAATCAATAAATGTAATCCAGCAT
ATAAACAGAGCCAAAGACAAAAACCACATGATTATCTCAATAGATGCAGAAAAAGCCTTTGACAAAATTCAA
CAACGCTTCATGCTAAAAACTCTCAATAAATTAGGTATTGATGGGACGTATTTCAAAATAATAAGAGCTATC
TATGACAAACCCACAGCCAATATCATACTGAATGGGCAAAAACTGGAAGCATTCCCTTTGAAAACTGGCACA
AGACAGGGATGCCCTCTCTCACCGCTCCTATTCAACATAGTGTTGGAAGTTCTGGCCAGGGCAATCAGGCAG
GAGAAGGAAATAAAGGGTATTCAATTAGGAAAAGAGGAAGTCAAATTGTCCCTGTTTGCAGACGACATGATT
GTTTATCTAGAAAACCCCATCGTCTCAGCCCAAAATCTCCTTAAGCTGATAAGCAACTTCAGCAAAGTCTCA
GGATACAAAATCAATGTACAAAAATCACAAGCATTCTTATACACCAACAACAGACAAACAGAGAGCCAAATC
ATGGGTGAACTCCCATTCACAATTGCTTCAAAGAGAATAAAATACCTAGGAATCCAACTTACAAGGGATGTG
AAGGACCTCTTCAAGGAGAACTACAAACCACTGCTCAAGGAAATAAAAGAGGACACAAACAAATGGAAGAAC
TGCTCATGGGTAGGAAGAATCAATATCGTGAAAATGGCCATACTGCCCAAGGTAATTTACAGATTCAATGCC
ATCCCCATCAAGCTACCAATGACTTTCTTCACAGAATTGGAAAAAACTACTTTAAAGTTCATATGGAACCAA
AAAAGAGCCCGCATTGCCAAGTCAATCCTAAGCCAAAAGAACAAAGCTGGAGGCATCACACTACCTGACTTC
AAACTATACTACAAGGCTCCAGTAACCAAAACAGCATGGTACTGGTACCAAAACAGAGATATAGATCAATGG
AACAGAACAGAGCCCTCAGAAATAATGCCGCATATCTACAACTATCTGATCTTTGACAAACCTGAGAAAAAC
AAGCAATGGGGAAAGGATTCCCTATTTAATAAATGGTGCTGGGAAAACTGGCTAGCCATATGTAGAAAGCTG
AAACTGGATCCCTTCCTTACACCTTATACAAAAATCAATTCAAGATGGATTAAAGATTTAAACGTTAAACCT
AAAACCATAAAAACCCTAGAAGAAAACCTAGGCATTACCATTCAGGACATAGGCGTGGGCAAGGACTTCATG
TCCAAAACACCAAAAGCAATGGCAACAAAAGACAAAATTGACAAATGGGATCTAATTAAACTAAAGAGCTTC
TGCACAGCAAAAGAAACTACCATCAGAGTGAACAGGCAACCTACAACATGGGAGAAAATTTTCGCAACCTAC
TCATCTGACAAAGGGCTAATATCCAGAATCTACAATGAACTCAAACAAATTTACAAGAAAAAAACAAACAAC
CCCATCAAAAAGTGGGCGAAGGACATGAACAGACACTTCTCAAAAGAAGACATTTATGCAGCCAAAAAACAC
ATGAAGAAATGCTCATCATCACTGGCCATCAGAGAAATGCAAATCAAAACCACTATGAGATATCATCTCACA
CCAGTTAGAATGGCAATCATTAAAAAGTCAGGAAACAACAGGTGCTGGAGAGGATGCGGAGAAATAGGAACA
CTTTTACACTGTTGGTGGGACTGTAAACTAGTTCAACCATTGTGGAAGTCAGTGTGGCGATTCCTCAGGGAT
CTAGAACTAGAAATACCATTTGACCCAGCCATCCCATTACTGGGTATATAACCAAATGAGTATAAATCATGC
TGCTATAAAGACACATGCACACGTATGTTTATTGCGGCACTATTCACAATAGCAAAGACTTGGAACCAACCC
AAATGTCCAACAATGATAGACTGGATTAAGAAAATGTGGCACATATACACCATGGAATACTATGCAGCCATA
AAAAATGATGAGTTCATATCCTTTGTAGGGACATGGATGAAATTGGAAACCATCATTCTCAGTAAACTATCG
CAAGAACAAAAAACCAAACACCGCATATTCTCACTCATAGGTGGGAATTGAACAATGAGATCACATGGACAC
AGGAAGGGGAATATCACACTCTGGGGACTGTGGTGGGGTCGGGGGAGGGGGGAGGGATAGCATTGGGAGATA
TACCTAATGCTAGATGACACATTAGTGGGTGCAGCGCACCAGCATGGCACATGTATACATATGTAACTAACC
TGCACAATGTGCACATGTACCCTAAAACTTAGAGTATAATAAAAAAAATAAAAAATAAAAAACAACTCTCAG
AAGCAAAAAAAAAAAAAAAAAAAAAAAAAAAAGCAATCTTGCAGATATCTGACGAGTCTAAGCTGTTCAAAG
ATATGTTGCATGGAGAAAATAGAATAGTAGAAACCTAGACAAAGACTGGGAAATAAAGATGGTCTTATCCCC
AATACTCTTTTACCTTTTTTGTCTTATGAAACATTAACCTTTTTCTCATAAATGACCAGAAGACCTTTATAT
TATAATTCGTCAACTCCCCTCATTTGTGTCTGCTTTAGGCTCCAAGTGAGCTCACTCATTCTCCATCTGGAA
AGAAAATATGGGCATGGCTTCCATTTGGACTTGTACAGACAGTGGCCCATAATGGGAACCAGGTGACACATC
ACAAGGGCAGGTTCTGACACCTCTTCCTTCCAGAAGCCCAGGGGTGCTGGCAGCTGCTTCTGAGGATCTCTC
TCTTCCTTGGCTCATATTTAGCAAAATCAAATTTAAAGAACCCCATTCCTCGCTATCCACCATCCCCCTATT
CATGTGCCAGCCACTCCTATTGGATCCTGTTGCTTTAGCTAATTTTTATGAAAATAATAGTCATTCACCTGT
TAGGTACTTATCTAAGGTTTGTTTCAAAGCAAGTTTGGTCCCCTTGCTGAGGGTCTCCAGCTTTTTCCCAGA
CTCTGCCTCTGACCCTGGATTCAACATTCCCTCAGGAAGCTTCGGAAGAGAGGAAAGCAAATTAGCCACAGA
AGCTGTGGGGGTCCGTGGCCTTGGTTGCTGCTCCTGCTGTTTTTTTGACCAGCAGGTGGCATGGATAGCTCC
CCTCCCGACATGTCACTGCAGGAGAGGAGTTTATATGGATGCTAAGTGGTCTGTGCACCTTGTCGTCGCTAA
AAAAGGGGCTTCCTCCATTAGCGAATTGGACGACAGATGTATCCTACGGTCTCTTGATTTCCTTTTTTGCTT
TCTTGTCATAGACCTGACAAGTTTCTTCAGTGTGTGAAAAATCCTGAGGATTCATCTTGCACATCTGAGATC
TGAGCCAGTCGCTGTGGTTGTTTTAGCTCCTTGACTCCTTGTGGTTTATGTCATCATACATGACTCAGCATA
CCTGCTGGTGCAGAGCTGAAGATTTTGGAGGGTCCTCCACAATAAGGTCAATGCCAGAGACGGAAGCCTTTT
TCCCCAAAGTCTTAAAATAACTTATATCATCAGCATACCTTTATTGTGATCTATCAATAGTCAAGAAAAATT
ATTGTATAAGATTAGAATGAAAATTGTATGTTAAGTTACTTCACTTTAATTCTCATGTGATCCTTTTATGTT
ATTTATATATTGGTAACATCCTTTCTATTGAAAAATCACCACACCAAACCTCTCTTATTAGAACAGGCAAGT
GAAGAAAAGTGAATGCTCAAGTTTTTCAGAAAGCATTACATTTCCAAATGAATGACCTTGTTGCATGATGTA
TTTTTGTACCCTTCCTACAGATAGTCAAACCATAAACTTCATGGTCATGGGTCATGTTGGTGAAAATTATTC
TGTAGGATATAAGCTACCCACGTACTTGGTGCTTTACCCCAACCCTTCCAACAGTGCTGTGAGGTTGGTATT
ATTTCATTTTTTAGATGAGAAAATGGGAGCTCAGAGAGGTTATATATTTAAGTTGGTGCAAAAGTAATTGCA
AGTTTTGCCACCGAAAGGAATGGCAAAACCACAATTATTTTTGAACCAACCTAATAATTTACCGTAAGTCCT
ACATTTAGTATCAAGCTAGAGACTGAATTTGAACTCAACTCTGTCCAACTCCAAAATTCATGTGCTTTTTCC
TTCTAGGCCTTTCATACCAAACTAATAGTAGTTTATATTCTCTTCCAACAAATGCATATTGGATTAAATTGA
CTAGAATGGAATCTGGAATATAGTTCTTCTGGATGGCTCCAAAACACATGTTTTTCTTCCCCCGTCTTCCTC
CTCCTCTTCATGCTCAGTGTTTTATATATGTAGTATACAGTTAAAATATACTTGTTGCTGGTACTGGCAGCT
TATATTTTCTCTCTTTTTTCATGGATTAACCTTGCTTGAGGGCTTTAACAATTGTATTACTTTTTCAAAGAA
CTAAGCTTTAGCTTCATTGATTTTTTTCTATTTAATTGGGTTTTGCTCTTCTCTTTAGCATTGGAAACATAG
AAATGCTTTCTGATTTCTTTGGGTAGATTTACGTATTCAGCTTCTTGAGATGGAAGTTTAGATCACTGATCC
TTCAGCTTGTTTTCTTTTTTGTATACATAGATTTTAGGACGATATATTTTCCCTTGAGTTCTGCTTTAGCTG
CAGCTCTTATGTTTTGATATGCCTCTCTTTATTATCCTTCAGTTAAAAATATCTTTCAATTCATTGTTATAT
AAAAATATGTGCCTAGTTTTTAACATCTGGAGATTTTCTAGTTTTGAAAAAAACATAAGCCAGGCATGGTGG
CTCACACCTGTATCCCCAGCACTTTGGGAGGCCGAGACGGGAGGATCGCCTGAGCTCAGGAGTTTTTACACC
AGCCTGGGAATAACAGTGAGACATTATCTCCAAAAAAATTACCTGGGTATGGTGTTGTGCACCTGTAGTCCC
AGCTACTCTGGAGACTGAGGTGGGAGGATTGTTTGAGCTTGGGAGGTTGAGGCTGCAGGGAGCTGTGATCAC
ACCACTGCACTCTGGCCTGAGTGACAGATTGAGACCCTGTCTCAATAAAAGCAAAAATAAAGAAAATAAACC
ATATGTGTTGAACAAAGGATTAATAAATTAATTTGAGACTCCTTCAGGGAATGACCACAATTTATTGAAAAT
AGCCTAAATGTTGGAGTCAGGCATTTCTGGATTCATATTTTGACATCATGCTGTCATCTTGAACAAAATGCC
TAACCTTTCTGAACTTCAACTTCCTTGCCACTCAAATAAGGATTACAAAACTTAAAATGTGGTAAGTACTAA
AGACGACAGCAAAAATTGAGTCCAGCACAGAGCTTCCTAAATAAGCAAGCACTCAACAGAGTTGGTTCCTTT
CTTCCTCCCCTGCTTGACAATCCAGTTTCCCACAGGAGCCTTTGTAGCTGTAGCCACCATGGTCAGTCCAGG
GATTCTTCACTAGCCCCTTCTCCCCTGGCAGACATCCTTGTGGGAGTTTAGTCTTGGCTCGACATGAGGATG
GGGGTTTGGGACCAGTTCTGAGTGAGAATCAGACTTGCCCCAAGTTGCCATTAGCTCCCCCTGCAGAATGTC
TTCAGAATCGGGGCCCGGTCAGTCTCCTGGGTGACCTGCTGTTTTCCTCTTAAGATCCTTTCCACTTTGGTT
GCTGCTTTCGGGACTCATCGAGTCCTTGCTCAACAGGATACCCCTTGAAGTGGCTGCCTGGGCCACATCCCC
TTCCAAACAAGAAATCAAAATATTAGAAATCAATTTTTGAAATTTCCCCTAGGAAGACTCATTTGAGTGTTC
AAGTTCAGAGCCAGTGGAGACCTTAGGGGAGGGTGGTCACAAGGATTTTGCACAGTGCTTTAGAGGGTCCCA
GGGAGCCACAGAGGTGGTGAGGGGCTGGGTGCTCTTTTCTCCGTGCATGACCTTGTGTGTCTATCTTCATTA
CCACAATGCCTCATCTCTACCTCCTTTCCCCCTGTAGTTCCAACGTGGGTATCTTTGCCATCTCTGGCCCGA
AGGACTTTCTGACCTACATGTATAAATACCCCCTCACAATATATATTACTTTTCCTATAAGTGACTTCTCTA
CTGGATTACTGGTTGCTCATACACCTCATATTTTACTCGTAAATCTACTACTCCCTGTCTGCCTACTCCATT
CTCATTTGCTGTAGAAAATTCTCTTACCATCCCAACTTTCACCCACCATCATGCTTACCCAAAGGCTGTGGG
AATGACCTGGGCCCTAATGCCCCTTTTCTAAATTCCTAAGGCTCACCATTTTCCTATTGTAATGGTTCTTGA
CCTTATAATGTTTGAGGCACCTTTTCAAATATAGTCCTTTGATTTCAGACTGAATACTTGAAAGGACACACA
CACACATACGTAAGTGCATATGACTGCATACACCCACACACACACACGTGCCTGTATACAGTCATATGATAC
ATACACAAACACACGCACACAAGCCTGCATACATCATATGCCAACAGTGGGGATATGTTCTGAGAAATGCAT
CATTAGATGATTTTGTCATTGTGTGAACATCATAGAGTGTACTTACACTAACCTAGATGGTCTAACCTACTA
CACACCCAGGCTACATGGTATCACCTATTCCTCCTAGGCTACAAGCCTGTACAGCGTGTGTCTGTACTAAAT
GCTGTGGGCAATTTTAACCTGATGGTAAATGTTTGTGTATCTAAACATATCTAAACATAGAAAAGGTACAGT
AAACATGCAGTATTATAATCTTATGAGACCGTCATCATATATGTGGTCCACTGTTTGGGCCATCATTGGCTG
AAAAGTGGTTATGCGACACATGACTGTATATATACTTTCCTGTTACAACAACAGTGTCTCTCAATCCACAGT
AATTGCAGCATCCAGTAGGTCTTACTTTAGCCCTGAGTCACCATTTGTGTCAACGTGTTTAGTGCCATGTCC
ACGTCTCTCATGTAACTGGCAGAGCTATCAAATATTTTGGCAAAACACATTGTTTCTTTGGCTTTGCCTTGG
TAACTTTCTGTGCCTTTTGTAGCTCTTGTTTGGAAGAAGCTCAACCCATGTCTGCACACTGTGATACAAGGG
GGACAGCATCGACATCGACTTACTTCTTGGTGCCTTATTCCTCCTTAGAACAATTCCTAAATCTGTAACTTA
AGTTTCTCAGGAAGATTCCATACTGCACAGAAAACTGCTTTTGTGGGTTTTTAAAAGGCAAGTTGTTATATG
TGCTGGATAGTTTTTAAGTATGACATAAAAATTGTATAAAGTAAAATATTAAAATACACCTAGAATACTGTA
TAACTTTAAGTCATTTTATCAACACATTGCTAATCCAGATATTTTCCCGCAGTTTTTCTTTGAATAACAGAG
CAATTAATTTACTTTTACTATGAAGAGTCATCATTTTAGTATGTATTTTAAGCAATCCACCAAGAACTCAGT
AGGCAGCTGAGAGGTGCTGCCCAGAGAAGTGGTGATTAGCTTGGCCTTAGCTCACCCACACAAAGCACAACA
GGCTTTGAACTATTCCCTAACGGGGCATTTATTCTTTTTTTTTTTTTTTTTTGGGAGACGGAGTCTCGCTGT
CGCCCAGGCTAGAGTGCAGTGGCGCGATCTCGGCTCACTGCAGGCTCCACCCCCTGGGGTTCACGCCATTCT
CCTGCCTCAGCCTCCCAAGTAGCTGGGACTGCAGGCGCCCGCCATCTCGCCCGGCTAATTTTTTGTATTTTT
AGTAGAGACGGGGTTTCACCGTGTTAGCCAGGATAGGGCATTTATTCTTGAACTTGATTCAGAGAGGCACAC
ATTACCATTCTCTAATCAGAATGCAAGTAGCGCAAGGCGGTGGAAACTATGGAATTCGGAGGCAGGTGATGC
ATTGGGCGAGTTTATTAACATCTGTGACTCTCTAGTTTGAAATTTATTTGTAACAGACAAAAATGAATTAAA
CAAACAATAAAAGTATAATAAAGAA

SEQ ID NO: 5: human CD38 amino acid sequence.

(SEQ ID NO: 5)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution T237A in the Daratumumab epitope. The substitution is in bold. The three-dimensional structure including this amino acid substitution is shown in FIG. 2

(SEQ ID NO: 6)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQALEAWVIHG
GREDSRDLCQDPTIKELESIISKRNIQFSCKNIYRPDKFLQCVKNPEDS
SCTSEI

Sequence of CD38 containing the substitution E239F in the Daratumumab epitope. The substitution is in bold. The three-dimensional structure including this amino acid substitution is shown in FIG. 2

(SEQ ID NO: 7)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLFAWVIHG
GREDSRDLCQDPTIKELESIISKRNIQFSCKNIYRPDKFLQCVKNPEDS
SCTSEI

Sequence of CD38 containing the substitution Q272R in the Daratumumab epitope. The substitution is in bold. The three-dimensional structure including this amino acid substitution is shown in FIG. 2.

(SEQ ID NO: 8)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIRFSCKNIYRPDKFLQCVKNPEDS
SCTSEI

Sequence of CD38 containing the substitution S274F in the Daratumumab epitope. The substitution is in bold. The three-dimensional structure including this amino acid substitution is shown in FIG. 2.

(SEQ ID NO: 9)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFFCKNIYRPDKFLQCVKNPEDS
SCTSEI

Sequence of CD38 containing the substitution K276F in the Daratumumab epitope. The substitution is in bold. The three-dimensional structure including this amino acid substitution is shown in FIG. 2.

(SEQ ID NO: 10)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCFNIYRPDKFLQCVKNPEDS
SCTSEI

Sequence of CD38 containing the substitution M77F in the Isatuximab epitope. The substitution is in bold.

(SEQ ID NO: 11)
MANCEFSPVSGDKPCCRLSRRAQLCLGVSILVLILVVVLAVVVPRWRQQ
WSGPGTTKRFPETVLARCVKYTEIHPEFRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution R78F in the Isatuximab epitope. The substitution is in bold.

(SEQ ID NO: 12)
MANCEFSPVSGDKPCCRLSRRAQLCLGVSILVLILVVVLAVVVPRWRQQ
WSGPGTTKRFPETVLARCVKYTEIHPEMFHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution H79F in the Isatuximab epitope. The substitution is in bold.

(SEQ ID NO: 13)
MANCEFSPVSGDKPCCRLSRRAQLCLGVSILVLILVVVLAVVVPRWRQQ
WSGPGTTKRFPETVLARCVKYTEIHPEMRFVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution V80F in the Isatuximab epitope. The substitution is in bold.

(SEQ ID NO: 14)
MANCEFSPVSGDKPCCRLSRRAQLCLGVSILVLILVVVLAVVVPRWRQQ
WSGPGTTKRFPETVLARCVKYTEIHPEMRHFDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution K111F in the Isatuximab epitope. The substitution is in bold.

(SEQ ID NO: 15)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMFLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution L12F in the Isatuximab epitope. The substitution is in bold.

(SEQ ID NO: 16)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKFGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution G113F in the Isatuximab epitope. The substitution is in bold.

(SEQ ID NO: 17)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLFTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution T114F in the Isatuximab epitope. The substitution is in bold.

(SEQ ID NO: 18)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGFQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution Q115F in the isatuximab epitope. The substitution is in bold.

(SEQ ID NO: 19)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTFTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution Ti 16F in the isatuximab epitope. The substitution is in bold.

(SEQ ID NO: 20)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQFVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution V117F in the isatuximab epitope. The substitution is in bold.

(SEQ ID NO: 21)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTFPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution P118F in the Isatuximab epitope. The substitution is in bold.

(SEQ ID NO: 22)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVFCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

Sequence of CD38 containing the substitution P232F in the Isatuximab epitope. The substitution is in bold.

(SEQ ID NO: 23)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQFEKVQTLEAWVIHG
GREDSRDLCQDPTIKELESIISKRNIQFSCKNIYRPDKFLQCVKNPEDS
SCTSEI

Sequence of CD38 containing the substitution E233F in the Isatuximab epitope. The substitution is in bold.

(SEQ ID NO: 24)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPFKVQTLEAWVIHG
GREDSRDLCQDPTIKELESIISKRNIQFSCKNIYRPDKFLQCVKNPEDS
SCTSEI

Sequence of CD38 containing the substitution K234F in the Isatuximab epitope. The substitution is in bold.

(SEQ ID NO: 25)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEFVQTLEAWVIHG
GREDSRDLCQDPTIKELESIISKRNIQFSCKNIYRPDKFLQCVKNPEDS
SCTSEI

NK cell CD38 knock out was generated by lentiviral transduction of Cas9 and gRNA targeting the first intron (5′ TGTACTTGACGCATCGCGCCAGG 3′ (SEQ ID NO: 52)). Other gRNAs have also been tested and can be used in this protocol.

NK92 CD38KO cells were further modified to add the different mutated version of CD38 (CD38-Codon optimized not recognized by gRNA (CD38-CO), CD38 S274F, E239F, Q272R, T237A, K276F with the first letter being the original amino acid, the number the position on the protein chain, and last letter the substitution of amino acid. TG2 was the empty vector used as control (GFP only). All plasmids were designed by the inventors, and the cloning was ordered and performed by GenScript.

Lentiviruses were generated by calcium-phosphate based transfection (Sigma, CAPHOS-1KT) according to the manufacturer's recommendations in HEK293FT cells between passage number 3-15. DMEM complete medium was used for the duration of the experiment, consisting of 500 ml DMEM (Gibco), containing 55 ml FBS (Gibco), 5.5 ml L-glutamine solution (Sigma), 5.5 ml Sodium pyruvate solution (Sigma), 5.5 ml Non-essential amino acid solution (Sigma), 11 ml HEPES solution (Gibco). Cells were seeded in Poly-D-Lysine coated 150 mm dish (BD Biosciences). After 6 h of incubation at 37° C. 5% CO₂, medium was replaced by 20 ml complete medium containing 25 μM of Chloroquine (Sigma), and 1 ml of 2×HeBS (Sigma) buffer with 1 ml of the plasmids of interest were co-transfected with the envelope plasmid pCMV-VSV-G and two packaging plasmids, pDMLg/pPRE and pRSV-Rev to produce VSV-G-pseudotyped lentiviruses.plasmid solution (30 μg of vector, 15 μg of Gag/pol, 10 μg envelope, 0.25M CaCl₂) (Sigma) and ddH₂O to 1000 μl). After 16 h of incubation change medium with 20 ml of fresh complete medium, and after 24 h collect medium for viral collection by filtering the supernatant in 0.45 μm filter (Milipore) using a 20 ml syringe. After another 24 h, a second harvest was made, and Lenti-X concentrator (Takara Bio) was added to total content of viral supernatant from both harvests in 1 volume of Lenti-X for 3 volumes of supernatant. After 6 h of in the fridge, viral solutions were centrifuged at 1500 g for 45 min (no brake) at 4° C. Viruses were resuspended in PBS 5% FBS at 1/10 or 1/50 of original volume.

Other viruses then Lentivirus, e.g. alpha Retrovirus, gamma Retrovirus, Adenovirus or AAV can be used for transduction.

Lentivirus or other viruses can be pseudotyped with envelope proteins other than VSV-G, e.g. Baboon env, RD114, GALV, or even engineered and chimeric envelope proteins.

The Virus titer was measured by transducing HEK293FT cells with different volumes of viral solution. HEK293FT cells were seeded at 50 000 cells per well in 24 well plate (Corning) in DMEM+ Glutamax medium (Gibco)+8 μg/ml of protamine sulfate (Sigma Aldrich). Viral solution was added and diluted in a serial solution followed by a centrifugation at 1000 g for 1 h (no brake) at 32° C. The plate was incubated for 6 h at 37° C. 5% CO₂ before changing the medium to DMEM without protamine sulfate. After 3 days, transduction efficiency was assessed by flowcytometry after fixation of cells in 1% PFA 5 min at room temperature, for GFP reporter carrying construct, or after staining and fixation when applicable.

$$Calculation of the titre was T = \frac{( \% construct expressing cells ⋆ Number of cells seeded )}{volume of viral solution added} .$$

Titer had been calculated from wells showing between 5% and 20% positive cells.

For the transduction of NK92 cells, 30 000 cells were seeded in a flat bottom 96 well plate (Corning) supplemented with 8 μg/ml of protamine sulfate (Sigma Aldrich) and 7 μg/ml of Vy-OZ (Vycellix) and viral solution at a MOI of 8. The plate was then centrifuged at 1000 g for 1 h (no brake) 32° C., followed by 5 h at 37° C. 5% CO₂ before media change with IL2 addition (500 U/ml final concentration). Every 2 days 1L2 was added, and media changed until assessment of expression by flow cytometry (3-5 days post transduction), and proliferation for sorting to get pure transduced cells.

PBMC and isolated primary NK cells were transduced as above but using MOI=15. Either 30000 cells in flat 96 well plate or 250000 cells in 24 well plate (Corning) were transduced.

Peripheral blood mononuclear cells (PBMCs) were isolated from buffy coats by gradient centrifugation, using Lymphoprep (Stemcell technologies) gently mixed with buffy coat (1 part of lymphoprep for 2 parts of buffy coat) followed by centrifugation at 800 g for 30 min (no brake) 20° C. and harvest of the white-blood-cell-band. Subsequently, 2 washes with phosphate-buffered saline (PBS; Gibco) have been performed, with centrifugation for 10 min at 300×g between the 2 washes, and 200×g, 10 min after the second wash. Cell viability was assessed by trypan blue exclusion. If necessary, PBMCs were directly frozen in human serum albumin containing 10% DMSO for subsequent phenotyping and cytotoxicity experiments. Primary NK were isolated using the Miltenyi Biotec NK cell isolation kit Human, according to the manufacturer's instructions. Phenotyping was done by surface staining using live dead marker (Invitrogen) and CD3, CD56 after 20 min of incubation in the fridge before wash in PBS and fixation in 1% PFA 5 min room temperature in the dark. Analysis was done by flowcytometry.

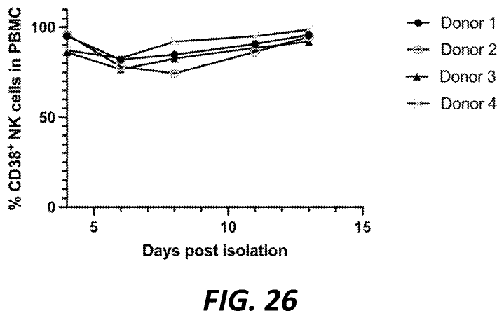

For in vitro expansion of NK cells in PBMCs, the PBMC were seeded directly after the Lymphoprep isolation at 0.5*10⁶ cells/ml in SCGM medium (CellGenix) supplemented with 5% Human Serum, 500 U/ml of IL2 and 10 ng/ml CD3 antibody. Every following day for 5 days IL2 500 U/ml was added and after day 5 IL-2 was added 3 times per week. FIG. 26 shows CD38 expression remains detectable during 14 days of expansion.

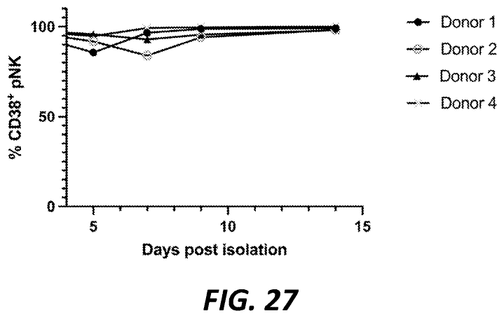

For ex vivo expansion of isolated primary NK cells, 1*10⁶ cells/ml were seeded in SCGM medium (CellGenix) supplemented with 10% Human Serum, 1000 U/ml of IL2 and 20 ng/ml IL21, directly after isolation. Every following day for 5 days 1L2 500 U/ml was added and after day 5 IL-2 was added 3 times per week. FIG. 27 shows CD38 expression remains detectable during 14 days of expansion.

To test which substitutions can be best used in therapy, several in vitro assays can be employed (FIG. 5). In order to be able to use cells with these modified CD38 molecules clinically, one has to determine that the cells are no longer recognized by daratumumab and that the cells are still functional in terms of degranulation/killing target cells. It can be assumed that avoidance of recognition by daratumumab leads to inertness to ADCC, as ADCC is only possible if binding of the antibody to the antigen epitope is a) happening and b) of sufficiently high affinity. Results of shielding from Daratumumab binding are shown in FIGS. 6-8 and 13-14, while assessment of functionality of the modified cells is shown in FIGS. 17-24.

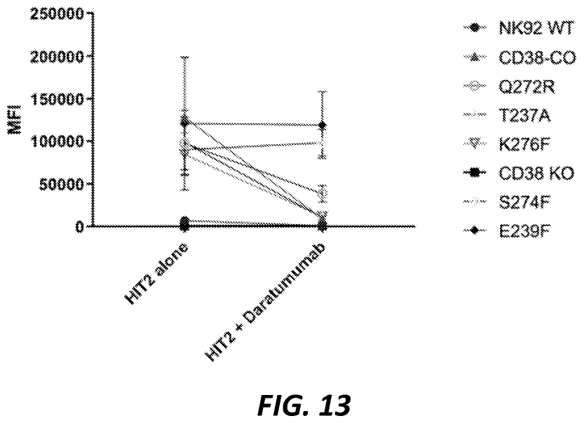

Recognition by daratumumab: Once the endogenous CD38 is removed and the mutated CD38 is expressed, binding studies using daratumumab and other CD38 antibodies have been performed to assess recognition of the mutated epitope by daratumumab. Results are shown in FIGS. 6-8 and 13-14 for Daratumumab, and 15-16 for Isatuximab. For this, the modified cells are incubated for 40 minutes with daratumumab (c=10 μg/ml) at 4° C. Excess daratumumab is removed by washing the cells 2 times with PBS+2% FBS. Then, the cells are incubated with a secondary fluorescently-labeled antibody recognizing the Fc-domain of daratumumab. This allows visualization of cells that are recognized by daratumumab. After removal of excess secondary antibody, the cells are fixed with 1% PFA for 5 minutes at 4° C., washed, and resuspended in PBS. Analysis of CD38 expression is done by flow cytometry. Alternatively, Daratumumab can be tagged with a fluorophore prior to CD38 staining of the cells. Alternatively, the cells are first incubated with Daratumumab, and subsequently stained with anti-CD38 antibodies that recognize a different epitope then daratumumab, to determine if the pre-incubation leads to a reduction of binding of the second antibody, thus proving binding competition (FIGS. 13-16). Optionally, the cells can be stained with several anti-CD38 antibodies, including daratumumab, simultaneously, to allow for detection of total CD38 versus mutated CD38. Sorted CD38 NK cells, wild type, and CD38KO cells were seeded at 80000 cells/well in a V-bottom 96 well plate (Corning). Daratumumab was added to cells at 55 μg/ml (decided upon titration) in 50 μl PBS (Sigma). Incubation was 40 minutes in the fridge followed by two washes with PBS and incubation with a secondary antibody anti-Fc (Cedarlane) used a 1/100. The commercial monoclonal anti CD38 HIT2 was used as control to assess the presence of CD38 surface expression. Cells were fixed in 1% PFA at room temperature and analysed by flowcytometry (CytoflexS Beckmann coulter). Isatuximab was also used to assess exclusivity of our mutants following the same protocol but at 4 μg/ml (decided upon titration).

Functionality of the modified NK cells: Cells that express the mutated CD38, will then be assessed for functionality. For this, several functional assays can be employed. The most common one is a flow cytometry-based in vitro responsiveness assay for determination of degranulation (measuring CD107 that is accessible during release of cytotoxic granules) and cytokine production (IFNgamma is commonly used as standard cytokine for NK cells). For the in vitro responsiveness assay, modified NK cells are co-incubated with target cells at an effector to target ratio (E:T ratio) of 1:1 or 1:3 for four to six hours or overnight. During the incubation, a fluorescently-labeled antibody targeting CD107 is present. CD107 is a membrane protein that resides in intracellular vesicles. When NK cells degranulate, the CD107 molecule can be bound by the CD107-targeting antibody, which enables subsequent detection of degranulated NK cells by flow cytometry. After the co-incubation, the modified NK cells are further stained for surface and intracellular markers, such as CD3, CD56, CD38, live-dead cell marker, as well as intracellular cytokines such as INFgamma. Flow cytometric analysis allows assessment of the percentage of NK cells that respond with either degranulation of cytokine response, shown in FIGURE S 17-23. In addition, the extent of the response can be measured, by comparing the mean fluorescent intensity (MFI) of CD107 or IFNgamma. NK cells (cell line, PBMCs or isolated NK cells) were coincubated with target cells at a ratio of 1:1 in a final volume of 200 μL in round-bottomed 96-well plates at 37° C. and 5% CO₂ for 4 hours. Fluorochrome-conjugated anti-CD107a mAb was added at the initiation of the assay. As controls, 100 000 effector cells were incubated alone or with phorbol 12-myristate 13-acetate (PMA, at 50 ng/ml, Sigma-Aldrich) and ionomycin (500 ng/mL, Sigma-Aldrich). After 1 hour of coincubation, Monensin (GolgiStop; BD Biosciences) was added at a 1:300 dilution to inhibit protein transportation. Surface staining was done 4 h after the beginning of the assay by incubating cells with selected antibodies 30 minutes for in the fridge followed by permeabilization by Cytofix/Cytoperm (BD) for intracellular staining of IFNgamma. The cells were then washed, resuspended in PBS, and fixed by 5 minute incubation with 1% PFA. The samples were analyzed with Beckman Coulter Cytoflex or BD Symphony flow cytometers.

Inertness of modified NK cells towards ADCC: Resistance of modified NK cells to antibody-mediated cytotoxicity (ADCC) by other immune effector cells, can be measured by using the modified NK cells as target cells. For this, the in vitro responsiveness assay described above can be modified to include the antibody of interest, daratumumab in this case, during the co-incubation of effector and target cells. By using unmodified NK cells or macrophages as effectors, and either modified or control-engineered NK cells as target cells, on can assess the level of ADCC the modified NK cell evoke in other immune cells, ie the level of resistance to NK-cell mediated ADCC or macrophage-mediated ADCC or ADCP. By assessing degranulation (CD107 expression) and cytokine response (IFNgamma expression) of unmodified effector NK cells towards modified target NK cells, we hope to show that the inserted CD38 mutations lead to resistance to daratumumab-mediated ADCC. In addition to measuring response of effector cells, one can also measure killing of modified target cells directly. For this, we will stain the modified target NK cells after co-incubation with propidium iodide (PI) and Annexin V (AnnV)-antibody, to distinguish apoptotic and necrotic cell death of the modified NK cells. Since inertness to ADCC is inherently dependent on evasion of antibody (daratumumab) recognition, we are certain that once the cells are shielded from recognition by daratumumab, they will also be inert to ADCC.

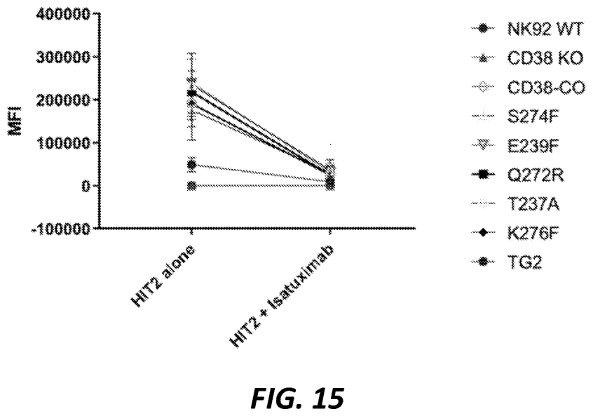

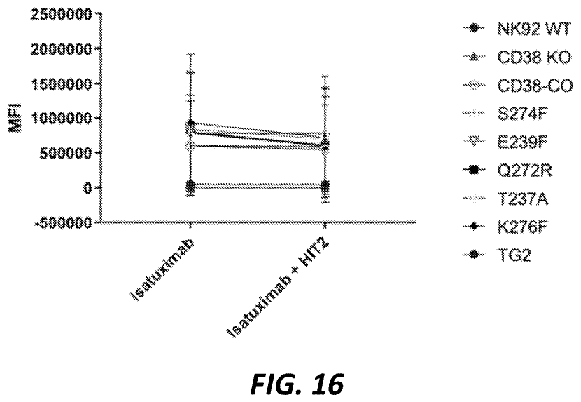

The criteria to what constitutes a good/optimal amino acid replacement in CD38 are 1) that the modified NK cells are not recognized by daratumumab, but can be stained with anti-CD38 antibodies that are specific for epitopes different from daratumumab-epitope (shown in FIGS. 6-8 and 13-14), 2) that the modified NK cells still recognized by other clinically relevant antibodies targeting the same antigen, such as Isatuximab (ie that the modification is specific for only one of the antibodies, Daratumumab in this case), as shown in FIGS. 15-16, and that the modified NK cells are functional in terms of degranulation (as surrogate marker for killing) and IFNgamma (shown in FIGS. 17-23). However, these criteria are not absolute, meaning that if modified NK cells are still recognized to a small degree by daratumumab, this does not exclude this specific modification, as they may be spared in the competitive environment, where cells with high expression of wt/endogenous CD38 will be preferentially killed (33), and the cells with low recognition may be ultimately spared in the patient. In addition, since inertness to ADCC is inherently dependent on evasion of antibody (daratumumab) recognition, we assume that once the cells are shielded from recognition by daratumumab, they will also be inert to ADCC. Hence ADCC does not need to be tested in every setting.

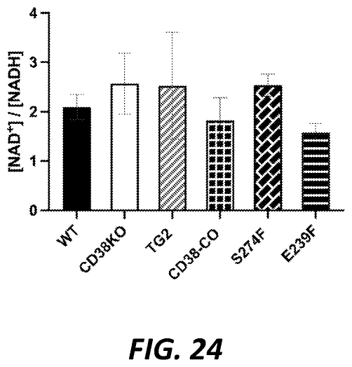

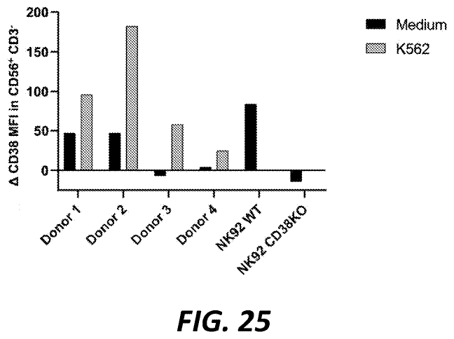

Modified CD38 molecules harbouring the amino acid changes, are also able to perform their enzymatic function. This is depicted in FIG. 24, showing that CD38KO and CD38KO+TG2 (empty vector control) use less NAD+ as substrate, thus elevating the NAD+/NADH ratio. The CD38-codon optimized (CD38-CO), and the E239F modified CD38 molecules on the other hand reduce the NAD+/NADH ratio, confirming enzymatic function of the cells harbouring the modified CD38 molecules. This assay is performed according to the manufacturer instruction (Abcam, ab65348). One million cells were collected, washed with PBS and followed by extraction of NAD+ and NADH with extraction buffer in 2 cycles of freeze/thaw (20 min dry ice, 10 min RT). Then after centrifugation the supernatant is divided in two 1.5 ml Eppendorf tubes, one for NADtotal, the other for NADH alone. NADH alone is heated at 60° C. on heating block for 30 min while NADtotal remains on ice in the dark. After 30 min, the samples should are transferred to a flat transparent 96 well plate (20 μl for NADtotal, 30 μl for NADH up to 50 μl with extraction buffer). Cycling enzyme was then added to the solution to convert NAD+ in NADH in NADtotal sample: after 10 min incubation, 10 μl of developer solution was added followed by 1 h30 of incubation at RT covered with aluminum foil. Analysis was done by absorbance at 450 nm on Tecan FT500. Absorbance of standard value at 0 pmol was subtracted from all samples. Standard curve was used to calculate the quantity in pmol of NADH in the samples. The quantity per well was divided by the volume added in the well to have the concentration in pmol/μl. NAD+ quantity was calculated as follow: NAD+=NADtotal− NADH.

### Example 2.1 CD47 and Magrolimab

CD47 is a target for immunotherapy in solid cancers and heamatological malignancies, including MDS and AML. There are >23 therapeutic agents targeting CD47, one of them being the monoclonal antibody magrolimab, which is currently being tested in 27 clinical trials against various cancers. CD47 is a transmembrane protein ubiquitously expressed on human cells and overexpressed in many types of cancer cells for which it is important for the development and progression of cancer. CD47 protects cells from phagocytosis by binding to SIRPalpha on macrophages, triggering a “don't-eat-me” signal, which inhibits phagocytosis. Many CD47 mAbs not only block CD47 from binding to SIRPalpha, but simultaneously trigger Fc Receptor gamma on macrophages, which acts as an “eat-me” signal, thus delivering a potent signal to the macrophages to destroy the tumor cells via ADCP.

cDNA Sequence of CD47
Nucleotide Sequence (918): cDNA

(SEQ ID NO: 26)
ATGTGGCCCCTGGTAGCGGCGCTGTTGCTGGGCTCGGCGTGCTGCGGATCAGCTCAGCTA
CTATTTAATAAAACAAAATCTGTAGAATTCACGTTTTGTAATGACACTGTCGTCATTCCA
TGCTTTGTTACTAATATGGAGGCACAAAACACTACTGAAGTATACGTAAAGTGGAAATTT
AAAGGAAGAGATATTTACACCTTTGATGGAGCTCTAAACAAGTCCACTGTCCCCACTGAC
TTTAGTAGTGCAAAAATTGAAGTCTCACAATTACTAAAAGGAGATGCCTCTTTGAAGATG
GATAAGAGTGATGCTGTCTCACACACAGGAAACTACACTTGTGAAGTAACAGAATTAACC
AGAGAAGGTGAAACGATCATCGAGCTAAAATATCGTGTTGTTTCATGGTTTTCTCCAAAT
GAAAATATTCTTATTGTTATTTTCCCAATTTTTGCTATACTCCTGTTCTGGGGACAGTTT
GGTATTAAAACACTTAAATATAGATCCGGTGGTATGGATGAGAAAACAATTGCTTTACTT
GTTGCTGGACTAGTGATCACTGTCATTGTCATTGTTGGAGCCATTCTTTTCGTCCCAGGT
GAATATTCATTAAAGAATGCTACTGGCCTTGGTTTAATTGTGACTTCTACAGGGATATTA
ATATTACTTCACTACTATGTGTTTAGTACAGCGATTGGATTAACCTCCTTCGTCATTGCC
ATATTGGTTATTCAGGTGATAGCCTATATCCTCGCTGTGGTTGGACTGAGTCTCTGTATT
GCGGCGTGTATACCAATGCATGGCCCTCTTCTGATTTCAGGTTTGAGTATCTTAGCTCTA
GCACAATTACTTGGACTAGTTTATATGAAATTTGTGGCTTCCAATCAGAAGACTATACAA
CCTCCTAGGAATAACTGA

Amino Acid sequence of CD47. The binding site (epitope) of Magrolimab is in bold.

### Translation (305 aa):

(SEQ ID NO: 27)
MWPLVAALLLGSACCGSAQLLFNKTKSVEFTFCNDTVVIPCFVTNMEAQNTTEVYVKWKF
KGRDIYTFDGALNKSTVPTDFSSAKIEVSQLLKGDASLKMDKSDAVSHTGNYTCEVTELT
REGETIIELKYRVVSWESPNENILIVIFPIFAILLFWGQFGIKTLKYRSGGMDEKTIALL
VAGLVITVIVIVGAILFVPGEYSLKNATGLGLIVTSTGILILLHYYVFSTAIGLTSFVIA
ILVIQVIAYILAVVGLSLCIAACIPMHGPLLISGLSILALAQLLGLVYMKFVASNQKTIQ
PPRNN

Sequence of CD47 containing the substitution Q1F in the Magrolimab epitope. The substitution is in bold. The position of amino acids is counted from the protein after removal of the leader peptide.

(SEQ ID NO: 28)
MWPLVAALLLGSACCGSAFLLFNKTKSVEFTFCNDTVVIPCFVTNMEAQNTTEVYVKWKF
KGRDIYTFDGALNKSTVPTDFSSAKIEVSQLLKGDASLKMDKSDAVSHTGNYTCEVTELT
REGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVFSTAIGLTSFVIA
ILVIQVIAYILAVVGLSLCIAACIPMHGPLLISGLSILALAQLLGLVYMKFVASNQKTIQ
PPRNN

Sequence of CD47 containing the substitution L2F in the Magrolimab epitope. The substitution is in bold. The position of amino acids is counted from the protein after removal of the leader peptide.

(SEQ ID NO: 29)
MWPLVAALLLGSACCGSAQFLFNKTKSVEFTFCNDTVVIPCFVTNMEAQNTTEVYVKWKF
KGRDIYTFDGALNKSTVPTDFSSAKIEVSQLLKGDASLKMDKSDAVSHTGNYTCEVTELT
REGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVFSTAIGLTSFVIA
ILVIQVIAYILAVVGLSLCIAACIPMHGPLLISGLSILALAQLLGLVYMKFVASNQKTIQ
PPRNN

Sequence of CD47 containing the substitution L3F in the Magrolimab epitope. The substitution is in bold. The position of amino acids is counted from the protein after removal of the leader peptide.

(SEQ ID NO: 30)
MWPLVAALLLGSACCGSAQLFFNKTKSVEFTFCNDTVVIPCFVTNMEAQNTTEVYVKWKF
KGRDIYTFDGALNKSTVPTDFSSAKIEVSQLLKGDASLKMDKSDAVSHTGNYTCEVTELT
REGETIIELKYRVVSWFSPNENILIVIFPIFAILLFWGQFGIKTLKYRSGGMDEKTIALL
VAGLVITVIVIVGAILFVPGEYSLKNATGLGLIVISTGILILLHYYVFSTAIGLTSFVIA
ILVIQVIAYILAVVGLSLCIAACIPMHGPLLISGLSILALAQLLGLVYMKFVASNQKTIQ
PPRNN

Sequence of CD47 containing the substitution T34F in the Magrolimab epitope. The substitution is in bold. The position of amino acids is counted from the protein after removal of the leader peptide.

(SEQ ID NO: 31)
MWPLVAALLLGSACCGSAQLLFNKTKSVEFTFCNDTVVIPCFVTNMEAQNTFEVYVKWKF
KGRDIYTFDGALNKSTVPTDFSSAKIEVSQLLKGDASLKMDKSDAVSHTGNYTCEVTELT
REGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVFSTAIGLTSFVIA
ILVIQVIAYILAVVGLSLCIAACIPMHGPLLISGLSILALAQLLGLVYMKEVASNQKTIQ
PPRNN

Sequence of CD47 containing the substitution E35F in the Magrolimab epitope. The substitution is in bold. The position of amino acids is counted from the protein after removal of the leader peptide.

(SEQ ID NO: 32)
MWPLVAALLLGSACCGSAQLLFNKTKSVEFTFCNDTVVIPCFVTNMEAQNTTFVYVKWKF
KGRDIYTFDGALNKSTVPTDFSSAKIEVSQLLKGDASLKMDKSDAVSHTGNYTCEVTELT
REGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVFSTAIGLTSFVIA
ILVIQVIAYILAVVGLSLCIAACIPMHGPLLISGLSILALAQLLGLVYMKFVASNQKTIQ
PPRNN

Sequence of CD47 containing the substitution V36F in the Magrolimab epitope. The substitution is in bold. The position of amino acids is counted from the protein after removal of the leader peptide.

(SEQ ID NO: 33)
MWPLVAALLLGSACCGSAQLLFNKTKSVEFTFCNDTVVIPCFVTNMEAQNTTEFYVKWKF
KGRDIYTFDGALNKSTVPTDFSSAKIEVSQLLKGDASLKMDKSDAVSHTGNYTCEVTELT
REGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVFSTAIGLTSFVIA
ILVIQVIAYILAVVGLSLCIAACIPMHGPLLISGLSILALAQLLGLVYMKFVASNQKTIQ
PPRNN

Sequence of CD47 containing the substitution E97F in the Magrolimab epitope. The substitution is in bold. The position of amino acids is counted from the protein after removal of the leader peptide.

(SEQ ID NO: 34)
MWPLVAALLLGSACCGSAQLLFNKTKSVEFTFCNDTVVIPCFVTNMEAQNTTEVYVKWKF
KGRDIYTFDGALNKSTVPTDFSSAKIEVSQLLKGDASLKMDKSDAVSHTGNYTCFVTELT
REGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVFSTAIGLTSFVIA
ILVIQVIAYILAVVGLSLCIAACIPMHGPLLISGLSILALAQLLGLVYMKFVASNQKTIQ
PPRNN

Sequence of CD47 containing the substitution V98F in the Magrolimab epitope. The substitution is in bold. The position of amino acids is counted from the protein after removal of the leader peptide.

(SEQ ID NO: 35)
MWPLVAALLLGSACCGSAQLLFNKTKSVEFTFCNDTVVIPCFVTNMEAQNTTEVYVKWKF
KGRDIYTFDGALNKSTVPTDFSSAKIEVSQLLKGDASLKMDKSDAVSHTGNYTCEFTELT
REGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVFSTAIGLTSFVIA
ILVIQVIAYILAVVGLSLCIAACIPMHGPLLISGLSILALAQLLGLVYMKFVASNQKTIQ
PPRNN

Sequence of CD47 containing the substitution T99F in the Magrolimab epitope. The substitution is in bold. The position of amino acids is counted from the protein after removal of the leader peptide.

(SEQ ID NO: 36)
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVFELTREGETIIELKYRVVSWFSPNENILIVIFPI
FAILLFWGQFGIKTLKYRSGGMDEKTIALLVAGLVITVIVIVGAILFVPG
EYSLKNATGLGLIVISTGILILLHYYVESTAIGLTSFVIAILVIQVIAYI
LAVVGLSLCIAACIPMHGPLLISGLSILALAQLLGLVYMKFVASNQKTIQ
PPRNN

Sequence of CD47 containing the substitution E100F in the Magrolimab epitope. The substitution is in bold. The position of amino acids is counted from the protein after removal of the leader peptide.

(SEQ ID NO: 37)
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTFLTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRNN

Sequence of CD47 containing the substitution L101F in the Magrolimab epitope. The substitution is in bold. The position of amino acids is counted from the protein after removal of the leader peptide.

(SEQ ID NO: 38)
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTEFTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRNN

Sequence of CD47 containing the substitution T102F in the Magrolimab epitope. The substitution is in bold. The position of amino acids is counted from the protein after removal of the leader peptide.

(SEQ ID NO: 39)
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELFREGETIIELKYRVVSWFSPNENILIVIFPI
FAILLFWGQFGIKTLKYRSGGMDEKTIALLVAGLVITVIVIVGAILFVPG
EYSLKNATGLGLIVISTGILILLHYYVFSTAIGLTSFVIAILVIQVIAYI
LAVVGLSLCIAACIPMHGPLLISGLSILALAQLLGLVYMKFVASNQKTIQ
PPRNN

Sequence of CD47 containing the substitution R103F in the Magrolimab epitope. The substitution is in bold. The position of amino acids is counted from the protein after removal of the leader peptide.

(SEQ ID NO: 40)
MWPLVAALLLGSACCGSAQLLFNKTKSVEFTFCNDTVVIPCFVTNMEAQN
TTEVYVKWKFKGRDIYTFDGALNKSTVPTDESSAKIEVSQLLKGDASLKM
DKSDAVSHTGNYTCEVTELTFEGETIIELKYRVVSWFSPNENILIVIFPI
FAILLFWGQFGIKTLKYRSGGMDEKTIALLVAGLVITVIVIVGAILFVPG
EYSLKNATGLGLIVISTGILILLHYYVFSTAIGLTSFVIAILVIQVIAYI
LAVVGLSLCIAACIPMHGPLLISGLSILALAQLLGLVYMKEVASNQKTIQ
PPRNN

Sequence of CD47 containing the substitution E104F in the Magrolimab epitope. The substitution is in bold. The position of amino acids is counted from the protein after removal of the leader peptide.

(SEQ ID NO: 41)
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTRFGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRNN

### Example 2.2: CD52 and Alemtuzumab

Alemtuzumab is an important antibody in the therapy of relapsing remittent multiple sclerosis (RRMS) under the trademark Lemtrada. Recently, it is being used under the trademark CAMPATH-1HH for chronic lymphocytic leukemia. It has also been used against T cell lymphoma, non-Hodgkin's lymphoma and rheumatoid arthritis. The mechanisms of action include NK cell-mediated ADCC and complement-dependent cytotoxicity (CDC) and a direct apoptotic effect. The antibody binds to CD52, which is expressed at high concentrations in lymphoma. However, it is also expressed on cells of the immune system, including B cells, T cells, NK cells, Monocytes, Maccrophages, despite its clear successes, alemtuzumab has also been shown to result in substantial toxicity due to attendant immunosuppression associated with its use, and in particular, increased risk of viral and other opportunistic infections, most likely due to a depletion of immune cells.

CD52 is a molecule of only 12 amino acids, which are linked to a glycosylphospatidylinositol (GPI) anchor to the membrane. The exact biological function of CD52 is so far unclear, but some evidence points to a function in T cell migration and co-stimulation. The critical amino acids for recognition by alemtuzumab are the C-terminal amino acids (QTSSPS). The protein is heavily glycosylated, but the glycosylations do not seem to impact binding of alemtuzumab.

Nucleotide Sequence (186 nt): cDNA

(SEQ ID NO: 42)
ATGAAGCGCTTCCTCTTCCTCCTACTCACCATCAGCCTCCTGGTTATGGT
ACAGATACAAACTGGACTCTCAGGACAAAACGACACCAGCCAAACCAGCA
GCCCCTCAGCATCCAGCAACATAAGCGGAGGCATTTTCCTTTTCTTCGTG
GCCAATGCCATAATCCACCTCTTCTGCTTCAGTTGA

Translation (61 Aa): (with Leader Peptide and Before Post-Translational Modifications)

(SEQ ID NO: 43)
MKRFLFLLLTISLLVMVQIQTGLSGQNDTSQTSSPSASSNISGGIFLFFV
ANAIIHLFCFS

Sequence of the 12-peptide final CD52 protein. These are amino acids 25-36 of the peptide before post-translational modifications and without the leader peptide. The Alemtuzumab epitope is in bold.

(SEQ ID NO: 44)
GQNDTSQTSSPS

Sequence of CD52 containing the substitution Q31F in the Alemtuzumab epitope. The substitution is in bold. The position of amino acids is counted from the protein containing the leader peptide and before post-translational modifications.

(SEQ ID NO: 45)
GQNDTSFTSSPS

Sequence of CD52 containing the substitution T32F in the Alemtuzumab epitope. The substitution is in bold. The position of amino acids is counted from the protein containing the leader peptide and before post-translational modifications.

(SEQ ID NO: 46)
GQNDTSQFSSPS

Sequence of CD52 containing the substitution S33F in the Alemtuzumab epitope. The substitution is in bold. The position of amino acids is counted from the protein containing the leader peptide and before post-translational modifications.

(SEQ ID NO: 47)
GQNDTSQTFSPS

Sequence of CD52 containing the substitution S34F in the Alemtuzumab epitope. The substitution is in bold. The position of amino acids is counted from the protein containing the leader peptide and before post-translational modifications.

(SEQ ID NO: 48)
GQNDTSQTSFPS

Sequence of CD52 containing the substitution P35F in the Alemtuzumab epitope. The substitution is in bold. The position of amino acids is counted from the protein containing the leader peptide and before post-translational modifications.

(SEQ ID NO: 49)
GQNDTSQTSSFS

Sequence of CD52 containing the substitution S36F in the Alemtuzumab epitope. The substitution is in bold. The position of amino acids is counted from the protein containing the leader peptide and before post-translational modifications.

(SEQ ID NO: 50)
GQNDTSQTSSPF

### Example 3: MM Patient Receiving Daratumumab and Autologous Expanded GEAR NK Cells (CD38-GEAR-NK)

Following the Examples 1-2, and referring to FIG. 10, a patient is diagnosed with MM. He/she receives the standard treatment of autologous stem cell transplantation (auto-SCT) where hematopoietic stem cells are harvested from the blood either by apheresis or by removing bone marrow from the pelvic bone (iliac crest) with a special needle. During apheresis, NK cells are also harvested from the blood. After harvesting the hematopoietic stem cells, the patient receives high-dose chemotherapy and is then re-infused with his/her own stem cells. These stem cells can repopulate all blood cell lineages. The harvested NK cells will be expanded in an enclosed, automated in vitro expansion procedure under GMP conditions and genetically modified with one of the constructs identified and described in Example 2 above. Successfully modified NK cells will express a CD38 variant that has a small change in the amino acid sequence and is thus no longer recognized by the therapeutic anti-CD38 mAb Daratumumab. The expanded NK cells are tested for activity, quality and sterility, and subsequently viably frozen.

Meanwhile, the patient undergoes standard therapy, which included auto-SCT and treatment with immunomodulatory drugs (IMiD) and proteasome inhibitors (PIs) and potentially mAbs.

Although the treatment of MM has experienced some groundbreaking advancements in the past decade, MM is still considered an incurable disease, as all patients relapse eventually (34, 35). When the relapse occurs the patient then goes on to treatment with approved monoclonal therapeutic antibodies or experimental immunotherapy approaches if he/she is admitted into the ongoing clinical trials. Recently, the mAb daratumumab (Dara) was introduced as front-line treatment in newly diagnosed MM patients. This mAb recognizes CD38 highly and ubiquitously expressed on MM cells. Dara induces tumor cell death by several mechanisms of action, including binding to the Fc receptor on NK cells, which kill the target cells via antibody-dependent cellular cytotoxicity (ADCC)(36).

Another treatment approach, which has been done in a completed phase I/IIa clinical trial at Karolinska University Hospital, is to infuse the patient's own ex vivo expanded NK cells (30). NK cells from cancer patients often have an altered profile of receptor surface expression, and a decrease in functionality, a phenomenon also observed in MM. However, the autologous NK cells that have been expanded in GMP-controlled conditions, have a normalized expression profile, with up-regulated expression of activating receptors, down-regulation of inhibitory receptors (37). When tested in an in vitro responsiveness assay measuring CD107 release, the functionality has also been shown to be restored (37). These NK cells are re-infused into the patient, and showed objective measurable responses to NK cell infusions in terms of reduction in M-component and/or minimal residual disease (MRD) which lead to an increase in overall survival and decrease of measurable disease parameters such as M-component in the plasma (30).

We propose to combine these two treatment approaches by infusing CD38-GEAR engineered NK cells and daratumumab. In intermittent cycles, the patient receives Daratumumab and his/her own expanded and genetically modified (antibody-resistant) NK cells. While Daratumumab depletes all NK cells with the endogenous CD38 via ADCC and activation-induced depletion, the genetically modified NK cells will not be recognized by the Fab-region of Daratumumab and hence will not be depleted. They can however bind to the Fc-region of Daratumumab via their Fc-receptors CD16a and CD32c and perform ADCC of Daratumumab-opsonized MM cells. This will lead to a prolonged activity of Daratumumab, which may eventually lead to a longer time for clearance, a higher efficacy and longer levels of Daratumumab needed.

The treatment can be repeated in several cycles, depending on the yield of patient-derived autologous expanded NK cells from the initial apheresis.

Infused autologous NK cells can be detected up to four weeks after the last infusion in the circulation of the patient by Flow Cytometry.

Efficacy of the treatment can be assessed by electrophoresis of M component in the plasma in patients that have measurable disease (=M component) at the onset of relapse. Furthermore, next-generation sequencing (NGS) for minimal residual disease (MRD) can be performed. In addition, flow cytometric detection of MRD (EuroFlow) can be performed. Ultimately, overall survival (OS) and progression free survival (PFS) based on clinical parameters are determined in every patient.

To assess engraftment, reconstitution and persistence of adoptively transferred, genetically modified, ex vivo expanded, autologous NK cells, Flow cytometry or PCR can be performed. For Flow cytometry, CD38 antibodies and daratumumab can be combined with a standard panel for blood cells (e.g. CD3, CD14, CD19, CD56, Gr-1) and an extended panel for marker-combinations specific for ex vivo expanded NK cells, such as Ki67 or HLA-DR (30). For PCR, primers targeting the specific engineered mutation can be designed. Alternatively, since the introduced transgene is codon-optimized (thus having a different nt sequence then the endogenous CD38, despite having only a minor change in amino acid sequence), universal primers for the engineered CD38 can be designed.

For this, blood shall be drawn and analyzed prior to infusion, and 1 day, 3 days, 1 week and 1 month after every infusion.

To avoid reactivation of varicella zoster virus, which has been observed after adoptive transfer of autologous unmodified NK cells, the patients will receive anti-viral prophylaxis treatment (Valacyclovir, 500 mg twice daily for six months)(30).

CD38 is also expressed in many other malignant hematological diseases, including, leukemias and lymphomas, such as T- and B-cell acute lymphocytic leukemia, B-cell chronic lymphocytic leukemia, primary systemic amyloidosis, Waldenstrom macroglobulinemia, mantle-cell lymphoma, pro-lymphocytic/myelocytic leukemia, acute myeloid leukemia, chronic myeloid leukemia, follicular lymphoma, Burkitt's lymphoma, large granular lymphocytic (LGL) leukemia, NK-cell leukemia and plasma-cell leukemia. The methods of this example can be applied to other disorders which are treated with an antibody to CD38.

The modification in the GEAR-38 is specific to Daratumumab, see FIGS. 6 and 13-14. To assure that the modified CD38 molecule is only shielding the NK cells from recognition by Daratumumab, but not other CD38-targeting antibodies, binding of Isatuximab was tested. FIGS. 6 and 15-16 show that these modifications are indeed specific to Daratumumab, as binding by Isatuximab was not perturbed.

### Example 4: GEAR-38 Hematopoietic Stein Cell

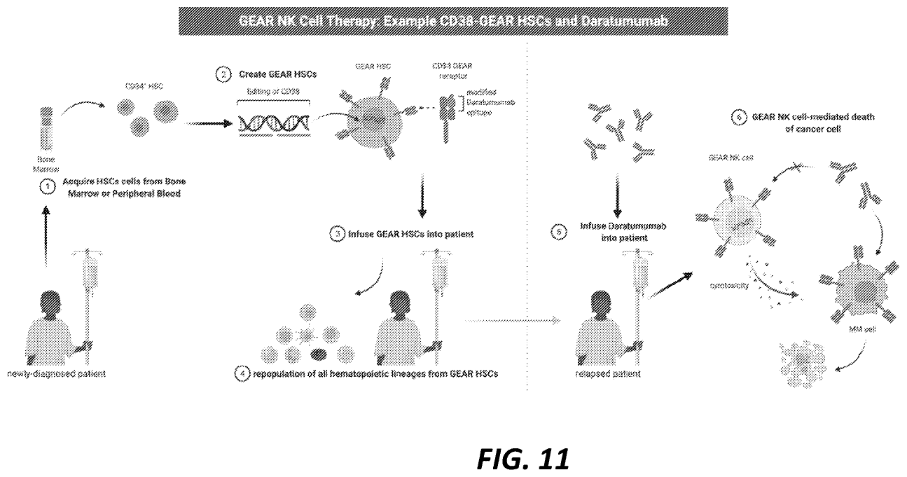

As in Example 3, and referring to FIG. 11, but here HSCs instead of mature NK cells will be modified. The result being that all hematopoietic cells derived from the transplant will be resistant to daratumumab-mediated killing/depletion. Autologous HSCs can be used for MM patients, where the malignant cell is a terminally differentiated cell. Allogeneic HSCs should be used in case of hematopoietic cancers where the malignant cell is a less differentiated cell population.

A patient is diagnosed with MM. He/she receives the standard treatment of autologous stem cell transplantation (auto-SCT) where hematopoietic stem cells are harvested from the blood either by apheresis or by removing bone marrow from the pelvic bone (iliac crest) with a special needle. During apheresis, HSCs are also harvested from the blood. HSCs are then genetically modified and quality controlled. These stem cells can repopulate all blood cell lineages. The harvested HSCs will be expanded in an enclosed, automated in vitro expansion procedure under GMP conditions and genetically modified with one of the constructs identified and described in Example 2 above. Successfully modified HS cells will express a CD38 variant that has a small change in the amino acid sequence and is thus no longer recognized by the therapeutic anti-CD38 mAb Daratumumab. The expanded HS cells are tested for activity, quality and sterility, and subsequently viably frozen. Once the HSCs are released according to standard release criteria, the patient receives high-dose chemotherapy and is then re-infused with his/her own stem cells.

We propose to combine two treatment approaches by infusing CD38-GEAR engineered HSCs and subsequently treating the patient with daratumumab. The patient is first transplanted with his own genetically engineered HSCs, which will repopulate all hematopoietic lineages. In cycles, the patient then receives Daratumumab. While Daratumumab depletes all CD38+ cells derived from non-modified HSCs via ADCC, ADCP, and activation-induced depletion, the genetically modified HSCs and their progeny will not be recognized by the Fab-region of Daratumumab and hence will not be depleted.

NK cells derived from modified HSCs can however bind to the Fc-region of Daratumumab via their Fc-receptors CD16a and CD32c and perform ADCC of Daratumumab-opsonized MM cells. This will lead to a prolonged activity of Daratumumab, which may eventually lead to a longer time for clearance, a higher efficacy and longer levels of Daratumumab needed. Furthermore, it will lead to a bigger population of mature NK cells able to use Daratumumab for ADCC-mediated killing of MM cells.

Infused autologous HSCs and their progeny can be detected by Flow Cytometry or PCR.

Efficacy of the treatment can be assessed by electrophoresis of M component in the plasma in patients that have measurable disease (=M component) at the onset of relapse.

Furthermore, next-generation sequencing (NGS) for minimal residual disease (MRD) can be performed. In addition, flow cytometric detection of MRD (EuroFlow) can be performed. Ultimately, overall survival (OS) and progression free survival (PFS) based on clinical parameters are determined in every patient.

To assess engraftment, reconstitution and persistence of adoptively transferred, genetically modified, ex vivo expanded, autologous HSCs, Flow cytometry or PCR can be performed on BM biopsies (for modified HSCs) or peripheral blood cells (for blood cells derived from modified HSCs). For Flow cytometry, CD38 antibodies and daratumumab can be combined with a standard panel for blood cells (e.g. CD3, CD14, CD19, CD56, Gr-1), while for PCR, primers targeting the specific engineered mutation can be designed. Alternatively, since the introduced transgene is codon-optimized (thus having a different nt sequence then the endogenous CD38, despite having only a minor change in amino acid sequence), universal primers for the engineered CD38 can be designed.

For this, blood shall be drawn and analyzed prior to infusion, and at several timepoints after infusion.

To avoid reactivation of varicella zoster virus, which has been observed after adoptive transfer of autologous unmodified NK cells, the patients will receive anti-viral prophylaxis treatment (Valacyclovir, 500 mg twice daily for six months).

In addition to the functional testing as outlined in examples 1-3, GEAR-CD38 HSCs should be furthermore assessed in their ability to develop into all hematopoietic cell lineages. This would be done by in vitro differentiation and then phenotyping for the different blood cell lineages, as well as differentiation in humanized mice.

### Example 5: GEAR-38 NK Cells Derived from Patient iPSCs

In this Example, we follow the methods of Examples 1, 2, 3 and 4 but here induced pluripotent stem cells (iPSCs) are modified and then differentiated into mature NK cells (or T cells), iPSCs have been used to generate many different cell types with distinct functions. Several protocols have been developed to derive NK cells from iPSCs. These iPSC-derived NK cells show cytotoxicity, target cell specificity, phenotype and proliferation capacity comparable to peripheral blood derived NK cells from healthy donors. During differentiation from iPSC to HSC, or from HSC to NK cell, genetic modifications can be introduced (38). By using the procedures described in examples 1, 2, 3, and 4, we can generate large quantities of modified NK cells necessary for clinical use. In addition to using fully mature iPSC-derived NK cells, several precursor stages, for example the immature NK cell (iNK) may be used as genetically modified clinical product.

In addition to the functional testing as outlined in Examples 1-3, GEAR-CD38 iPSCs should be furthermore assessed in their ability to develop into functional NK cells. This can be done by the previously described functional assays, and by phenotyping for NK cell surface markers, such as CD56, NKp46, DNAM-1 and other surface markers.

### Example 6: GEAR-38 NK Cell Lines

In this Example, we follow the methods of Examples 1, 2, 3 and 4 but here NK cell lines such as NK-92, KHYG-1 or others are modified and used as cellular product. The use of cell lines as NK cell source offers several advantages, such as unlimited growth capacity, and the potential to use these cells as off-the-shelf product. The cell line NK-92 has been used as unmodified or genetically engineered cellular product in many clinical trials, primarily in the context of hematological malignancies, but also some solid cancers. Other NK cell lines, such as KHYG-1 are being tested for clinical use. Genetic modification of NK cell lines is feasible and similar procedures as described in Examples 1, 2, 3, and 4 can be employed to generate CD38-GEAR NK-92 cells. As most currently available NK cell lines lack expression of the Fc-receptor CD16, these cells need to be modified to express CD16 for clinical use in antibody therapy. A naturally occurring high affinity variant, and a non-cleavable version of CD16 may be introduced (38), to increase ADCC capacity of the modified NK cell line product.

### Example 7: GEAR-19 Hematopoietic Stem Cell

Following the process of Examples 1-5, and referring to FIG. 12, we will genetically modify hematopoietic stem cells for bone marrow transplantation so that the blood cells developing from the stem cell graft will not be recognized by certain mAbs that can be used in therapy. In this way, the cells that will comprise the new blood system of the patient will be resistant to antibody-mediated effects such as antibody-dependent cellular cytotoxicity (ADCC), antibody-dependent complement activation or antibody-dependent cellular phagocytosis (ADCP). To achieve this goal, the cells of the cellular product will be modified for one or several specific surface proteins. These proteins are potential targets for subsequent antibody therapies. As an example, HSC transplantation and subsequent treatment with CD19-CAR T cells will be discussed.

For the treatment of B cell malignancies, such as acute myeloid leukemia (AML), acute lymphoid leukemia (ALL), chronic myeloid leukemia (CML), chronic lymphoid leukemia (CLL), multiple myeloma (MM) and other diseases, antibody therapies have become critical. These include monoclonal antibodies, antibody-drug conjugates, bi- or tri-specific antibodies, or CAR cells, where the antibody-recognition domain is genetically introduced into T cells or NK cells. Often, these antibodies target not only the antigen on malignant cells, but also on healthy bystander cells, a process termed on-target-off-tumor effects.

HSC transplantation is a standard therapy for these patients, which prolongs the survival and can be curative in some cases. However, many patients relapse, as some cancer cells remain. These patients are treated with a variety of different therapies, including chemotherapy, radiotherapy and lately immunotherapy approaches.

In the case of anti-CD19 treatment, such as CD19-CAR T cells, the malignant cells expressing high levels of CD19 are targeted and eliminated. However, the patients will suffer from B cell aplasia, the complete loss of CD19-positive cells, for as long as the CD19-CAR T cells remain in the patient's body. This could be a life-long condition, which leaves the patient immune-compromised and susceptible to recurring infections (39). It is therefore not done in MM patients. Currently approved CAR T therapies are marketed under the brand names: Abecma® (BCMA, idecabtagene vicleuce), Carvykti® (BCMA, ciltacabtagene autoleucel) Breyanzi® (CD19, lisocabtagene maraleucel), Kymriah® (CD19, tisagenlecleucel), Tecartus® (CD19, Brexucabtagene autoleucel) or Yescarta® (CD19, axicabtagene ciloleucel).

To decrease this particular drawback of CD19-CAR T cells, the HSC graft will be genetically engineered to be resistant to anti-CD19-mediated depletion. This will be achieved by changing one or several amino acids in the anti-CD19-binding site of CD19, so that the antibody or the CAR T cell cannot recognize CD19 on the B cells that develop from the genetically engineered HSC graft Autologous HSCs can be used for MM patients, where the malignant cell is a terminally differentiated cell. Allogeneic HSCs should be used in case of hematopoietic cancers where the malignant cell is a less differentiated cell population, such as CD19+ malignancies.

Genetic modifications can be introduced by different techniques, such as knock-out (KO) of the native CD19, with knock-in (KI) of the modified CD19, CRISPR (clustered regularly interspaced short palindromic repeats) editing of the native CD19 at the desired nucleotides, editing using TALENs (transcription activator-like effector nucleases) or ZFNs (Zinc Finger Nucleases). These nucleases can be delivered by electroporation, viral vector gene transfer, piggy-back or sleeping beauty delivery systems, engineered or biological nanoparticles, extracellular vesicles and many more technologies.

The sequence of editing steps can vary, by codon-optimizing the nucleotide sequence of the modified CD19 it can be assured that the newly introduced gene will not be targeted by knock-out or editing strategies.

FIG. 12 shows the steps required to develop and administer a CD19 cell. HSC apheresis is now the primary method for obtaining HSCs for stem cell transplants. The HSCs are mobilized from the bone marrow by treatment with cytokines, primarily G-CSF, so that they are more abundant in the blood. The HSCs are quality controlled, and either cryopreserved in liquid nitrogen or directly used for transplant. In addition, the HSCs can be evaluated for CFU-GM, which is currently the most reliable indicator of functionality.

Harvesting HSCs from bone marrow instead of apheresis is preferred by some transplantation centers, as the risk for severe GvHD seems to be less, especially in the context of haplo-identical transplantation. This method is mandatory in children and patients with aplastic anemias.

Genetic modification can be performed immediately after the harvest of HSCs. It could either be done very early during the procedure, to avoid prolonged culture time of the graft, or later, to target CD19+ B cells and their precursors specifically. The modifications of the CD19 antigen comprise all changes of one or several amino acids that would change the recognition of and the biding to CAR-CD19. These are predicted to be in the epitope of the anti-CD19 antibody used for generating the ScFv of the CAR construct. The substitutions would be from the native amino acids to those that have different physico-chemical properties, e.g different electric charge or structure, which both could abolish/compromise binding to the CAR.

Once these substitutions have been introduced into the HSCs, an assessment of binding to CAR-19 cells and other CD19 antibodies will be done. In addition, functionality will be assessed by differentiation of the HSCs into cells of the hematopoietic system to check of the modified cells can develop into all lineages. In addition, differentiated B cells should be assessed in terms of differentiation, BCR rearrangement, and antibody production. Cells will be expanded under standard expansion conditions, and quality and release criteria assessed as for unmodified cell product.

The CD19-GEAR HSC product can then be administered to the patient, using the same procedures and follow-up criteria as for any HSC graft.

Following the teachings of Example 1 we will identify the CD19-scFv epitope in the CD19 gene and suitable substitutions to eliminate or reduce antibody binding. Once we have determined what needs to be modified in the CD19 gene we will develop a strategy to either replace or edit the CD19 gene by either Knockout-knockin or targeted editing strategies.

For the Knockout-Knockin approach, the CD19 gene will be disrupted using CRISPR, ZFNs or TALENs. Subsequently, modified CD19 coding sequence will be introduced by electroporation, viral vector gene transfer, piggy-back or sleeping beauty delivery systems, engineered or biological nanoparticles, extracellular vesicles and many more technologies.

For CRISPR editing strategy, gRNAs that target the Cas9 towards the DNA sequence in the CD19 gene that encodes the CD19-CAR-binding epitope will be designed. These gRNAs will be tested for targeting efficiency (use them to generate a CD19-ko) in B cells. Homology-directed repair (HDR) templates will be designed for those gRNAs that show Cas9 cutting activity to perform editing of several amino acids that are in the vicinity of the induced double-strand break in HSCs. The resulting cells will be tested using functional assays, i.e., binding of CD19 antibody, CD19-CAR T/NK cells, proliferation, potential to generate all hematopoietic lineages with functional capacity, primarily B cell development.

The use of base editors (mutated Cas9 variants), which in theory can induce edits without generating double-strand breaks will be assessed.

The engineered cells are then expanded as needed for treatment. Appropriate quality controls are in place to ensure sterility, phenotype and overall safety of the cells.

### Example 8: ALL Patient Receiving CD19-CAR T Cells and Allogeneic GEAR Hematopoietic Stem Cells (CD19-GEAR-HSC)

A patient is diagnosed with ALL. He/she receives the standard treatment of allogeneic stem cell transplantation (SCT) where hematopoietic stem cells are harvested from the blood of a relative or unrelated donor either by apheresis or by removing bone marrow from the pelvic bone (iliac crest) with a special needle. After harvesting the hematopoietic stem cells, these cells are genetically modified with a construct as outlined in Examples 4 and 7. Successfully modified HSCs will harbor the gene of CD19 with a variant that has a small change in the amino acid sequence and is thus no longer recognized by the therapeutic antibodies and the single chain variable fragment (scFv) of the CD19-CAR-T cells or CD19-CAR-NK cells. The modified HSCs are tested for quality, and subsequently re-infused to the patient after he/she has undergone high-dose chemotherapy. These stem cells can repopulate all blood cell lineages.

In case of relapse, this patient can now be eligible for treatment with autologous CD19-CAR-T cells or autologous or off-the-shelf CD19-CAR-NK cells (derived from the allogeneic HSC graft). Currently approved CD19-CAR-T cells are marketed under the brand names Abecma® (BCMA, idecabtagene vicleuce), Carvykti® (BCMA, ciltacabtagene autoleucel) Breyanzi® (CD19, lisocabtagene maraleucel), Kymriah® (CD19, tisagenlecleucel), Tecartus® (CD19, Brexucabtagene autoleucel) or Yescarta® (CD19, axicabtagene ciloleucel). These cells are transduced to express a CAR consisting of the scFv of anti-CD19 mAb, a transmembrane domain and an intracellular signaling domain, and upon recognition of the CD19 antigen, they start killing the antigen-expressing cell. The healthy cells derived from the patients' modified GEAR-HSCs, however, harbor the CD19 variant that is not recognized by the CD19-CAR cells, and are thus resistant. This will lead to a situation where the malignant CD19+ ALL cells can be efficiently killed by the CD19-CAR cells, while the healthy cells remain. This patient may be less susceptible to long-term side-effects of CD19-CAR cell therapy, such as recurrent infections due to loss of all antibody-producing cells (39).

### Example 9: GEAR-19/38 Hematopoietic Stem Cell

Following the process of Examples 1-8, we will genetically modify HSCs for bone marrow transplantation so that the blood cells developing from the stem cell graft will not be recognized by certain mAbs that can be used in therapy. In this way, the cells that will comprise the new blood system of the patient will be resistant to antibody-mediated effects such as antibody-dependent cellular cytotoxicity (ADCC), antibody-dependent complement activation or antibody-dependent cellular phagocytosis (ADCP). To achieve this goal, the cells of the cellular product will be modified for one or several specific surface proteins. These proteins are potential targets for subsequent antibody therapies. As an example, HSC transplantation and subsequent treatment with CD19-CAR T cells and CD38 targeting antibodies such as Daratumumab will be discussed.

HSC transplantation is a standard therapy for these patients, which prolongs the survival and can be curative in some cases. However, many patients relapse, as some cancer cells remain. These patients are treated with a variety of different therapies, including chemotherapy, radiotherapy and lately immunotherapy approaches. Recently, CD19-CAR T cells have been approved for adult relapsed refractory (R/R) Diffuse Large B-cell Lymphoma (DLBCL) and pediatric and young adult R/R Acute Lymphoblastic Leukemia (ALL), large B-cell lymphoma or follicular lymphoma, high grade B cell lymphoma, primary mediastinal large B-cell lymphoma, R/R mantle cell lymphoma, adult R/R ALL. It can be expected that these cell products will be approved for many more indications soon. Furthermore, CAR cells with different specificity than CD19, e.g. BCMA or CD38 are currently being tested and approvals seem to be coming soon.

In the case of anti-CD19 treatment, such as CD19-CAR T cells, the malignant cells expressing high levels of CD19 are targeted and eliminated. However, the patients will suffer from B cell aplasia, the complete loss of CD19-positive cells, for as long as the CD19-CAR T cells remain in the patient's body. This could be a life-long condition, which leaves the patient immune-compromised and susceptible to recurring infections (39).

We propose to combine two treatment approaches by infusing CD19/38-GEAR engineered HSCs and subsequently treating the patient with daratumumab and CD19-CAR T cells sequentially. The patient is first transplanted with his own genetically engineered HSCs, which will repopulate all hematopoietic lineages.

In cycles, the patient then receives Daratumumab. While Daratumumab depletes all CD38+ cells derived from non-modified HSCs via ADCC, ADCP, and activation-induced depletion, the genetically modified HSCs and their progeny will not be recognized by the Fab-region of Daratumumab and hence will not be depleted. NK cells derived from modified HSCs can however bind to the Fc-region of Daratumumab via their Fc-receptors CD16a and CD32c and perform ADCC of Daratumumab-opsonized MM cells. This will lead to a prolonged activity of Daratumumab, which may eventually lead to a longer time for clearance, a higher efficacy and longer levels of Daratumumab needed. Furthermore, it will lead to a bigger population of mature NK cells able to use Daratumumab for ADCC-mediated killing of MM cells.

In case the patient relapses or becomes refractory to Daratumumab-treatment, we can consider treating this patient now with CD19-CAR T cells. The hematopoietic cell derived from the modified stem cell transplant will be resistant to CD19-CAR T cell-mediated elimination, thus preventing B cell aplasia. The malignant MM B cells are not derived from the modified transplant, and thus will be effectively killed by the CD19-CAR T cells.

Autologous HSCs can be used for MM patients, where the malignant cell is a terminally differentiated cell. Allogeneic HSCs should be used in case of hematopoietic cancers where the malignant cell is a less differentiated cell population, such as CD19+ malignancies.

Harvesting of HSCs, introduction of the genetic modifications, and functional and sterility testing will be done analogous to the Examples 1-8.

### Example 10: Non-Hodgkin Lymphoma Patient Receiving Anti-CD20 mAb (Rituximab) and Allogeneic GEAR Hematopoietic Stem Cells (CD20-GEAR-HSC)

Following the methods of Example 1-4 and 7-9 we can design an HSCs resistant to an anti-CD20 antibody. Rituximab is a currently approved therapeutic antibody. The Rituximab package insert is incorporated herein in its entirety. The HSC graft can be modified to differentiate into cells, especially B cells, that are resistant to recognition by anti-CD20 mAbs such as rituximab. The generation of this CD20-GEAR HSC product is analogous to the CD19-GEAR HSC product of Examples 7-9.

Apart from rituximab, many other CD20-specific antibodies are approved for therapy. Examples are Ocrelizumab, Veltuzumab, Obinutuzumab, Ofatumumab and many more.

The modifications of the CD20 antigen comprise all changes of one or several amino acids that would change the recognition of and the biding to anti-CD20 mAbs such as rituximab. These are predicted to be in the epitope of the anti-CD20 antibody. The substitutions would be from the native amino acids to those that have different physico-chemical properties, e.g. different electric charge or structure, which both could abolish/compromise binding to the mAb.

Following the teachings of Example 1 we will identify the CD20 antibody epitope in the CD20 gene and suitable substitutions to eliminate or reduce antibody binding. Once we have determined what needs to be modified in the CD20 gene we will develop a strategy to either replace or edit the CD20 gene by either Knockout-knockin or targeted editing strategies.

For the Knockout-Knockin approach, the CD20 gene will be disrupted using CRISPR, ZFNs or TALENs. Subsequently, modified CD20 coding sequence will be introduced by electroporation, viral vector gene transfer, piggy-back or sleeping beauty delivery systems, engineered or biological nanoparticles, extracellular vesicles and many more technologies.

For CRISPR editing strategy, gRNAs that target the Cas9 towards the DNA sequence in the CD20 gene that encodes the CD19-CAR-binding epitope will be designed. These gRNAs will be tested for targeting efficiency (use them to generate a CD20-ko) in B cells. Homology-directed repair (HDR) templates will be designed for those gRNAs that show Cas9 cutting activity to perform editing of several amino acids that are in the vicinity of the induced double-strand break in HSCs. The resulting cells will be tested using functional assays, i.e., binding of CD20 antibody, CD20-CAR T/NK cells, proliferation, potential to generate all hematopoietic lineages with functional capacity, primarily B cell development.

The use of base editors (mutated Cas9 variants), which in theory can induce edits without generating double-strand breaks will be assessed.

The engineered cells are then expanded as needed for treatment. Appropriate quality controls are in place to ensure sterility, phenotype and overall safety of the cells.

A patient is diagnosed with Non-Hodgkin lymphoma. He/she receives the standard treatment of allogeneic stem cell transplantation (SCT) where hematopoietic stem cells (HSCs) are harvested from the blood either by apheresis or by removing bone marrow from the pelvic bone (iliac crest) with a special needle. After harvesting the hematopoietic stem cells, these cells are genetically modified with one of the constructs identified and described in the current invention. Successfully modified HSCs will harbor the gene of CD20 with a variant that has a small change in the amino acid sequence and is thus no longer recognized by the therapeutic antibodies such as Rituximab, Ocrelizumab, Ofatumumab, Obinutuzomab. The modified HSCs are tested for quality, and subsequently re-infused to the patient after he/she has undergone high-dose chemotherapy. These stem cells can repopulate all blood cell lineages.

In case of relapse, this patient can now be eligible for treatment with therapeutic antibodies targeting CD20+ cells. The doctors decide that the optimal treatment would be rituximab. This antibody treatment eradicates the malignant cells, but also depletes all cells expressing the endogenous CD20 antigen. The healthy cells derived from the patients' modified GEAR-HSCs, however, harbor the CD20 variant that is not recognized by rituximab, and are thus resistant. This will lead to a situation where the malignant CD20+ cells can be efficiently eradicated, while the healthy cells remain. This patient may be less susceptible to long-term side-effects of rituximab therapy, such as recurrent infections due to loss of all antibody-producing cells.

### Example 11: Acute Myelogenous Lymphoma Patient Receiving Anti-CD117 (Anti-cKIT) mAb (KITMAB) or Anti-cKIT-ADC (LOP628) and Allogeneic GEAR Hematopoietic Stem Cells (CD117-GEAR-HSC)

Following the methods of Example 1-4 and 7-9 we can design an HSCs resistant to an anti-CD117 (cKIT) antibody. LOP628 is a currently tested therapeutic antibody for gastrointestinal stromal tumors (GIST), small cell lung cancer (SCLC), non-small cell lung cancer (NSCLC), melanoma, and acute myelogenous leukemia (AML). The HSC graft can be modified to differentiate into hematopoietic cells, that are resistant to recognition by anti-CD117 mAbs such as LOP628, KITMAB and others. The generation of this CD117-GEAR HSC product is analagous to the CD19-GEAR HSC product of Examples 7-9.

Apart from LOP628, KITMAB, many other CD117-specific antibodies are currently tested for therapy. The biggest obstacle to getting them approved, is that these antibodies do not only bind to malignant cells, but all developing hematopoietic cells express the antigen at some cell stage. The current invention could substantially improve the development to a clinical product.

The modifications of the CD 117 antigen comprise all changes of one or several amino acids that would change the recognition of and the binding to anti-CD117 mAbs. These are predicted to be in the epitope of the anti-CD117 antibody. The substitutions would be from the native amino acids to those that have different physico-chemical properties, e.g different electric charge or structure, which both could abolish/compromise binding to the mAb.

Following the teachings of Example 1 we will identify the CD 117 antibody epitope in the CD117 gene and suitable substitutions to eliminate or reduce antibody binding. Once we have determined what needs to be modified in the CD117 gene we will develop a strategy to either replace or edit the CD117 gene by either Knockout-knockin or targeted editing strategies.

For the Knockout-Knockin approach, the CD 117 gene will be disrupted using CRISPR, ZFNs or TALENs. Subsequently, modified CD117 coding sequence will be introduced by electroporation, viral vector gene transfer, piggy-back or sleeping beauty delivery systems, engineered or biological nanoparticles, extracellular vesicles and many more technologies.

For CRISPR editing strategy, gRNAs that target the Cas9 towards the DNA sequence in the CD 117 gene that encodes the CD117-binding epitope will be designed. These gRNAs will be tested for targeting efficiency (use them to generate a CD117-ko) in B cells. Homology-directed repair (HDR) templates will be designed for those gRNAs that show Cas9 cutting activity to perform editing of several amino acids that are in the vicinity of the induced double-strand break in HSCs. The resulting cells will be tested using functional assays, i.e., binding of CD117 antibody, CD117-ADC, CD117-bispecifics, KITMAB is currently tested for imatinib-resistant GIST. CD117-CAR T/NK cells, proliferation, potential to generate all hematopoietic lineages with functional capacity, primarily B cell development.

The use of base editors (mutated Cas9 variants), which in theory can induce edits without generating double-strand breaks will be assessed.

The engineered cells are then expanded as needed for treatment. Appropriate quality controls are in place to ensure sterility, phenotype and overall safety of the cells.

A patient is diagnosed with AML. He/she receives the standard treatment of allogeneic stem cell transplantation (SCT) where hematopoietic stem cells are harvested from the blood either by apheresis or by removing bone marrow from the pelvic bone (iliac crest) with a special needle. After harvesting the hematopoietic stem cells, these cells are genetically modified with one of the constructs identified and described in the current invention. Successfully modified HSCs will harbor the gene of CD117 with a variant that has a small change in the amino acid sequence and is thus no longer recognized by the therapeutic antibodies. The modified HSCs are tested for quality, and subsequently re-infused to the patient after he/she has undergone high-dose chemotherapy. These stem cells can repopulate all blood cell lineages.

In case of relapse, this patient can now be eligible for treatment with therapeutic antibodies targeting CD117+ cells. The doctors decide that the optimal treatment would be anti-CD117 antibody. This antibody treatment eradicates the malignant cells, but also depletes all cells expressing the endogenous CD117 antigen. The healthy cells derived from the patients' modified GEAR-HSCs, however, harbor the CD117 variant that is not recognized by the antibody, and are thus resistant. This will lead to a situation where the malignant CD117+ cells can be efficiently eradicated, while the healthy cells remain. This patient may be less susceptible to long-term side-effects of CD117-antibody therapy, such as recurrent infections due to loss of hematopoietic cells.

### Example 12: Acute Myelogenous Lymphoma Patient Receiving Anti-CD34 BiTE and Allogeneic GEAR Hematopoietic Stem Cells (CD34-GEAR-HSC)

Following the methods of Example 1-4 and 7-9 we can design an HSCs resistant to an anti-CD34 antibody or anti-CD34 bispecific T cell engager (BiTE). A novel anti-CD34 BiTE has recently been published for the depletion of AML and leukemic stem cells (40).

This BiTE can be given in addition to non-myeloablative conditioning treatment to kill/deplete remaining leukemic stem cells. After that, the patient is infused with stem cell graft, which also expresses CD34, hence the BiTE cannot be used anymore or else it would also deplete the new HSCs. If we instead infuse CD34-GEAR-HSCs as graft, then this BiTE could be used longer and more than once, as the new GEAR-HSCs would be resistant to CD34 targeting of the BiTE.

The modifications of the CD34 antigen comprise all changes of one or several amino acids that would change the recognition of and the biding to anti-CD34 mAbs. These are predicted to be in the epitope of the anti-CD34 antibody used for generating the CD34-BiTE. The substitutions would be from the native amino acids to those that have different physico-chemical properties, e.g. different electric charge or structure, which both could abolish/compromise binding to the mAb.

Following the teachings of Example 1 we will identify the CD34 antibody epitope in the CD34 gene and suitable substitutions to eliminate or reduce BiTE binding. Once we have determined what needs to be modified in the CD34 gene we will develop a strategy to either replace or edit the CD34 gene by either Knockout-knockin or targeted editing strategies.

For the Knockout-Knockin approach, the CD34 gene will be disrupted using CRISPR, ZFNs or TALENs. Subsequently, modified CD34 coding sequence will be introduced by electroporation, viral vector gene transfer, piggy-back or sleeping beauty delivery systems, engineered or biological nanoparticles, extracellular vesicles and many more technologies.

For CRISPR editing strategy, gRNAs that target the Cas9 towards the DNA sequence in the CD34 gene that encodes the CD34-binding epitope will be designed. These gRNAs will be tested for targeting efficiency (use them to generate a CD34-ko) in B cells. Homology-directed repair (HDR) templates will be designed for those gRNAs that show Cas9 cutting activity to perform editing of several amino acids that are in the vicinity of the induced double-strand break in HSCs. The resulting cells will be tested using functional assays, i.e., binding of CD34 antibody, CD34-ADC, CD34-bispecifics. CD34-CAR T/NK cells, proliferation, potential to generate all hematopoietic lineages with functional capacity.

The use of base editors (mutated Cas9 variants), which in theory can induce edits without generating double-strand breaks will be assessed.

The engineered cells are then expanded as needed for treatment. Appropriate quality controls are in place to ensure sterility, phenotype and overall safety of the cells.

A patient is diagnosed with AML. He/she receives the standard treatment of allogeneic stem cell transplantation (SCT) where hematopoietic stem cells are harvested from the blood either by apheresis or by removing bone marrow from the pelvic bone (iliac crest) with a special needle. After harvesting the hematopoietic stem cells, these cells are genetically modified with one of the constructs identified and described in the current invention. Successfully modified HSCs will harbor the gene of CD34 with a variant that has a small change in the amino acid sequence and is thus no longer recognized by the therapeutic antibodies. The modified HSCs are tested for quality, and subsequently re-infused to the patient after he/she has undergone high-dose chemotherapy. These stem cells can repopulate all blood cell lineages.

In case of relapse, this patient can now be eligible for treatment with therapeutic antibodies targeting CD34+ cells. The doctors decide that the optimal treatment would be anti-CD34 antibody. This antibody treatment eradicates the malignant cells, but also depletes all cells expressing the endogenous CD34 antigen. The healthy cells derived from the patients' modified GEAR-HSCs, however, harbor the CD34 variant that is not recognized by the antibody, and are thus resistant. This will lead to a situation where the malignant CD34+ cells can be efficiently eradicated, while the healthy cells remain. This patient may be less susceptible to long-term side-effects of CD34-antibody therapy, such as recurrent infections due to loss of hematopoietic cells.

### Example 13: T1D Patient Receiving Pancreatic Islet Transplantation of Autologous or Allogeneic GEAR Pancreatic Islet Cells (GEAR-Autoislets/Alloislets)

Following the teachings of Example 1 we will identify the epitope binding regions for Islet Cell Antibodies (ICA, against cytoplasmic proteins in the beta cell) antibodies to Glutamic Acid Decarboxylase (GAD-65), Insulin Autoantibodies (IAA), and IA-2A, to protein tyrosine phosphatase [2]

We will identify antibodies responsible for the binding epitope to the corresponding antibodies and suitable substitutions to eliminate or reduce antibody binding.

Once we have determined what needs to be modified in relevant gene, we will develop a strategy to either replace or edit that gene using the teachings of Example 1.

The expanded cells after appropriate quality control are administered to a patient in need thereof. The cells may be administered once or multiple times.

Patients can be tested during therapy to determine the presence of engineered cells and dosing adjusted based on test results.

### Example 14: Treatment of Colorectal Cancer with Theralizumab (Anti-CD28 Agonistic Antibody, CD28-SuperMAB) and Autologous GEAR Hematopoietic Stem Cells (CD28-GEAR-HSC)

Following the methods of Example 1 we can design an antibody-resistant cell type for which prior trials have failed due to on-target off-tumor activities leading to severe side-effects.

One such failed antibody is anti-CD28 antibody theralizumab (TGN1412). TGN1412 is a humanized IgG4 agonistic anti-CD28 monoclonal antibody designed to stimulate T cells by activating CD28 signaling without the need for prior activation of the T-cell antigen receptor. It was originally intended for the treatment of B cell chronic lymphocytic leukemia (B-CLL) and rheumatoid arthritis. In the first and only in-human study in 2006, it caused severe inflammatory reactions and chronic organ failure. A phase I and II clinical trial have been completed for arthritis and clinical trials for cancer are underway. The antibody binds and is an agonist of CD28, a co-stimulatory molecule expresses by T cells, NK cells and eosinophil granulocytes. These cells can release many pro-inflammatory cytokines when activated by TGN1412, which has been suggested to be the cause for the observed adverse events.

Following the methods of Example 1 we can design an HSC resistant to an anti-CD28 antibody.

The generation of this CD28GEAR HSC product is in analogous to the CD19GEAR HSC product of Examples 7-9.

The modifications of the CD28 antigen comprise all changes of one or several amino acids that would change the recognition of and the biding to anti-CD28 mAbs such as theralizumab. These are predicted to be in the epitope of the anti-CD28 antibody. The substitutions would be from the native amino acids to those that have different physico-chemical properties, e.g different electric charge or structure, which both could abolish/compromise binding to the mAb.

Following the teachings of Example 1 identification of the CD28-scFv epitope in the CD28 gene and suitable substitutions to eliminate or reduce antibody binding are necessary.

Once we have determined what needs to be modified in the CD28 gene we will develop a strategy to either replace or edit the CD28 gene.

The resulting cells are tested with functional assays, ie, cytotoxicity and cytokine production, proliferation, exhaustion, coping with metabolic stress etc as are relevant for the particular clinical application.

The engineered cells are then expanded as needed for treatment. Appropriate quality controls are in place to ensure sterility, phenotype and overall safety of the cells.

The expanded cells after appropriate quality control are administered to a patient in need thereof. therapeutic antibody administered at any timepoint thereafter will not affect the hematopoietic cells that developed from the modified HSCs.

Patients can be tested during therapy to determine the presence of engineered cells and dosing adjusted based on test results.

The patient receives a transplant with this edited HSCs. In case of a relapse, he/she can be treated with theralizumab, because all hematopoietic cells, including memory T cells, express an edited version of CD28 that will not be recognized by the mAb. In parallel, he/she receives the other half of the frozen HSCs, that remain unedited and can be activated by the agonistic mAb theralizumab.

### Example 15: IL-2 and CD25 and Tregs

Following the methods of Examples 1, 2 and 3 we can design cells resistant to IL-2, Tregs, SLAMF7, CD19, CD20, CD22, CD25, CD28, CD30, CD33, CD47, CD52, CD 117 and/or PDGFRA.

### Example 16: Cell Replacement

Using the present methods, any undesired cell population can be removed and replaced with engineered cells that are resistant to the antibody.

### Example 17: Tissue Replacement

Using the present methods, any undesired tissue can be treated to engineer cells that are resistant to the antibody.

One of skill in the art will appreciate that the invention described herein can be used to design cells resistant to any therapeutic antibody and such cells used therapeutically to help treat patients undergoing treatment with that therapeutic antibody.

### REFERENCES

1. H. Nahi et al., Incidence, characteristics, and outcome of solitary plasmacytoma and plasma cell leukemia. Population-based data from the Swedish Myeloma Register. Eur J Haematol 99, 216-222 (2017).
2. M. V. Mateos, J. F. San Miguel, Management of multiple myeloma in the newly diagnosed patient. Hematology Am Soc Hematol Educ Program 2017, 498-507 (2017).
3. S. K. Kumar et al., Continued improvement in survival in multiple myeloma: changes in early mortality and outcomes in older patients. Leukemia 28, 1122-1128 (2014).
4. H. M. Lokhorst et al., Targeting CD38 with Daratumumab Monotherapy in Multiple Myeloma. N Engl J Med 373, 1207-1219 (2015).
5. T. Plesner, J. Krejcik, Daratumumab for the Treatment of Multiple Myeloma. Front Immunol 9, 1228 (2018).
6. M. Attal et al., Isatuximab plus pomalidomide and low-dose dexamethasone versus pomalidomide and low-dose dexamethasone in patients with relapsed and refractory multiple myeloma (ICARIA-MM): a randomised, multicentre, open-label, phase 3 study. Lancet 394, 2096-2107 (2019).
7. J. Mikhael et al., A dose-finding Phase 2 study of single agent isatuximab (anti-CD38 mAb) in relapsed/refractory multiple myeloma. Leukemia 34, 3298-3309 (2020).
8. S. Ibrahim et al., CD38 expression as an important prognostic factor in B-cell chronic lymphocytic leukemia. Blood 98, 181-186 (2001).
9. A. Funaro et al., Involvement of the multilineage CD38 molecule in a unique pathway of cell activation and proliferation. J Immunol 145, 2390-2396 (1990).
10. F. Bonello et al., CD38 as an immunotherapeutic target in multiple myeloma. Expert Opin Biol Ther 18, 1209-1221 (2018).
11. M. Naeimi Kararoudi et al., CD38 deletion of human primary NK cells eliminates daratumumab-induced fratricide and boosts their effector activity. Blood 136, 2416-2427 (2020).
12. K. V. Woan et al., Harnessing features of adaptive NK cells to generate iPSC-derived NK cells for enhanced immunotherapy. Cell Stem Cell, (2021).
13. D. Viola et al., Daratumumab induces mechanisms of immune activation through CD38+NK cell targeting. Leukemia, (2020).
14. S. Y. Rah, J. Y. Kwak, Y. J. Chung, U. H. Kim, ADP-ribose/TRPM2-mediated Ca2+ signaling is essential for cytolytic degranulation and antitumor activity of natural killer cells. Sci Rep 5, 9482 (2015).
15. G. Sconocchia et al., CD38 triggers cytotoxic responses in activated human natural killer cells. Blood 94, 3864-3871 (1999).
16. T. Lischke et al., CD38 controls the innate immune response against Listeria monocytogenes. Infect Immun 81, 4091-4099 (2013).
17. R. Mallone et al., Signaling through CD38 induces NK cell activation. Int Immunol 13, 397-409 (2001).
18. S. Deaglio et al., Human CD38 and CD16 are functionally dependent and physically associated in natural killer cells. Blood 99, 2490-2498 (2002).
19. E. Glaria, A. F. Valledor, Roles of CD38 in the Immune Response to Infection. Cells 9, (2020).
20. Q. Liu et al., Crystal structure of human CD38 extracellular domain. Structure 13, 1331-1339 (2005).
21. H. Nahi et al., Infectious complications and NK cell depletion following daratumumab treatment of Multiple Myeloma. PloS one 14, e0211927 (2019).
22. T. Casneuf et al., Effects of daratumumab on natural killer cells and impact on clinical outcomes in relapsed or refractory multiple myeloma. Blood Adv 1, 2105-2114 (2017).
23. J. S. Miller et al., Successful adoptive transfer and in vivo expansion of human haploidentical NK cells in patients with cancer. Blood 105, 3051-3057 (2005).
24. M. C. Hareth Nahi, Stephan Meinke, Charlotte Gran, Nicole Marquardt, Garniel Afram, Tolga Sutlu, Mari Gilljam, Birgitta Stellan, Arnika K. Wagner, Pontus Blomberg, Per-Henrik Holmqvist, Lillian Walther-Jallow, Karin Mellstrom, Johan Liwing, Charlotte Gustafsson, Robert Minsson, Emma Watz, Monika Klimkowska, Gösta Gahrton, Johan Lund, Per Ljungman, Hans-Gustaf Ljunggren, Evren Alici, Autologous Activated and Expanded NK Cells as Consolidation Therapy Following Autologous Stem Cell Transplantation in Multiple Myeloma. Cell Reports Medicine accepted january 2022, (2022).
25. A. T. Bjorklund et al., Complete Remission with Reduction of High-Risk Clones following Haploidentical NK-Cell Therapy against MDS and AML. Clin Cancer Res 24, 1834-1844 (2018).
26. C. J. Denman et al., Membrane-bound IL-21 promotes sustained ex vivo proliferation of human natural killer cells. PloS one 7, e30264 (2012).
27. E. Liu et al., Use of CAR-Transduced Natural Killer Cells in CD19-Positive Lymphoid Tumors. N Engl J Med 382, 545-553 (2020).
28. R. Romee et al., Cytokine-induced memory-like natural killer cells exhibit enhanced responses against myeloid leukemia. Sci Transl Med 8, 357ra123 (2016).
29. F. Cichocki et al., iPSC-derived NK cells maintain high cytotoxicity and enhance in vivo tumor control in concert with T cells and anti-PD-1 therapy. Sci Transl Med 12, (2020).
30. H. Nahi et al., Autologous NK cells as consolidation therapy following stem cell transplantation in multiple myeloma. Cell Rep Med 3, 100508 (2022).
31. G. Alter, J. M. Malenfant, M. Altfeld, CD107a as a functional marker for the identification of natural killer cell activity. J Immunol Methods 294, 15-22 (2004).
32. Y. T. Bryceson et al., Functional analysis of human NK cells by flow cytometry. Methods Mol Biol 612, 335-352 (2010).
33. S. Sarkar et al., The CD38 (low) natural killer cell line KHYG1 transiently expressing CD16(F158V) in combination with daratumumab targets multiple myeloma cells with minimal effector NK cell fratricide. Cancer immunology, immunotherapy: CII 69, 421-434 (2020).
34. S. K. Kumar et al., Natural history of relapsed myeloma, refractory to immunomodulatory drugs and proteasome inhibitors: a multicenter IMWG study. Leukemia 31, 2443-2448 (2017).
35. J. Laubach et al., Management of relapsed multiple myeloma: recommendations of the International Myeloma Working Group. Leukemia 30, 1005-1017 (2016).
36. N. van de Donk, S. Z. Usmani, CD38 Antibodies in Multiple Myeloma: Mechanisms of Action and Modes of Resistance. Front Immunol 9, 2134 (2018).
37. E. Alici et al., Autologous antitumor activity by NK cells expanded from myeloma patients using GMP-compliant components. Blood 111, 3155-3162 (2008).
38. H. Zhu et al., Pluripotent stem cell-derived NK cells with high-affinity noncleavable CD16a mediate improved antitumor activity. Blood 135, 399-410 (2020).
39. S. L. Maude et al., Chimeric antigen receptor T cells for sustained remissions in leukemia. N Engl J Med 371, 1507-1517 (2014).
40. L. C. M. Arruda et al., A novel CD34-specific T-cell engager efficiently depletes acute myeloid leukemia and leukemic stem cells in vitro and in vivo. Haematologica, (2022).

We claim:

1. A cell comprising an otherwise wild-type protein having at least one mutation in the binding site of a therapeutic agent, the at least one mutation being configured to disrupt specific binding of the therapeutic agent to the protein while retaining the physiological function of the wild-type protein.

2. The cell of claim 1 wherein the therapeutic agent is a therapeutic antibody.

3. The cell of claim 1 wherein the at least one mutation is induced with gene editing.

4. The cell of claim 1 wherein the at least one mutation is at least one amino acid substitution for a naturally occurring amino acid in the binding site.

5. The cell of claim 1, which is a primate cell, preferably a human cell, more preferably a primary human cell.

6. The cell of claim 1, which is selected from an immunocyte (e.g., plasma cell, B cell, macrophage, NK cell, dendritic cell, neutrophil, monocyte, T cell), stem cell (e.g, hematopoietic stem cell, induced pluripotent stem cell), or a somatic cell.

7. The cell of claim any of the preceding claims which is allogeneic and/or otherwise configured for adoptive therapy in a subject, preferably a human subject.

8. The cell of claim any of the preceding claims wherein the therapeutic agent binding site is present in a protein expressed on the surface of the cell, the protein selected from CD38, SLAMF7, CD19, CD20, CD22, CD25, CD28, CD30, CD33, CD47, CD52, or PDGFRA.

9. The cell of claim 8 wherein the therapeutic agent binding site is present in CD38, the therapeutic agent is an antibody, and the antibody is daratumumab or isatuximab.

10. The cell of claim 8 wherein the therapeutic agent binding site is present in SLAMF7, the therapeutic agent is an antibody, and the antibody is elotuzumab.

11. The cell of claim 8 wherein the therapeutic agent binding site is present in CD19, the therapeutic agent is an antibody, and the antibody is blinatumomab.

12. The cell of claim 8 wherein the therapeutic agent binding site is present in CD19, and the therapeutic agent is a CD19-CAR-T cell or NK cell, such as Abecma, Breyanzi, Kymriah, Tecartus or Yescarta.

13. The cell of claim 8 wherein the therapeutic agent binding site is present in CD20, the therapeutic agent is an antibody, and the antibody is ibritumomab, tiuxetan, obinutuzumab, ocrelizumab, ofatumumab, or rituximab.

14. The cell of claim 8 wherein the therapeutic agent binding site is present in CD22, the therapeutic agent is an antibody, and the antibody is inotuzumab.

15. The cell of claim 8 wherein the therapeutic agent binding site is present in CD30, the therapeutic agent is an antibody, and the antibody is brentuximab.

16. The cell of claim 8 wherein the therapeutic agent binding site is present in CD33, the therapeutic agent is an antibody, and the antibody is gemtuzumab or ozogamicin.

17. The cell of claim 8 wherein the therapeutic agent binding site is present in CD52, the therapeutic agent is an antibody, and the antibody is alemtuzumab or ANT1034.

18. The cell of claim 8 wherein when the therapeutic agent binding site is present in CD47, the therapeutic agent is an antibody, and the antibody is Ti-061 or CC-90002 or magrolimab or AK117 or AO-176 or CPO107 JMT601 (CPO107) or DSP107 or Evorpacept (ALX148) or HX009 or IBI188 or IBI322 or IMC-002 or IMM0306 or OSE-172PF-07257876 or SHR-1603 or SHR2150 or SRF231 or STI-6643 or TG-1801 or TJ011133 or TTI-621 or ZL-1201.

19. The cell of claim 8 wherein the therapeutic agent binding site is present in PDGFRA, the therapeutic agent is an antibody, and the antibody is olaratumab.

20. A method of treating a patient comprising administering to the patient:
(i) a cell comprising an otherwise wild-type protein having at least one mutation in the binding site of a therapeutic agent, the at least one mutation being configured to disrupt specific binding of the therapeutic agent to the protein while retaining the physiological function of the wild-type protein; and
(ii) the therapeutic agent.

21. The method of claim 20 wherein the at least one mutation comprises at least one amino acid substitution in the binding site.

22. The method of claim 20 wherein the cell is selected from an immunocyte (e.g., plasma cell, B cell, macrophage, NK cell, dendritic cell, neutrophil, monocyte, T cell), stem cell (e.g, hematopoietic stem cell, induced pluripotent stem cell), or a somatic cell.

23. The method of claim 21 wherein the therapeutic agent binding site is present in a protein expressed on the surface of the cell selected from CD38, SLAMF7, CD19, CD20, CD22, CD25, CD28, CD30, CD33, CD47, CD52, or PDGFRA.

24. The method of claim 23 wherein the therapeutic agent binding site is present in CD38, the therapeutic agent is an antibody, and the antibody is Daratumumab or Isatuximab or TAK-079.

25. The method cell of claim 23 wherein the therapeutic agent binding site is present in SLAMF7, the therapeutic agent is an antibody, and the antibody is Elotuzumab.

26. The method cell of claim 23 wherein the therapeutic agent binding site is present in CD19, the therapeutic agent is an antibody, and the antibody is Blinatumomab.

27. The method of claim 23 wherein the therapeutic agent binding site is present in CD20, the therapeutic agent is an antibody, and the antibody is ibritumomab tiuxetan, obinutuzumab, ocrelizumab, ofatumumab, rituximab, or rituximab/hyaluronidase.

28. The method of claim 23 wherein the therapeutic agent binding site is present in CD22, the therapeutic agent is an antibody, and the antibody is inotuzumab

29. The method of claim 23 wherein the therapeutic agent binding site is present in CD30, the therapeutic agent is an antibody, and the antibody is Brentuximab.

30. The method of claim 23 wherein the therapeutic agent binding site is present in CD33, the therapeutic agent is an antibody, and the antibody is gemtuzumab ozogamicin.

31. The method of claim 23 wherein the therapeutic agent binding site is present in CD52, the therapeutic agent is an antibody, and the antibody is alemtuzumab or ANT1034

32. The method of claim 18 wherein the therapeutic agent binding site is present in CD47, the therapeutic agent is an antibody, and the antibody is Ti-061 or CC-90002 or magrolimab or AK117 or AO-176 or CPO107 JMT601 (CPO107) or DSP107 or Evorpacept (ALX148) or HX009 or IBI188 or IBI322 or IMC-002 or IMM0306 or OSE-172PF-07257876 or SHR-1603 or SHR2150 or SRF231 or STI-6643 or TG-1801 or TJ011133 or TTI-621 or ZL-1201.

33. The method of claim 23 wherein the therapeutic agent binding site is present in PDGFRA, the therapeutic agent is an antibody, and the antibody is olaratumab.

34. The method of claim 23 wherein protein expressed on the surface of the cell is functional for all purposes except therapeutic agent binding.

35. The method of claim 23 wherein the CD38 on the surface of the cell is functional for all purposes except daratumumab binding.

36. The method of claim 35 wherein the binding site of daratumumab on the cell is modified so that it is no longer recognized by daratumumab.

37. The method of claim 36 having at least one amino acid substitution in the daratumumab binding site.

38. The method of claim 37 wherein the at least one amino acid substitution is made to amino acids 233-246 or 267-286 of SEQ ID NO 5.

39. The method of claim 38 wherein the at least one amino substitution is made to amino acids 237, 239, 272, 274 and/or 276 of SEQ ID NO: 5.

40. The method of claim 38 wherein the at least one amino acid substitution is selected from the following: T237A, E239F, Q272R, S274F, and/or K276F.

41. The method of claim 35 wherein the CD38 on the surface of the cell comprises SEQ ID NO: 6, 7, 8, 9, or 10.

42. The method of claim 23 wherein the CD38 on the surface of the cell is functional for all purposes except isatuximab binding.

43. The method of claim 42 wherein the binding site of isatuximab on the cell is modified so that it is no longer recognized by isatuximab.

44. The method of claim 43 having at least one amino acid substitution in the isatuximab binding site.

45. The method of claim 44 wherein the amino acid substitution is made to one or more of amino acids 77-80, 111-118, or 232-234 of SEQ ID NO 5.

46. The method of claim 45 wherein the amino acid substitution is made to amino acids 77, 78, 79, 80, 111, 112, 113, 114, 115, 116, 117, 118, 232, 233 and/or 234 of SEQ ID NO: 5.

47. The method of claim 46 wherein the amino acid substitution is selected from the following: M77F, R78F, H79F, V80F, K11F, L112F, G113F, T114F, Q115F, T 16F, V117F, P118F, P232F, E233F and/or K234F.

48. The method of claim 42 wherein the CD38 on the surface of the cell comprises SEQ ID NO: 11, 12, 13, 14, 15, 16, 17, 18, 19, 20, 21, 22, 23, 24, or 25.

49. The method of claim 23 wherein the therapeutic agent binding site is present in CD47, the therapeutic agent is an antibody, and the antibody is Magrolimab.

50. The method of claim 49 wherein the amino acid substitution is made to a region of CD47 comprising amino acids 1-3, 34-36 and/or 97-104 of SEQ ID NO: 27.

51. The method of claim 50 wherein the amino acid substitution is made to one or more of amino acids, 1, 2, 3, 34, 35, 36, 97, 98, 99, 100, 101, 102, 103, and/or 104 of SEQ ID NO: 27.

52. The method of claim 51 wherein the amino acid substitution is selected from the following: Q1F, L2F, L3F, T34F, E35F, V36F, E97F, V98F, T99F, E100F, L101F, T102F, R103F and/or E104F.

53. The method of claim 52 wherein the CD47 on the surface of the cell comprises SEQ ID NO: 28, 29, 30, 31, 32, 33, 34, 35, 36, 37, 38, 39, 40, and/or 41.

54. The method of claim 23 wherein the therapeutic agent binding site is present in CD52, the therapeutic agent is an antibody, and the antibody is alemtuzumab.

55. The method of claim 54 wherein the amino acid substitution is made to a region of CD52 comprising amino acids 31-36 of SEQ ID NO: 43.

56. The method of claim 55 wherein the amino acid substitution is made to amino acids 31, 32, 33, 34, 35 and/or 36 of SEQ ID NO: 43.

57. The method of claim 56 wherein the amino acid substitution is selected from the following: Q31F, T32F, S33F, S34F, P35F and/or S36F.

58. The method of claim 57 wherein the CD52 on the surface of the cell comprises SEQ ID NO: 45, 46, 47, 48, 49 and/or 50.

59. An adoptive cell therapy method comprising administering the cell of claim 1 to patient in need thereof.

60. The adoptive cell therapy of claim 59 wherein the therapeutic agent binding site is present in CD38 expressed on the surface of the cell and the cell is functional for all purposes except daratumumab binding.

61. The adoptive cell therapy of claim 60 wherein the therapeutic agent binding site is a binding site of daratumumab and the daratumumab binding site is modified so that it is no longer recognized by daratumumab.

62. The adoptive cell therapy of claim 61 wherein the daratumumab binding site comprises at least one amino acid substitution.

63. The adoptive cell therapy of claim 62 wherein the at least one amino acid substitution is made within an extracellular region subsequence of amino acids present in SEQ ID NO: 5, preferably within amino acids 233-246 or 267-286 of SEQ ID NO 5.

64. The adoptive cell therapy of claim 63 wherein the at least one amino acid substitution is made to amino acids 237, 239, 272, 274 and/or 276 of SEQ ID NO: 5.

65. The adoptive cell therapy of claim 64 wherein the amino acid substitution is selected from the following: T237A, E239F, Q272R, S274F, K276F.

66. The adoptive cell therapy of claim 59 wherein the therapeutic agent binding site is present in CD38 expressed on the surface of the cell and the cell is functional for all purposes except isatuximab binding.

67. The adoptive cell therapy of claim 66 wherein the therapeutic agent binding site is a binding site of isatuximab and the isatuximab binding site is modified so that it is no longer recognized by isatuximab.

68. The adoptive cell therapy of claim 67 wherein the isatuximab binding site comprises at least one amino acid substitution.

69. The adoptive cell therapy of claim 62 wherein the at least one amino acid substitution is made within an extracellular region subsequence of amino acids present in SEQ ID NO: 5.

70. The adoptive cell therapy of claim 69 wherein the amino acid substitution is made to a region of CD38 comprising amino acids 77-80, 111-118, or 232-234 of SEQ ID NO 5.

71. The adoptive cell therapy of claim 70 wherein the amino acid substitution is made to amino acids 77, 78, 79, 80, 111, 112, 113, 114, 115, 116, 117, 118, 232, 233 and/or 234.

72. The adoptive cell therapy of claim 71 wherein the amino acid substitution is selected from the following: M77F, R78F, H79F, V80F, K111F, L112F, G113F, T114F, Q115F, T 16F, V117F, P118F, P232F, E233F and/or K234F.

73. The adoptive cell therapy of claim 72 wherein the CD38 on the surface of the cell comprises SEQ ID NO: 11, 12, 13, 14, 15, 16, 17, 18, 19, 20, 21, 22, 23, 24, or 25.

74. A cell configured for adoptive therapy, which comprises a cell surface and/or transmembrane protein having at least one mutation in the binding site of a therapeutic antibody, the at least one mutation being configured to disrupt specific binding of the therapeutic antibody to the cell surface and/or transmembrane protein.

75. The cell of claim 74 comprising SEQ ID NO: 6, 7, 8, 9, and/or 10.

76. The cell of claim 74 comprising SEQ ID NO: 11, 12, 13, 14, 15, 16, 17, 18, 19, 20, 21, 22, 23, 24, and/or 25.

77. The cell of claim 74 comprising SEQ ID NO: 28, 29, 30, 31, 32, 33, 34, 35, 36, 37, 38, 39, 40, and/or 41.

78. The cell of claim 74 comprising SEQ ID NO: 45, 46, 47, 48, 49, and/or 50.

79. The cell of any of claims 74-78 wherein the at least one mutation is introduced by knock-out (KO) of the native protein, with knock-in (KI) of the modified native protein, CRISPR editing of the native protein at the desired nucleotides, and/or editing using TALENs (transcription activator-like effector nucleases) or ZFNs (Zinc Finger Nucleases).

80. The cell of claim 79 wherein the CRISPR editing comprises introducing a guide RNA represented by SEQ ID NO: 1, 2, 51, and/or 52.

81. The cell of claim 74, which comprises an otherwise wild-type protein having at least one mutation in the binding site of a first therapeutic antibody, the at least one mutation being configured to disrupt specific binding of the therapeutic antibody to the protein while retaining the physiological function of the wild-type protein.

82. The cell of claim 81, which is configured to retain binding sites to a second therapeutic antibody.

83. A therapeutic method comprising administering an antibody to a subject in need thereof, wherein the cell of claim 1 has been administered to the subject prior to administering the antibody, and wherein the antibody is a monoclonal antibody, CAR T, a BIKE or a TRIKE.

84. The method of claim 83 wherein the method is a treatment for a malignant hematological disease.

85. The method of claim 84 wherein the malignant hematological disease is selected from, multiple myeloma, leukemias and lymphomas, such as T- and B-cell acute lymphocytic leukemia, B-cell chronic lymphocytic leukemia, primary systemic amyloidosis, Waldenstrom macroglobulinemia, mantle-cell lymphoma, pro-lymphocytic/myelocytic leukemia, acute myeloid leukemia, chronic myeloid leukemia, follicular lymphoma, Burkitt's lymphoma, large granular lymphocytic (LGL) leukemia, NK-cell leukemia or plasma-cell leukemia.

86. A therapy cell comprising at least one mutation to more than one therapeutic antibody target site such that the therapeutic antibodies no longer bind to the cell.

87. The therapy cell of claim 86 wherein the mutation is induced with gene editing.

88. The therapy cell of claim 86 wherein the antibody target sites are selected from sites present in CD38, SLAMF7, CD19, CD20, CD22, CD25, CD28, CD30, CD33, CD47, CD52, and/or PDGFRA.

89. A cell comprising an exogenous nucleotide sequence which encodes a mutant form of a human cell-surface or transmembrane protein, the mutant form engineered to lack an epitope that allows for specific binding to a therapeutic antibody but otherwise having all the same functional capabilities as the corresponding wild-type cell-surface or transmembrane protein.

90. The cell of claim 89, in which the exogenous nucleotide sequence encodes a mutant form of human CD38, SLAMF7, CD19, CD20, CD22, CD25, CD28, CD30, CD33, CD47, CD52, or PDGFRA, or a variant thereof having at least 80% sequence identity thereto.

91. A method of producing a cell for adoptive therapy comprising:
(a) obtaining an allogeneic and/or stem cell comprising a nucleic acid sequence encoding a protein expressed on the surface of the allogenic and/or stem cell, wherein the protein comprises a therapeutic antibody binding site;
(b) identifying the amino acid residues of the therapeutic antibody binding site; and
(c) introducing one or more mutations into the nucleic acid sequence encoding the protein so that specific binding of a therapeutic antibody to the binding site is disrupted while maintaining all other functions of the expressed protein.

92. The method of claim 91 wherein the therapeutic antibody binding site is selected from a site present in CD38, SLAMF7, CD19, CD20, CD22, CD25, CD28, CD30, CD33, CD47, CD52, or PDGFRA.

93. The method of claim 92 wherein the therapeutic antibody is daratumumab, and an amino acid substitution is made to amino acids 233-246 or 267-286 of CD38 as represented by SEQ ID NO 5.

94. The method of claim 93 wherein the amino substitution is made to amino acids 237, 239, 272, 274 and/or 276 of SEQ ID NO: 5.

95. The method of claim 94 wherein the amino acid substitution is selected from the following: T237A, E239F, Q272R, S274F, and/or K276F.

96. The method of claim 92 wherein the therapeutic antibody is isatuximab, and an amino acid substitution is made to a region of CD38 comprising amino acids 77-80, 111-118, or 232-234 of SEQ ID NO 5.

97. The method of claim 96 wherein the amino acid substitution is made to amino acids 77, 78, 79, 80, 111, 112,113, 114, 115, 116, 117, 118, 232, 233 and/or 234.

98. The method of claim 97 wherein the amino acid substitution is selected from the following: M77F, R78F, H79F, V80F, K11 IF, I 112F, GI 13F, T114F, Q115F, T116F, V117F, P118F, P232F, E233F and/or K234F.
